(19) United States
(12) Patent Application Publication
(10) Pub. No.: US 2025/0388688 A1
(43) Pub. Date: Dec. 25, 2025

(54) ANTI-TSHR ANTIBODIES AND USES THEREOF

(71) Applicant: ETHYREAL BIO, INC., Cambridge, MA (US)

(72) Inventors: Lauren Pepper, Belmont, MA (US); Melissa Geddie, Arlington, MA (US); Alexey Alexandrovich Lugovskoy, Belmont, MA (US); Tinashe Chandauka, New York, NY (US); Charles Gordon Grummitt, Bagunte (PT); Simon Lempriere Westbrook, Dubai (AE); Stephen Edward Rapecki, Burnham (GB)

(21) Appl. No.: 19/170,463

(22) Filed: Apr. 4, 2025

Related U.S. Application Data
(60) Provisional application No. 63/574,825, filed on Apr. 4, 2024.
(60) Provisional application No. 63/768,464, filed on Mar. 7, 2025.

(51) Int. Cl. C07K 16/28; A61K 40/31; A61K 40/42; A61K 47/68; C40B 40/08

(52) U.S. Cl. CPC C07K 16/2869; A61K 40/31; A61K 40/4202; A61K 47/6849; C40B 40/08; C07K 2317/52; C07K 2317/565; C07K 2317/567; C07K 2317/92

(57) ABSTRACT

Provided herein are antigen binding proteins that bind to thyroid stimulating hormone receptor (TSHR) and methods of using the same.

### RELATED APPLICATIONS

This application claims the benefit of U.S. Provisional Application Ser. No. 63/574,825, filed Apr. 4, 2024, and U.S. Provisional Application Ser. No. 63/768,464, filed Mar. 7, 2025, the entire disclosures of each are incorporated herein by reference.

### SEQUENCE LISTING

The instant application contains a Sequence Listing which has been submitted electronically in XML format and is hereby incorporated by reference in its entirety. Said XML file, created on Sep. 10, 2025, is named 763100_TB9-003_ST26.xml and is 139,443 bytes in size.

### BACKGROUND

Thyroid-stimulating hormone receptor (TSHR) is a G-protein coupled receptor (GPCR) that is mainly expressed on thyroid epithelial cells as well as periorbital fibroblasts and adipose tissue and has a key role in the regulation of thyroid function. Activation of TSHR by thyroid-stimulating hormone (TSH) results in the growth and proliferation of thyrocytes and thyroid hormone production, in particular the production of thyroxine (T₄) and triidothyronine (T₃). These hormones have several critical functions in the human body, as they support human metabolic and cardiovascular systems, and regulate the metabolism of fats, proteins, and carbohydrates. There are presently a number of known but unresolved problems relating to the thyroid including general thyroid disease and thyroid-related disorders (e.g., Graves' disease) as well as other diseases where TSHR plays a pathophysiological role (e.g., thyroid eye disease and thyroid cancer). Therefore, there is a need for therapeutics that prevent or treat these diseases and disorders associated with inappropriate TSHR activation.

### SUMMARY

In an aspect, provided herein is an antigen binding protein or an antigen binding fragment thereof which specifically binds thyroid-stimulating hormone receptor (TSHR), wherein the antigen binding protein or an antigen binding fragment thereof comprises a heavy chain variable (VH) domain and a light chain variable (VL) domain, wherein: the VH domain comprises a HCDR1 sequence comprising the amino acid sequence of SEQ ID NO: 44, a HCDR2 sequence comprising the amino acid sequence of SEQ ID NO: 49 or SEQ ID NO: 39, and a HCDR3 sequence comprising the amino acid sequence of SEQ ID NO: 50; a HCDR1 sequence comprising the amino acid sequence of SEQ ID NO: 45, a HCDR2 sequence comprising the amino acid sequence of SEQ ID NO: 49 or SEQ ID NO: 39, and a HCDR3 sequence comprising the amino acid sequence of SEQ ID NO: 50; a HCDR1 sequence comprising the amino acid sequence of SEQ ID NO: 46, a HCDR2 sequence comprising the amino acid sequence of SEQ ID NO: 49 or SEQ ID NO: 39, and a HCDR3 sequence comprising the amino acid sequence of SEQ ID NO: 50; a HCDR1 sequence comprising the amino acid sequence of SEQ ID NO: 47, a HCDR2 sequence comprising the amino acid sequence of SEQ ID NO: 49 or SEQ ID NO: 39, and a HCDR3 sequence comprising the amino acid sequence of SEQ ID NO: 50; a HCDR1 sequence comprising the amino acid sequence of SEQ ID NO: 48, a HCDR2 sequence comprising the amino acid sequence of SEQ ID NO: 49 or SEQ ID NO: 39, and a HCDR3 sequence comprising the amino acid sequence of SEQ ID NO: 50; a HCDR1 sequence comprising the amino acid sequence of SEQ ID NO: 48, a HCDR2 sequence comprising the amino acid sequence of SEQ ID NO: 49 or SEQ ID NO: 39, and a HCDR3 sequence comprising the amino acid sequence of SEQ ID NO: 51; a HCDR1 sequence comprising the amino acid sequence of SEQ ID NO: 47, a HCDR2 sequence comprising the amino acid sequence of SEQ ID NO: 49 or SEQ ID NO: 39, and a HCDR3 sequence comprising the amino acid sequence of SEQ ID NO: 51; a HCDR1 sequence comprising the amino acid sequence of SEQ ID NO: 48, a HCDR2 sequence comprising the amino acid sequence of SEQ ID NO: 39, and a HCDR3 sequence comprising the amino acid sequence of SEQ ID NO: 52; a HCDR1 sequence comprising the amino acid sequence of SEQ ID NO: 48, a HCDR2 sequence comprising the amino acid sequence of SEQ ID NO: 39, and a HCDR3 sequence comprising the amino acid sequence of SEQ ID NO: 53; a HCDR1 sequence comprising the amino acid sequence of SEQ ID NO: 38, a HCDR2 sequence comprising the amino acid sequence of SEQ ID NO: 39, and a HCDR3 sequence comprising the amino acid sequence of SEQ ID NO: 60; a HCDR1 sequence comprising the amino acid sequence of SEQ ID NO: 38, a HCDR2 sequence comprising the amino acid sequence of SEQ ID NO: 39, and a HCDR3 sequence comprising the amino acid sequence of SEQ ID NO: 61; a HCDR1 sequence comprising the amino acid sequence of SEQ ID NO: 62, a HCDR2 sequence comprising the amino acid sequence of SEQ ID NO: 39, and a HCDR3 sequence comprising the amino acid sequence of SEQ ID NO: 40; a HCDR1 sequence comprising the amino acid sequence of SEQ ID NO: 63, a HCDR2 sequence comprising the amino acid sequence of SEQ ID NO: 39, and a HCDR3 sequence comprising the amino acid sequence of SEQ ID NO: 40; a HCDR1 sequence comprising the amino acid sequence of SEQ ID NO: 64, a HCDR2 sequence comprising the amino acid sequence of SEQ ID NO: 39, and a HCDR3 sequence comprising the amino acid sequence of SEQ ID NO: 40; a HCDR1 sequence comprising the amino acid sequence of SEQ ID NO: 65, a HCDR2 sequence comprising the amino acid sequence of SEQ ID NO: 39, and a HCDR3 sequence comprising the amino acid sequence of SEQ ID NO: 40; a HCDR1 sequence comprising the amino acid sequence of SEQ ID NO: 66, a HCDR2 sequence comprising the amino acid sequence of SEQ ID NO: 39, and a HCDR3 sequence comprising the amino acid sequence of SEQ ID NO: 40; a HCDR1 sequence comprising the amino acid sequence of SEQ ID NO: 38, a HCDR2 sequence comprising the amino acid sequence of SEQ ID NO: 67, and a HCDR3 sequence comprising the amino acid sequence of SEQ ID NO: 40; a HCDR1 sequence comprising the amino acid sequence of SEQ ID NO: 38, a HCDR2 sequence comprising the amino acid sequence of SEQ ID NO: 39, and a HCDR3 sequence comprising the amino acid sequence of SEQ ID NO: 68; and the VL domain comprises: a LCDR1 sequence comprising the amino acid sequence of SEQ ID NO: 54, a LCDR2 sequence comprising the amino acid sequence of SEQ ID NO: 42, and a LCDR3 sequence comprising the amino acid sequence of SEQ ID NO: 58 or SEQ ID NO: 59; a LCDR1 sequence comprising the amino acid sequence of SEQ ID NO: 55, a LCDR2 sequence comprising the amino acid sequence of SEQ ID NO: 42, and a LCDR3 sequence comprising the amino acid sequence of SEQ ID NO: 58; a LCDR1 sequence comprising the amino acid sequence of SEQ ID NO: 55, a LCDR2 sequence comprising the amino acid sequence of SEQ ID NO: 42, and a LCDR3 sequence comprising the amino acid sequence of SEQ ID NO: 59; a LCDR1 sequence comprising the amino acid sequence of SEQ ID NO: 56, a LCDR2 sequence comprising the amino acid sequence of SEQ ID NO: 42, and a LCDR3 sequence comprising the amino acid sequence of SEQ ID NO: 59; a LCDR1 sequence comprising the amino acid sequence of SEQ ID NO: 55, a LCDR2 sequence comprising the amino acid sequence of SEQ ID NO: 57, and a LCDR3 sequence comprising the amino acid sequence of SEQ ID NO: 59; a LCDR1 sequence comprising the amino acid sequence of SEQ ID NO: 41, a LCDR2 sequence comprising the amino acid sequence of SEQ ID NO: 42, and a LCDR3 sequence comprising the amino acid sequence of SEQ ID NO: 97; a LCDR1 sequence comprising the amino acid sequence of SEQ ID NO: 41, a LCDR2 sequence comprising the amino acid sequence of SEQ ID NO: 42, and a LCDR3 sequence comprising the amino acid sequence of SEQ ID NO: 69; a LCDR1 sequence comprising the amino acid sequence of SEQ ID NO: 70, a LCDR2 sequence comprising the amino acid sequence of SEQ ID NO: 42, and a LCDR3 sequence comprising the amino acid sequence of SEQ ID NO: 71; a LCDR1 sequence comprising the amino acid sequence of SEQ ID NO: 72, a LCDR2 sequence comprising the amino acid sequence of SEQ ID NO: 42, and a LCDR3 sequence comprising the amino acid sequence of SEQ ID NO: 71; a LCDR1 sequence comprising the amino acid sequence of SEQ ID NO: 74, a LCDR2 sequence comprising the amino acid sequence of SEQ ID NO: 42, and a LCDR3 sequence comprising the amino acid sequence of SEQ ID NO: 73; a LCDR1 sequence comprising the amino acid sequence of SEQ ID NO: 75, a LCDR2 sequence comprising the amino acid sequence of SEQ ID NO: 42, and a LCDR3 sequence comprising the amino acid sequence of SEQ ID NO: 73; a LCDR1 sequence comprising the amino acid sequence of SEQ ID NO: 76, a LCDR2 sequence comprising the amino acid sequence of SEQ ID NO: 42, and a LCDR3 sequence comprising the amino acid sequence of SEQ ID NO: 73; or a LCDR1 sequence of SEQ ID NO: 41, a LCDR2 sequence comprising the amino acid sequence of SEQ ID NO: 42, and a LCDR3 sequence comprising the amino acid sequence of SEQ ID NO: 77.

In some embodiments, the VH comprises an amino acid sequence at least 90% identical to SEQ ID NO: 1 and the VL comprises an amino acid sequence at least 90% identical to SEQ ID NO: 2. In some embodiments, the VH comprises an amino acid sequence at least 95% identical to SEQ ID NO: 3 and the VL comprises an amino acid sequence at least 90% identical to SEQ ID NO: 4.

In some embodiments, the VH comprises the amino acid sequence of SEQ ID NO: 3, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 78, SEQ ID NO: 79, SEQ ID NO: 80, SEQ ID NO: 81, SEQ ID NO: 82, SEQ ID NO: 83, SEQ ID NO: 84, SEQ ID NO: 85, SEQ ID NO: 86, SEQ ID NO: 87, or SEQ ID NO: 88; and the VL comprises the amino acid sequence of SEQ ID NO: 4, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 89, SEQ ID NO: 90, SEQ ID NO: 91, SEQ ID NO: 92, SEQ ID NO: 93, SEQ ID NO: 94, SEQ ID NO: 95, or SEQ ID NO: 96.

In some embodiments, the VH comprises an amino acid sequence of SEQ ID NO: 78 and the VL comprises an amino acid sequence of SEQ ID NO: 25; the VH comprises an amino acid sequence of SEQ ID NO: 78 and the VL comprises an amino acid sequence of SEQ ID NO: 32; the VH comprises an amino acid sequence of SEQ ID NO: 16 and the VL comprises an amino acid sequence of SEQ ID NO: 25; the VH comprises an amino acid sequence of SEQ ID NO: 16 and the VL comprises an amino acid sequence of SEQ ID NO: 32; the VH comprises an amino acid sequence of SEQ ID NO: 7 and the VL comprises an amino acid sequence of SEQ ID NO: 29; the VH comprises an amino acid sequence of SEQ ID NO: 7 and the VL comprises an amino acid sequence of SEQ ID NO: 35; the VH comprises an amino acid sequence of SEQ ID NO: 7 and the VL comprises an amino acid sequence of SEQ ID NO: 30; the VH comprises an amino acid sequence of SEQ ID NO: 7 and the VL comprises an amino acid sequence of SEQ ID NO: 36; the VH comprises an amino acid sequence of SEQ ID NO: 8 and the VL comprises an amino acid sequence of SEQ ID NO: 29; the VH comprises an amino acid sequence of SEQ ID NO: 8 and the VL comprises an amino acid sequence of SEQ ID NO: 35; the VH comprises an amino acid sequence of SEQ ID NO: 8 and the VL comprises an amino acid sequence of SEQ ID NO: 30; the VH comprises an amino acid sequence of SEQ ID NO: 8 and the VL comprises an amino acid sequence of SEQ ID NO: 36; the VH comprises an amino acid sequence of SEQ ID NO: 9 and the VL comprises an amino acid sequence of SEQ ID NO: 29; the VH comprises an amino acid sequence of SEQ ID NO: 9 and the VL comprises an amino acid sequence of SEQ ID NO: 35; the VH comprises an amino acid sequence of SEQ ID NO: 9 and the VL comprises an amino acid sequence of SEQ ID NO: 30; the VH comprises an amino acid sequence of SEQ ID NO: 9 and the VL comprises an amino acid sequence of SEQ ID NO: 36; the VH comprises an amino acid sequence of SEQ ID NO: 10 and the VL comprises an amino acid sequence of SEQ ID NO: 29; the VH comprises an amino acid sequence of SEQ ID NO: 10 and the VL comprises an amino acid sequence of SEQ ID NO: 35; the VH comprises an amino acid sequence of SEQ ID NO: 10 and the VL comprises an amino acid sequence of SEQ ID NO: 30; the VH comprises an amino acid sequence of SEQ ID NO: 10 and the VL comprises an amino acid sequence of SEQ ID NO: 36; the VH comprises an amino acid sequence of SEQ ID NO: 11 and the VL comprises an amino acid sequence of SEQ ID NO: 29; the VH comprises an amino acid sequence of SEQ ID NO: 11 and the VL comprises an amino acid sequence of SEQ ID NO: 35; the VH comprises an amino acid sequence of SEQ ID NO: 11 and the VL comprises an amino acid sequence of SEQ ID NO: 30; the VH comprises an amino acid sequence of SEQ ID NO: 11 and the VL comprises an amino acid sequence of SEQ ID NO: 36; the VH comprises an amino acid sequence of SEQ ID NO: 12 and the VL comprises an amino acid sequence of SEQ ID NO: 29; the VH comprises an amino acid sequence of SEQ ID NO: 12 and the VL comprises an amino acid sequence of SEQ ID NO: 35; the VH comprises an amino acid sequence of SEQ ID NO: 12 and the VL comprises an amino acid sequence of SEQ ID NO: 30; the VH comprises an amino acid sequence of SEQ ID NO: 12 and the VL comprises an amino acid sequence of SEQ ID NO: 36; the VH comprises an amino acid sequence of SEQ ID NO: 13 and the VL comprises an amino acid sequence of SEQ ID NO: 29; the VH comprises an amino acid sequence of SEQ ID NO: 12 and the VL comprises an amino acid sequence of SEQ ID NO: 35; the VH comprises an amino acid sequence of SEQ ID NO: 12 and the VL comprises an amino acid sequence of SEQ ID NO: 30; the VH comprises an amino acid sequence of SEQ ID NO: 12 and the VL comprises an amino acid sequence of SEQ ID NO: 36; the VH comprises an amino acid sequence of SEQ ID NO: 16 and the VL comprises an amino acid sequence of SEQ ID NO: 29; the VH comprises an amino acid sequence of SEQ ID NO: 16 and the VL comprises an amino acid sequence of SEQ ID NO: 35; the VH comprises an amino acid sequence of SEQ ID NO: 16 and the VL comprises an amino acid sequence of SEQ ID NO: 30; the VH comprises an amino acid sequence of SEQ ID NO: 16 and the VL comprises an amino acid sequence of SEQ ID NO: 36; the VH comprises an amino acid sequence of SEQ ID NO: 17 and the VL comprises an amino acid sequence of SEQ ID NO: 29; the VH comprises an amino acid sequence of SEQ ID NO: 17 and the VL comprises an amino acid sequence of SEQ ID NO: 35; the VH comprises an amino acid sequence of SEQ ID NO: 17 and the VL comprises an amino acid sequence of SEQ ID NO: 30; the VH comprises an amino acid sequence of SEQ ID NO: 17 and the VL comprises an amino acid sequence of SEQ ID NO: 36; the VH comprises an amino acid sequence of SEQ ID NO: 18 and the VL comprises an amino acid sequence of SEQ ID NO: 29; the VH comprises an amino acid sequence of SEQ ID NO: 18 and the VL comprises an amino acid sequence of SEQ ID NO: 35; the VH comprises an amino acid sequence of SEQ ID NO: 18 and the VL comprises an amino acid sequence of SEQ ID NO: 30; the VH comprises an amino acid sequence of SEQ ID NO: 18 and the VL comprises an amino acid sequence of SEQ ID NO: 36; the VH comprises an amino acid sequence of SEQ ID NO: 19 and the VL comprises an amino acid sequence of SEQ ID NO: 29; the VH comprises an amino acid sequence of SEQ ID NO: 19 and the VL comprises an amino acid sequence of SEQ ID NO: 35; the VH comprises an amino acid sequence of SEQ ID NO: 19 and the VL comprises an amino acid sequence of SEQ ID NO: 30; the VH comprises an amino acid sequence of SEQ ID NO: 19 and the VL comprises an amino acid sequence of SEQ ID NO: 36; the VH comprises an amino acid sequence of SEQ ID NO: 20 and the VL comprises an amino acid sequence of SEQ ID NO: 29; the VH comprises an amino acid sequence of SEQ ID NO: 20 and the VL comprises an amino acid sequence of SEQ ID NO: 35; the VH comprises an amino acid sequence of SEQ ID NO: 20 and the VL comprises an amino acid sequence of SEQ ID NO: 30; the VH comprises an amino acid sequence of SEQ ID NO: 20 and the VL comprises an amino acid sequence of SEQ ID NO: 36; the VH comprises an amino acid sequence of SEQ ID NO: 21 and the VL comprises an amino acid sequence of SEQ ID NO: 29; the VH comprises an amino acid sequence of SEQ ID NO: 21 and the VL comprises an amino acid sequence of SEQ ID NO: 35; the VH comprises an amino acid sequence of SEQ ID NO: 21 and the VL comprises an amino acid sequence of SEQ ID NO: 30; the VH comprises an amino acid sequence of SEQ ID NO: 21 and the VL comprises an amino acid sequence of SEQ ID NO: 36; the VH comprises an amino acid sequence of SEQ ID NO: 22 and the VL comprises an amino acid sequence of SEQ ID NO: 29; the VH comprises an amino acid sequence of SEQ ID NO: 22 and the VL comprises an amino acid sequence of SEQ ID NO: 35; the VH comprises an amino acid sequence of SEQ ID NO: 22 and the VL comprises an amino acid sequence of SEQ ID NO: 30; the VH comprises an amino acid sequence of SEQ ID NO: 22 and the VL comprises an amino acid sequence of SEQ ID NO: 36; the VH comprises an amino acid sequence of SEQ ID NO: 11 and the VL comprises an amino acid sequence of SEQ ID NO: 26; the VH comprises an amino acid sequence of SEQ ID NO: 20 and the VL comprises an amino acid sequence of SEQ ID NO: 26; the VH comprises an amino acid sequence of SEQ ID NO: 12 and the VL comprises an amino acid sequence of SEQ ID NO: 26; the VH comprises an amino acid sequence of SEQ ID NO: 21 and the VL comprises an amino acid sequence of SEQ ID NO: 26; the VH comprises an amino acid sequence of SEQ ID NO: 11 and the VL comprises an amino acid sequence of SEQ ID NO: 27; the VH comprises an amino acid sequence of SEQ ID NO: 20 and the VL comprises an amino acid sequence of SEQ ID NO: 27; the VH comprises an amino acid sequence of SEQ ID NO: 12 and the VL comprises an amino acid sequence of SEQ ID NO: 27; the VH comprises an amino acid sequence of SEQ ID NO: 21 and the VL comprises an amino acid sequence of SEQ ID NO: 27; the VH comprises an amino acid sequence of SEQ ID NO: 11 and the VL comprises an amino acid sequence of SEQ ID NO: 28; the VH comprises an amino acid sequence of SEQ ID NO: 20 and the VL comprises an amino acid sequence of SEQ ID NO: 28; the VH comprises an amino acid sequence of SEQ ID NO: 12 and the VL comprises an amino acid sequence of SEQ ID NO: 28; the VH comprises an amino acid sequence of SEQ ID NO: 21 and the VL comprises an amino acid sequence of SEQ ID NO: 28; the VH comprises an amino acid sequence of SEQ ID NO: 11 and the VL comprises an amino acid sequence of SEQ ID NO: 31; the VH comprises an amino acid sequence of SEQ ID NO: 20 and the VL comprises an amino acid sequence of SEQ ID NO: 31; the VH comprises an amino acid sequence of SEQ ID NO: 12 and the VL comprises an amino acid sequence of SEQ ID NO: 31; the VH comprises an amino acid sequence of SEQ ID NO: 21 and the VL comprises an amino acid sequence of SEQ ID NO: 31; the VH comprises an amino acid sequence of SEQ ID NO: 14 and the VL comprises an amino acid sequence of SEQ ID NO: 30; the VH comprises an amino acid sequence of SEQ ID NO: 23 and the VL comprises an amino acid sequence of SEQ ID NO: 30; the VH comprises an amino acid sequence of SEQ ID NO: 15 and the VL comprises an amino acid sequence of SEQ ID NO: 30; the VH comprises an amino acid sequence of SEQ ID NO: 24 and the VL comprises an amino acid sequence of SEQ ID NO: 30; the VH comprises an amino acid sequence of SEQ ID NO: 11 and the VL comprises an amino acid sequence of SEQ ID NO: 32; the VH comprises an amino acid sequence of SEQ ID NO: 20 and the VL comprises an amino acid sequence of SEQ ID NO: 32; the VH comprises an amino acid sequence of SEQ ID NO: 12 and the VL comprises an amino acid sequence of SEQ ID NO: 32; the VH comprises an amino acid sequence of SEQ ID NO: 21 and the VL comprises an amino acid sequence of SEQ ID NO: 32; the VH comprises an amino acid sequence of SEQ ID NO: 11 and the VL comprises an amino acid sequence of SEQ ID NO: 33; the VH comprises an amino acid sequence of SEQ ID NO: 20 and the VL comprises an amino acid sequence of SEQ ID NO: 33; the VH comprises an amino acid sequence of SEQ ID NO: 12 and the VL comprises an amino acid sequence of SEQ ID NO: 33; the VH comprises an amino acid sequence of SEQ ID NO: 21 and the VL comprises an amino acid sequence of SEQ ID NO: 33; the VH comprises an amino acid sequence of SEQ ID NO: 11 and the VL comprises an amino acid sequence of SEQ ID NO: 34; the VH comprises an amino acid sequence of SEQ ID NO: 20 and the VL comprises an amino acid sequence of SEQ ID NO: 34; the VH comprises an amino acid sequence of SEQ ID NO: 12 and the VL comprises an amino acid sequence of SEQ ID NO: 34; the VH comprises an amino acid sequence of SEQ ID NO: 21 and the VL comprises an amino acid sequence of SEQ ID NO: 34; the VH comprises an amino acid sequence of SEQ ID NO: 11 and the VL comprises an amino acid sequence of SEQ ID NO: 37; the VH comprises an amino acid sequence of SEQ ID NO: 20 and the VL comprises an amino acid sequence of SEQ ID NO: 37; the VH comprises an amino acid sequence of SEQ ID NO: 12 and the VL comprises an amino acid sequence of SEQ ID NO: 37; the VH comprises an amino acid sequence of SEQ ID NO: 21 and the VL comprises an amino acid sequence of SEQ ID NO: 37; the VH comprises an amino acid sequence of SEQ ID NO: 14 and the VL comprises an amino acid sequence of SEQ ID NO: 36; the VH comprises an amino acid sequence of SEQ ID NO: 23 and the VL comprises an amino acid sequence of SEQ ID NO: 36; the VH comprises an amino acid sequence of SEQ ID NO: 15 and the VL comprises an amino acid sequence of SEQ ID NO: 36; or the VH comprises an amino acid sequence of SEQ ID NO: 24 and the VL comprises an amino acid sequence of SEQ ID NO: 36.

In an aspect, provided herein is an antigen binding protein or the antigen binding fragment thereof which specifically binds thyroid-stimulating hormone receptor (TSHR), wherein the antigen binding protein or an antigen binding fragment thereof comprising the VH comprises an amino acid sequence of SEQ ID NO: 78 and the VL comprises an amino acid sequence of SEQ ID NO: 25; the VH comprises an amino acid sequence of SEQ ID NO: 78 and the VL comprises an amino acid sequence of SEQ ID NO: 32; the VH comprises an amino acid sequence of SEQ ID NO: 16 and the VL comprises an amino acid sequence of SEQ ID NO: 25; the VH comprises an amino acid sequence of SEQ ID NO: 16 and the VL comprises an amino acid sequence of SEQ ID NO: 32; the VH comprises an amino acid sequence of SEQ ID NO: 7 and the VL comprises an amino acid sequence of SEQ ID NO: 29; the VH comprises an amino acid sequence of SEQ ID NO: 7 and the VL comprises an amino acid sequence of SEQ ID NO: 35; the VH comprises an amino acid sequence of SEQ ID NO: 7 and the VL comprises an amino acid sequence of SEQ ID NO: 30; the VH comprises an amino acid sequence of SEQ ID NO: 7 and the VL comprises an amino acid sequence of SEQ ID NO: 36; the VH comprises an amino acid sequence of SEQ ID NO: 8 and the VL comprises an amino acid sequence of SEQ ID NO: 29; the VH comprises an amino acid sequence of SEQ ID NO: 8 and the VL comprises an amino acid sequence of SEQ ID NO: 35; the VH comprises an amino acid sequence of SEQ ID NO: 8 and the VL comprises an amino acid sequence of SEQ ID NO: 30; the VH comprises an amino acid sequence of SEQ ID NO: 8 and the VL comprises an amino acid sequence of SEQ ID NO: 36; the VH comprises an amino acid sequence of SEQ ID NO: 9 and the VL comprises an amino acid sequence of SEQ ID NO: 29; the VH comprises an amino acid sequence of SEQ ID NO: 9 and the VL comprises an amino acid sequence of SEQ ID NO: 35; the VH comprises an amino acid sequence of SEQ ID NO: 9 and the VL comprises an amino acid sequence of SEQ ID NO: 30; the VH comprises an amino acid sequence of SEQ ID NO: 9 and the VL comprises an amino acid sequence of SEQ ID NO: 36; the VH comprises an amino acid sequence of SEQ ID NO: 10 and the VL comprises an amino acid sequence of SEQ ID NO: 29; the VH comprises an amino acid sequence of SEQ ID NO: 10 and the VL comprises an amino acid sequence of SEQ ID NO: 35; the VH comprises an amino acid sequence of SEQ ID NO: 10 and the VL comprises an amino acid sequence of SEQ ID NO: 30; the VH comprises an amino acid sequence of SEQ ID NO: 10 and the VL comprises an amino acid sequence of SEQ ID NO: 36; the VH comprises an amino acid sequence of SEQ ID NO: 11 and the VL comprises an amino acid sequence of SEQ ID NO: 29; the VH comprises an amino acid sequence of SEQ ID NO: 11 and the VL comprises an amino acid sequence of SEQ ID NO: 35; the VH comprises an amino acid sequence of SEQ ID NO: 11 and the VL comprises an amino acid sequence of SEQ ID NO: 30; the VH comprises an amino acid sequence of SEQ ID NO: 11 and the VL comprises an amino acid sequence of SEQ ID NO: 36; the VH comprises an amino acid sequence of SEQ ID NO: 12 and the VL comprises an amino acid sequence of SEQ ID NO: 29; the VH comprises an amino acid sequence of SEQ ID NO: 12 and the VL comprises an amino acid sequence of SEQ ID NO: 35; the VH comprises an amino acid sequence of SEQ ID NO: 12 and the VL comprises an amino acid sequence of SEQ ID NO: 30; the VH comprises an amino acid sequence of SEQ ID NO: 12 and the VL comprises an amino acid sequence of SEQ ID NO: 36; the VH comprises an amino acid sequence of SEQ ID NO: 13 and the VL comprises an amino acid sequence of SEQ ID NO: 29; the VH comprises an amino acid sequence of SEQ ID NO: 12 and the VL comprises an amino acid sequence of SEQ ID NO: 35; the VH comprises an amino acid sequence of SEQ ID NO: 12 and the VL comprises an amino acid sequence of SEQ ID NO: 30; the VH comprises an amino acid sequence of SEQ ID NO: 12 and the VL comprises an amino acid sequence of SEQ ID NO: 36; the VH comprises an amino acid sequence of SEQ ID NO: 16 and the VL comprises an amino acid sequence of SEQ ID NO: 29; the VH comprises an amino acid sequence of SEQ ID NO: 16 and the VL comprises an amino acid sequence of SEQ ID NO: 35; the VH comprises an amino acid sequence of SEQ ID NO: 16 and the VL comprises an amino acid sequence of SEQ ID NO: 30; the VH comprises an amino acid sequence of SEQ ID NO: 16 and the VL comprises an amino acid sequence of SEQ ID NO: 36; the VH comprises an amino acid sequence of SEQ ID NO: 17 and the VL comprises an amino acid sequence of SEQ ID NO: 29; the VH comprises an amino acid sequence of SEQ ID NO: 17 and the VL comprises an amino acid sequence of SEQ ID NO: 35; the VH comprises an amino acid sequence of SEQ ID NO: 17 and the VL comprises an amino acid sequence of SEQ ID NO: 30; the VH comprises an amino acid sequence of SEQ ID NO: 17 and the VL comprises an amino acid sequence of SEQ ID NO: 36; the VH comprises an amino acid sequence of SEQ ID NO: 18 and the VL comprises an amino acid sequence of SEQ ID NO: 29; the VH comprises an amino acid sequence of SEQ ID NO: 18 and the VL comprises an amino acid sequence of SEQ ID NO: 35; the VH comprises an amino acid sequence of SEQ ID NO: 18 and the VL comprises an amino acid sequence of SEQ ID NO: 30; the VH comprises an amino acid sequence of SEQ ID NO: 18 and the VL comprises an amino acid sequence of SEQ ID NO: 36; the VH comprises an amino acid sequence of SEQ ID NO: 19 and the VL comprises an amino acid sequence of SEQ ID NO: 29; the VH comprises an amino acid sequence of SEQ ID NO: 19 and the VL comprises an amino acid sequence of SEQ ID NO: 35; the VH comprises an amino acid sequence of SEQ ID NO: 19 and the VL comprises an amino acid sequence of SEQ ID NO: 30; the VH comprises an amino acid sequence of SEQ ID NO: 19 and the VL comprises an amino acid sequence of SEQ ID NO: 36; the VH comprises an amino acid sequence of SEQ ID NO: 20 and the VL comprises an amino acid sequence of SEQ ID NO: 29; the VH comprises an amino acid sequence of SEQ ID NO: 20 and the VL comprises an amino acid sequence of SEQ ID NO: 35; the VH comprises an amino acid sequence of SEQ ID NO: 20 and the VL comprises an amino acid sequence of SEQ ID NO: 30; the VH comprises an amino acid sequence of SEQ ID NO: 20 and the VL comprises an amino acid sequence of SEQ ID NO: 36; the VH comprises an amino acid sequence of SEQ ID NO: 21 and the VL comprises an amino acid sequence of SEQ ID NO: 29; the VH comprises an amino acid sequence of SEQ ID NO: 21 and the VL comprises an amino acid sequence of SEQ ID NO: 35; the VH comprises an amino acid sequence of SEQ ID NO: 21 and the VL comprises an amino acid sequence of SEQ ID NO: 30; the VH comprises an amino acid sequence of SEQ ID NO: 21 and the VL comprises an amino acid sequence of SEQ ID NO: 36; the VH comprises an amino acid sequence of SEQ ID NO: 22 and the VL comprises an amino acid sequence of SEQ ID NO: 29; the VH comprises an amino acid sequence of SEQ ID NO: 22 and the VL comprises an amino acid sequence of SEQ ID NO: 35; the VH comprises an amino acid sequence of SEQ ID NO: 22 and the VL comprises an amino acid sequence of SEQ ID NO: 30; the VH comprises an amino acid sequence of SEQ ID NO: 22 and the VL comprises an amino acid sequence of SEQ ID NO: 36; the VH comprises an amino acid sequence of SEQ ID NO: 11 and the VL comprises an amino acid sequence of SEQ ID NO: 26; the VH comprises an amino acid sequence of SEQ ID NO: 20 and the VL comprises an amino acid sequence of SEQ ID NO: 26; the VH comprises an amino acid sequence of SEQ ID NO: 12 and the VL comprises an amino acid sequence of SEQ ID NO: 26; the VH comprises an amino acid sequence of SEQ ID NO: 21 and the VL comprises an amino acid sequence of SEQ ID NO: 26; the VH comprises an amino acid sequence of SEQ ID NO: 11 and the VL comprises an amino acid sequence of SEQ ID NO: 27; the VH comprises an amino acid sequence of SEQ ID NO: 20 and the VL comprises an amino acid sequence of SEQ ID NO: 27; the VH comprises an amino acid sequence of SEQ ID NO: 12 and the VL comprises an amino acid sequence of SEQ ID NO: 27; the VH comprises an amino acid sequence of SEQ ID NO: 21 and the VL comprises an amino acid sequence of SEQ ID NO: 27; the VH comprises an amino acid sequence of SEQ ID NO: 11 and the VL comprises an amino acid sequence of SEQ ID NO: 28; the VH comprises an amino acid sequence of SEQ ID NO: 20 and the VL comprises an amino acid sequence of SEQ ID NO: 28; the VH comprises an amino acid sequence of SEQ ID NO: 12 and the VL comprises an amino acid sequence of SEQ ID NO: 28; the VH comprises an amino acid sequence of SEQ ID NO: 21 and the VL comprises an amino acid sequence of SEQ ID NO: 28; the VH comprises an amino acid sequence of SEQ ID NO: 11 and the VL comprises an amino acid sequence of SEQ ID NO: 31; the VH comprises an amino acid sequence of SEQ ID NO: 20 and the VL comprises an amino acid sequence of SEQ ID NO: 31; the VH comprises an amino acid sequence of SEQ ID NO: 12 and the VL comprises an amino acid sequence of SEQ ID NO: 31; the VH comprises an amino acid sequence of SEQ ID NO: 21 and the VL comprises an amino acid sequence of SEQ ID NO: 31; the VH comprises an amino acid sequence of SEQ ID NO: 14 and the VL comprises an amino acid sequence of SEQ ID NO: 30; the VH comprises an amino acid sequence of SEQ ID NO: 23 and the VL comprises an amino acid sequence of SEQ ID NO: 30; the VH comprises an amino acid sequence of SEQ ID NO: 15 and the VL comprises an amino acid sequence of SEQ ID NO: 30; the VH comprises an amino acid sequence of SEQ ID NO: 24 and the VL comprises an amino acid sequence of SEQ ID NO: 30; the VH comprises an amino acid sequence of SEQ ID NO: 11 and the VL comprises an amino acid sequence of SEQ ID NO: 32; the VH comprises an amino acid sequence of SEQ ID NO: 20 and the VL comprises an amino acid sequence of SEQ ID NO: 32; the VH comprises an amino acid sequence of SEQ ID NO: 12 and the VL comprises an amino acid sequence of SEQ ID NO: 32; the VH comprises an amino acid sequence of SEQ ID NO: 21 and the VL comprises an amino acid sequence of SEQ ID NO: 32; the VH comprises an amino acid sequence of SEQ ID NO: 11 and the VL comprises an amino acid sequence of SEQ ID NO: 33; the VH comprises an amino acid sequence of SEQ ID NO: 20 and the VL comprises an amino acid sequence of SEQ ID NO: 33; the VH comprises an amino acid sequence of SEQ ID NO: 12 and the VL comprises an amino acid sequence of SEQ ID NO: 33; the VH comprises an amino acid sequence of SEQ ID NO: 21 and the VL comprises an amino acid sequence of SEQ ID NO: 33; the VH comprises an amino acid sequence of SEQ ID NO: 11 and the VL comprises an amino acid sequence of SEQ ID NO: 34; the VH comprises an amino acid sequence of SEQ ID NO: 20 and the VL comprises an amino acid sequence of SEQ ID NO: 34; the VH comprises an amino acid sequence of SEQ ID NO: 12 and the VL comprises an amino acid sequence of SEQ ID NO: 34; the VH comprises an amino acid sequence of SEQ ID NO: 21 and the VL comprises an amino acid sequence of SEQ ID NO: 34; the VH comprises an amino acid sequence of SEQ ID NO: 11 and the VL comprises an amino acid sequence of SEQ ID NO: 37; the VH comprises an amino acid sequence of SEQ ID NO: 20 and the VL comprises an amino acid sequence of SEQ ID NO: 37; the VH comprises an amino acid sequence of SEQ ID NO: 12 and the VL comprises an amino acid sequence of SEQ ID NO: 37; the VH comprises an amino acid sequence of SEQ ID NO: 21 and the VL comprises an amino acid sequence of SEQ ID NO: 37; the VH comprises an amino acid sequence of SEQ ID NO: 14 and the VL comprises an amino acid sequence of SEQ ID NO: 36; the VH comprises an amino acid sequence of SEQ ID NO: 23 and the VL comprises an amino acid sequence of SEQ ID NO: 36; the VH comprises an amino acid sequence of SEQ ID NO: 15 and the VL comprises an amino acid sequence of SEQ ID NO: 36; or the VH comprises an amino acid sequence of SEQ ID NO: 24 and the VL comprises an amino acid sequence of SEQ ID NO: 36; the VH comprises an amino acid sequence of SEQ ID NO: 79 and the VL comprises an amino acid sequence of SEQ ID NO: 2; the VH comprises an amino acid sequence of SEQ ID NO: 80 and the VL comprises an amino acid sequence of SEQ ID NO; 2; the VH comprises an amino acid sequence of SEQ ID NO: 81 and the VL comprises an amino acid sequence of SEQ ID NO: 2; the VH comprises an amino acid sequence of SEQ ID NO: 82 and the VL comprises an amino acid sequence of SEQ ID NO: 2; the VH comprises an amino acid sequence of SEQ ID NO: 83 and the VL comprises an amino acid sequence of SEQ ID NO: 2; the VH comprises an amino acid sequence of SEQ ID NO: 84 and the VL comprises an amino acid sequence of SEQ ID NO: 2; the VH comprises an amino acid sequence of SEQ ID NO: 85 and the VL comprises an amino acid sequence of SEQ ID NO: 2; the VH comprises an amino acid sequence of SEQ ID NO: 86 and the VL comprises an amino acid sequence of SEQ ID NO: 2; the VH comprises an amino acid sequence of SEQ ID NO: 87 and the VL comprises an amino acid sequence of SEQ ID NO: 2; the VH comprises an amino acid sequence of SEQ ID NO: 88 and the VL comprises an amino acid sequence of SEQ ID NO: 2; the VH comprises an amino acid sequence of SEQ ID NO: 1 and the VL comprises an amino acid sequence of SEQ ID NO: 89; the VH comprises an amino acid sequence of SEQ ID NO: 1 and the VL comprises an amino acid sequence of SEQ ID NO: 90; the VH comprises an amino acid sequence of SEQ ID NO: 1 and the VL comprises an amino acid sequence of SEQ ID NO: 91; the VH comprises an amino acid sequence of SEQ ID NO: 1 and the VL comprises an amino acid sequence of SEQ ID NO: 92; the VH comprises an amino acid sequence of SEQ ID NO: 1 and the VL comprises an amino acid sequence of SEQ ID NO: 93; the VH comprises an amino acid sequence of SEQ ID NO: 1 and the VL comprises an amino acid sequence of SEQ ID NO: 94; the VH comprises an amino acid sequence of SEQ ID NO: 1 and the VL comprises an amino acid sequence of SEQ ID NO: 95; or the VH comprises an amino acid sequence of SEQ ID NO: 1 and the VL comprises an amino acid sequence of SEQ ID NO: 96.

In one aspect, the disclosure provides an antigen binding protein or the antigen binding fragment thereof which specifically binds thyroid-stimulating hormone receptor (TSHR), wherein the antigen binding protein or an antigen binding fragment thereof comprises: a. a VH comprising an amino acid sequence of SEQ ID NO: 120 and a VL comprising an amino acid sequence of SEQ ID NO: 2; b. a VH comprising an amino acid sequence of SEQ ID NO: 121 and a VL comprising an amino acid sequence of SEQ ID NO: 2; c. a VH comprising an amino acid sequence of SEQ ID NO: 122 and a VL comprising an amino acid sequence of SEQ ID NO: 2; d. a VH comprising an amino acid sequence of SEQ ID NO: 123 and a VL comprising an amino acid sequence of SEQ ID NO: 2; e. a VH comprising an amino acid sequence of SEQ ID NO: 124 and a VL comprising an amino acid sequence of SEQ ID NO: 2; f. a VH comprising an amino acid sequence of SEQ ID NO: 125 and a VL comprising an amino acid sequence of SEQ ID NO: 2; g. a VH comprising an amino acid sequence of SEQ ID NO: 126 and a VL comprising an amino acid sequence of SEQ ID NO: 2; or h. a VH comprising an amino acid sequence of SEQ ID NO: 127 and a VL comprising an amino acid sequence of SEQ ID NO: 2.

In one aspect, the disclosure provides an antigen binding protein or the antigen binding fragment thereof which specifically binds thyroid-stimulating hormone receptor (TSHR), wherein the antigen binding protein or an antigen binding fragment thereof comprises: a. a VH comprising an amino acid sequence of SEQ ID NO: 128 and a VL comprising an amino acid sequence of SEQ ID NO: 129; b. a VH comprising an amino acid sequence of SEQ ID NO: 136 and a VL comprising an amino acid sequence of SEQ ID NO: 137; c. a VH comprising an amino acid sequence of SEQ ID NO: 138 and a VL comprising an amino acid sequence of SEQ ID NO: 129; d. a VH comprising an amino acid sequence of SEQ ID NO: 139 and a VL comprising an amino acid sequence of SEQ ID NO: 129; e. a VH comprising an amino acid sequence of SEQ ID NO: 140 and a VL comprising an amino acid sequence of SEQ ID NO: 129; f. a VH comprising an amino acid sequence of SEQ ID NO: 141 and a VL comprising an amino acid sequence of SEQ ID NO: 137; g. a VH comprising an amino acid sequence of SEQ ID NO: 142 and a VL comprising an amino acid sequence of SEQ ID NO: 137; h. a VH comprising an amino acid sequence of SEQ ID NO: 143 and a VL comprising an amino acid sequence of SEQ ID NO: 129; or i. a VH comprising an amino acid sequence of SEQ ID NO: 143 and a VL comprising an amino acid sequence of SEQ ID NO: 137.

In some embodiments, the protein further comprises an immunoglobulin Fc domain or variant thereof. In some embodiments, the Fc domain or variant thereof comprises a first Fc heavy chain and a second Fc heavy chain. In some embodiments, at least one of the Fc heavy chains comprises one or more mutations to promote increased half-life. In some embodiments, the at least one Fc heavy chain comprises one or more substitutions at amino acid positions 252, 254, and/or 256, according to EU numbering. In some embodiments, the substitution at amino acid position 252 is a tyrosine (Y), the substitution at amino acid position 254 is a threonine (T), and the substitution at amino acid position 256 is a glutamic acid (E). In some embodiments, the Fc heavy chain comprises an amino acid sequence of SEQ ID NO: 5. In some embodiments, the at least one Fc heavy chain comprises one or more substitutions at amino acid positions 428 and/or 434, according to EU numbering. In some embodiments, the substitution at amino acid position 428 is a leucine (L), and the substitution at amino acid position 434 is a serine(S).

In some embodiments, the at least one of the Fc heavy chains comprises heterodimerization mutations. In some embodiments, the heterodimerization mutations are charge stabilization mutations. In some embodiments, the heterodimerization mutations comprise an engineered disulfide bond.

In some embodiments, the antigen binding protein or antigen binding fragment thereof has reduced aggregation compared to an antigen binding protein comprising a VH domain of SEQ ID NO: 1 and a VL domain of SEQ ID NO: 2.

In some embodiments, the antigen binding protein or the antigen binding fragment thereof has increased solubility compared to an antigen binding protein comprising a VH domain of SEQ ID NO: 1 and a VL domain of SEQ ID NO: 2.

In some embodiments, the antigen binding protein or the antigen binding fragment thereof has a higher melting temperature compared to an antigen binding protein comprising a VH domain of SEQ ID NO: 1 and a VL domain of SEQ ID NO: 2.

In some embodiments, the antigen binding protein or the antigen binding fragment thereof blocks TSHR autoantibodies from binding to TSHR.

In some embodiments, the antigen binding protein or the antigen binding fragment thereof decreases an autoimmune antibody response compared to an antigen binding protein comprising a VH domain of SEQ ID NO: 1 and a VL domain of SEQ ID NO: 2.

In some embodiments, the antigen binding protein or the antigen binding fragment thereof has a reduced anti-drug antibody response compared to an antigen binding protein comprising a VH domain of SEQ ID NO: 1 and a VL domain of SEQ ID NO: 2.

In another aspect, provided herein is an antigen binding protein or an antigen binding fragment thereof which specifically binds thyroid-stimulating hormone receptor (TSHR), wherein the antigen binding protein or an antigen binding fragment thereof comprises a heavy chain variable (VH) domain with at least 90% identity to SEQ ID NO: 78 with a glutamic acid (E) at position 16, a serine(S) at position 77, and a glutamine (Q) at position 111 relative to SEQ ID NO: 78; and a light chain variable (VL) domain with at least 90% identity to SEQ ID NO: 25 with a leucine (L) amino acid at position 40 and a glycine (G) amino acid at position 58 relative to SEQ ID NO: 25.

In another aspect, provided herein is an antigen binding protein or an antigen binding fragment thereof which specifically binds thyroid-stimulating hormone receptor (TSHR), wherein the antigen binding protein or an antigen binding fragment thereof comprises: a heavy chain variable (VH) domain with at least 90% identity to SEQ ID NO: 78 and comprising a heavy chain framework region 1 (HFR1) amino acid sequence of SEQ ID NO: 98, a heavy chain framework region 2 (HFR2) amino acid sequence of SEQ ID NO: 99, a heavy chain framework region 3 (HFR3) amino acid sequence of SEQ ID NO: 100, and a heavy chain framework region 4 (HFR4) amino acid sequence of SEQ ID NO: 101; and a light chain variable (VL) domain with at least 90% identity to SEQ ID NO: 25 and comprising a light chain framework region 1 (LFR1) amino acid sequence of SEQ ID NO: 102, a light chain framework region 2 (LFR2) amino acid sequence of SEQ ID NO: 103, a light chain framework region 3 (LFR3) amino acid sequence of SEQ ID NO: 104, and a light chain framework region 4 (LFR4) amino acid sequence of SEQ ID NO: 105.

In some embodiments, the antigen binding protein or an antigen binding fragment thereof comprises an HCDR1 sequence of SEQ ID NO: 38, an HCDR2 sequence of SEQ ID NO: 39, an HCDR3 sequence of SEQ ID NO: 40, an LCDR1 sequence of SEQ ID NO: 41, an LCDR2 sequence of SEQ ID NO: 42, and an LCDR3 sequence of SEQ ID NO: 43.

In another aspect, provided herein is an antigen binding protein or an antigen binding fragment thereof which specifically binds thyroid-stimulating hormone receptor (TSHR), wherein the antigen binding protein or an antigen binding fragment thereof comprises: a heavy chain variable (VH) domain with at least 90% identity to SEQ ID NO: 3 and comprising a heavy chain framework region 1 (HFR1) amino acid sequence of SEQ ID NO: 112, a heavy chain framework region 2 (HFR2) amino acid sequence of SEQ ID NO: 113, a heavy chain framework region 3 (HFR3) amino acid sequence of SEQ ID NO: 114, and a heavy chain framework region 4 (HFR4) amino acid sequence of SEQ ID NO: 115; and a light chain variable (VL) domain with at least 90% identity to SEQ ID NO: 4 and comprising a light chain framework region 1 (LFR1) amino acid sequence of SEQ ID NO: 116, a light chain framework region 2 (LFR2) amino acid sequence of SEQ ID NO: 117, a light chain framework region 3 (LFR3) amino acid sequence of SEQ ID NO: 118, and a light chain framework region 4 (LFR4) amino acid sequence of SEQ ID NO: 119.

In some embodiments, the antigen binding protein or an antigen binding fragment thereof comprises an HCDR1 sequence of SEQ ID NO: 106, an HCDR2 sequence of SEQ ID NO: 107, an HCDR3 sequence of SEQ ID NO: 108, an LCDR1 sequence of SEQ ID NO: 109, an LCDR2 sequence of SEQ ID NO: 110, and an LCDR3 sequence of SEQ ID NO: 111.

In another aspect, provided herein is a pharmaceutical composition comprising the antigen binding protein or the antigen binding fragment thereof of the present disclosure.

In some embodiments, the composition comprises a concentration of the antigen binding protein or antigen binding fragment thereof of any one of claims 1-22 in a concentration ≥150 mg/mL.

In an aspect, provided herein is an isolated nucleic acid molecule encoding the antigen binding protein or the antigen binding fragment of the present disclosure.

In yet another aspect, provided herein is an expression vector comprising a nucleic acid molecule of the present disclosure.

In an aspect, provided herein is a host cell comprising an expression vector comprising a nucleic acid molecule of the present disclosure.

In another aspect, provided herein is a method of treating or preventing a thyroid stimulating hormone receptor (TSHR)-related disease in a subject, comprising administering to a subject in need thereof the antigen binding protein or antigen binding fragment of the present disclosure.

In some embodiments, the TSHR-related disease is an autoimmune disease. In some embodiments, the autoimmune disease is Graves' disease. In some embodiments, the TSHR-related disease is cancer.

In another aspect, provided herein is a method of treating an autoimmune disease associated with autoantibodies to thyroid stimulating hormone receptor (TSHR) in a subject, comprising administering to a subject in need thereof the antigen binding protein or antigen binding fragment thereof of the present disclosure.

In another aspect, provided herein is a chimeric antigen receptor (CAR) comprising a TSHR-binding domain comprising the antigen binding protein or antigen binding fragment thereof of the present disclosure.

In an aspect, provided herein is a cell comprising the CAR of the present disclosure.

In an aspect, provided herein is an antibody drug conjugate (ADC) comprising the antigen binding protein or an antigen binding fragment thereof which specifically binds thyroid-stimulating hormone receptor (TSHR) of the present disclosure.

In some embodiments, the ADC is conjugated to a radioisotope or to a therapeutic small molecule.

In an aspect, provided herein is a method of treating or preventing a disease or disorder comprising administration of the ADC of the present disclosure.

In another aspect, provided herein is a method of diagnosing or detecting a disease or disorder comprising administration of the ADC of the present disclosure.

In some embodiments, the disease or disorder is associated with thyroid-stimulating hormone receptor (TSHR) expression.

In one aspect, the disclosure provides a nucleic acid library comprising a plurality of polynucleotide sequences, each polynucleotide sequence in the plurality encoding for a variant anti-TSHR antigen binding protein comprising one or both of: a variant variable heavy chain (VH) comprising one or more amino acid substitutions in the amino acid sequence of SEQ ID NO: 1 or 128, and a variant variable light chain (VL) comprising one or more amino acid substitutions in the amino acid sequence of SEQ ID NO: 2 or 129.

In some embodiments, each variant VH encoded the polynucleotide sequence in the plurality comprises or consists of one acid substitution in the amino acid sequence of SEQ ID NO: 1 or 128.

In some embodiments, each variant VL encoded the polynucleotide sequence in the plurality comprises or consists of one acid substitution in the amino acid sequence of SEQ ID NO: 2 or 129.

In some embodiments, the library comprises a diversity of at least about 100, 200, 300, 400, 500, 600, 700, 800, 900, 1000, 1500, or 2000 unique polynucleotide sequences.

In some embodiments, the library comprises a diversity of about 100 to about 2000 unique polynucleotide sequences encoding variant VH amino acid sequences of SEQ ID NO: 1 or 128.

In some embodiments, the library comprises a diversity of about 1920 unique polynucleotide sequences encoding variant VH amino acid sequences of SEQ ID NO: 1 or 128.

In some embodiments, the library comprises a diversity of about 100 to about 2000 unique polynucleotide sequences encoding variant VL amino acid sequences of SEQ ID NO: 2 or 129.

In some embodiments, the library comprises a diversity of about 1660 unique polynucleotide sequences encoding variant VL amino acid sequences of SEQ ID NO: 2 or 129.

### BRIEF DESCRIPTION OF THE DRAWINGS

FIG. 1 depicts a summary of the DMS library fluorescent activated cell sorting strategy. Expression of the light or heavy chain is depicted on the y-axis and relative binding to human TSHR is depicted on the x-axis.

FIG. 2 depicts FACS analysis of TSHR variant antibody fab domains based on expression and TSHR binding. The top two graphs correspond to incubation at pH 7.5 with TSHR at 1 nM. The bottom two graphs correspond to incubation at pH 5.5 with TSHR at 1 nM.

FIG. 3A-3D (SEQ ID NOs: 145-146) depict enrichment heatmaps from FACS analysis of TSHR variant antibody fab domains based on expression and TSHR binding. FIG. 3A depicts log enrichment of specific mutations in the VH paratope under pH 7.5 conditions. FIG. 3B depicts log enrichment of specific mutations in the VH paratope under pH 5.5 conditions. FIG. 3C depicts log enrichment of specific mutations in the VL paratope under pH 7.5 conditions. FIG. 3D depicts log enrichment of specific mutations in the VL paratope under pH 5.5 conditions.

FIG. 4A-4H (SEQ ID NOs: 145-146) depict enrichment heatmaps from FACS analysis of TSHR variant antibody fab domains based on expression and TSHR binding. FIG. 4A depicts raw enrichment of specific mutations in the VH paratope under pH 7.5 conditions under the negative gating. FIG. 4B depicts raw enrichment of specific mutations in the VH paratope under pH 7.5 conditions under MP gating. FIG. 4C depicts raw enrichment of specific mutations in the VH paratope under pH 7.5 conditions under positive gating. FIG. 4D depicts raw enrichment of specific mutations in the VH paratope under pH 7.5 conditions under pospos gating. FIG. 4E depicts raw enrichment of specific mutations in the VL paratope under pH 7.5 conditions under the negative gating. FIG. 4F depicts raw enrichment of specific mutations in the VL paratope under pH 7.5 conditions under MP gating. FIG. 4G depicts raw enrichment of specific mutations in the VL paratope under pH 7.5 conditions under positive gating. FIG. 4H depicts raw enrichment of specific mutations in the VL paratope under pH 7.5 conditions under pospos gating.

FIG. 5A-5H (SEQ ID NOs: 145-146) depict enrichment heatmaps from FACS analysis of TSHR variant antibody fab domains based on expression and TSHR binding. FIG. 5A depicts raw enrichment of specific mutations in the VH paratope under pH 5.5 conditions under the negative gating. FIG. 5B depicts raw enrichment of specific mutations in the VH paratope under pH 5.5 conditions under MP gating. FIG. 5C depicts raw enrichment of specific mutations in the VH paratope under pH 5.5 conditions under positive gating. FIG. 5D depicts raw enrichment of specific mutations in the VH paratope under pH 5.5 conditions under pospos gating. FIG. 5E depicts raw enrichment of specific mutations in the VL paratope under pH 5.5 conditions under the negative gating. FIG. 5F depicts raw enrichment of specific mutations in the VL paratope under pH 5.5 conditions under MP gating. FIG. 5G depicts raw enrichment of specific mutations in the VL paratope under pH 5.5 conditions under positive gating. FIG. 5H depicts raw enrichment of specific mutations in the VL paratope under pH 5.5 conditions under pospos gating.

FIG. 6 depicts FACS analysis of TSHR variant antibody fab domains based on expression and TSHR binding. A thermal gate was employed to select for variants with the same or better antigen binding signal as parental after heating at 55° C. (VH) or 60° C. (VL) during 10 min incubation at pH 7.5 with a human TSHR concentration of 25 nM.

FIG. 7A-7B (SEQ ID NOs: 145-146) depict enrichment heatmaps from FACS analysis of TSHR variant antibody fab domains based on expression and TSHR binding. FIG. 7A depicts log enrichment of specific mutations in the VH paratope under thermal conditions of 10 min at 55° C.

FIG. 7B depicts log enrichment of specific mutations in the VL paratope under thermal conditions of 10 min at 55° C.

FIG. 8 depicts FACS analysis of TSHR variant antibody fab domains based on expression and NSB reagent binding corresponding to human cell lysate for non-specific binding. A positive and negative binding gate was employed.

FIG. 9A-9D (SEQ ID NOs: 145-146) depict enrichment heatmaps from FACS analysis of TSHR variant antibody fab domains based on expression and NSB binding. FIG. 9A depicts log enrichment of specific mutations in the VH paratope under negative gating. FIG. 9B depicts log enrichment of specific mutations in the VH paratope under positive gating. FIG. 9C depicts log enrichment of specific mutations in the VL paratope under negative gating. FIG. 9D depicts log enrichment of specific mutations in the VL paratope under positive gating.

FIG. 10 depicts a scheme for selecting TSHR variant antibodies with differential biding at pH 7.5 and 5.5. MP refers to a “minus pos” population meaning a population of yeast cells expressing a Fab with slightly altered affinity compared to parental. Pos refers to a parental like yeast population expressing Fabs with comparable affinity compared to parental.

FIG. 11A-FIG. 11B depicts FACS analysis of TSHR variant antibody fab domains based on expression and TSHR binding. A MP and positive binding gate was employed. The top two graphs correspond to incubation at pH 7.5 with TSHR at 1 nM. The bottom two graphs correspond to incubation at pH 5.5 with TSHR at 1 nM. FIG. 11A corresponds to the VH library and FIG. 11B corresponds to the VL library.

FIG. 12A-12D (SEQ ID NOs: 145-146) depict enrichment heatmaps from FACS analysis of TSHR variant antibody fab domains based on expression and TSHR binding. FIG. 12A depicts raw enrichment of specific mutations in the VH paratope under the pH switch condition under the MP gating. FIG. 12B depicts raw enrichment of specific mutations in the VH paratope under the pH switch condition under the positive gating. FIG. 12C depicts raw enrichment of specific mutations in the VL paratope under the pH switch condition under the MP gating. FIG. 12D depicts raw enrichment of specific mutations in the VL paratope under the pH switch condition under the positive gating.

FIG. 13A-13B depicts binding fluorescence at pH 7.5 divided by expression at the yeast surface (FIG. 13A) and binding at pH 5.5 compared to binding at pH 7.5 (FIG. 13B). Select TSHR variant antibodies are depicted. For each data point, the left bar corresponds to the parental antibody and the right bar corresponds to the variant.

FIG. 14 depicts binding fluorescence at pH 7.5 and pH 5.5 relative to WT binding fluorescence for several T3 antibody variants.

### DETAILED DESCRIPTION

Before the present disclosure is described, it is to be understood that this disclosure is not limited to particular methods and experimental conditions described, as such methods and conditions may vary. It is also to be understood that the terminology used herein is for the purpose of describing particular embodiments only, and is not intended to be limiting, since the scope of the present disclosure will be limited only by the appended claims.

Unless defined otherwise, all technical and scientific terms used herein have the same meaning as commonly understood by one of ordinary skill in the art to which this disclosure belongs.

Although any methods and materials similar or equivalent to those described herein can be used in the practice of the present disclosure, exemplary methods and materials are now described. All publications mentioned herein are incorporated herein by reference to describe in their entirety.

As used herein, the terms “protein”, “peptide” and “polypeptide” are used interchangeably to designate a series of amino acid residues connected to each other by peptide bonds between the alpha-amino and carboxy groups of adjacent residues. The terms “protein”, “peptide” and “polypeptide” refer to a polymer of amino acids, including modified amino acids (e.g., phosphorylated, glycated, glycosylated, etc.) and amino acid analogs, regardless of its size or function. “Protein” and “polypeptide” are often used in reference to relatively large polypeptides, whereas the term “peptide” is often used in reference to small polypeptides, but usage of these terms in the art overlaps. The terms “protein”, “peptide” and “polypeptide” are used interchangeably herein when referring to a gene product and fragments thereof. These terms encompass, e.g., native and artificial proteins, protein fragments and polypeptide analogs (such as muteins, variants, and fusion proteins) of a protein sequence as well as post-translationally, or otherwise covalently or non-covalently, modified proteins. A peptide, polypeptide, or protein may be monomeric or polymeric. A polypeptide can have the amino acid sequence of naturally occurring polypeptide from any mammal. Such native sequence polypeptide can be isolated from nature or can be produced by recombinant or synthetic means. In some embodiments, the polypeptide is a “variant”. “Variant” means a biologically active polypeptide having at least about 80% amino acid sequence identity with the native sequence polypeptide after aligning the sequences and introducing gaps, if necessary, to achieve the maximum percent sequence identity, and not considering any conservative substitutions as part of the sequence identity. Such variants include, for instance, polypeptides wherein one or more amino acid residues are added, or deleted, at the N- or C-terminus of the polypeptide. In some embodiments, a variant will have at least about 80% amino acid sequence identity. In some embodiments, a variant will have at least about 90% amino acid sequence identity. In some embodiments, a variant will have at least about 95% amino acid sequence identity with the native sequence polypeptide. A “derivative” of a polypeptide is a polypeptide (e.g., an antibody) that has been chemically modified, e.g., via conjugation to another chemical moiety (such as, for example, polyethylene glycol or albumin, e.g., human serum albumin), phosphorylation, and glycosylation

As used herein, the terms “antibody” and “antibodies” include full-length antibodies, antigen binding fragments of full-length antibodies, and molecules comprising antibody CDRs, VH regions, and/or VL regions. Examples of antibodies include, without limitation, monoclonal antibodies, recombinantly produced antibodies, monospecific antibodies, multispecific antibodies (including bispecific antibodies), human antibodies, humanized antibodies, chimeric antibodies, immunoglobulins, synthetic antibodies, tetrameric antibodies comprising two heavy chain and two light chain molecules, an antibody light chain monomer, an antibody heavy chain monomer, an antibody light chain dimer, an antibody heavy chain dimer, an antibody light chain-antibody heavy chain pair, intrabodies, heteroconjugate antibodies, antibody-drug conjugates, single domain antibodies, monovalent antibodies, single chain antibodies or single-chain Fvs (scFv), camelized antibodies, affibodies, common light chain antibodies, Fab fragments, F(ab′)2 fragments, disulfide-linked Fvs (sdFv), anti-idiotypic (anti-Id) antibodies (including, e.g., anti-anti-Id antibodies), and antigen-binding fragments of any of the above. In certain embodiments, antibodies described herein refer to polyclonal antibody populations. Antibodies can be of any type (e.g., IgG, IgE, IgM, IgD, IgA or IgY), any class (e.g., IgG1, IgG2, IgG3, IgG4, IgA1 or IgA2), or any subclass (e.g., IgG2a or IgG2b) of immunoglobulin molecule. In certain embodiments, antibodies described herein are IgG antibodies, or a class (e.g., human IgG1 or IgG4) or subclass thereof. As used herein, the terms “VH” and “VL” refer to antibody heavy and light chain variable domain, respectively, as described in Kabat et al., (1991) Sequences of Proteins of Immunological Interest (NIH Publication No. 91-3242, Bethesda), which is herein incorporated by reference in its entirety.

As used herein, the term “antigen binding protein” or “binding domain” or “binding specificity” refers to a molecule that specifically binds to an antigen as such binding is understood by one skilled in the art. For example, an antigen binding protein that specifically binds to an antigen may bind to other molecules, generally with lower affinity as determined by, e.g., immunoassays (e.g., ELISA), BIAcore®, KinExA 3000 instrument (Sapidyne Instruments, Boise, ID), surface plasmon resonance (SPR) analysis, biolayer interferometry (BLI), or other assays known in the art. In certain embodiments, an antigen-binding moiety that specifically binds to an antigen binds to the antigen with a Ka that is at least 2 logs (e.g., factors of 10), 2.5 logs, 3 logs, 4 logs or greater than the Ka when the molecule binds non-specifically to another antigen.

As used herein, the term “VH/VL pair” refers to a combination of a VH and a VL that together form the binding site for an antigen.

As used herein, the term “heavy chain” when used in reference to an antibody can refer to any distinct type, e.g., alpha (α), delta (δ), epsilon (¿), gamma (γ), and mu (μ), based on the amino acid sequence of the constant domain, which give rise to IgA, IgD, IgE, IgG, and IgM classes of antibodies, respectively, including subclasses of IgG, e.g., IgG1, IgG2, IgG3, and IgG4.

As used herein, the term “full-length antibody heavy chain” refers to an antibody heavy chain comprising, from N to C terminal, a VH, a CH1 region, a hinge region, a CH2 domain and a CH3 domain.

As used herein, the term “light chain” when used in reference to an antibody can refer to any distinct type, e.g., kappa (κ) or lambda (λ) based on the amino acid sequence of the constant domains. Light chain amino acid sequences are well known in the art. In specific embodiments, the light chain is a human light chain. As used herein, the term “complementarity determining region” or “CDR” refers to sequences of amino acids within antibody variable regions, which confer antigen specificity and binding affinity. In general, there are three CDRs in each heavy chain variable region (HCDR1, HCDR2, HCDR3) and three CDRs in each light chain variable region (LCDR1, LCDR2, LCDR3). Exemplary hypervariable loops occur at amino acid residues 26-32 (L1), 50-52 (L2), 91-96 (L3), 26-32 (H1), 53-55 (H2), and 96-101 (H3). (Chothia and Lesk, J. Mol. Biol. 196:901-917 (1987)). Exemplary CDRs (CDR-L1, CDR-L2, CDR-L3, CDR-H1, CDR-H2, and CDR-H3) occur at amino acid residues 24-34 of L1, 50-56 of L2, 89-97 of L3, 31-35B of H1, 50-65 of H2, and 95-102 of H3 (Kabat et al., Sequences of Proteins of Immunological Interest, 5th ed. (1991)). Thus, the VHs may be comprised within the corresponding CDRs and references herein to the “hypervariable loops” of VH and VL domains should be interpreted as also encompassing the corresponding CDRs, and vice versa, unless otherwise indicated. “Framework regions” or “FR” are known in the art to refer to the non-CDR portions of the variable regions of the heavy and light chains. In general, there are four FRs in each heavy chain variable region (FR-H1, FR-H2, FR-H3, and FR-H4), and four FRs in each light chain variable region (FR-L1, FR-L2, FR-L3, and FR-L4).

The precise amino acid sequence boundaries of a given CDR or FR can be readily determined using any of a number of well-known schemes, including those described by Kabat et al. (1991), “Sequences of Proteins of Immunological Interest,” 5th Ed. Public Health Service, National Institutes of Health, Bethesda, MD. (“Kabat” numbering scheme), Al-Lazikani et al., (1997) JMB 273, 927-948 (“Chothia” numbering scheme), MacCallum et al., J. Mol. Biol. 262:732-745 (1996), “Antibody-antigen interactions: Contact analysis and binding site topography,” J. Mol. Biol. 262, 732-745. (“Contact” numbering scheme), Lefranc M. P. et al., “IMGT unique numbering for immunoglobulin and T cell receptor variable domains and Ig superfamily V-like domains,” Dev. Comp. Immunol., 2003 January; 27(1):55-77 (“IMGT” numbering scheme), and Honegger A. and Pluckthun A., “Yet another numbering scheme for immunoglobulin variable domains: an automatic modeling and analysis tool,” J. Mol. Biol., 2001 Jun. 8; 309(3):657-70, (Aho numbering scheme).

The boundaries of a given CDR or FR may vary depending on the scheme used for identification. For example, the Kabat scheme is based on sequence alignments, while the Chothia scheme is based on structural information. Numbering for both the Kabat and Chothia schemes is based upon the most common antibody region sequence lengths, with insertions accommodated by insertion letters, for example, “30a,” and deletions appearing in some antibodies. The two schemes place certain insertions and deletions (“indels”) at different positions, resulting in differential numbering. The Contact scheme is based on analysis of complex crystal structures and is similar in many respects to the Chothia numbering scheme.

As used herein, the term “single chain variable fragment” (scFv) refers to a fusion protein comprising at least one antibody fragment comprising a variable region of a light chain and at least one antibody fragment comprising a variable region of a heavy chain, wherein the light and heavy chain variable regions are contiguously linked via a short flexible polypeptide linker, and capable of being expressed as a single chain polypeptide, and wherein the scFv retains the specificity of the intact antibody from which it is derived. Unless specified, as used herein an scFv may have the VL and VH variable regions in either order, e.g., with respect to the N-terminal and C-terminal ends of the polypeptide, the scFv may comprise VL-linker-VH or may comprise VH-linker-VL.

The term “human antibody,” as used herein, is intended to include antibodies having variable and Fc domains derived from human germline immunoglobulin sequences. The human mAbs of the disclosure may include amino acid residues not encoded by human germline immunoglobulin sequences (e.g., mutations introduced by random or site-specific mutagenesis in vitro or by somatic mutation in vivo), for example in the CDRs and in particular CDR3. However, the term “human antibody,” as used herein, is not intended to include mAbs in which CDR sequences derived from the germline of another mammalian species (e.g., mouse), have been grafted onto human FR sequences. The term includes antibodies recombinantly produced in a non-human mammal, or in cells of a non-human mammal. The term is not intended to include antibodies isolated from or generated in a human subject.

The term “multispecific antigen-binding molecules,” as used herein refers to bispecific, tri-specific or multi-specific antigen-binding molecules, and antigen-binding fragments thereof. Multispecific antigen-binding molecules may be specific for different epitopes of one target polypeptide or may contain antigen-binding domains specific for epitopes of more than one target polypeptide. In certain embodiment, the multispecific antigen binding molecules of the disclosure comprises at least a first binding specificity for a subunit of a receptor and at least a second binding specificity for another receptor subunit. A multispecific antigen-binding molecule can be a single multifunctional polypeptide, or it can be a multimeric complex of two or more polypeptides that are covalently or non-covalently associated with one another. The term “multispecific antigen-binding molecules” includes antibodies of the present disclosure that may be linked to or co-expressed with another functional molecule, e.g., another peptide or protein. For example, an antibody or fragment thereof can be functionally linked (e.g., by chemical coupling, genetic fusion, non-covalent association or otherwise) to one or more other molecular entities, such as a protein or fragment thereof to produce a bi-specific or a multi-specific antigen-binding molecule with a second binding specificity. According to the present disclosure, the term “multispecific antigen-binding molecules” also includes bispecific, trispecific or multispecific antibodies or antigen-binding fragments thereof. In certain exemplary embodiments, an antibody of the present disclosure is functionally linked to another antibody or antigen-binding fragment thereof to produce a bispecific antibody with a second binding specificity.

In exemplary embodiments, the heteromeric antibodies of the present disclosure are bispecific antibodies. Bispecific antibodies can be monoclonal, e.g., human or humanized, antibodies that have binding specificities for at least two different antigens. In certain embodiments, the bispecific antibodies of the disclosure comprises at least a first binding domain for a receptor subunit and at least a second binding domain for another receptor subunit.

Methods for making bispecific antibodies are well-known. Traditionally, the recombinant production of bispecific antibodies was based on the co-expression of two immunoglobulin heavy chain/light chain pairs, where the two heavy chains have different specificities (Milstein et al., Nature 305:537 (1983)). Because of the random assortment of immunoglobulin heavy and light chains, the hybridomas (quadromas) produce a potential mixture of ten different antibody molecules, of which only one has the correct bispecific structure. The purification of the correct molecule is usually accomplished by affinity chromatography steps. More modern techniques for generating bispecific antibodies employ heterodimerization domains that favor desired pairing of heavy chain from the antibody with a first specificity to the heavy chain of an antibody with a second specificity.

Antibody variable domains with the desired binding specificities can be fused to immunoglobulin constant domain sequences. The fusion typically is with an immunoglobulin heavy chain constant domain, comprising at least part of the hinge, CH2, and CH3 regions. It may have the first heavy chain Fc domain (CH1) containing the site necessary for light chain binding present in at least one of the fusions. DNAs encoding the immunoglobulin heavy chain fusions and, if desired, the immunoglobulin light chain, are inserted into separate expression vectors, and are co-transformed into a suitable host organism. For further details of generating bispecific antibodies see, for example Suresh et al., Meth. Enzymol. 121:210 (1986).

As used herein, the term “Fc” refers to a polypeptide comprising a CH2 domain and a CH3 domain, wherein the C-terminus of the CH2 domain is linked (directly or indirectly) to the N-terminus of the CH3 domain. The term “Fc polypeptide” includes an antibody heavy chain linked to an antibody light chain by disulfide bonds (e.g., to form a half-antibody).

In certain embodiments, an Fc chain begins in the hinge region just upstream of the papain cleavage site and ends at the C-terminus of the antibody. Accordingly, a complete Fc chain comprises at least a hinge domain, a CH2 domain, and a CH3 domain. In certain embodiments, an Fc chain comprises at least one of: a hinge (e.g., upper, middle, and/or lower hinge region) domain, a CH2 domain, a CH3 domain, a CH4 domain, or a variant, portion, or fragment thereof. In certain embodiments, an Fc domain comprises a complete Fc chain (i.e., a hinge domain, a CH2 domain, and a CH3 domain). In certain embodiments, an Fc chain comprises a hinge domain (or portion thereof) fused to a CH3 domain (or portion thereof). In certain embodiments, an Fc chain comprises a CH2 domain (or portion thereof) fused to a CH3 domain (or portion thereof). In certain embodiments, an Fc chain consists of a CH3 domain or portion thereof. In certain embodiments, an Fc chain consists of a hinge domain (or portion thereof) and a CH3 domain (or portion thereof). In certain embodiments, an Fc chain consists of a CH2 domain (or portion thereof) and a CH3 domain. In certain embodiments, an Fc chain consists of a hinge domain (or portion thereof) and a CH2 domain (or portion thereof). In certain embodiments, an Fc chain lacks at least a portion of a CH2 domain (e.g., all or part of a CH2 domain). An Fc chain herein generally refers to a polypeptide comprising all or part of the Fc chain of an immunoglobulin heavy-chain. This includes, but is not limited to, polypeptides comprising the entire CH1, hinge, CH2, and/or CH3 domains as well as fragments of such peptides comprising only, e.g., the hinge, CH2, and CH3 domain. The Fc chain may be derived from an immunoglobulin of any species and/or any subtype, including, but not limited to, a human IgG1, IgG2, IgG3, IgG4, IgD, IgA, IgE, or IgM antibody. The Fc domain encompasses native Fc and Fc variant molecules. As with Fc variants and native Fc's, the term Fc chain includes molecules in monomeric or multimeric form, whether digested from whole antibody or produced by other means. In some embodiment, the Fc chain comprises the carboxy-terminal portions of both heavy chains held together by disulfides. In certain embodiments, an Fc chain consists of a CH2 domain and a CH3 domain.

In some embodiments, an Fc polypeptide comprises part or all of a wild-type hinge sequence (generally at its N-terminal). In some embodiments, an Fc polypeptide does not comprise a functional or wild-type hinge sequence.

As used herein, the term “CH1 domain” refers to the first constant domain of an antibody heavy chain (e.g., amino acid positions 118-215 of human IgG1, according to the EU index). The term includes naturally occurring CH1 domains and engineered variants of naturally occurring CH1 domains (e.g., CH1 domains comprising one or more amino acid insertions, deletions, substitutions, or modifications relative to a naturally occurring CH1 domain).

As used herein, the term “CH2 domain” refers to the second constant domain of an antibody heavy chain (e.g., amino acid positions 231-340 of human IgG1, according to the EU index). The term includes naturally occurring CH2 domains and engineered variants of naturally occurring CH2 domains (e.g., CH2 domains comprising one or more amino acid insertions, deletions, substitutions, or modifications relative to a naturally occurring CH2 domain).

As used herein, the term “CH3 domain” refers to the third constant domain of an antibody heavy chain (e.g., amino acid positions 341-447 of human IgG1, according to the EU index). The term includes naturally occurring CH3 domains and engineered variants of naturally occurring CH3 domains (e.g., CH3 domains comprising one or more amino acid insertions, deletions, substitutions, or modifications relative to a naturally occurring CH3 domain).

As used herein, the term “EU index” refers to the EU numbering convention for the Fc domains of an antibody, as described in Edelman, G M. et al., Proc. Natl. Acad. USA, 63, 78-85 (1969) and Kabat et al., Sequences of Proteins of Immunological Interest, U.S. Dept. Health and Human Services, 5th edition, 1991, each of which is herein incorporated by reference in its entirety. All numbering of amino acid positions of the Fc polypeptides, or fragments thereof, used herein is according to the EU index.

As used herein, the term “specifically binds,” “specifically binding,” “binding specificity” or “specifically recognized” refers that an antigen binding protein or antigen-binding fragment thereof that exhibits appreciable affinity for an antigen (e.g., a TSHR antigen, e.g., thyroid stimulating hormone (TSH)) and does not exhibit significant cross reactivity to a different target protein. As used herein, the term “affinity” refers to the strength of the interaction between an antigen binding protein or antigen-binding fragment thereof antigen binding site and the epitope to which it binds. Methods to determine such specific binding are also well known in the art. In certain embodiments, the antigen binding protein or antigen binding fragment thereof can bind to a human thyroid stimulating hormone receptor (TSHR), but not to TSHR from other species. Alternatively, in some embodiments, the antigen binding proteins or antigen binding fragments bind to human TSHR and to TSHR from one or more non-human species. In certain exemplary embodiments, affinity is measured by surface plasmon resonance (SPR), e.g., in a Biacore instrument. As readily understood by those skilled in the art, an antigen binding protein affinity may be reported as a dissociation constant (KD) in molarity (M). The antigen binding protein or antigen-binding fragment thereof of the disclosure have KD values in the range of about 10-5 M to about 10-12 M (i.e., low micromolar to picomolar range), about 10-7 M to 10-11 M, about 10-8 M to about 10-10 M, about 10-9 M. In certain embodiments, the antigen binding protein or antigen-binding fragment thereof has a binding affinity of about 10-5 M, 10-6 M, 10-7 M, 10-8 M, 10-9 M, 10-10 M, 10-11 M, or 10-12 M. In certain embodiments, the antigen binding protein or antigen-binding fragment thereof has a binding affinity of about 10-7 M to about 10-9 M (nanomolar range).

Specific binding can be determined according to any art-recognized means for determining such binding. In some embodiments, specific binding is determined by competitive binding assays (e.g., ELISA) or Biacore assays. In certain embodiments, the assay is conducted at about 20° C., 25° C., 30° C., or 37° C. In certain embodiments, the assay is conducted at physiological pH, at an acidic pH (e.g., a pH more acidic than physiological pH), or at a basic pH (e.g., a pH more basic than physiological pH).

As used herein, “administer” or “administration” refers to the act of injecting or otherwise physically delivering a substance as it exists outside the body (e.g., an isolated binding polypeptide provided herein) into a patient, such as by, but not limited to, subcutaneous, pulmonary (e.g., inhalation), mucosal (e.g., intranasal), intradermal, intravenous, intramuscular delivery and/or any other method of physical delivery described herein or known in the art. When a disease, or a symptom thereof, is being managed or treated, administration of the substance typically occurs after the onset of the disease or symptoms thereof. When a disease, or symptom thereof, is being prevented, administration of the substance typically occurs before the onset of the disease or symptoms thereof and may be continued chronically to defer or reduce the appearance or magnitude of disease-associated symptoms.

As used herein, the term “composition” is intended to encompass a product containing the specified ingredients (e.g., an isolated binding polypeptide provided herein) in, optionally, the specified amounts, as well as any product which results, directly or indirectly, from combination of the specified ingredients in, optionally, the specified amounts.

“Effective amount” means the amount of active pharmaceutical agent (e.g., an isolated binding polypeptide of the present disclosure) sufficient to effectuate a desired physiological outcome in an individual in need of the agent. The effective amount may vary among individuals depending on the health and physical condition of the individual to be treated, the taxonomic group of the individuals to be treated, the formulation of the composition, assessment of the individual's medical condition, and other relevant factors.

As used herein, the terms “subject” and “patient” are used interchangeably. As used herein, a subject can be a mammal, such as a non-primate (e.g., cows, pigs, horses, cats, dogs, rats, mice, etc.) or a primate (e.g., monkey and human). In certain embodiments, the term “subject,” as used herein, refers to a vertebrate, such as a mammal. Mammals include, without limitation, humans, non-human primates, wild animals, feral animals, farm animals, sport animals, and pets.

As used herein, the term “therapy” refers to any protocol, method and/or agent that can be used in the prevention, management, treatment and/or amelioration of a disease or a symptom related thereto. In some embodiments, the term “therapy” refers to any protocol, method and/or agent that can be used in the modulation of an immune response to an infection in a subject or a symptom related thereto. In some embodiments, the terms “therapies” and “therapy” refer to a biological therapy, supportive therapy, and/or other therapies useful in the prevention, management, treatment and/or amelioration of a disease or a symptom related thereto, known to one of skill in the art such as medical personnel. In other embodiments, the terms “therapies” and “therapy” refer to a biological therapy, supportive therapy, and/or other therapies useful in the modulation of an immune response to an infection in a subject or a symptom related thereto known to one of skill in the art such as medical personnel.

As used herein, the terms “treat,” “treatment” and “treating” refer to the reduction or amelioration of the progression, severity, and/or duration of a disease or a symptom related thereto, resulting from the administration of one or more therapies (including, but not limited to, the administration of one or more prophylactic or therapeutic agents, such as an isolated binding polypeptide provided herein). The term “treating,” as used herein, can also refer to altering the disease course of the subject being treated. Therapeutic effects of treatment include, without limitation, preventing occurrence or recurrence of disease, alleviation of symptom(s), diminishment of direct or indirect pathological consequences of the disease, decreasing the rate of disease progression, amelioration or palliation of the disease state, and remission or improved prognosis.

The term “about” or “approximately” means within about 20%, such as within about 10%, within about 5%, or within about 1% or less of a given value or range.

### TSHR-Related Diseases

As used herein, the terms “thyroid stimulating hormone receptor” or “TSHR” refer to full length human TSHR protein or the proteolytically cleaved TSHR protein made up of two subunits. The full-length TSHR protein comprises an amino acid sequence of 764 amino acids in length. Although it may exist as a single polypeptide chain under some situations on thyroid cells or in extra-thyroid cells, most of the TSHR on thyroid cells is cleaved and divided into two subunits, A and B. A is considered an extracellular subunit and B is a large intracellular portion. The two subunits can be crosslinked by disulfide bonds. To achieve full functionality, TSHR also undergoes N-linked glycosylation, palmitoylation and other types of post-translational modifications. TSHR is a member of the G-protein coupled receptor (GPCR) family and consists of three domains: the extracellular leucine-rich repeat domain (LRD), the hinge region and a transmembrane domain (TMD) with an intracellular C-terminus (Nunez Miguel, et al. (2004) Thyroid 14:991-1011).

In the thyroid, the TSHR is present on the basal membrane of thyroid follicular epithelial cells. Binding of TSH to the TSHR starts activation of the TSHR signaling cascade which involves binding of G-proteins to the TSHR followed by stimulation of the cyclic AMP pathway and synthesis of thyroid hormones, including thyroxine (T4) and triiodothyronine (T3) (Sanders J et al (1997) Balliere's Clinical Endocrinology and Metabolism. Ed T F Davies 11:451-479; pub Balliere Tindall, London and Latif R et al (2009) Endocrinology and Metabolism Clinics of North America 38:319-341). In certain embodiments, TSHR is a cell surface receptor that is anchored to the cell membrane. In certain embodiments, a subunit of TSHR may be shed from the cell surface as a soluble protein. In certain embodiments, the subunit of TSHR that is shed from the cell surface is the A subunit.

In some instances, GPCRs are known to be in close physical or functional proximity to other types of cell surface receptors, leading to signaling or “crosstalk” between the GPCRs and other cell surface receptors, for example ion channels (Davies et al. (2023) Prog Mol Biol Transl Sci 195:101-120), tyrosine kinase receptors (Krieger et al. (2020) Pharmacol Ther 209:107502) and integrins (Teoh et al. (2012) Journal of Allergy, vol. 2012, Article ID: 341282). IGF-1R, a tyrosine kinase receptor, and TSHR exhibit crosstalk in periorbital fibroblasts, which can lead to amplification of signaling pathways associated with thyroid eye disease. Teprotumumab, a fully human monoclonal antibody to IGF-1R, attenuates signaling initiated at either TSHR or IGF-1R, thereby blocking pathologic immune responses in active thyroid eye disease (Douglas et al. (2020) N Engl J Med 382:341-52). Thus, functional antagonism of TSHR signaling may have a broader therapeutic potential by impacting functional signaling of other receptors that may be involved in TSHR mediated diseases. In certain embodiments, the antigen binding proteins or the antigen binding fragment thereof as disclosed herein may block or inhibit signaling between TSHR and other cell surface receptors (e.g., IGF-1R). In certain embodiments, the antigen binding proteins or the antigen binding fragment thereof as disclosed herein may enhance or stimulate signaling between TSHR and other cell surface receptors (e.g., IGF-1R).

In some embodiments, the thyroid associated disease, disorder or condition is a thyroid cancer metastases, goiter, multinodular goiter, congenital hypothyroidism, hyperthyroidism, Graves' disease, ophthalmic Graves' disease, neonatal hyperthyroidism, hypothyroidism, thyroid disease, autoimmune thyroid disease, Hashimoto's thyroiditis, Painless thyroiditis (PT), Postpartum thyroiditis (PPT), and Subacute thyroiditis (SAT), Graves' ophthalmopathy and pre-tibial myxoedema, Graves' eye disease, thyroid eye disease (TED) (e.g. Acute mild TED, Acute moderate to severe TED, Acute severe sight threatening TED, Recurrent TED, Chronic TED, TED secondary to treatment with radioactive iodine), Graves' disease (Acute moderate to severe graves' disease, management, Chronic Grave's disease management, Thyrotoxicosis), Graves' disease associated disorders (e.g., thyroid acropathy, Graves dermopathy), thyroid cancer (e.g., differentiated thyroid carcinoma (DTC), recurrent radioactive iodine resistant DTC, metastatic DTC), or follicular thyroid carcinoma. In some embodiments, the follicular thyroid carcinoma is iodine resistant or exhibits reduced iodine uptake. In some embodiments, the thyroid associated disease, disorder or condition is an acute sight threatening thyroid eye disease (Dysthyroid optic neuropathy or DON), acute moderate to severe thyroid eye disease, chronic thyroid eye disease, acute severe thyrotoxicosis, Graves' disease or acute severe Graves' disease.

### Autoimmune Thyroid Diseases

Autoimmune thyroid diseases (AITD) are one of the most prevalent autoimmune conditions. The major thyroid autoantigens targeted by the autoimmune system are thyroid peroxidase (TPO), thyroglobulin (Tg) and TSHR. TPO autoantibodies and thyroglobulin autoantibodies are serological markers of thyroid autoimmunity in different forms of autoimmune thyroid diseases including Hashimoto's thyroiditis, Graves' disease and postpartum thyroiditis (PPT) (Rees Smith B ef al (2007) Thyroid 17:923-938). TSHR autoantibodies are markers of TSHR autoimmunity, and in particular, Graves' disease. Furthermore, these TSHR autoantibodies are responsible for potentially initiating and driving the pathology of Graves' disease. Types of TSHR autoantibodies may include those commonly referred to as stimulating autoantibodies, blocking autoantibodies and neutral autoantibodies.

Thyroid stimulating autoantibodies bind to the TSHR and mimic the actions of TSH, thereby stimulating the thyroid to produce high levels of T3 and T4 and are thereby considered agonistic antibodies. The feedback control mechanism of thyroid function is no longer effective in the presence of thyroid stimulating autoantibodies and the patients present with clinical symptoms of a hyperactive thyroid characterized by an excess of thyroid hormones in serum and their metabolic consequences. This condition is known as Graves' disease or in some geographies, as Basedow's disease. These TSHR-activating autoantibodies may also interact with TSHR found in the retro-orbital tissue and contribute to the development of eye-related symptoms of Graves' disease, known as Graves' ophthalmopathy, Graves orbitopathy or thyroid eye disease (TED). Other signs and symptoms of Graves' disease include hyperthyroidism, goiter, and pretibial myxedema. Symptoms of hyperthyroidism are mainly insomnia, hand tremors, hyperactivity, hair loss, excessive sweating, oligomenorrhea, itching, heat intolerance, weight loss, diarrhea, frequent defecation, palpitations, periodic partial muscle weakness or paralysis, and skin warmth and moistness.

### Cancer

In some embodiments, the antigen binding protein or the antigen binding fragment thereof that specifically binds to TSHR can be used to treat a cancer in a subject. In some embodiments, the cancer is a thyroid cancer. In some embodiments, the cancer is a thyroid cancer that has disseminated from its primary site in the thyroid to other tissues and organs. In some embodiments, the cancer is an extra-thyroid cancer (e.g., a cancer not associated with the thyroid).

Expression of TSHR has been recognized in benign or malignant thyroid cells (thyrocytes), serving as the receptor for TSH. Activation of the signaling cascade through TSHR has been shown to serve as oncogenic pathways in thyroid cancer. Since the seminal publication by Ichikawa et al ((1976) Journal of Clinical Endocrinology and Metabolism 42:395-398), several independent studies have demonstrated significant continued expression of TSHR in the majority of differentiated thyroid carcinomas (Rowe et al. (2017) Endocr Relat Cancer 24(6):R191-R202). The targeting of TSHR is particularly useful in the context of recurrent or metastatic differentiated thyroid carcinoma. In this setting, antagonistic blockade of TSHR would prevent binding of TSH to TSHR. Suppression of TSH has been demonstrated to aid in reduction of thyroid tumor size and prevention of thyroid cancer recurrence (Mazzaferri et al. (1994) American Journal of Medicine 97:418-428). It is also thought that this would sensitize thyroid cancers to radioiodine exposure, which would be of use in the setting of recurrent differentiated thyroid carcinoma where cancers often develop adaptive resistance to radioiodine therapy. Furthermore, thyroid autoimmunity is associated with increased risk of thyroid cancer, in particular differentiated thyroid cancers.

Thus, clinically useful strategies have been proposed to treat well-differentiated thyroid cancer, which harbors a higher density of TSHR. Targeted TSHR therapies would be of great value in reducing the size of thyroid tumors prior to surgical intervention which requires margins that may include important tissues such as trachea, larynx, nerves, lymphatic vessels, blood vessels and bone. In addition, targeted therapies would be of great value in lengthening survival time or improving overall survival in the setting of unresectable or disseminated thyroid cancers. In settings where thyroid cancer patients have underlying autoimmune thyroid disease, targeted therapeutics may have a role in antagonizing the proliferative effect of autoantibodies or reducing lymphocytic thyroiditis that is associated with thyroid autoantibody positivity (Viola et al. (2023) Endocr Relat Cancer 30(7):e230042). Finally, targeted therapeutics conjugated with radiolabeled isotopes could improve diagnosis and detection of TSHR expressing thyroid tumors. Expression of TSHR in extra-thyroid cancer cells is also documented. For instance, expression of TSHR has been reported in human ovarian tissue (Aghajanova et al. (2009)). Functionality of TSHR in primary human ovarian tissues under TSH or thyroid hormone stimulation was also demonstrated through assessing downstream signaling pathways or metabolites. TSHR activation has been shown to activate canonical G-protein coupled signaling pathways and transregulated activation of epithelial growth factor receptor (EGFR) to promote ovarian cancer cell proliferation (Huang, et al. (2016) Science Reports 6:27471). TSHR has also been shown to be expressed in liver cancer, glioma (low grade and Grade IV glioblastoma), and breast cancer.

### TSHR-Targeting Binding Proteins

In an aspect of the present disclosure, provided herein are antigen binding proteins or antigen binding fragments thereof that bind specifically to thyroid stimulating hormone receptor (TSHR). In some embodiments, the TSHR binding proteins block the binding of TSHR autoantibodies to TSHR.

One component of an antigen binding protein or an antigen binding fragment thereof of the present disclosure is one or more antigen binding domains or binding specificity which binds one or more cell surface targets (e.g., membrane bound TSHR) or one or more soluble targets (e.g., soluble TSHR).

Any type of binding moiety that specifically binds to a specific receptor subunit can be employed in the antigen binding protein or the antigen binding fragment thereof disclosed herein. In certain embodiments, the binding moiety comprises an antibody variable domain. Exemplary binding moieties comprising an antibody variable domain include, without limitation, a VH, a VL, a VHH, a VH/VL pair, an scFv, a diabody, or a Fab. Other suitable binding moiety formats include, without limitation, lipocalins (see e.g., Gebauer M. et al., 2012, Method Enzymol. 503:157-188, which is incorporated by reference herein in its entirety), adnectins (see e.g., Lipovsek D., 2011, Protein Eng. Des. Sel. 24:3-9, which is incorporated by reference herein in its entirety), avimers (see e.g., Silverman J, et al., 2005, Nat. Biotechnol. 23:1556-1561, which is incorporated by reference herein in its entirety), fynomers (see e.g., Schlatter D, et al., 2012, mAbs 4:497-508, which is incorporated by reference herein in its entirety), kunitz domains (see e.g., Hosse R. J. et al., 2006, Protein Sci. 15:14-27, which is incorporated by reference herein in its entirety), knottins (see e.g., Kintzing J. R. et al., 2016, Curr. Opin. Chem. Biol. 34:143-150, which is incorporated by reference herein in its entirety), affibodies (see e.g., Feldwisch J. et al., 2010 J. Mol. Biol. 398:232-247, which is incorporated by reference herein in its entirety), and DARPins (see e.g., Pluckthun A., 2015, Annu. Rev. Pharmacol. Toxicol. 55:489-511, which is incorporated by reference herein in its entirety).

In certain embodiments, the binding domain comprises the heavy and/or light chain variable regions of a conventional antibody or antigen binding fragment thereof (e.g., a Fab or scFv), wherein the term “conventional antibody” is used herein to describe heterotetrameric antibodies containing heavy and light immunoglobulin chains arranged according to the “Y” configuration. Such conventional antibodies may derive from any suitable species including but not limited to antibodies of llama, alpaca, camel, mouse, rat, rabbit, goat, hamster, chicken, monkey, or human origin. In certain exemplary embodiments, the conventional antibody comprises a heavy chain variable domain (VH) and a light chain variable domain (VL) wherein the VH and/or VL domains or one or more complementarity determining regions (CDRs) thereof are derived from the same antibodies. In certain embodiments, the conventional antibody antigen binding region may be referred to as a “Fab” (Fragment antigen-binding). The Fab comprises one constant and one variable domain from each of heavy chain and light chain. The variable heavy and light chains contain the CDRs responsible for antigen binding.

In certain embodiments, the antigen binding protein or the antigen binding fragment thereof of the present disclosure comprises a VH domain that comprises an amino acid sequence that is at least about 80%, 85%, 90%, 91%, 92%, 93%, 94%, 95%, 96%, 97%, 98%, 99%, or 100% identical to an amino acid sequence set forth in SEQ ID NOs: 1, 3, 4, 7, 8, 9, 10, 11, 12, 13, 14, 15, 16, 17, 18, 19, 20, 21, 22, 23, 24, 78, 79, 80, 81, 82, 83, 84, 85, 86, 87, or 88.

In certain embodiments, the antigen binding protein or the antigen binding fragment thereof of the present disclosure comprises a VL domain that comprises an amino acid sequence that is at least about 80%, 85%, 90%, 91%, 92%, 93%, 94%, 95%, 96%, 97%, 98%, 99%, or 100% identical to an amino acid sequence set forth in SEQ ID NOs: 2, 4, 25, 26, 27, 28, 29, 30, 31, 32, 33, 34, 35, 36, 37, 89, 90, 91, 92, 93, 94, 95, 96, or 97.

In other embodiments, the specific receptor subunit binding subunit comprises at least a CDR or VHH domain of a VHH antibody or Nanobody. VHH antibodies, which are camelid-derived heavy chain antibodies, are composed of two heavy chains and are devoid of light chains (Hamers-Casterman, et al. Nature. 1993; 363; 446-8). Each heavy chain of the VHH antibody has a variable domain at the N-terminus, and these variable domains are referred to in the art as “VHH” domains in order to distinguish them from the variable domains of the heavy chains of the conventional antibodies i.e., the VH domains. Similar to conventional antibodies, the VHH domains of the molecule comprise HCDR1, HCDR2 and HCDR3 regions which confer antigen binding specificity and therefore VHH antibodies or fragments such as isolated VHH domains, are suitable as components of the multispecific binding proteins of the present disclosure.

In some embodiments, the TSHR-binding protein is a multispecific antigen binding protein. The term “multispecific antigen binding protein” as used herein refers to bispecific, tri-specific or multi-specific antigen-binding molecules, and antigen-binding fragments thereof. Multispecific antigen-binding molecules may be specific for different epitopes of one target polypeptide or may contain antigen-binding domains specific for epitopes of more than one target polypeptide. In certain embodiment, the multispecific antigen binding molecules of the disclosure comprises at least a first binding specificity for a subunit of a receptor and at least a second binding specificity for a subunit. A multispecific antigen-binding molecule can be a single multifunctional polypeptide, or it can be a multimeric complex of two or more polypeptides that are covalently or non-covalently associated with one another. The term “multispecific antigen-binding molecules” includes antibodies of the present disclosure that may be linked to or co-expressed with another functional molecule, e.g., another antibody, antibody fragment, peptide or protein. For example, an antibody or fragment thereof can be functionally linked (e.g., by chemical coupling, genetic fusion, non-covalent association or otherwise) to one or more other molecular entities, such as a protein or fragment thereof to produce a bi-specific or a multi-specific antigen-binding molecule with a second binding specificity. According to the present disclosure, the term “multispecific antigen-binding molecules” also includes bispecific, trispecific or multispecific antibodies or antigen-binding fragments thereof. In certain exemplary embodiments, an antibody of the present disclosure is functionally linked to another antibody or antigen-binding fragment thereof to produce a bispecific antibody with a second binding specificity.

Methods for making multispecific binding proteins are well known. For example, traditionally, the recombinant production of bispecific antibodies was based on the co-expression of two immunoglobulin heavy chain/light chain pairs, where the two heavy chains have different specificities (Milstein et al., Nature 305:537 (1983)). Because of the random assortment of immunoglobulin heavy and light chains, the hybridomas (quadromas) produce a potential mixture of ten different antibody molecules, of which only one has the correct bispecific structure. The purification of the correct molecule is usually accomplished by affinity chromatography steps. More modern techniques for generating bispecific antibodies employ heterodimerization domains that favor desired pairing of heavy chain from the antibody with a first specificity to the heavy chain of an antibody with a second specificity.

Antibody variable domains with the desired binding specificities can be fused to immunoglobulin constant domain sequences. The fusion typically is with an immunoglobulin heavy chain constant domain, comprising at least part of the hinge, CH2, and CH3 regions. It may have the first heavy chain Fc domain (CH1) containing the site necessary for light chain binding present in at least one of the fusions. DNAs encoding the immunoglobulin heavy chain fusions and, if desired, the immunoglobulin light chain, are inserted into separate expression vectors, and are co-transformed into a suitable host organism. For further details of generating bispecific antibodies see, for example Suresh et al., Meth. Enzymol. 121:210 (1986).

### Effector Function Mutations

As discussed above, the antigen binding proteins of the disclosure can be provided in various isotypes and with different Fc domains. The Fc region of the antigen binding protein primarily determines its effector function in terms of Fc binding, antibody-dependent cell-mediated cytotoxicity (ADCC) activity, complement dependent cytotoxicity (CDC) activity, and antibody-dependent cell phagocytosis (ADCP) activity. These “cellular effector functions”, as distinct from effector T cell function, involve the recruitment of cells bearing Fc receptors to the site of the target cells, resulting in killing of the antibody-bound cell.

An antigen binding protein or an antigen binding fragment thereof according to the present invention may be one that exhibits reduced effector function. In certain embodiments, the one or more mutations reduces one or more of antibody dependent cellular cytotoxicity (ADCC), antibody dependent cellular phagocytosis (ADCP), or complement dependent cytotoxicity (CDC). In certain embodiments, an antibody according to the present invention may lack ADCC, ADCP, and/or CDC activity. In either case, an antibody according to the present invention may comprise, or may optionally lack, an Fc region that binds to one or more types of Fc receptor. Use of different antibody formats, and the presence or absence of FcR binding and cellular effector functions, allow the antibody to be tailored for use in particular therapeutic purposes as discussed elsewhere herein.

In certain embodiments, the first and the second Fc domain comprises one or more mutations that reduces Fc effector function. In certain embodiments, the first Fc domain and the second Fc domain each comprise a L234A and L235A mutation. These IgG1 mutations are also known as the “LALA” mutations and are described in further detail in Xu et al. (Cell Immunol. 2000; 200:16-26). In certain embodiments, the first Fc domain and the second Fc domain each comprise a L234A, L235A, G237A, and/or P329G mutations. The Fc domain amino acid positions referred to herein are based on EU antibody numbering. Alternatively, an antibody may have a Fc domain which is effector null. An antibody may have a heavy chain Fc domain that does not bind Fcγ receptors, for example the Fc domain may comprise a L235E mutation. Another optional mutation for a heavy chain Fc domain is S228P, which increases stability. A heavy chain Fc domain may be an IgG4 comprising both the L235E mutation and the S228P mutation. This “IgG4-PE” heavy chain Fc domain is effector null. A disabled IgG1 heavy chain Fc domain may contain alanine at position 234, 235, and/or 237 (EU index numbering), e.g., it may be an IgG1 sequence comprising the L234A, L235A, and/or G237A mutations (“LALAGA”).

Human IgG1 Fc domains containing specific mutations or altered glycosylation on residue Asn297 (e.g., N297Q, N297D, and N297K, according to EU index numbering) have been shown to reduce binding to Fc receptors.

In other embodiments, it may be desirable to enhance the binding of the Fc region of an antigen binding protein to human Fc gamma receptor IIIA (FcγRIIIA) relative to that of the Fc region of a corresponding naturally occurring antibody. In certain embodiments, a Fc domain may be engineered for enhanced ADCC and/or CDC and/or ADCP. The potency of Fc-mediated effects may be enhanced by engineering the Fc domain by various established techniques. Such methods increase the affinity for certain Fc-receptors, thus creating potential diverse profiles of activation enhancement. This can be achieved by modification of one or several amino acid residues. Example mutations are one or more of the residues selected from 239, 332 and 330 for human IgG1 Fc domains (or the equivalent positions in other IgG isotypes). An antibody may thus comprise a human IgG1 Fc domain having one or more mutations independently selected from S239D, 1332E and A330L (EU index numbering).

Increased affinity for Fc receptors can also be achieved by altering the natural glycosylation profile of the Fc domain by, for example, generating under fucosylated or de-fucosylated variants. Non-fucosylated antibodies harbor a tri-mannosyl core structure of complex-type N-glycans of Fc without fucose residue. These glycoengineered antibodies that lack core fucose residue from the Fc N-glycans may exhibit stronger ADCC than fucosylated equivalents due to enhancement of FcγRIIIA binding capacity. For example, to increase ADCC, residues in the hinge region can be altered to increase binding to FcγRIIIA. Thus, an antibody may comprise a human IgG heavy chain Fc domain that is a variant of a wild-type human IgG heavy chain Fc domain. In certain embodiments, the variant human IgG heavy chain Fc domain binds to human Fcγ receptors selected from the group consisting of FcγRIIB and FcγRIIA with higher affinity than the wild type human IgG heavy chain Fc domain binds to the human FcγRIIIA. The antibody may comprise a human IgG heavy chain Fc domain that is a variant of a wild type human IgG heavy chain Fc domain, wherein the variant human IgG heavy chain Fc domain binds to human FcγRIIB with higher affinity than the wild type human IgG heavy chain Fc domain binds to human FcγRIIB. The variant human IgG heavy chain Fc domain can be a variant human IgG1, a variant human IgG2, or a variant human IgG4 heavy chain Fc domain. In one embodiment, the variant human IgG heavy chain Fc domain comprises one or more amino acid mutations selected from G236D, P238D, S239D, S267E, L328F, and L328E (EU index numbering system). in another embodiment, the variant human IgG heavy chain Fc domain comprises a set of amino acid mutations selected from the group consisting of: S267E and L328F; P238D and L328E; P238D and one or more substitutions selected from the group consisting of E233D, G237D, H268D, P271G, and A330R; P238D, E233D, G237D, H268D, P271G, and A330R; G236D and S267E; S239D and S267E; V262E, S267E, and L328F; and V264E, S267E, and L328F (EU index numbering system).

The enhancement of CDC may be achieved by amino acid changes that increase affinity for C1q, the first component of the classic complement activation cascade. Another approach is to create a chimeric Fc domain created from human IgG1 and human IgG3 segments that exploit the higher affinity of IgG3 for C1q. Antibodies of the present invention may comprise mutated amino acids at residues 329, 331 and/or 322 to alter the C1q binding and/or reduced or abolished CDC activity. In another embodiment, the antibodies or antibody fragments disclosed herein may contain Fc regions with modifications at residues 231 and 239, whereby the amino acids are replaced to alter the ability of the antibody to fix complement. In one embodiment, the antibody or fragment has a Fc domain comprising one or more mutations selected from E345K, E430G, R344D and D356R, in particular a double mutation comprising R344D and D356R (EU index numbering system).

The functional properties of the antigen binding proteins may be further tuned by combining amino acid substitutions that alter Fc binding affinity with amino acid substitutions that affect binding to FcRn. Binding proteins with amino acid substitutions that affect binding to FcRn (also referred to herein as “FcRn variants”) may in certain situations also increase serum half-life in vivo as compared to an unmodified binding protein. As it will be appreciated, any combination of Fc and FcRn variants may be used to tune clearance of the antigen-antibody complex. Suitable FcRn variants that may be combined with any of the Fc variants described herein that include without limitation N434A, N434S, M428L, V308F, V259I, M428L/N434S, V259I/V308F, Y436I/M428L, Y436I/N434S, Y436V/N434S, Y436V/M428L, M252Y, M252Y/S254T/T256E, and V259I/V308F/M428L.

### Heterodimerization Motifs

In certain exemplary embodiments, the first and second Fc domains of the TSHR antigen binding protein as disclosed herein are further engineered to enhance heterodimerization of the first specific and second specific binding domains and minimize the effects of incorrect chain pairing.

Any art-recognized approach that addresses the problem of incorrect chain pairing can be employed to improve desired antigen binding protein production. For example, for bispecific antibodies, US2010/0254989 A1 describes the construction of bispecific cMet-ErbB1 antibodies, where the VH and VL of the individual antibodies are fused genetically via a GlySer linker. For bispecific antibodies including an Fc domain, mutations may be introduced into the Fc to promote the correct heterodimerization of the Fc portion. Several such approaches are reviewed in Klein et al. (mAbs (2012) 4:6, 1-11), the contents of which are incorporated herein by reference in their entirety.

In certain embodiments, the binding specificities of a multispecific antibody are heterodimerized through knobs-into-holes (KiH) pairing of Fc domains. This dimerization technique utilizes “protuberances” or “knobs” with “cavities” or “holes” engineered into the interface of CH3 domains. Where a suitably positioned and dimensioned knob or hole exists at the interface of either the first or second CH3 domain, it is only necessary to engineer a corresponding hole or knob, respectively, at the adjacent interface, thus promoting and strengthening Fc domain pairing in the CH3/CH3 domain interface. The IgG Fc domain that is fused to the binding region is provided with a knob, and the IgG Fc domain of the conventional antibody is provided with a hole designed to accommodate the knob, or vice-versa. A “knob” refers to an at least one amino acid side chain, typically a larger side chain, that protrudes from the interface of the CH3 portion of a first Fc domain. The protrusion creates a “knob” which is complementary to and received by a “hole” in the CH3 portion of a second Fc domain. The “hole” is an at least one amino acid side chain, typically a smaller side chain, which recedes from the interface of the CH3 portion of the second Fc domain. This technology is described, for example, in U.S. Pat. Nos. 5,821,333; 5,731,168 and 8,216,805; Ridgway et al. Protein Engineering (1996) 9:617-621); and Carter P. J. Immunol. Methods (2001) 248:7-15, which are herein incorporated by reference. Exemplary amino acid residues that may act as the knob include arginine (R), phenylalanine (F), tyrosine (Y) or tryptophan (W). An existing amino acid residue in the CH3 domain may be replaced or substituted with a knob amino acid residue. Preferred amino acids to substitute may include any amino acids with a small side chain, such as alanine (A), asparagine (N), aspartic acid (D), glycine (G), serine(S), threonine (T), or valine (V).

Exemplary amino acid residues that may act as the hole include alanine (A), serine(S), threonine (T), or valine (V). An existing amino acid residue in the CH3 domain may be replaced or substituted with a hole amino acid residue. Preferred amino acids to substitute may include any amino acids with a large side chain, such as arginine (R), phenylalanine (F), tyrosine (Y) or tryptophan (W). The CH3 domain is preferably derived from a human IgG1 antibody. Exemplary amino acid substitutions to the CH3 domain include Y349C, S354C, T366S, T366Y, T366W, F405A, F405W, Y407T, Y407A, Y407V, T394S, or combinations thereof. A preferred exemplary combination is S354C, T366Y or T366W for the knob mutation on a first CH3 domain and Y349C, T366S, L368A, Y407T or Y407V for the hole mutation on a second CH3 domain.

In certain embodiments, the two Fc domains of the antigen binding construct are heterodimerized through Fab arm exchange (FAE). A human IgG1 possessing a P228S hinge mutation may contain an F405L or K409R CH3 domain mutation. Mixing of the two antibodies with a reducing agent leads to FAE. This technology is described in U.S. Pat. No. 9,212,230 and Labrijn A. F. PNAS (2013) 110(13):5145-5150, which are incorporated herein by reference.

In other embodiments, the two Fc domains of the antigen binding construct are heterodimerized through electrostatic steering effects. This dimerization technique utilizes electrostatic steering to promote and strengthen Fc domain pairing in the CH3/CH3 domain interface. The charge complementarity between two CH3 domains is altered to favor heterodimerization (opposite charge paring) over homodimerization (same charge pairing). In this method, the electrostatic repulsive forces prevent homodimerization. Certain exemplary amino acid residue substitutions which confer electrostatic steering effects include K409D, K392D, and/or K370D in a first CH3 domain and D399K, E356K, and/or E357K in a second CH3 domain. This technology is described in US Patent Publication No. 2014/0154254 A1 and Gunasekaran K. JBC (2010) 285(25):19637-19646, which are incorporated herein by reference.

In other embodiments, the charge complementarity is formed by a first Fc domain comprising a N297K and/or a T299K mutation, and a second Fc domain comprising a N297D and/or a T299D mutation.

In certain embodiments, the two Fc domains of the antigen binding construct are heterodimerized through hydrophobic interaction effects. This dimerization technique utilizes hydrophobic interactions instead of electrostatic ones to promote and strengthen Fc domain pairing in the CH3/CH3 domain interface. Exemplary amino acid residue substitution may include K409W, K360E, Q347E, Y349S, and/or S354C in a first CH3 domain and D399V, F405T, Q347R, E357W, and/or Y349C in a second CH3 domain. Preferred pairs of amino acid residue substitutions between a first CH3 domain and a second CH3 domain include K409W:D399V, K409W:F405T, K360E:Q347R, Y349S:E357W, and S354C:Y349C. This technology is described in US Patent Publication No. 2015/0307628 A1.

In certain embodiments, heterodimerization can be mediated through the use of leucine zipper fusions. Leucine zipper domains fused to the C terminus of each CH3 domain of the antibody chains force heterodimerization. This technology is described in Wranik B. JBC (2012) 287(52):43331-43339.

In certain embodiments, heterodimerization can be mediated through the use of a Strand Exchange Engineered Domain (SEED) body. CH3 domains derived from an IgG and IgA format force heterodimerization. This technology is described in Muda M. PEDS (2011) 24(5): 447-454.

In certain embodiments, the heterodimerization motif may comprise non-native, disulfide bonds formed by engineered cysteine residues. In certain embodiments, the first set of disulfide may comprise a Y349C mutation in the first Fc domain and a S354C mutation in the second Fc domain. In other embodiment, an engineered disulfide bond may be introduced by fusion a C-terminal extension peptide with an engineered cysteine residue to the C-terminus of each of the two Fc domains. In certain embodiments, the first Fc domain may comprise the substitution of the carboxyl-terminal as “PGK” with “GEC”, and the second Fc domain may comprise the substitution of the carboxyl terminal amino acids “PGK” with “KSCDKT (SEQ ID NO: 147)”.

In certain embodiments, the antigen binding proteins may employ the CrossMab principle (as reviewed in Klein et al.), which involves domain swapping between heavy and light chains so as to promote the formation of the correct pairings. Yet another approach involves engineering the interfaces between the paired VH-VL domains or paired CH1-CL domains of the heavy and light chains to increase the affinity between the heavy chain and its cognate light chain (Lewis et al. Nature Biotechnology (2014) 32:191-198).

An alternative approach to the production of an antigen binding protein preparations having the correct antigen specificity has been the development of methods that enrich for antibodies having the correct heavy chain-light chain pairings. For example, Spiess et al. (Nature Biotechnology (2013) 31: 753-758) describe a method for the production of a MET-EGFR bispecific antibody from a co-culture of bacteria expressing two distinct half-antibodies. Methods have also been described wherein the Fc domain of at least one of the heavy chains of a bispecific antibody is mutated so as to alter its binding affinity for an affinity agent, for example Protein A. This allows correctly paired heavy chain heterodimers to be isolated based on a purification technique that exploits the differential binding of the two heavy chains to an affinity agent (see US2010/0331527, WO2013/136186).

International patent application no. PCT/EP2012/071866 (WO2013/064701) addresses the problem of incorrect chain pairing using a method for multispecific antibody isolation based on the use of anti-idiotypic binding agents, in particular anti-idiotypic antibodies. The anti-idiotype binding agents are employed in a two-step selection method in which a first agent is used to capture antibodies having a VH-VL domain pairing specific for a first antigen and a second agent is subsequently used to capture antibodies also having a second VH-VL domain pairing specific for a second antigen.

In certain other embodiments, the antigen binding protein described herein further comprises a common light chain. The term “common light chain” as used herein refers to a light chain which is capable of pairing with a first heavy chain of an antibody which binds to a first antigen in order to form a binding site specifically binding to said first antigen and which is also capable of pairing with a second heavy chain of an antibody which binds to a second antigen in order to form a binding site specifically binding to said second antigen. A common light chain is a polypeptide comprising in N-terminal to C-terminal direction an antibody light chain variable domain (VL), and an antibody light chain constant domain (CL), which is herein also abbreviated as “VL-CL”. Multispecific binding proteins with a common light chain require heterodimerization of the distinct heavy chains. In certain embodiments, the heterodimerization methods listed above may be used with a common light chain. In certain exemplary embodiments, the heterodimerization motif may comprise non-native, disulfide bonds formed by engineered cysteine residues. Adding disulfide bonds, both between the heavy and light chain of an antibody has been shown to improve stability. Additionally, disulfide bonds have also been used as a solution to improve light-chain pairing within bispecific antibodies (Geddie M. L. et al, mABs (2022) 14(1)).

Unless otherwise stated, all antibody Fc domain numbering employed herein corresponds to the EU numbering scheme, as described in Edelman et al. (Proc. Natl. Acad. Sci. 63(1): 78-85. 1969).

Additional methods of heterodimerization of heavy and/or light chains and the generation and purification of asymmetric antibodies are known in the art. See, for example, Klein C. mAbs (2012) 4(6): 653-663, and U.S. Pat. No. 9,499,634, each of which is incorporated herein by reference.

### Antibody Conjugates

In certain embodiments, the antigen binding protein or the antigen binding fragment thereof that specifically binds TSHR of the present disclosure can be further conjugated to a payload. In certain embodiments, the payload comprises a small molecule. In certain embodiments, the payload comprises a protein or a peptide. In certain embodiments, the payload comprises a polynucleotide molecule. In certain embodiments, the payload comprises a radioisotope. In certain embodiments, the payload comprises a detectable label.

In certain embodiments, the antigen binding protein or antigen binding fragment thereof of the present disclosure is linked to a payload via a linker.

In certain embodiments, the payload can be conjugated to a Fc region of the antibody (e.g., carbohydrate moieties in the Fc region of an antibody can be used to conjugate a therapeutic agent). In certain embodiments, the payload is conjugated to a variable region of the antibody. The engineered carbohydrate moiety is then used to attach a payload. In addition, those of skill in the art will recognize numerous possible variations of the conjugation methods. For example, the carbohydrate moiety can be used to attach polyethyleneglycol in order to extend the half-life of an intact antibody, or antigen-binding fragment thereof, in blood, lymph, or other extracellular fluids. Moreover, it is possible to construct a “divalent conjugate” by attaching therapeutic agents to a carbohydrate moiety and to a free sulfhydryl group. Such a free sulfhydryl group may be located in the hinge region of the antibody component. In some embodiments, the payload can be conjugated to the Fab region of an antibody disclosed herein. In some embodiments, the payload is conjugated to a variable region, for example, of a light chain and/or a heavy chain. In some embodiments, the payload is conjugated to a constant domain, of a light chain and/or a heavy chain.

In certain embodiments, provided herein are antibody-drug conjugates (ADCs). ADCs comprise an antibody conjugated, i.e., covalently attached by a linker, to a drug moiety. The ADCs of the present disclosure may selectively deliver an effective dose of an agent to a tissue whereby greater selectivity, i.e., a lower efficacious dose may be achieved. In some embodiments, the bioavailability of the ADC, or an intracellular metabolite of the ADC, is improved in a subject when compared to the corresponding drug moiety.

In certain embodiments, the drug moiety of the ADC is not cleaved from the antibody until the ADC binds to a cell-surface receptor, i.e., TSHR, or enters a cell with a cell surface receptor specific for the antibody of the ADC. Alternatively, the drug moiety may be cleaved from the antibody after the ADC enters the cell. The drug moiety may be intracellularly cleaved in a subject from the antibody of the compound, or an intracellular metabolite of the compound, by enzymatic action, hydrolysis, oxidation, or other mechanisms.

In certain embodiments, provided herein are radioconjugates, i.e., a radioisotope conjugated with an antigen binding protein or antigen binding fragment thereof of the present disclosure. The radioisotope can serve as a therapeutic agent. The radioisotope can serve as an imaging molecule. Provided herein, in one aspect is a radiotherapeutic that comprises an antibody disclosed herein conjugated with a radioisotope. As used herein, a “radioisotope” and “radionuclide” may be used interchangeably, and may be an alpha particle emitting isotope, a beta particle emitting isotope, and/or a gamma-emitting isotope. Accordingly, an antibody disclosed herein can be conjugated with a beta particle emitter, an alpha particle emitter, and/or a gamma ray emitter. Non-limiting examples of radioisotopes that may be used include the following: 131I, 125I, 123I, 90Y, 177Lu, 186Re, 188Re, 89Sr, 153Sm, 32P, 225Ac, 213Bi, 213Po, 211At, 212Bi, 213Bi, 223Ra, 227Th, 149Tb, 161Tb, 47Sc, 67Cu, 134Ce, 137Cs, 212Pb, and 103Pd. Methods for affixing a radionuclide to an antibody or antibody fragment (i.e., “labeling” the targeting agent such an antibody with a radioisotope) are well known in the art. Specific methods for labeling are described, for example, in U.S. Pat. Nos. 9,603,954, 10,420,851, International Pub. No. WO 2017/155937 and U.S. Provisional Patent Application No. 63/042,651 filed Dec. 9, 2019 and titled “Compositions and methods for preparation of site-specific radioconjugates,” each of which is incorporated by reference herein.

In some embodiments, the radioisotope is selected from the group consisting of Iodine-125, Iodine-123, Iodine-126, Iodine-131, Iodine-133, Bromine-77, Indium-111, Indium-113m, Gallium-67, Gallium-68, Ruthenium-95, Ruthenium-97, Ruthenium-103, Ruthenium-105, Mercury-197, Mercury-203, Rhenium-99m, Rhenium-105, Rhenium-101, Tellurium-121m, Tellurium-122m, Tellurium-125m, Thulium-165, Thulium-167, Thulium-168, Technetium-99m, Fluorine-18, Rhenium-186, Rhenium-188, Silver-Ill, Platinum-197, Palladium-109, Copper-67, Phosphorus-32, Phosphorus-33, Yttrium-90, Scandium-47, Samarium-153, Lutetium-177, Rhodium-105, Praseodymium-142, Praseodymium-143, Terbium-161, Holmium-166 and Gold-199.

### Chimeric Antigen Receptors and T-Cell Receptors

In certain embodiments, the binding domain of the antigen binding protein or the antigen binding fragment thereof that recognizes and binds to TSHR of the present disclosure may be incorporated into a chimeric antigen receptor (CAR) that is to be expressed by an immune cell (e.g., T-cell, NK cell). In certain embodiments, the binding domain is a scFv that is incorporated into a CAR. First generation CARs typically had the intracellular domain from the CD35 chain, which is the primary transmitter of signals from endogenous TCRs. Second-generation CARs possess additional intracellular signaling domains from various costimulatory protein receptors (e.g., CD28, 4-1BB, ICOS, etc.) to the cytoplasmic tail of the CAR in order to provide additional signals to the T-cell. Third generation CARs combine multiple signaling domains in order to further augment potency.

CARs are able to redirect immune cell specificity and reactivity toward a selected target exploiting the ligand-binding domain properties. Immune cells, e.g., T-cells, expressing CARs provide a way of treating various cancers. More recently, CAR-T cells have shown therapeutic promise as a treatment for autoimmune diseases.

In certain embodiments, the binding domain of the antigen binding protein or the antigen binding fragment thereof that recognizes and binds to TSHR of the present disclosure may be incorporated into a T-cell receptor (TCR). TCRs are able to interact with immunogenic peptides (epitopes) bound to major histocompatibility complex (MHC) molecules and presented on the surface of target cells. Specific binding of a TCR triggers a signal cascade inside the T cell leading to the proliferation and differentiation into a maturated effector T cell. Endogenous TCRs are diverse in order to be able to target a vast variety of antigens, and this diversity is obtained by genetic rearrangement of different discontinuous segments of genes which code for the different structure regions of TCRs. TCRs are composed of one α chain and one β chain or of one & chain and one γ chain. The a/B TCR chains are composed of an N-terminal highly polymorphic variable region involved in antigen recognition and an invariant constant region. Exogenous TCRs can be engineered to specifically target an antigen of interest by replacing the variable region of a TCR with an antigen binding domain of an antigen binding protein or antibody.

### Expression of Antigen Binding Proteins

In one aspect, polynucleotides encoding the binding proteins (e.g., antigen binding proteins and antigen binding fragments thereof that specifically bind TSHR) disclosed herein are provided. Methods of making binding proteins comprising expressing these polynucleotides are also provided.

Polynucleotides encoding the TSHR binding proteins disclosed herein are typically inserted in an expression vector for introduction into host cells that may be used to produce the desired quantity of the binding proteins. Accordingly, in certain aspects, the disclosure provides expressions vectors comprising polynucleotides disclosed herein and host cells comprising these vectors and polynucleotides.

The term “vector” or “expression vector” is used herein to mean vectors used in accordance with the present disclosure as a vehicle for introducing into an expressing a desired gene in a cell. As known to those skilled in the art, such vectors may readily be selected from the group consisting of plasmids, phages, viruses and retroviruses. In general, vectors compatible with the disclosure will comprise a selection marker, appropriate restriction sites to facilitate cloning of the desired gene and the ability to enter and/or replicate in eukaryotic or prokaryotic cells.

Numerous expression vector systems may be employed for the purposes of this disclosure. For example, one class of vector utilizes DNA elements which are derived from animal viruses such as bovine papilloma virus, polyoma virus, adenovirus, vaccinia virus, baculovirus, retroviruses (RSV, MMTV or MOMLV), or SV40 virus. Others involve the use of polycistronic systems with internal ribosome binding sites. Additionally, cells which have integrated the DNA into their chromosomes may be selected by introducing one or more markers which allow selection of transfected host cells. The marker may provide for prototrophy to an auxotrophic host, biocide resistance (e.g., antibiotics) or resistance to heavy metals such as copper. The selectable marker gene can either be directly linked to the DNA sequences to be expressed, or introduced into the same cell by co-transformation. Additional elements may also be needed for optimal synthesis of mRNA. These elements may include signal sequences, splice signals, as well as transcriptional promoters, enhancers, and termination signals. In some embodiments, the cloned variable region genes are inserted into an expression vector along with the heavy and light chain Fc domain genes (e.g., human Fc domain genes) synthesized as discussed above.

In other embodiments, the binding proteins may be expressed using polycistronic constructs. In such expression systems, multiple gene products of interest such as heavy and light chains of antibodies may be produced from a single polycistronic construct. These systems advantageously use an internal ribosome entry site (IRES) to provide relatively high levels of polypeptides in eukaryotic host cells. Compatible IRES sequences are disclosed in U.S. Pat. No. 6,193,980, which is incorporated by reference herein in its entirety for all purposes. Those skilled in the art will appreciate that such expression systems may be used to effectively produce the full range of polypeptides disclosed in the instant application.

More generally, once a vector or DNA sequence encoding a binding protein, e.g. an antibody or fragment thereof, has been prepared, the expression vector may be introduced into an appropriate host cell. That is, the host cells may be transformed. Introduction of the plasmid into the host cell can be accomplished by various techniques well known to those of skill in the art. These include, but are not limited to, transfection (including electrophoresis and electroporation), protoplast fusion, calcium phosphate precipitation, cell fusion with enveloped DNA, microinjection, and infection with intact virus. See, Ridgway, A. A. G. “Mammalian Expression Vectors” Chapter 24.2, pp. 470-472 Vectors, Rodriguez and Denhardt, Eds. (Butterworths, Boston, Mass. 1988). Plasmid introduction into the host can be by electroporation. The transformed cells are grown under conditions appropriate to the production of the light chains and heavy chains, and assayed for heavy and/or light chain protein synthesis. Exemplary assay techniques include enzyme-linked immunosorbent assay (ELISA), radioimmunoassay (RIA), or fluorescence-activated cell sorter analysis (FACS), immunohistochemistry and the like.

As used herein, the term “transformation” shall be used in a broad sense to refer to the introduction of DNA into a recipient host cell that changes the genotype.

Along those same lines, “host cells” refers to cells that have been transformed with vectors constructed using recombinant DNA techniques and encoding at least one heterologous gene. In descriptions of processes for isolation of polypeptides from recombinant hosts, the terms “cell” and “cell culture” are used interchangeably to denote the source of antibody unless it is clearly specified otherwise. In other words, recovery of polypeptide from the “cells” may mean either from spun down whole cells, from supernatant of lysed cells culture, or from the cell culture containing both the medium and the suspended cells.

In one embodiment, a host cell line used for antibody expression is of mammalian origin. Those skilled in the art can determine particular host cell lines which are best suited for the desired gene product to be expressed therein. Exemplary host cell lines include, but are not limited to, GS-CHO and CHO-K1 (Chinese Hamster Ovary lines), DG44 and DUXB11 (Chinese Hamster Ovary lines, DHFR minus), HELA (human cervical carcinoma), CV-1 (monkey kidney line), COS (a derivative of CV-1 with SV40 T antigen), R1610 (Chinese hamster fibroblast) BALBC/3T3 (mouse fibroblast), HEK (human kidney line), SP2/O (mouse myeloma), BFA-1c1BPT (bovine endothelial cells), RAJI (human lymphocyte), 293 (human kidney). In one embodiment, the cell line provides for altered glycosylation, e.g., afucosylation, of the antibody expressed therefrom (e.g., PER.C6® (Crucell) or FUT8-knock-out CHO cell lines (POTELLIGENT® cells) (Biowa, Princeton, N.J.)). In one embodiment, NS0 cells may be used. CHO cells are particularly useful. Host cell lines are typically available from commercial services, e.g., the American Tissue Culture Collection, or from authors of published literature.

In vitro production allows scale-up to give large amounts of the desired polypeptides. Techniques for mammalian cell cultivation under tissue culture conditions are known in the art and include homogeneous suspension culture, e.g., in an airlift reactor or in a continuous stirrer reactor, or immobilized or entrapped cell culture, e.g., in hollow fibers, microcapsules, on agarose microbeads or ceramic cartridges. If necessary and/or desired, the solutions of polypeptides can be purified by the customary chromatography methods, for example gel filtration, ion-exchange chromatography, chromatography over DEAE-cellulose and/or (immuno-) affinity chromatography.

Genes encoding the binding proteins featured in the disclosure can also be expressed in non-mammalian cells such as bacteria or yeast or plant cells. In this regard, it will be appreciated that various unicellular non-mammalian microorganisms such as bacteria can also be transformed, i.e., those capable of being grown in cultures or fermentation. Bacteria, which are susceptible to transformation, include members of the enterobacteriaceae, such as strains of Escherichia coli or Salmonella; Bacillaceae, such as Bacillus subtilis; Pneumococcus; Streptococcus, and Haemophilus influenzae. It will further be appreciated that, when expressed in bacteria, the binding proteins can become part of inclusion bodies. In some embodiments, the binding proteins are then isolated, purified and assembled into functional molecules. In some embodiments, the binding proteins of the disclosure are expressed in a bacterial host cell. In some embodiments, the bacterial host cell is transformed with an expression vector comprising a nucleic acid molecule encoding a binding protein of the disclosure.

In addition to prokaryotes, eukaryotic microbes may also be used. Saccharomyces cerevisiae, or common baker's yeast, is the most commonly used among eukaryotic microbes, although a number of other strains are commonly available. For expression in Saccharomyces, the plasmid Yrp7, for example (Stinchcomb et al., Nature, 282:39 (1979); Kingsman et al., Gene, 7:141 (1979); Tschemper et al., Gene, 10:157 (1980)), is commonly used. This plasmid already contains the TRP1 gene which provides a selection marker for a mutant strain of yeast lacking the ability to grow in tryptophan, for example ATCC No. 44076 or PEP4-1 (Jones, Genetics, 85:12 (1977)). The presence of the trpl lesion as a characteristic of the yeast host cell genome then provides an effective environment for detecting transformation by growth in the absence of tryptophan.

### Formulations/Pharmaceutical Compositions

In certain embodiments, a pharmaceutical composition comprising a pharmaceutically acceptable carrier and a therapeutically effective amount of an antigen-binding protein described herein is provided. Some embodiments include pharmaceutical compositions comprising a therapeutically effective amount of any one of the binding proteins as described herein, or a binding protein-drug conjugate, in admixture with a pharmaceutically or physiologically acceptable formulation agent selected for suitability with the mode of administration.

Acceptable formulation materials are typically non-toxic to recipients at the dosages and concentrations employed.

In some embodiments, the pharmaceutical composition can contain formulation materials for modifying, maintaining, or preserving, for example, the pH, osmolarity, viscosity, clarity, color, isotonicity, odor, sterility, stability, rate of dissolution or release, adsorption, or penetration of the composition. Suitable formulation materials include, but are not limited to, amino acids (such as glycine, glutamine, asparagine, arginine, or lysine), antimicrobials, antioxidants (such as ascorbic acid, sodium sulfite, or sodium hydrogen-sulfite), buffers (such as borate, bicarbonate, Tris-HCl, citrates, phosphates, or other organic acids), bulking agents (such as mannitol or glycine), chelating agents (such as ethylenediamine tetraacetic acid (EDTA)), complexing agents (such as caffeine, polyvinylpyrrolidone, beta-cyclodextrin, or hydroxypropyl-beta-cyclodextrin), fillers, monosaccharides, disaccharides, and other carbohydrates (such as glucose, mannose, or dextrins), proteins (such as serum albumin, gelatin, or immunoglobulins), coloring, flavoring and diluting agents, emulsifying agents, hydrophilic polymers (such as polyvinylpyrrolidone), low molecular weight polypeptides, salt-forming counterions (such as sodium), preservatives (such as benzalkonium chloride, benzoic acid, salicylic acid, thimerosal, phenethyl alcohol, methylparaben, propylparaben, chlorhexidine, sorbic acid, or hydrogen peroxide), solvents (such as glycerin, propylene glycol, or polyethylene glycol), sugar alcohols (such as mannitol or sorbitol), suspending agents, surfactants or wetting agents (such as pluronics; PEG; sorbitan esters; polysorbates such as polysorbate 20 or polysorbate 80; triton; tromethamine; lecithin; cholesterol or tyloxapal), stability enhancing agents (such as sucrose or sorbitol), tonicity enhancing agents (such as alkali metal halides, e.g., sodium or potassium chloride, or mannitol sorbitol), delivery vehicles, diluents, excipients and/or pharmaceutical adjuvants (see, e.g., REMINGTON'S PHARMACEUTICAL SCIENCES (18th Ed., A. R. Gennaro, ed., Mack Publishing Company 1990), and subsequent editions of the same, incorporated herein by reference for any purpose).

In some embodiments the optimal pharmaceutical composition will be determined by a skilled artisan depending upon, for example, the intended route of administration, delivery format, and desired dosage. Such compositions can influence the physical state, stability, rate of in vivo release, and rate of in vivo clearance of the binding protein.

In some embodiments the primary vehicle or carrier in a pharmaceutical composition can be either aqueous or non-aqueous in nature. For example, a suitable vehicle or carrier for injection (e.g., subcutaneous) can be water, physiological saline solution, or artificial cerebrospinal fluid, possibly supplemented with other materials common in compositions for parenteral administration. Neutral buffered saline or saline mixed with serum albumin are further exemplary vehicles. Other exemplary pharmaceutical compositions comprise Tris buffer of about pH 7.0-8.5, or acetate buffer of about pH 4.0-5.5, which can further include sorbitol or a suitable substitute. In some embodiments, the pharmaceutical composition for subcutaneous administration can be aqueous in nature. In some embodiments, the pharmaceutical composition for subcutaneous administration can be non-aqueous in nature.

In some embodiments, the pharmaceutical compositions of the disclosure can be selected for parenteral delivery or subcutaneous delivery. Alternatively, the compositions can be selected for inhalation or for delivery through the digestive tract, such as orally. The preparation of such pharmaceutically acceptable compositions is within the skill of the art.

In some embodiments, the formulation components are present in concentrations that are acceptable to the site of administration. For example, buffers are used to maintain the composition at physiological pH or at a slightly lower pH, typically within a pH range of from about 5.0 to about 8.0.

Additional pharmaceutical compositions of the disclosure will be evident to those skilled in the art, including formulations involving binding proteins in sustained- or controlled-delivery formulations. Techniques for formulating a variety of other sustained- or controlled-delivery means, such as liposome carriers, bio-erodible microparticles or porous beads and depot injections, are also known to those skilled in the art. Additional examples of sustained-release preparations include semipermeable polymer matrices in the form of shaped articles, e.g., films, or microcapsules. Sustained release matrices can include polyesters, hydrogels, polylactides, copolymers of L-glutamic acid and gamma ethyl-L-glutamate, poly(2-hydroxyethyl-methacrylate), ethylene vinyl acetate, or poly-D (−)-3-hydroxybutyric acid. Sustained-release compositions can also include liposomes, which can be prepared by any of several methods known in the art.

The disclosure also encompasses kits for producing a single dose administration unit. The kits can each contain both a first container having a dried antigen binding protein and a second container having an aqueous formulation. Also included within the scope of this disclosure are kits containing single and multi-chambered pre-filled syringes (e.g., liquid syringes and lyosyringes).

The effective amount of a binding protein pharmaceutical composition to be employed therapeutically will depend, for example, upon the therapeutic context and objectives. One skilled in the art will appreciate that the appropriate dosage levels for treatment will thus vary depending, in part, upon the molecule delivered, the indication for which the binding protein is being used, the route of administration, and the size (body weight, body surface, or organ size) and condition (the age and general health) of the patient. Accordingly, the clinician can titer the dosage and modify the route of administration to obtain the optimal therapeutic effect.

Dosing frequency will depend upon the pharmacokinetic parameters of the binding protein in the formulation being used. Typically, a clinician will administer the composition until a dosage is reached that achieves the desired effect. The composition can therefore be administered as a single dose, as two or more doses (which may or may not contain the same amount of the desired molecule) over time, or as a continuous infusion via an implantation device or catheter. Further refinement of the appropriate dosage is routinely made by those of ordinary skill in the art and is within the ambit of tasks routinely performed by them. Appropriate dosages can be ascertained through use of appropriate dose-response data.

The route of administration of the pharmaceutical composition is in accord with known methods, e.g., orally; through injection by subcutaneous, intravenous, intraperitoneal, intracerebral (intraparenchymal), intracerebroventricular, intramuscular, intraocular, intraarterial, intraportal, or intralesional routes; by sustained release systems; or by implantation devices. Where desired, the compositions can be administered by bolus injection or continuously by infusion, or by implantation device.

### Method of Treatment/Use

Another aspect of the disclosure is an antigen binding protein or an antigen binding fragment thereof that specifically binds to TSHR as described herein for use as a medicament.

In a particular embodiment, a method of treating a disease or disorder through antagonistic activity is provided, the method comprising administering to a subject in need thereof an effective amount of an antigen binding protein or an antigen binding fragment thereof as described herein. In certain embodiments, a method of treating a disease or disorder through the blocking activity of the antigen binding protein or the antigen binding fragment thereof that specifically binds to TSHR is provided herein. In certain embodiments, the antigen binding protein or the antigen binding fragment thereof that specifically binds to TSHR blocks agonistic TSHR autoantibodies. In certain embodiments, the antigen binding protein or the antigen binding fragment thereof that specifically binds to TSHR may block natural ligands of TSHR. In certain embodiments, the antigen binding protein may not block natural ligands of TSHR. In certain embodiments, the natural ligand of TSHR is thyroid stimulating hormone (TSH).

The antigen binding proteins can be employed in any known assay method, such as competitive binding assays, direct and indirect sandwich assays, and immunoprecipitation assays for the detection and quantitation of one or more target antigens. The binding proteins will bind the one or more target antigens with an affinity that is appropriate for the assay method being employed.

For diagnostic applications, in some embodiments, antigen binding proteins can be labeled with a detectable moiety. The detectable moiety can be any one that is capable of producing, either directly or indirectly, a detectable signal. For example, the detectable moiety can be a radioisotope, such as ³H, ¹⁴C, ³²P, ³⁵S, ¹²⁵I, ⁹⁹Tc, ¹¹¹ In, or ⁶⁷Ga; a fluorescent or chemiluminescent compound, such as fluorescein isothiocyanate, rhodamine, or luciferin; or an enzyme, such as alkaline phosphatase, β-galactosidase, or horseradish peroxidase.

The antigen binding proteins are also useful for in vivo imaging. An antigen binding protein labeled with a detectable moiety can be administered to an animal, e.g., into the bloodstream, and the presence and location of the labeled antibody in the host assayed. The binding protein can be labeled with any moiety that is detectable in an animal, whether by nuclear magnetic resonance, radiology, or other detection means known in the art.

The disclosure also relates to a kit comprising a binding protein and other reagents useful for detecting target antigen levels in biological samples. Such reagents can include a detectable label, blocking serum, positive and negative control samples, and detection reagents. In some embodiments, the kit comprises a composition comprising any binding protein, polynucleotide, vector, vector system, and/or host cell described herein. In some embodiments, the kit comprises a container and a label or package insert on or associated with the container. Suitable containers include, for example, bottles, vials, syringes, IV solution bags, etc. The containers may be formed from a variety of materials such as glass or plastic. The container holds a composition which is by itself or combined with another composition effective for treating, preventing and/or diagnosing a condition and may have a sterile access port (for example the container may be an intravenous solution bag or a vial having a stopper that can be pierced by a hypodermic injection needle). In some embodiments, the label or package insert indicates that the composition is used for preventing, diagnosing, and/or treating the condition of choice. Alternatively, or additionally, the article of manufacture or kit may further comprise a second (or third) container comprising a pharmaceutically acceptable buffer, such as bacteriostatic water for injection (BWFI), phosphate-buffered saline, Ringer's solution and dextrose solution. It may further include other materials desirable from a commercial and user standpoint, including other buffers, diluents, filters, needles, and syringes.

In some embodiments, the present disclosure relates to a method of preventing and/or treating a disease or disorder (e.g., thyroid-related diseases, cancer). In some embodiments, the method comprises administering to a patient a therapeutically effective amount of at least one of the binding proteins, or pharmaceutical compositions related thereto, described herein. In some embodiments, the patient is a human.

The contents of the articles, patents, and patent applications, and all other documents and electronically available information mentioned or cited herein, are hereby incorporated by reference in their entirety to the same extent as if each individual publication was specifically and individually indicated to be incorporated by reference. Applicants reserve the right to physically incorporate into this application any and all materials and information from any such articles, patents, patent applications, or other physical and electronic documents.

While the present disclosure has been described with reference to the specific embodiments thereof, it should be understood by those skilled in the art that various changes may be made and equivalents may be substituted without departing from the true spirit and scope of the disclosure. It will be readily apparent to those skilled in the art that other suitable modifications and adaptations of the methods described herein may be made using suitable equivalents without departing from the scope of the embodiments disclosed herein. In addition, many modifications may be made to adapt a particular situation, material, composition of matter, process, process step or steps, to the objective, spirit and scope of the present disclosure. All such modifications are intended to be within the scope of the claims appended hereto. Having now described certain embodiments in detail, the same will be more clearly understood by reference to the following examples, which are included for purposes of illustration only and are not intended to be limiting.

### EXAMPLES

The following examples are put forth so as to provide those of ordinary skill in the art with a complete disclosure and description of how to make and use methods and compositions featured in the invention and are not intended to limit the scope of what the inventors regard as their invention. Efforts have been made to ensure accuracy with respect to numbers used (e.g., amounts, temperature, etc.) but some experimental errors and deviations should be accounted for. Unless indicated otherwise, parts are parts by weight, molecular weight is average molecular weight, temperature is in degrees Centigrade, and pressure is at or near atmospheric.

### Example 1. Generation of Anti-TSHR Antibodies

Antibody variants were generated based on the sequence of a parental TSHR-binding antibody (SEQ ID NOs: 1 and 2) (Núñez et al. (2022) J Mol Endocrinol 70(1):e220120 and Sanders et al. (2011) J Mole Endocrinol 46(2): 81-99). Mutations were designed to alter surface charge or disrupt hydrophobic patches to modulate solubility, aggregation propensity, or other biophysical properties of the antibody without decreasing binding affinity for the target TSHR protein. Mutations were also designed with the aim of altering kinetic binding parameters to the target TSHR protein. After a round of testing, mutations that maintained binding affinity within approximately three-fold of the parental antibody were combined to create additional TSHR-binding antibodies.

### Example 2. Determination of the Activity and Potency of TSHR Antibodies

Select TSHR antibody variants were tested for their ability to bind TSHR in an ELISA. Briefly, soluble TSHR protein was coated onto a plate at at 2 μg/ml in phosphate buffered saline (PBS). The plate was washed with PBS and blocked with 4% skim milk in PBS.

Three concentrations of test antibody (300, 100 and 33 nM) were plated including the parental antibody and human IgG1 isotype control antibody. Following wash steps with 1×PBS-Tween 0.05%, bound human IgG was detected with peroxidase conjugated goat anti-human IgG (Fc specific)-HRP at 1:5000 dilution and the ELISA was developed with TMB reagent followed by H2SO4. The plate was read by O.D. at 450 nm measured using a microplate spectrophotometer ELISA data was compared against the parental antibody sequence from which the TSHR antibody variants are derived. ELISA values are reported below in Table 1.

TABLE 1
ELISA Values

| — | — | — | — | — | — | ELISA (OD 450) | | |
|---|---|---|---|---|---|---|---|---|
| — | VH | VL | ka (1/s*M) | kd (1/s) | KD (nM) | 300 nM | 100 nM | 33.3 nM |
| parental | parental VH | parental VL | 1.42E+05 | 3.30E−04 | 2.32 | 0.461 | 0.43 | 0.406 |
| TSHB-1 | GL_5-51 VH | parental VL | 1.61E+05 | 2.73E−04 | 1.7 | 0.641 | 0.506 | 0.442 |
| TSHB-2 | GL_5- | parental VL | 1.98E+05 | 2.73E−03 | 13.8 | 0.081 | 0.072 | 0.069 |
| | 51_VH_MUT1 | | | | | | | |
| TSHB-3 | GL_5- | parental VL | 1.77E+05 | 5.33E−04 | 3.01 | 0.467 | 0.407 | 0.351 |
| | 51_VH_MUT2 | | | | | | | |
| TSHB-4 | GL_5- | parental VL | 1.50E+05 | 3.62E−04 | 2.41 | 0.542 | 0.453 | 0.374 |
| | 51_VH_MUT3 | | | | | | | |
| TSHB-5 | GL_5- | parental VL | 1.56E+05 | 4.40E−04 | 2.82 | 0.578 | 0.498 | 0.414 |
| | 51_VH_MUT4 | | | | | | | |
| TSHB-6 | GL_5- | parental VL | 1.42E+05 | 5.32E−04 | 3.73 | 0.548 | 0.493 | 0.45 |
| | 51_VH_MUT5 | | | | | | | |
| TSHB-7 | GL_5- | parental VL | 1.16E+05 | 4.77E−04 | 4.11 | 0.555 | 0.491 | 0.443 |
| | 51_VH_MUT6 | | | | | | | |
| TSHB-8 | GL_5- | parental VL | 1.53E+05 | 3.44E−04 | 2.25 | 0.586 | 0.551 | 0.471 |
| | 51_VH_MUT7 | | | | | | | |
| TSHB-9 | GL_5- | parental VL | 1.04E+05 | 6.76E−04 | 6.53 | 0.478 | 0.452 | 0.355 |
| | 51_VH_MUT8 | | | | | | | |
| TSHB-10 | GL_5- | parental VL | 1.15E+05 | 3.38E−04 | 2.94 | 0.557 | 0.496 | 0.44 |
| | 51_VH_MUT9 | | | | | | | |
| TSHB-11 | GL_5- | parental VL | 1.21E+04 | 6.43E−04 | 53.3 | 0.289 | 0.22 | 0.159 |
| | 51_VH_MUT10 | | | | | | | |
| TSHB-12 | 1-46_VH | parental VL | 1.40E+05 | 5.08E−04 | 3.62 | 0.52 | 0.475 | 0.402 |
| TSHB-13 | parental VH | GL_1-51_VL | 1.41E+05 | 2.59E−04 | 1.84 | 0.561 | 0.495 | 0.459 |
| TSHB-14 | parental VH | GL_1- | 1.40E+05 | 3.52E−04 | 2.52 | 0.572 | 0.522 | 0.477 |
| | | 51_VL_MUT1 | | | | | | |
| TSHB-15 | parental VH | GL_1- | 1.21E+05 | 3.72E−04 | 3.08 | 0.569 | 0.517 | 0.443 |
| | | 51_VL_MUT2 | | | | | | |
| TSHB-16 | parental VH | GL_1- | 1.42E+05 | 3.85E−04 | 2.7 | 0.566 | 0.487 | 0.443 |
| | | 51_VL_MUT3 | | | | | | |
| TSHB-17 | parental VH | GL_1- | 4.94E+04 | 4.07E−04 | 8.25 | 0.475 | 0.402 | 0.357 |
| | | 51_VL_MUT4 | | | | | | |
| TSHB-18 | parental VH | GL_1- | 1.25E+05 | 3.68E−04 | 2.94 | 0.55 | 0.495 | 0.42 |
| | | 51_VL_MUT5 | | | | | | |
| TSHB-19 | parental VH | GL_1- | 1.18E+05 | 3.69E−04 | 3.12 | 0.547 | 0.47 | 0.405 |
| | | 51_VL_MUT6 | | | | | | |
| TSHB-20 | parental VH | GL_1- | 1.09E+05 | 3.01E−04 | 2.77 | 0.54 | 0.465 | 0.411 |
| | | 51_VL_MUT7 | | | | | | |
| TSHB-21 | parental VH | GL_1- | 1.15E+05 | 3.57E−04 | 3.1 | 0.552 | 0.473 | 0.395 |
| | | 51_VL_MUT8 | | | | | | |
| TSHB-22 | parental VH | 1-40_VL | 1.29E+05 | 3.23E−04 | 2.5 | 0.53 | 0.493 | 0.447 |
| TSHB-23 | GL_5-51 VH | GL_1-51_VL | 1.41E+05 | 3.44E−04 | 2.45 | 0.538 | 0.466 | 0.512 |
| TSHB-24 | GL_5-51 VH | 1-40_VL | 1.29E+05 | 3.48E−04 | 2.7 | 0.54 | 0.49 | 0.425 |
| TSHB-25 | 1-46_VH | 1-40_VL | 1.38E+05 | 6.16E−04 | 4.48 | 0.367 | 0.409 | 0.38 |
| hIgG1 | — | — | — | — | — | 0.055 | 0.057 | 0.063 |
| isotype | | | | | | | | |
| control | | | | | | | | |
| blank | — | — | — | — | — | 0.055 | 0.052 | 0.051 |

Affinity of the TSHR antibodies to their target antigen was also investigated using surface plasmon resonance (SPR), Table 1. Measurements were taken on a Biacore T200 at 25 C using a CM5 chip coated with anti-human Fc. Test antibodies were captured at 0.1 ug/mL in HBS-EP+ (10 mM HEPES pH7.4, 150 mM NaCl, 3 mM EDTA, 0.05% Tween 20) assay buffer. Soluble TSHR (22-260) protein was flowed over the chip at 0 nM, 0.41 nM, 1.24 nM, 3.07 nM, 11.11 nM, 33.33 nM, 100 nM, 300 nM, 900 nM at a flow rate of 30 μL/min with an association time of 90s and dissociation time of 200s. A 1:1 binding kinetics fit was used to calculate kinetic parameters.

Affinity for TSHR by the antibodies can be screened using a kinetic exclusion assay (KinExA). KD determinations are most accurate when the concentration of the receptor (e.g., TSHR) in the sample is near or below the K_{D}. Binding curves can also be generated for the antibodies.

Graves' disease is an autoimmune disorder that presents with the production of autoantibodies that can bind to TSHR, thereby activating the receptor and causing hyperthyroidism. The antibodies of the present disclosure may block the autoantibodies produced by the subject without activating the receptor. Therefore, the extent to which the TSHR antibodies of the present disclosure block binding of isolated TSHR autoantibodies or autoantibodies in the sera of Graves' disease patients will be assessed.

The blocking of an autoimmune TSHR antibody by the antibodies of the present disclosure can be measured using flow cytometry and/or SPR/BLI or ELISA. Furthermore, patient sera from Graves' disease patients comprising autoimmune antibodies may be used to further measure the ability of the antibodies of the present disclosure to block the autoantibodies binding to TSHR-overexpressing cells. This can be measured with methods including flow cytometry and/or ELISA.

### Example 3. Determining In Vitro Activity of TSHR Antibodies Against TSHR Signaling

The antibodies will be screened for agonist activity using a TSHR activity reporter cell line. This cell line will allow for the assessment of ligand (TSH) based activation of TSHR or activation by other binding molecules to TSHR. Intracellular cAMP levels will be measured as an indicator of the ability of the antibodies to block autoimmune antibodies or TSH from activating TSHR. cAMP levels will also be used as a measurement of agonistic activity and blocking of an agonist TSHR antibody or Graves' disease patient sera or antibodies purified from that sera-induced production of cAMP.

An assay involving orbital fibroblasts will also be used as a way to interrogate the activity of the antibodies of the present disclosure. Orbital fibroblasts isolated from retro-orbital connective tissue obtained from orbital decompression surgery or normal primary human orbital fibroblasts expressing surface TSHR will be cultured. The cells will be treated with the antibodies and then incubated with M22, a potent human monoclonal TSHR stimulating antibody, other isolated autoantibodies, or TSH. Hyaluronic acid levels will be measured as a readout of TSHR activation or inhibition. Other markers such as cAMP, hyaluronan synthase, protein kinase A, pERK, and pAKT may be measured as well.

### Example 4. Measuring Various Characteristics of TSHR Antibodies for Developability

The anti-TSHR antibodies of the present disclosure will be further characterized for traits important to the developability of the antibodies for therapeutic use. The melting temperatures, serum stability, and stability at a low pH of the antibodies will be measured.

The antibodies' solubility and propensity for aggregation will also be tested. This can be tested employing hydrophobic interaction chromatography, dynamic light scattering (DLS), size-exclusion chromatography (SEC), or self-interaction (AC-SINS). The viscosity of the antibody in solution at a range of concentrations will also be measured.

Specificity of the antibodies will be tested by measuring binding to targets other than TSHR. Binding other proteins such as insulin, DNA baculovirus particles, or a cell lysate can be an indicator of the degree of polyreactivity of antibodies, and thus will be tested for the TSHR antibodies. The binding will be measured via ELISA, flow cytometry, BLI, or other methods as a predictor for off-target binding that could also lead to poor PK. Undesired binding to specific off-target proteins can be measured, as detected via a screen of binding to fixed cells expressing a single membrane or tethered secreted protein from a human protein library using a library from Retrogenix (Charles River Laboratories) or other similar technology. This assay can be followed up with measuring the binding to the off-target protein by a number of different assays such as SPR, BLI, ELISA, and/or flow cytometry.

Antibody degradation due to temperature, freeze-thaw cycles, agitation, pH, and other common conditions will be measured. The antibody sequences will be interrogated for liabilities where amino acid substitutions may improve any of the above conditions or characteristics.

### Example 5. Testing the Immunogenicity of TSHR Antibodies

An effort to reduce the immunogenicity of the TSHR antibodies may be made. Anti-drug antibodies (ADAs) may affect the risk profile and efficacy of a biological drug. Therefore, the level of ADAs in normal human serum that may bind to the TSHR antibodies of the present disclosure may be tested using ELISA or similar methodology. Additionally, the sera of Graves' disease patients may also be tested for ADA in the same manner. Computational predictions of T cell epitopes and/or primary human T cell or peripheral blood mononuclear cell (PBMC) assays may also be used to assess the predicted immunogenicity of the TSHR antibodies.

Additional anti-drug antibody assays are e.g., detailed in WO2007101661A1 (Hoffmann La Roche), WO2018178307A1 (Ablynx), WO2021046316A2 (Adverum Biotechnologies, Charles River), and US20180088140A1 (Genzyme Corporation), each of which is incorporated herein by reference.

### Example 6. Determining the Pharmacokinetics/Pharmacodynamics (PK/PD) in an In Vivo Model

Variable region (Fv) amino acid sequences in either CDRs and/or framework regions may affect cellular uptake and recycling (PK) of the anti-TSHR antibodies due to faster non-specific clearance due to undesired electrostatic interactions between the charged Fv and negatively charged components of vascular and/or tissue cells or by modulation of FcRn binding affinity and subsequent recycling of the antibody. Assessment of potential PK differences in a relevant pre-clinical animal model is desired prior to therapeutic administration in human subjects.

Potential differences in PK parameters (e.g., AUC, half-life, Cmax, Tmax, CI, and F) may be measured in a mouse, rat, and/or non-human primate (NHP) models. In mice, a transgenic FcRn mouse (Tg32) may be used in addition. Different dose levels (e.g. high, med, and low) will be administered using various routes (IV, SC) that will generate a range of appropriate exposures that are expected to be achieved in humans. The concentration of anti-TSHR antibody in serum will be measured using ELISA on blood samples taken at various timepoints before and after dosing. An appropriate anti-human IgG ELISA and standard curve will be employed to detect and quantitate human IgG in mouse, rat, or NHP sera. Pharmacokinetic analyses (noncompartmental) will be performed using appropriate tools such as Phoenix WinNonlin software or equivalent.

Potential differences in pharmacodynamic activity (e.g. receptor occupancy, potency, efficacy) may also be measured in rat and NHP models.

Measurements to observe T3, T4, and elevated TSH serum levels may be made to detect and quantitate the ability of anti-TSHR antibodies to inhibit TSH binding and subsequent signaling through the TSHR thus inhibiting thyroid function (biochemical hypothyroidism) in these preclinical models. Different dose levels (e.g. high, med, and low) will be administered using various routes (IV, SC) that will generate a range of appropriate exposures that are expected to be achieved in humans. The concentration of thyroid hormones (T3, T4, TSH) in serum will be measured in blood samples taken at various timepoints before and after dosing. An appropriate anti-rat or anti-NHP thyroid hormone ELISA and standard curve will be employed to detect and quantitate levels in rat or NHP sera. Other immunoassays formats may also be employed depending on species and sensitivity considerations.

### Example 7. Thermostability and AC-SINS Data of Select TSHR Antibodies

The TSHR antibodies designated ETY-2 and ETY-3 were compared against the parental antibody for thermostability and affinity-capture self-interaction nanoparticle spectroscopy (AC-SINS). ETY-3 has the 1-46 VH domain (SEQ ID NO: 3) and the 1-40 VL domain (SEQ ID NO: 4). ETY-2 has the GL_5-51 VH domain (SEQ ID NO: 78) and the GL_1-51 VL domain (SEQ ID NO: 25).

The thermostability was measured with differential scanning fluorimetry (DSF) and compare to the parental antibody. The results of the second melting temperature (Tm2) are reported below in Table 2.

TABLE 2
Melting Temperatures

| Antibody | DSF Tm2 (° C.) |
|---|---|
| Parental | 72.5 |
| ETY-2 | 74.0 |
| ETY-3 | 79.5 |

As shown in Table 2, both ETY-2 and ETY-3 had higher melting temperatures compared to the parental antibody, with ETY-3 being 7° C. higher.

Separately, AC-SINS was determined. In AC-SINS, a low value represents less antibody self-association, hence assessing its colloidal stability. While the parental antibody had a value of 4, ETY-2 had a value of only 1 and ETY-2 had a value of only 2. Thus, each antibody had a reduced self-association score compared to the parental antibody.

### Example 8. Paratope Mapping of a Parental Anti-TSHR Antibody

Paratope mapping of the parental anti-TSHR antibody having a VH domain of SEQ ID NO: 1 and a VL domain of SEQ ID NO: 2 was performed. Specifically, paratope mapping was performed to identify amino acid substitutions that affected 1) affinity to TSHR, 2) thermal stability, 3) polyreactivity, and 4) pH-dependent affinity to TSHR. Deep mutational scanning (DMS) was employed.

Paratope mapping aims at identifying all the residues of the variable regions of an antibody that interact with the antigen. The identification of these residues of interest was performed on the parental VH and VL using the DMS method coupled to a functional screening by flow cytometry. DMS is a mutagenesis method which aims to perform all possible mono-substitutions on all selected residues within a given protein sequence (e.g., the VH and VL paratopes). The DMS library was obtained in the form of DNA coding for the variable parts of the antibody under study. In this library, each DNA strand has a codon mutated from the parental sequence. This DMS library was then integrated into an expression plasmid specifically designed to express Fab antibodies on the surface of yeast. Yeast cells were then transformed and induced to allow the expression of the single mutated antibodies on their surface. This new library (called display library) was screened by flow cytometry using fluorescent reporters to reveal the expression of the antibody as well as the binding of the antibody with its antigen.

In general, two different sorts are performed: the “positive” sort and the “negative” sort. Positive sorting consisted of isolating the yeast population expressing a mutated and functional antibody (with no loss of binding compared to the parental antibody). Negative sorting consisted of isolating yeast cells expressing a complete mutated antibody (light chain+heavy chain) that had lost its ability to bind the antigen. The plasmids contained in these yeast populations were extracted and sequenced by high-throughput sequencing. Analysis of the sequencing data allowed the identification of the impact of each mutation on the functionality of the antibody and the production of positive and negative DMS maps of the antibody studied.

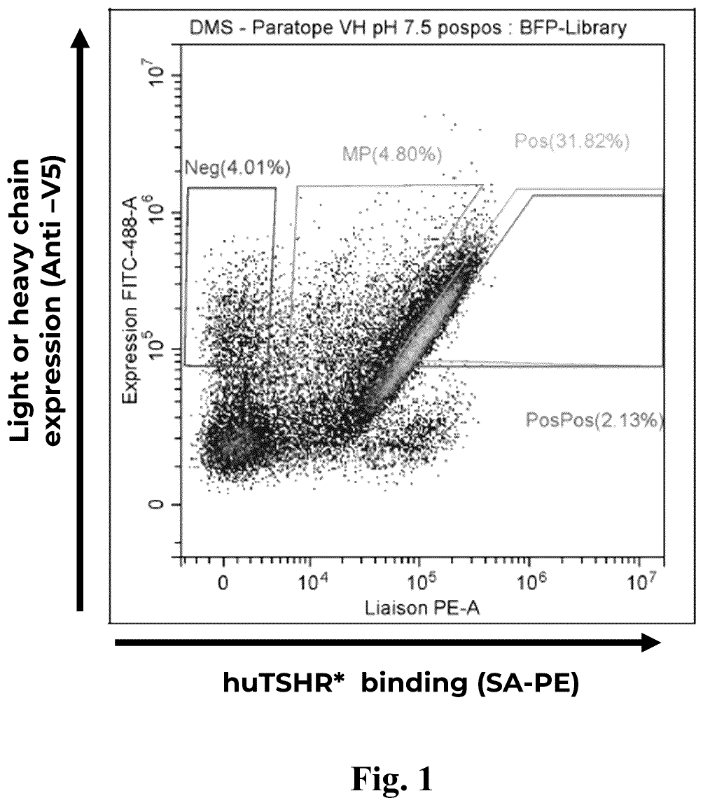

For the parental TSHR antibody, two libraries were made, one for the VH and one for the VL. The two DMS libraries were successfully generated and cloned into yeast cells. Each library contained approximately between 1400 and 1900 single mutants. For a FACS gating strategy, the following gates were used: Minus Positive (MP) gate: identifying variant antibodies with lower antigen affinity than parental, while maintaining antigen binding to the yeast surface. Negative gate: no antigen binding, but maintenance of expression of variant antibodies on the yeast surface. Double negative population gate: No antigen binding and no HC/LC pairing of the antibody at the yeast surface, which indicates de-structuring mutations or STOP codons. Positive gate: same or better antigen affinity of the variant compared to parental. PosPos gate: better affinity for antigen than parental. The gating strategy is described further in FIG. 1.

To determine the effect of single mutations, either a log 2 enrichment score was determined or a raw enrichment score was determined. Specifically, log 2 enrichment of the single mutants after sorting compared to before sorting divided by the enrichment of the parental was performed based on the following formula:

$$Enrichment = {log}_{2} [ \frac{Frequency of the mutant in the sorted population}{Frequency of the mutant in the unsorted population} / \frac{Mean frequency of the synonymous wild types in the sorted}{\text{?}}$$

$$\text{?} \text{indicates text missing or illegible when filed}$$

The raw enrichment values described herein were calculated as follows:

$$Enrichment = \frac{Frequency of the mutant in the sorted population}{Frequency of the mutant in the unsorted population}$$

### Affinity to TSHR

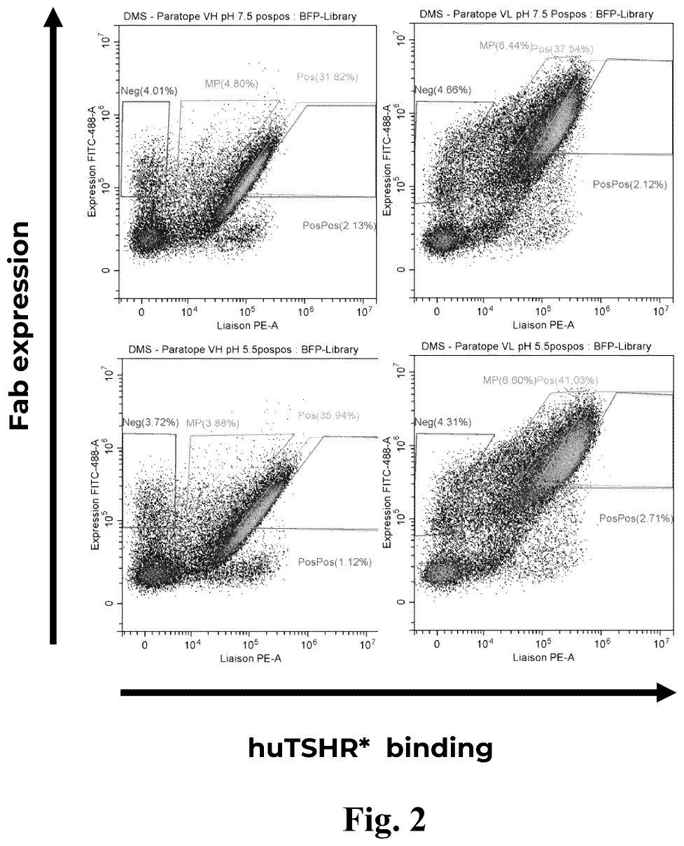

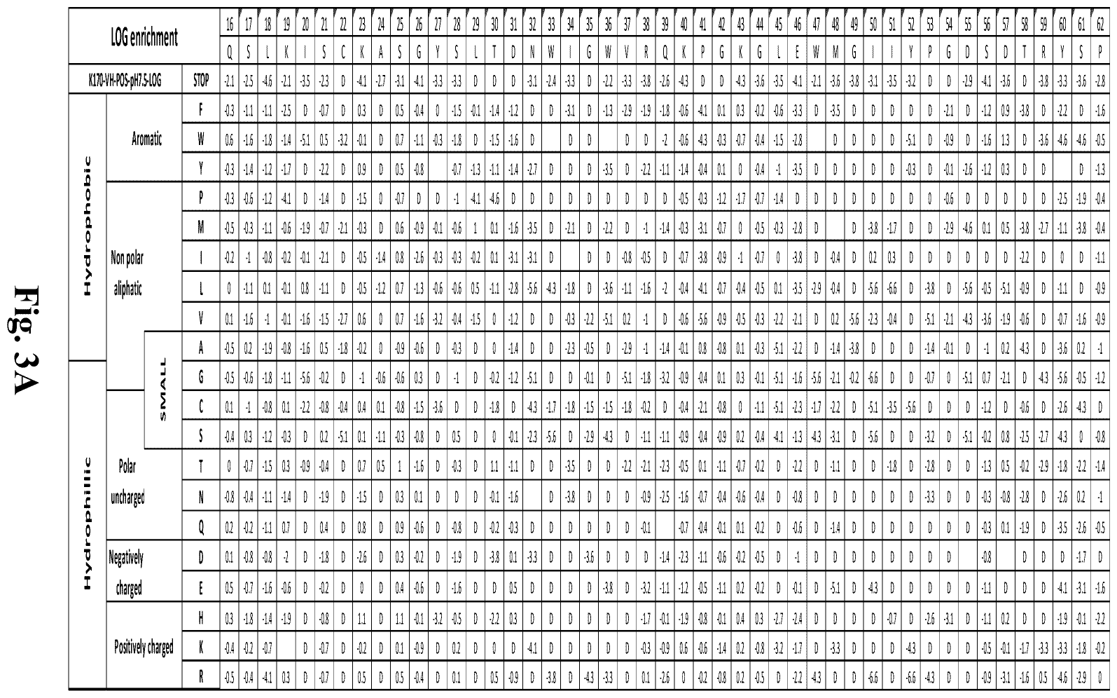

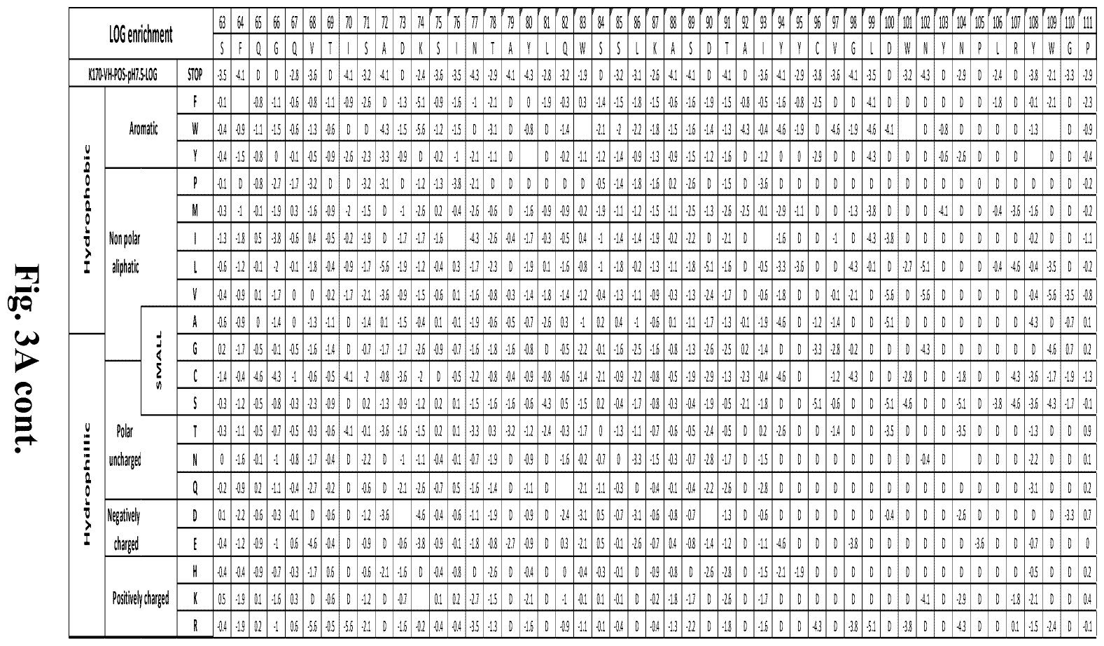

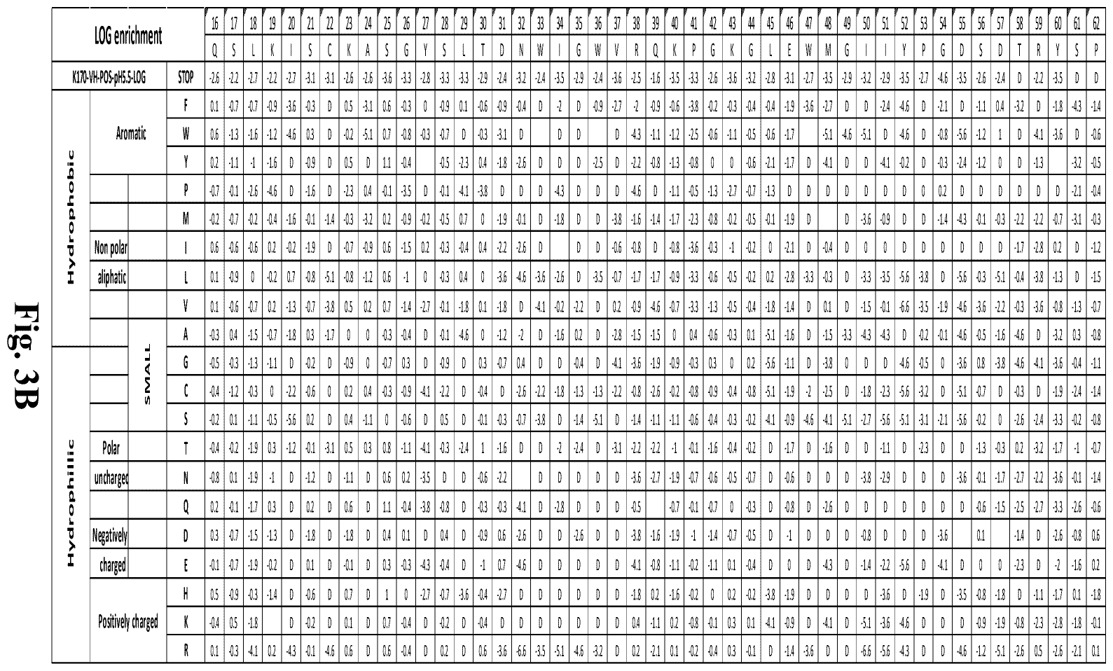

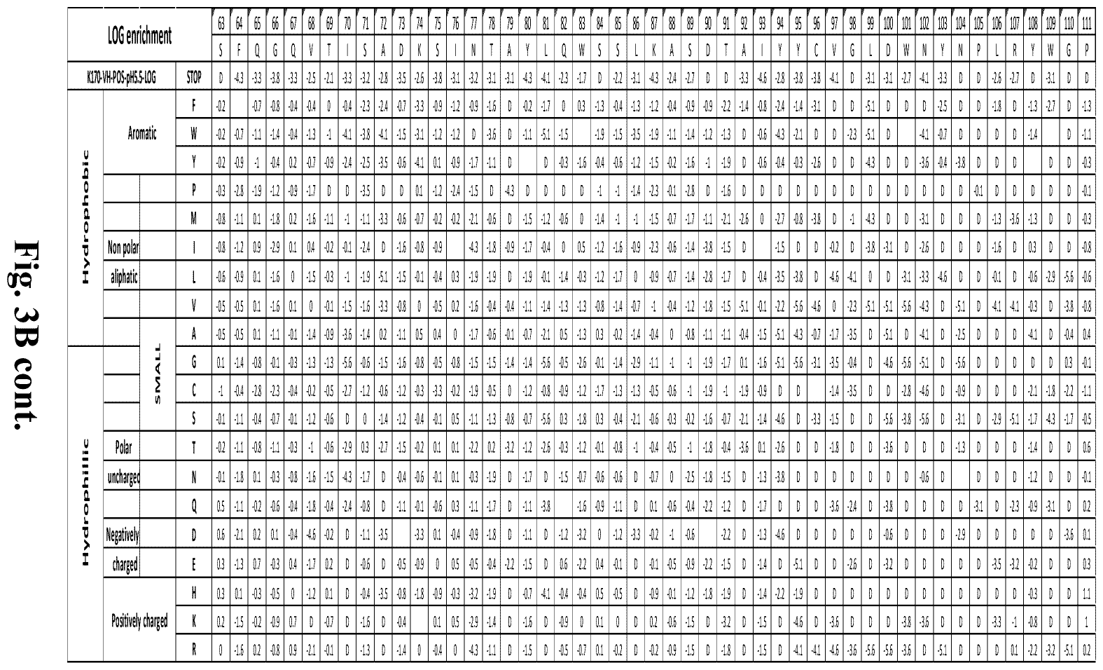

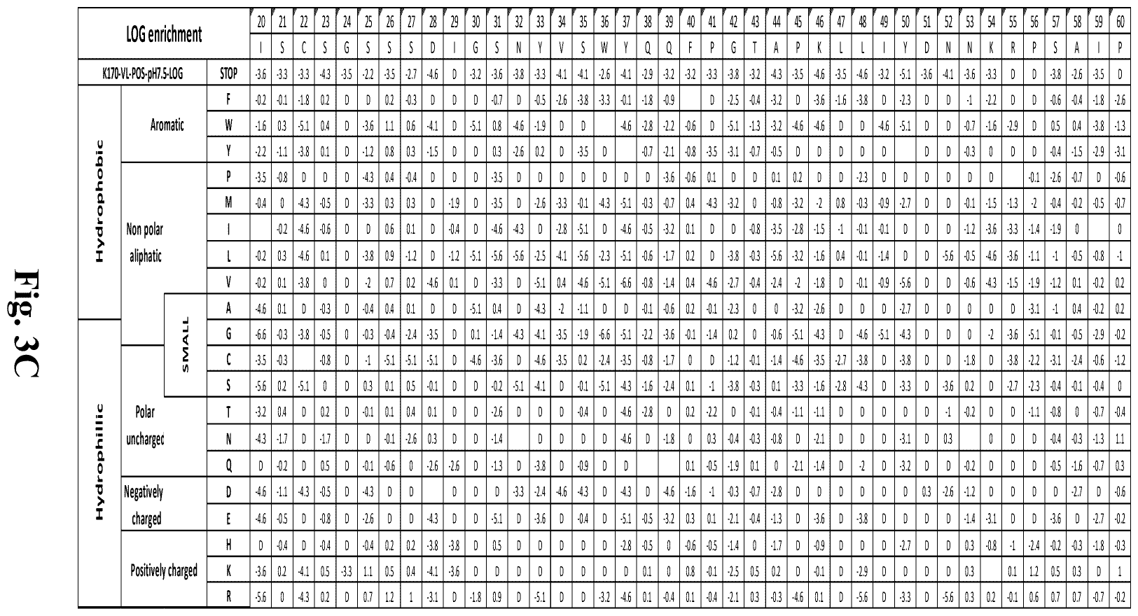

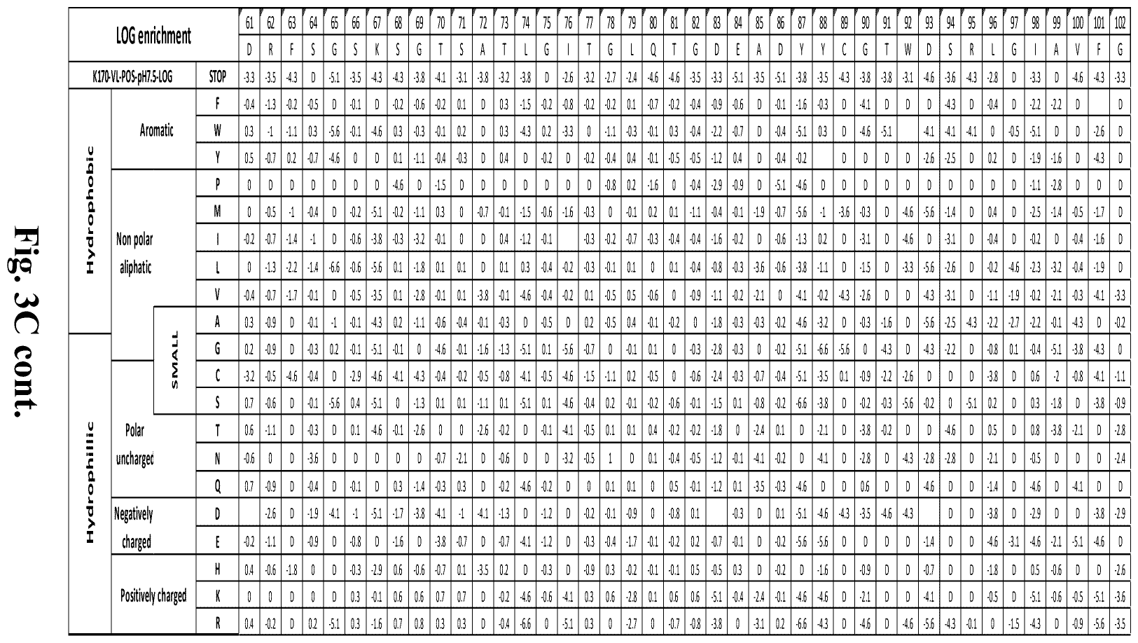

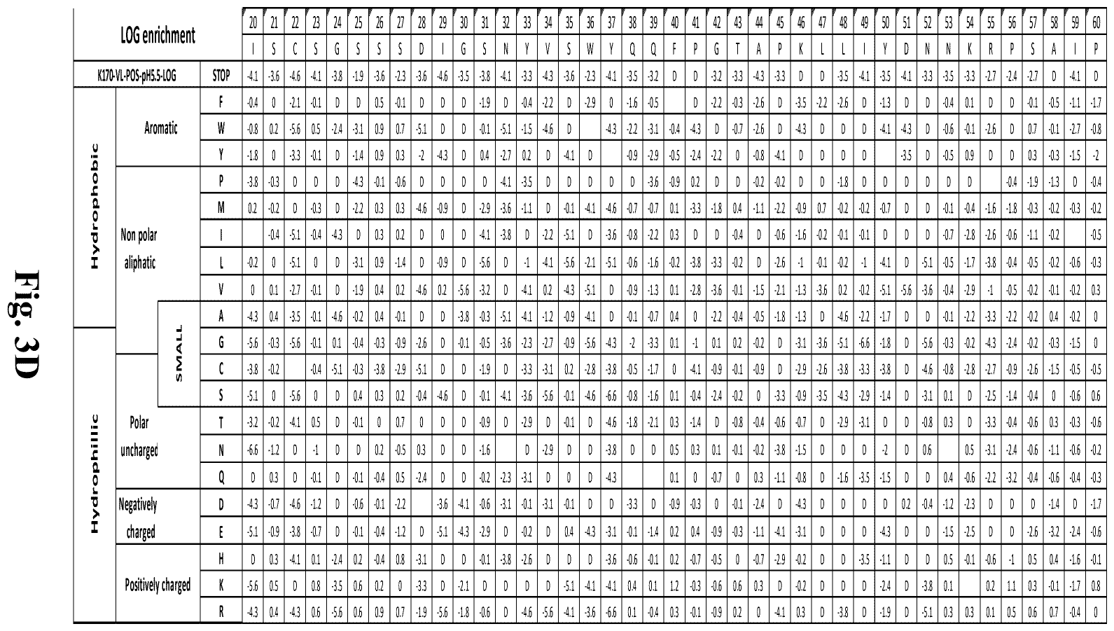

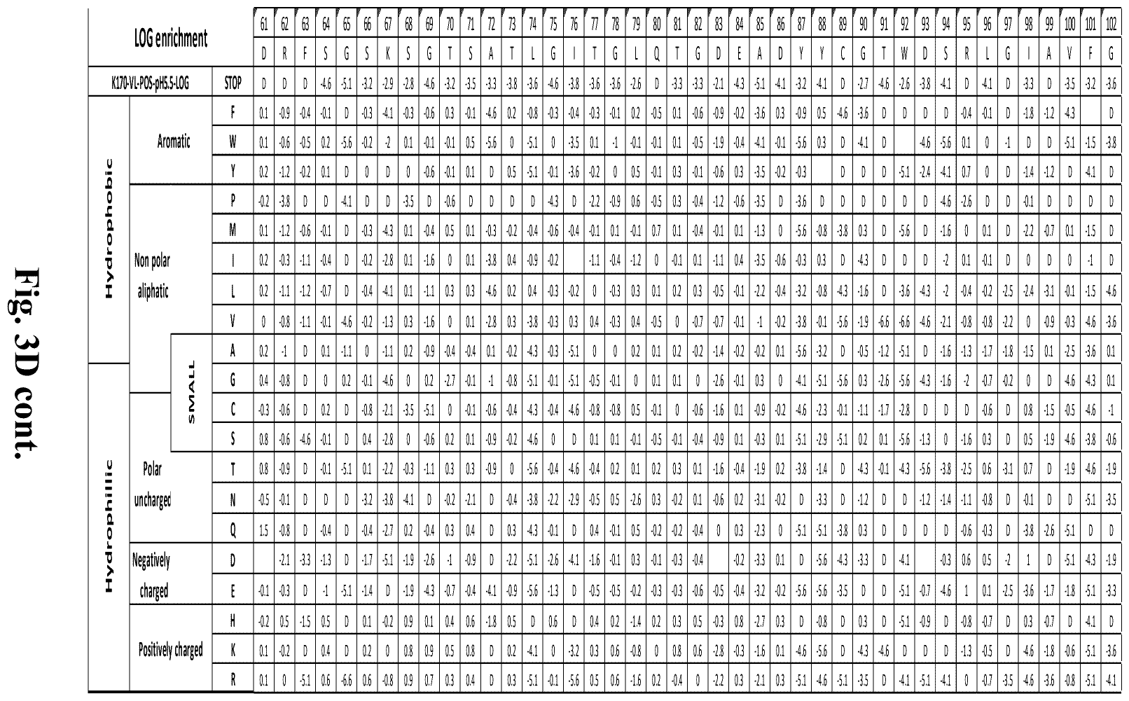

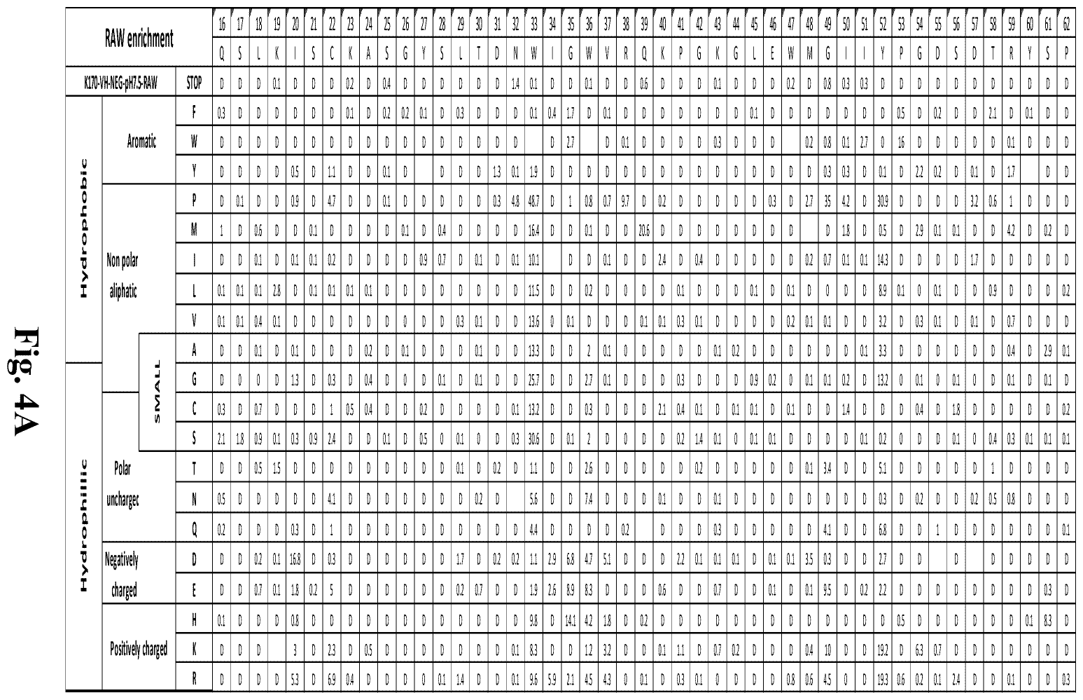

The VH paratope library and VL paratope library were screened for affinity changes to human TSHR relative to the parental antibody. The yeast cells expressing either the VH or VL library were incubated with 1 nM human TSHR at pH 7.5 or pH 5.5. A depiction of the FACS gating is shown in FIG. 2. Log2 enrichment for single amino acid substitutions were determined and reported in FIG. 3A-FIG. 3D.

In FIG. 3A-FIG. 3D, a value of “D” represents a mutant that is present in the unsorted library but absent in the sorted library and may indicate a loss of antigen binding. A value of “A” represents a mutant that is present in the sorted library but absent in the unsorted library.

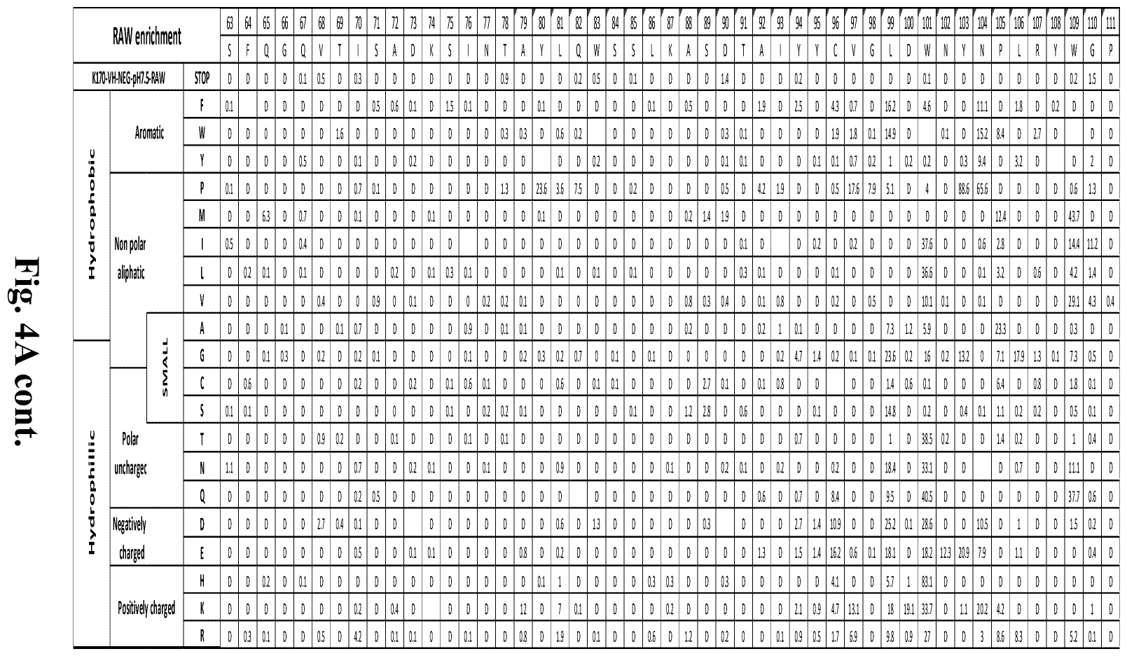

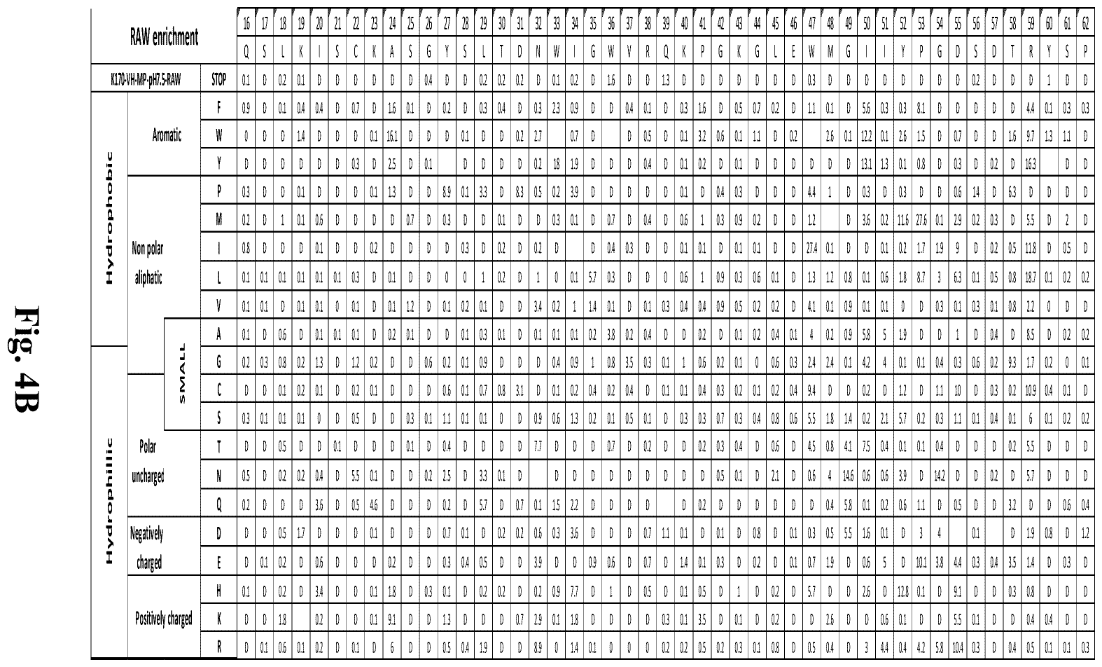

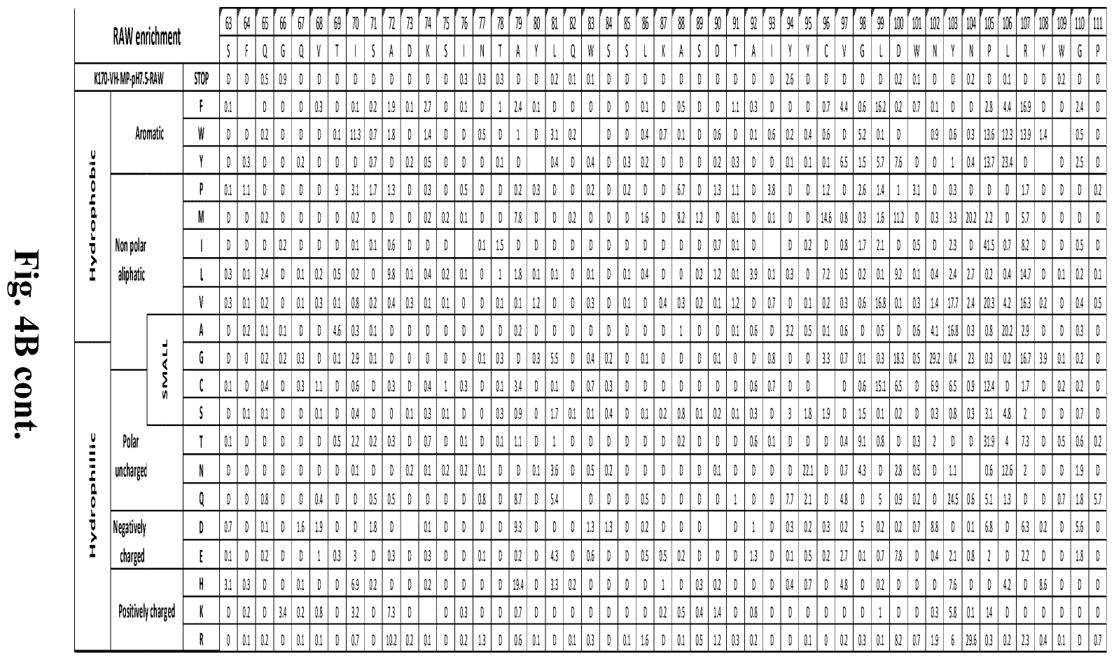

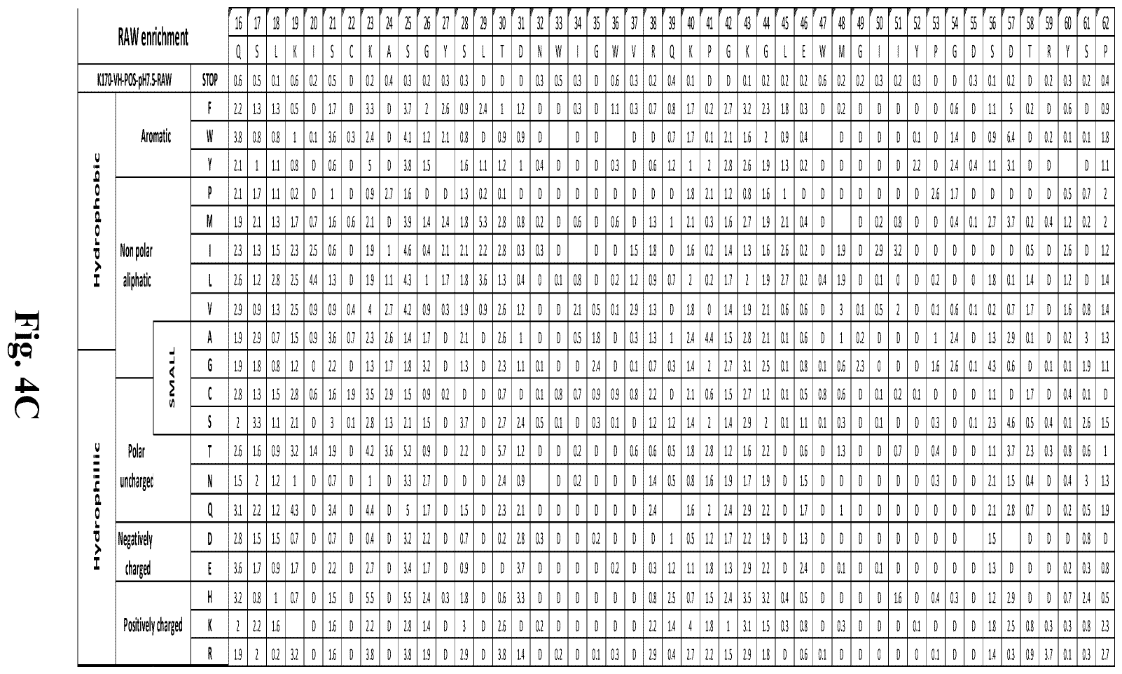

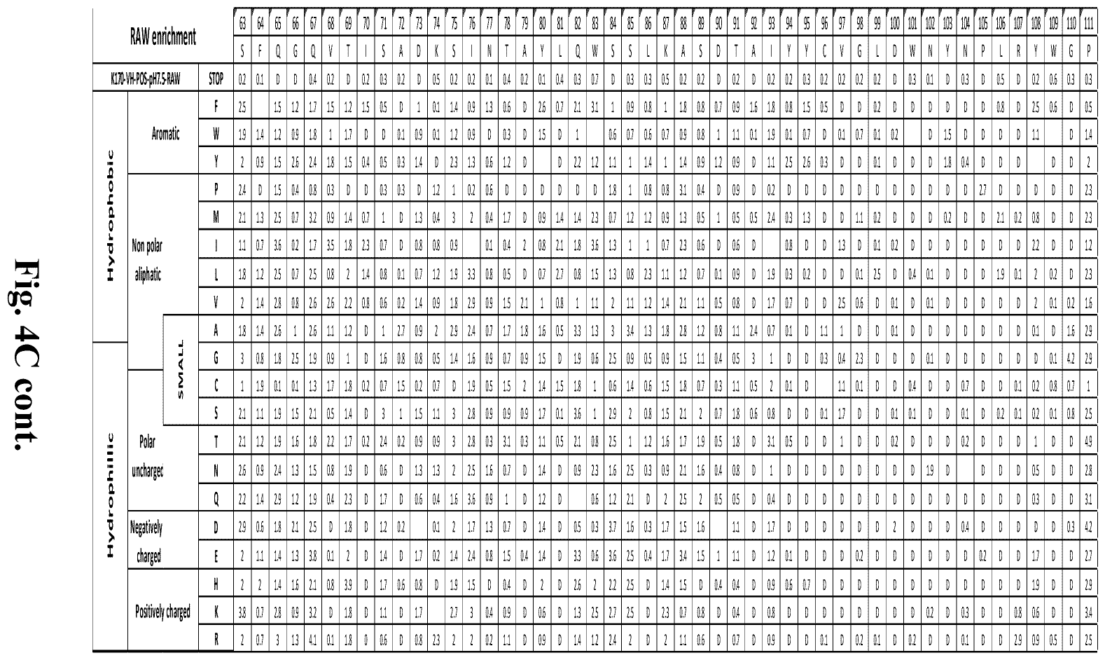

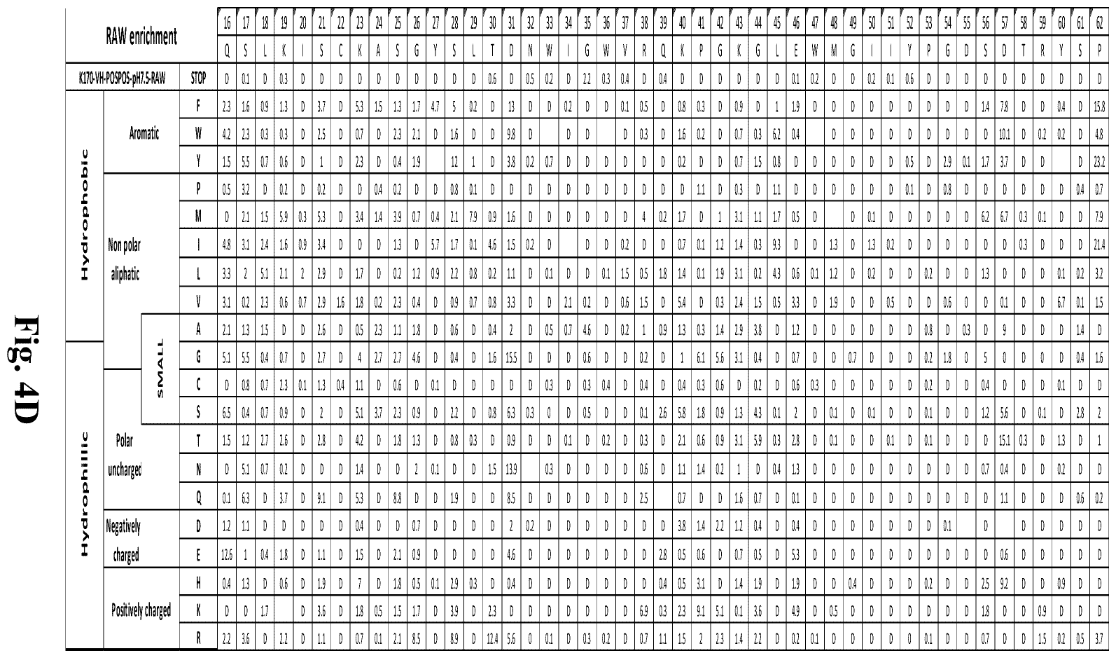

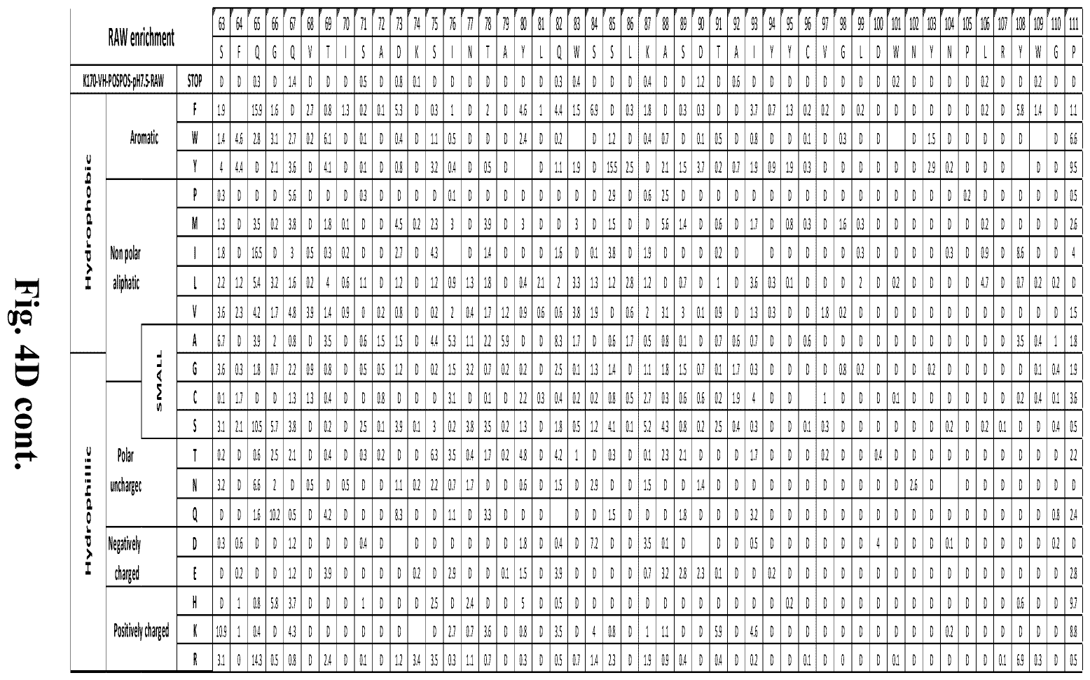

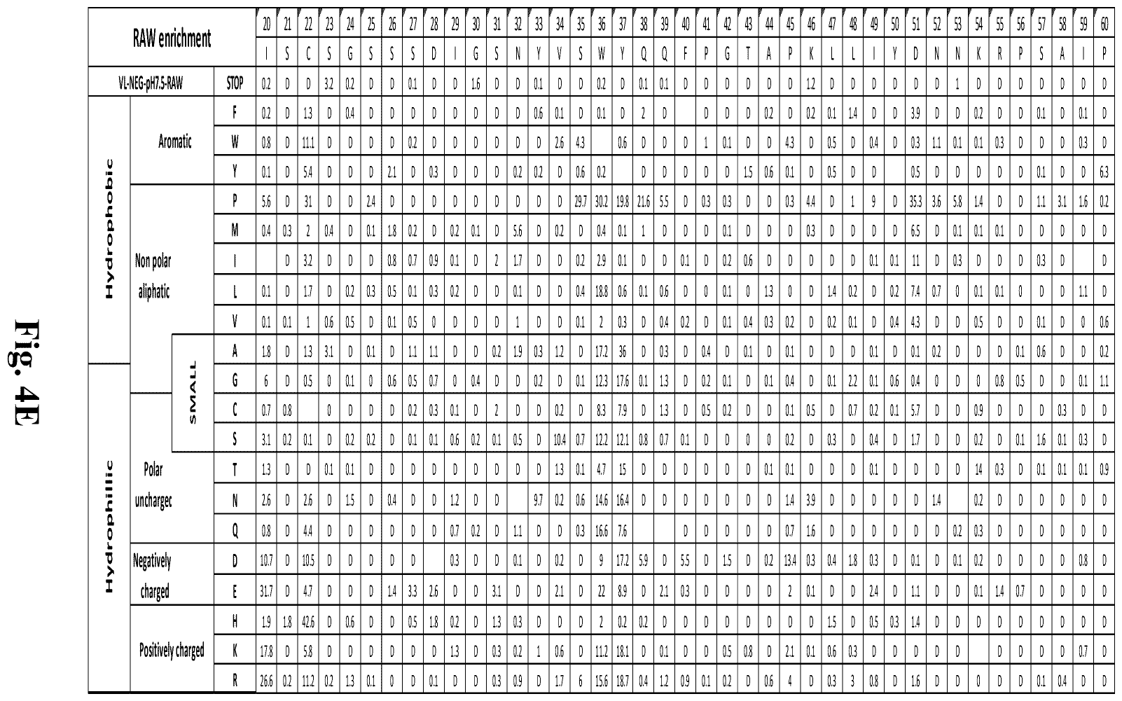

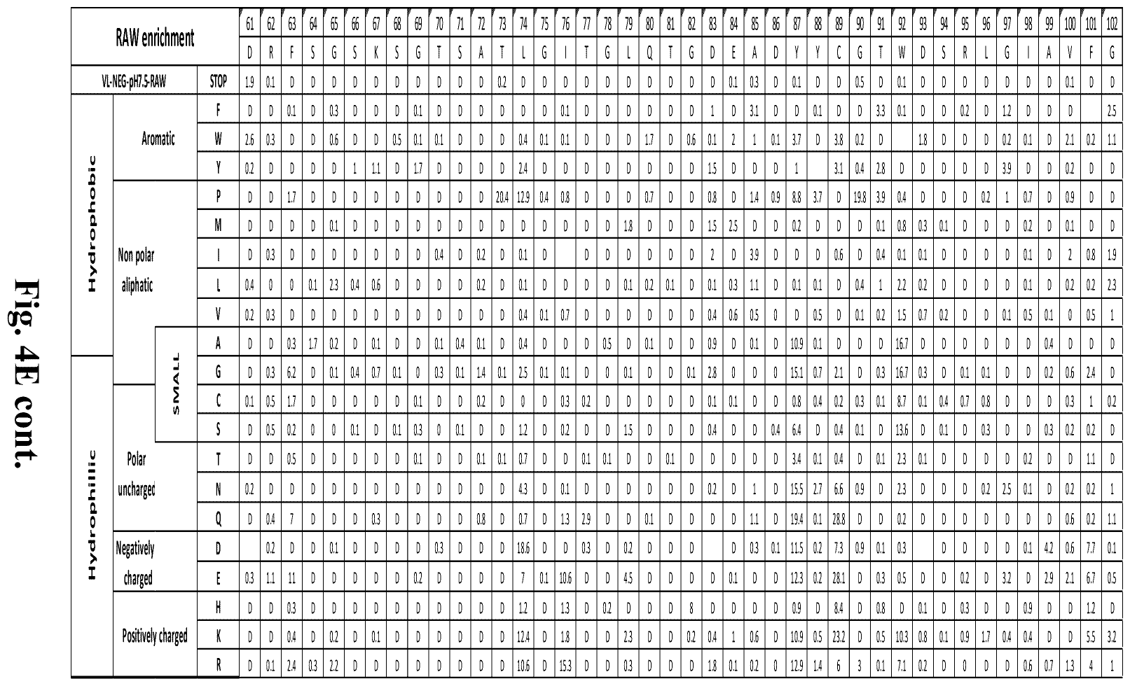

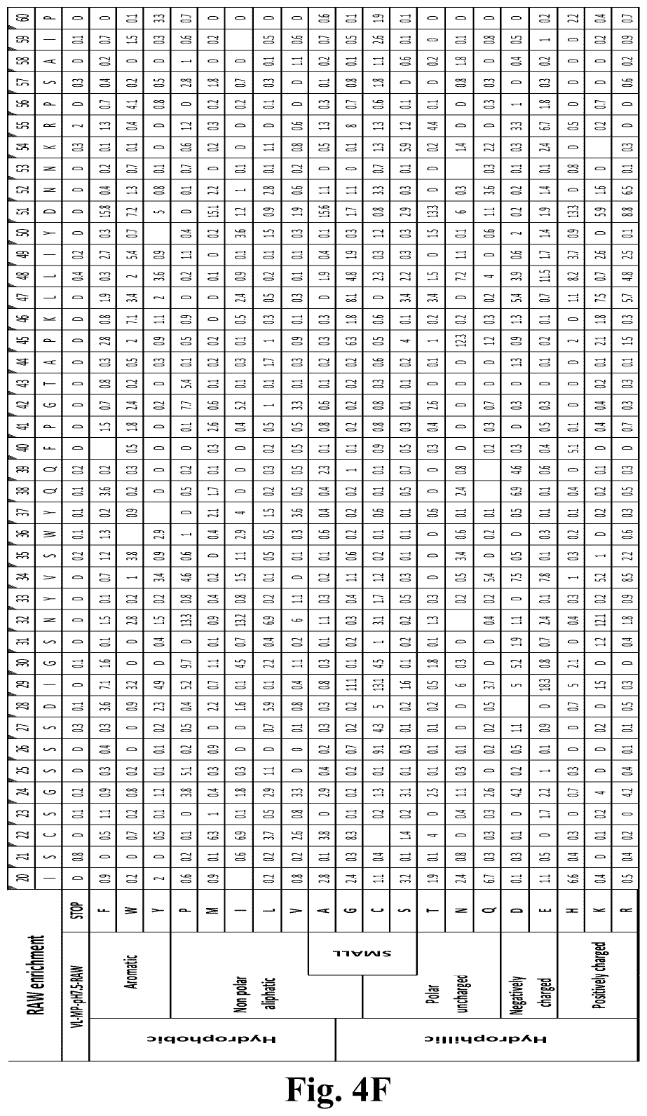

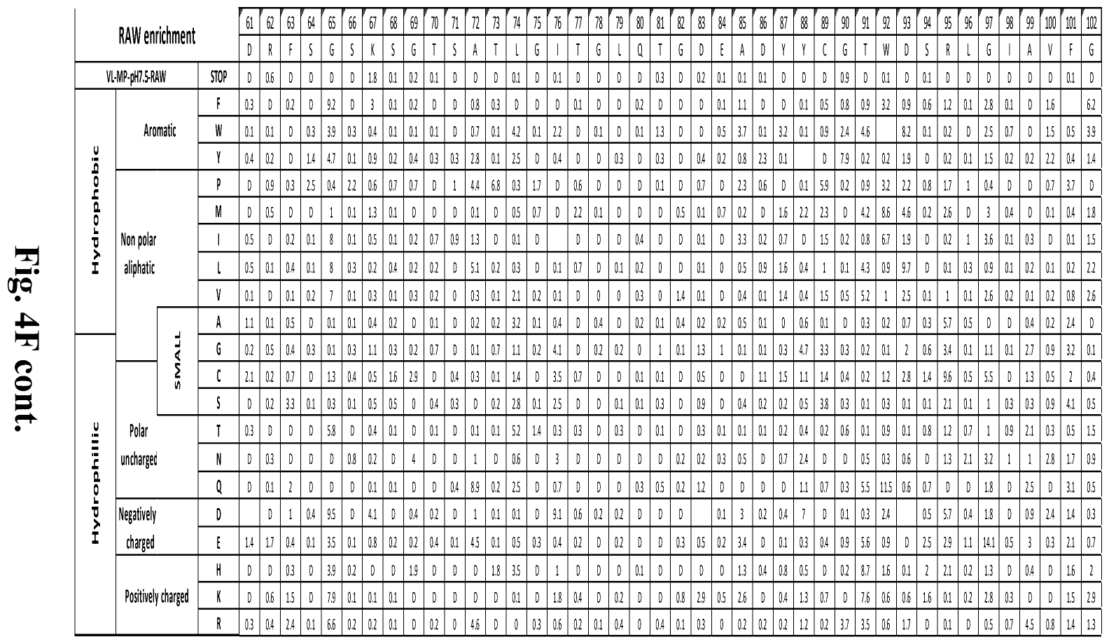

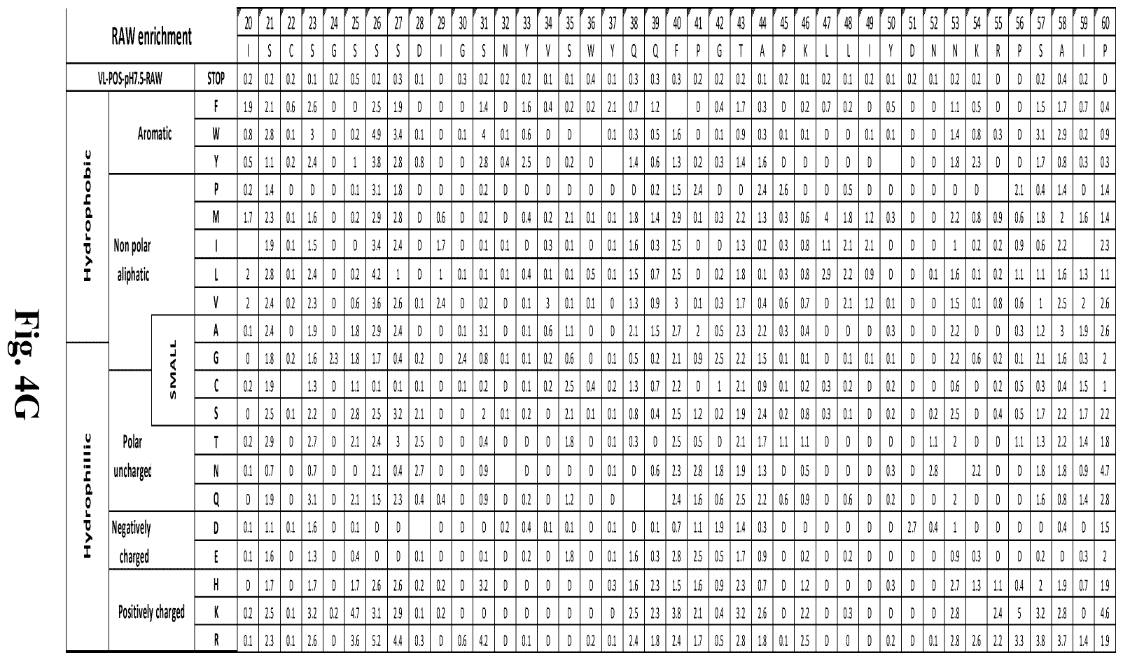

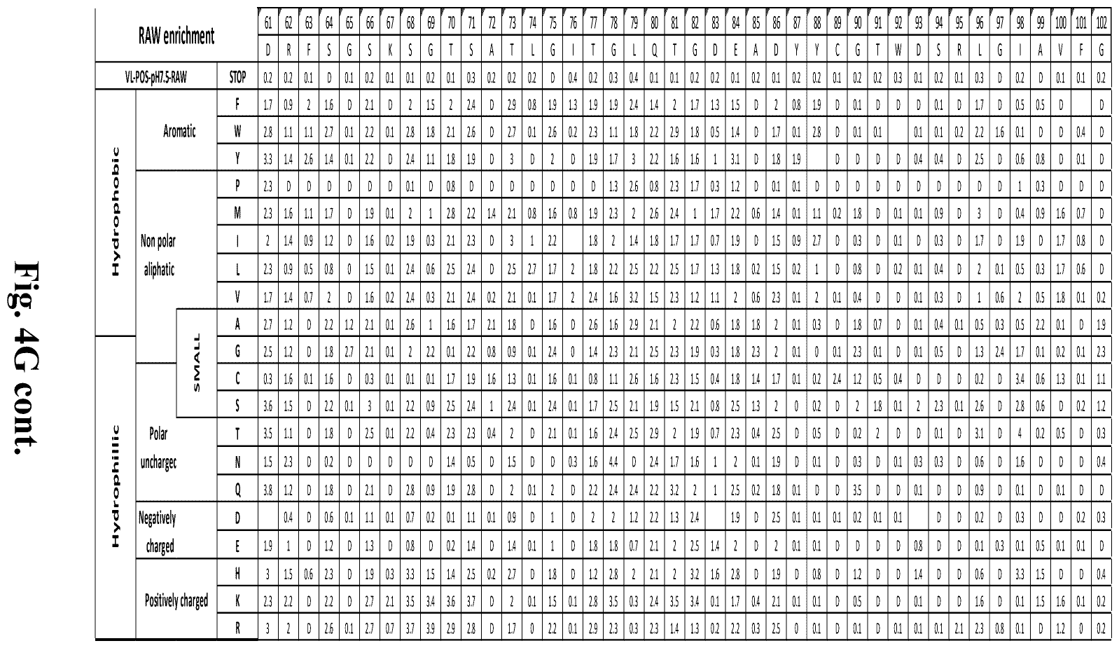

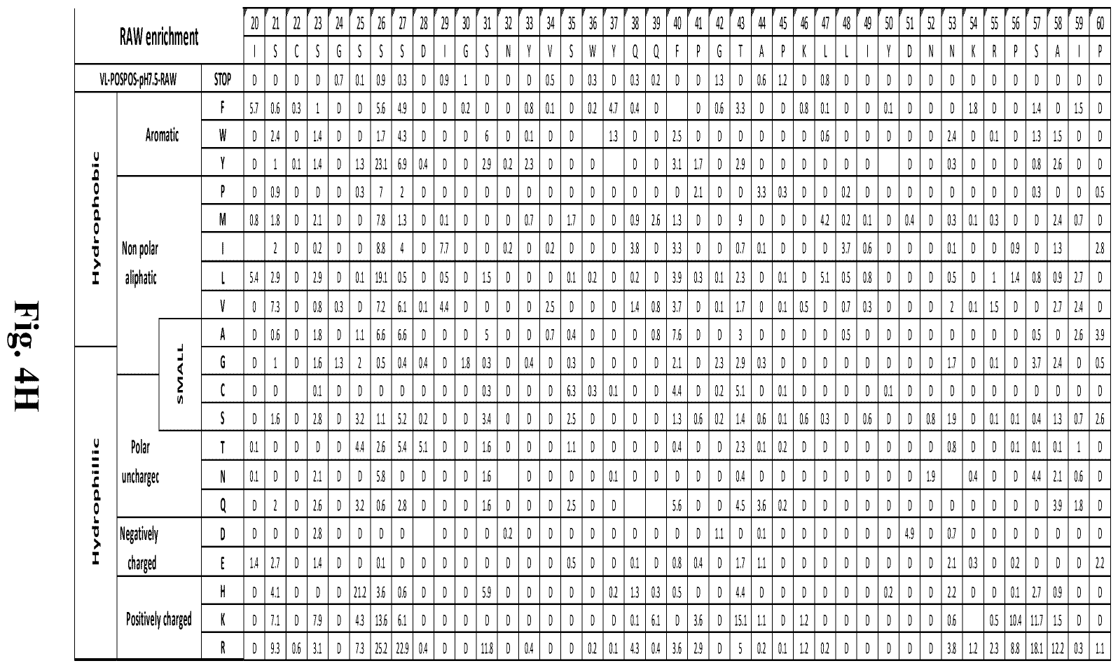

The raw enrichment of select variants was measured at pH 7.5 based on the different gating strategies described above. FIG. 4A and FIG. 4E depicts raw enrichment of specific mutations in the VH (FIG. 4A) and VL (FIG. 4E) paratope under pH 7.5 conditions under the negative gating. Higher enrichment values indicate that the mutation causes a strong loss of Fab recognition for its antigen or has a strong negative effect on the VH/VL pairing. FIG. 4B and FIG. 4F depicts raw enrichment of specific mutations in the VH (FIG. 4B) and VL (FIG. 4F) paratope under pH 7.5 conditions under MP gating. Higher enrichment values indicate that the mutation may considerably lower but not abolish affinity. FIG. 4C and FIG. 4G depicts raw enrichment of specific mutations in the VH (FIG. 4C) and VL (FIG. 4G) paratope under pH 7.5 conditions under positive gating. Higher enrichment values indicate that the mutation may improve affinity. Values at 3.5 or higher more strongly associate with improved affinity. FIG. 4D and FIG. 4H depicts raw enrichment of specific mutations in the VH (FIG. 4D) and VL (FIG. 4H) paratope under pH 7.5 conditions under pospos gating. Higher enrichment values indicate that the mutation may considerably improve affinity. Values at 5.0 or higher more strongly associate with improved affinity.

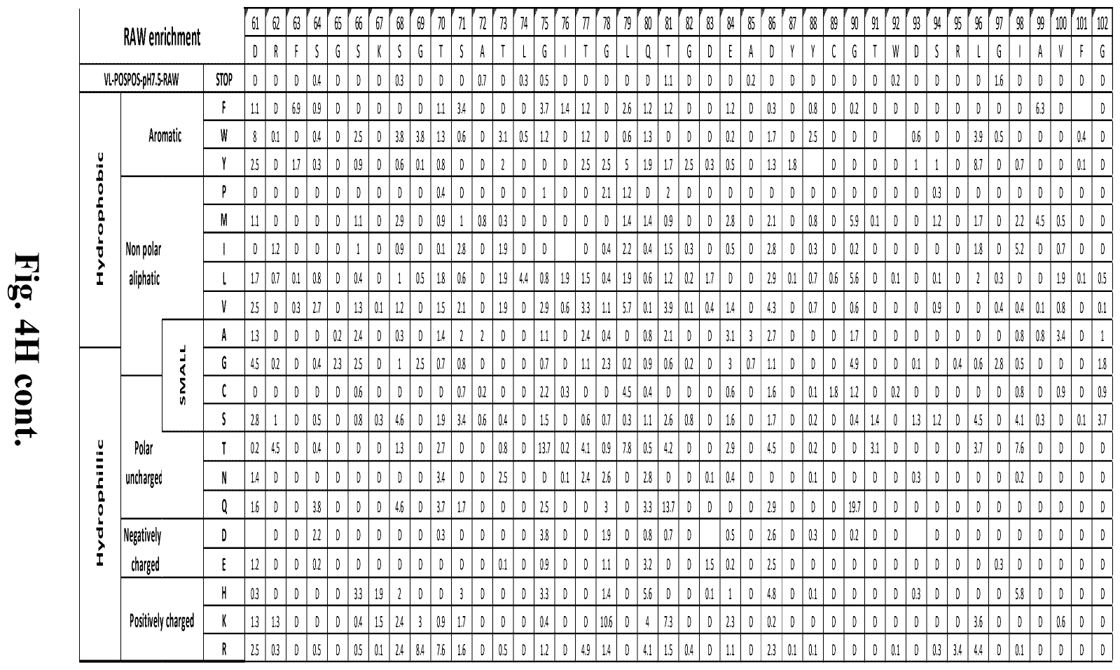

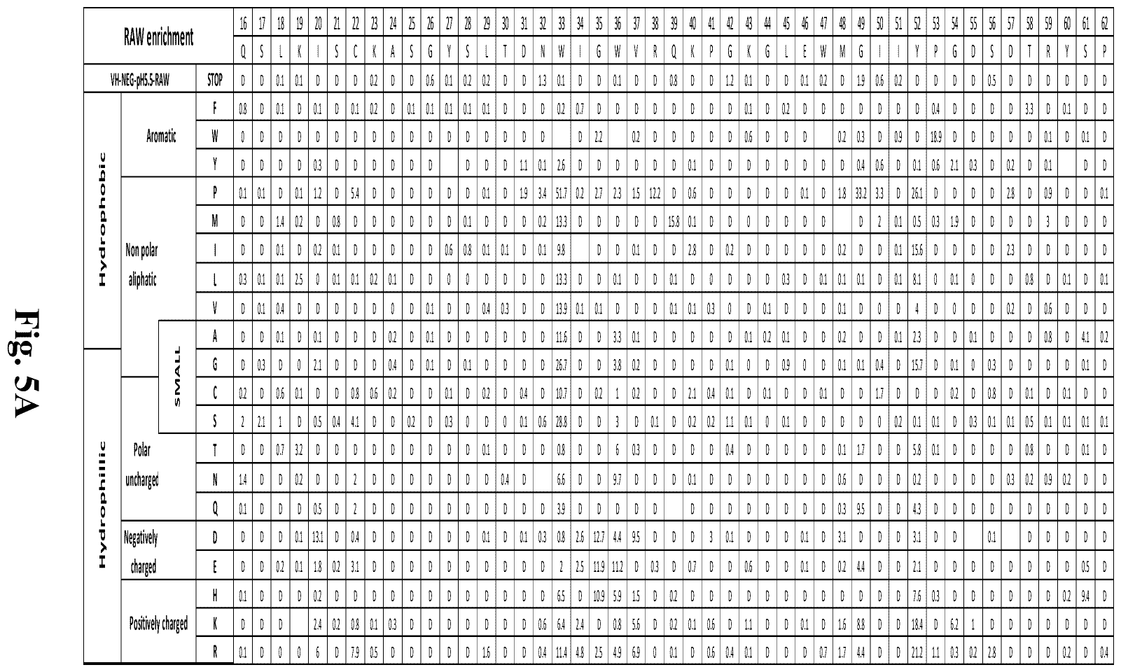

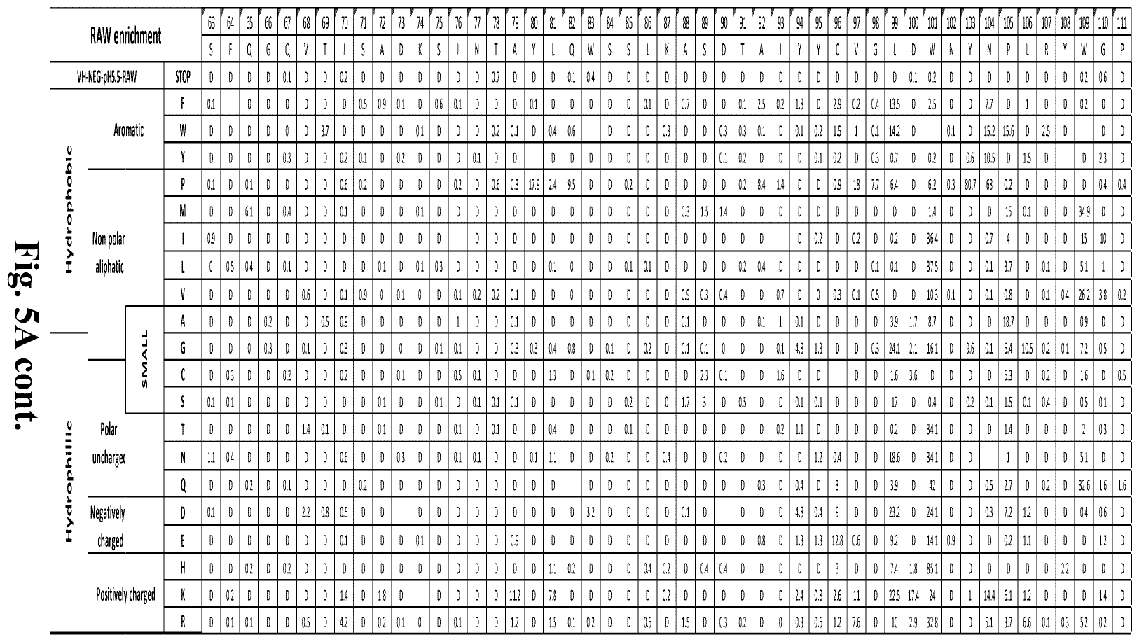

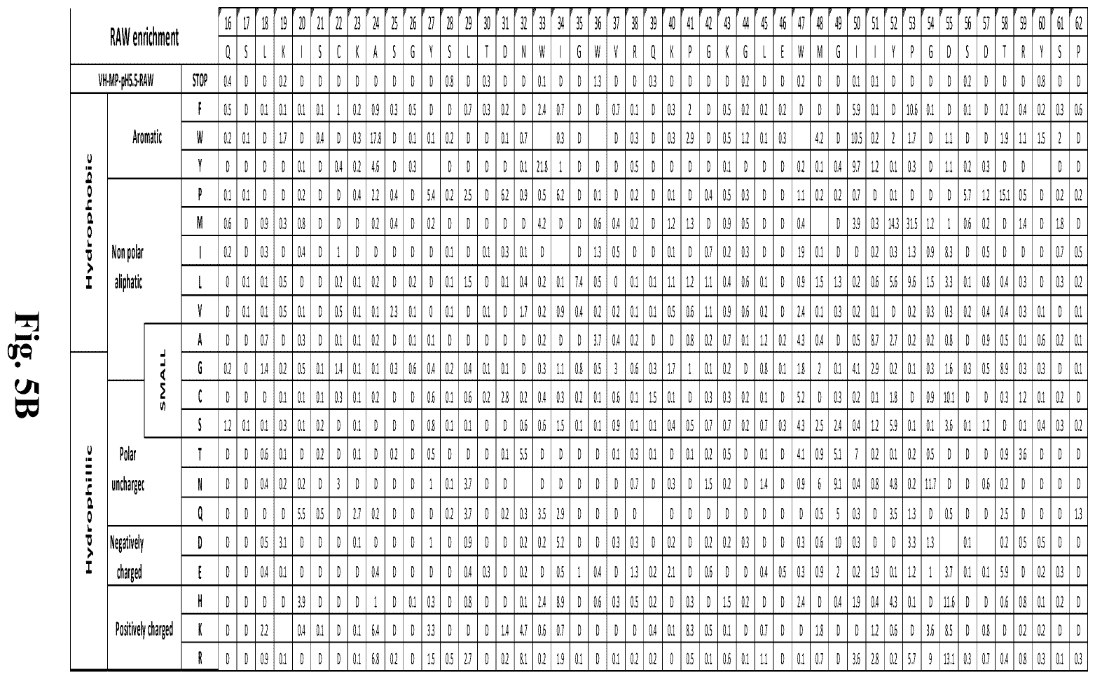

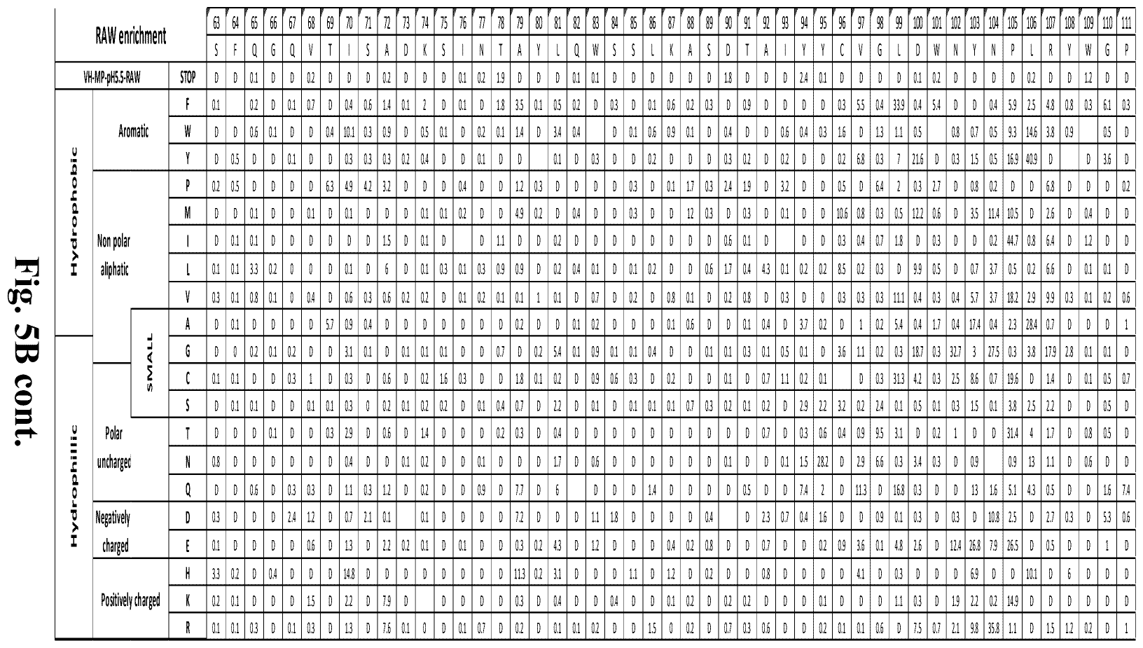

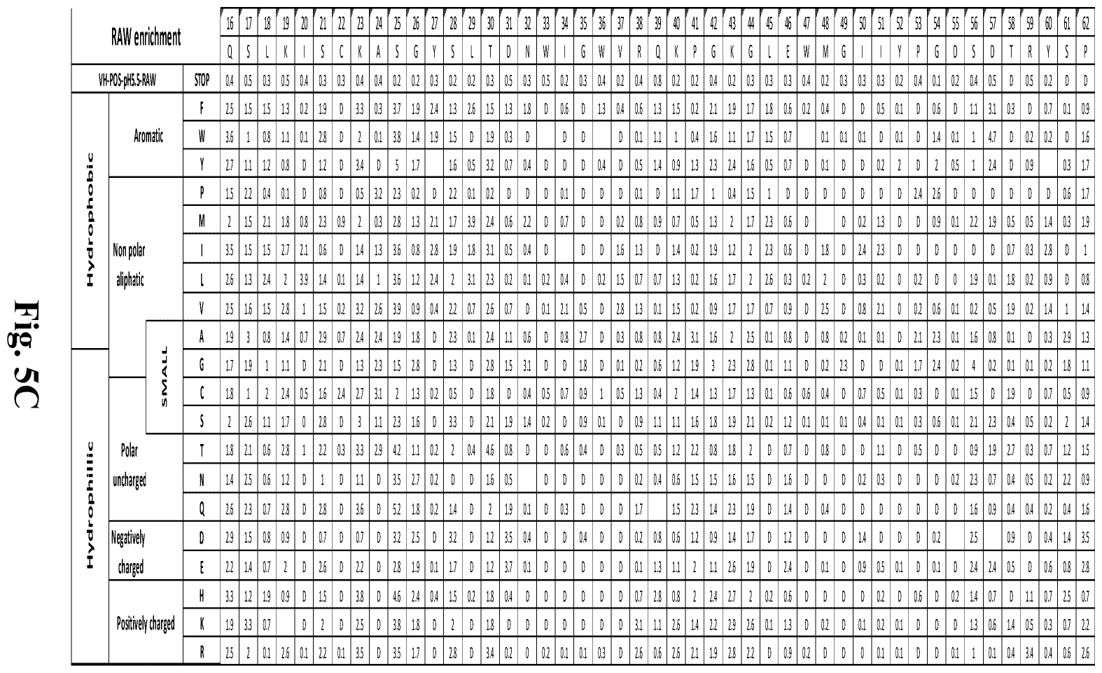

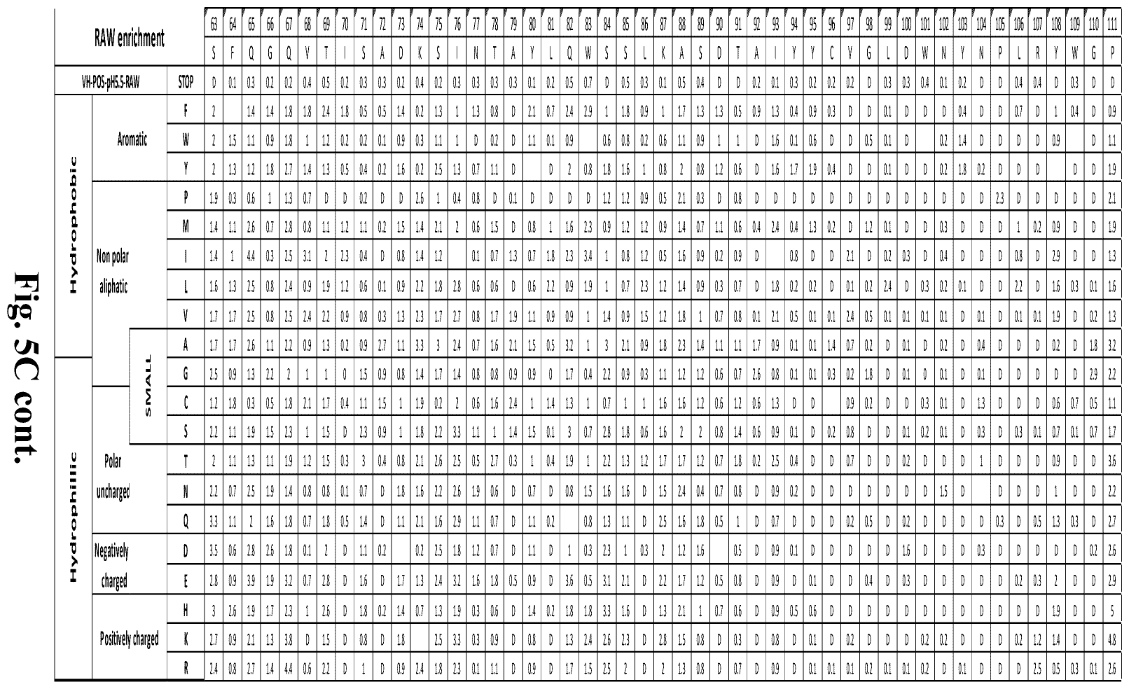

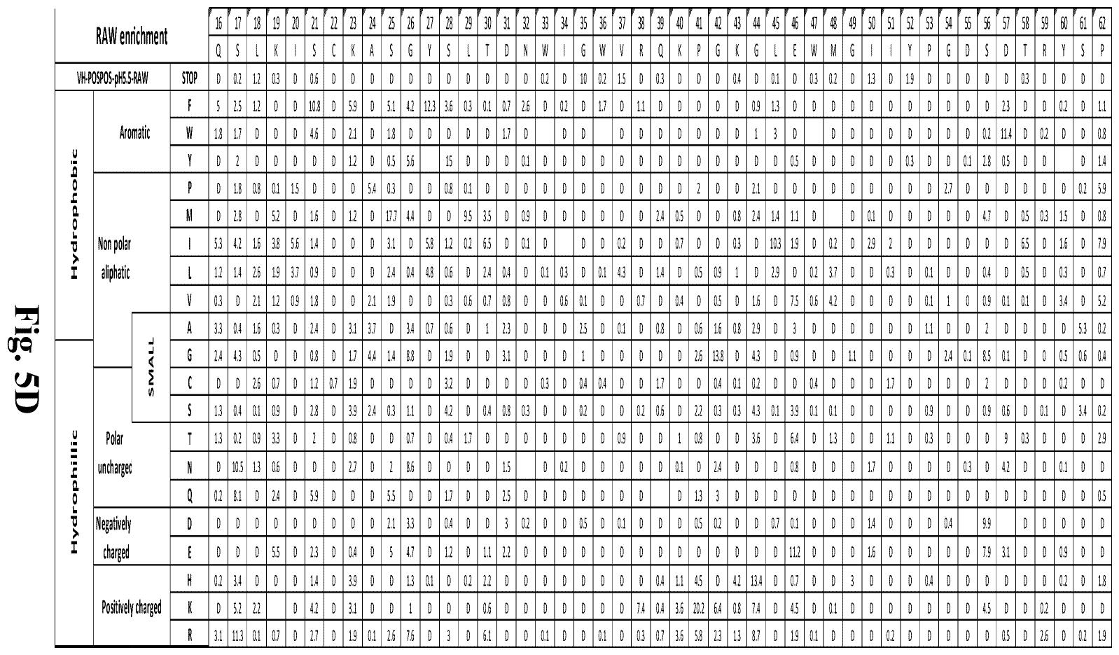

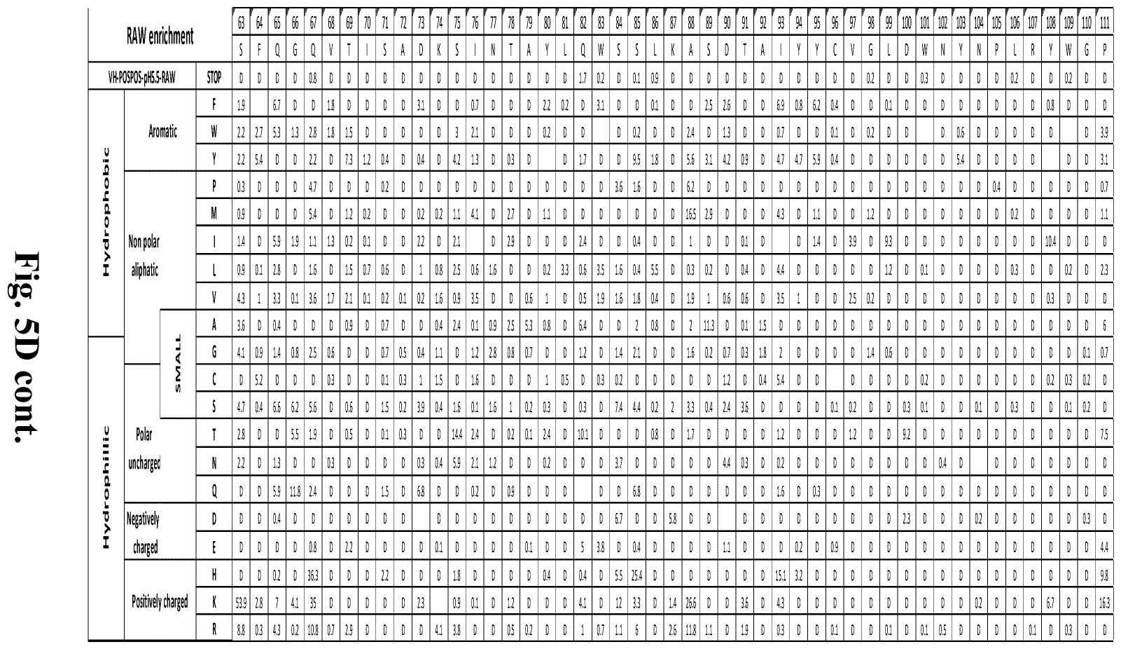

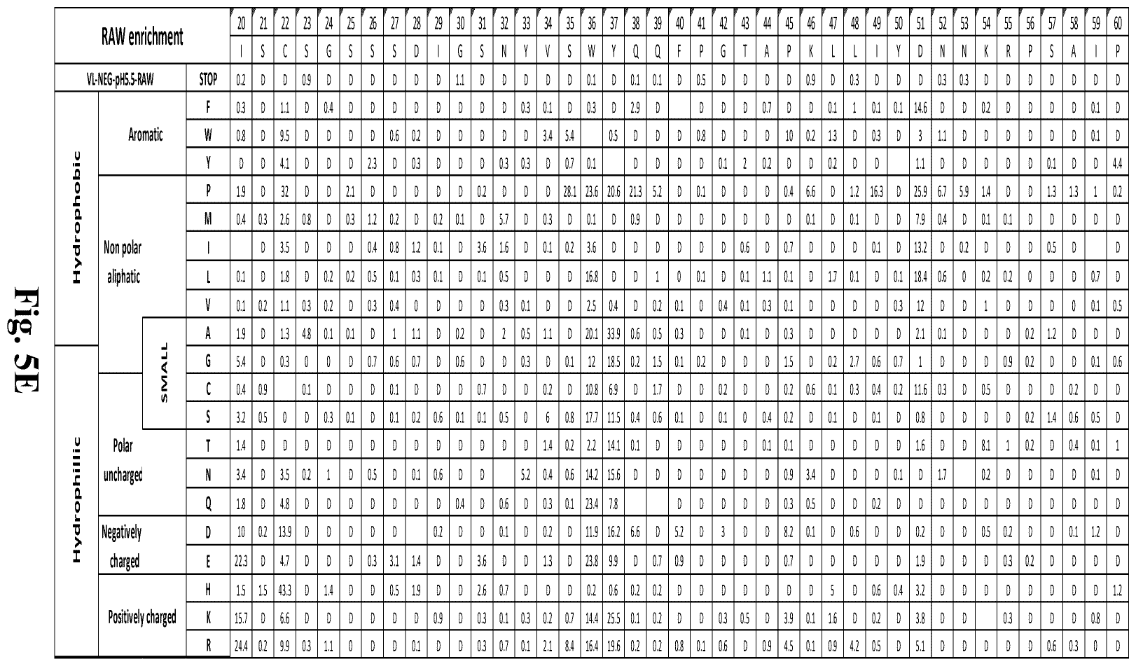

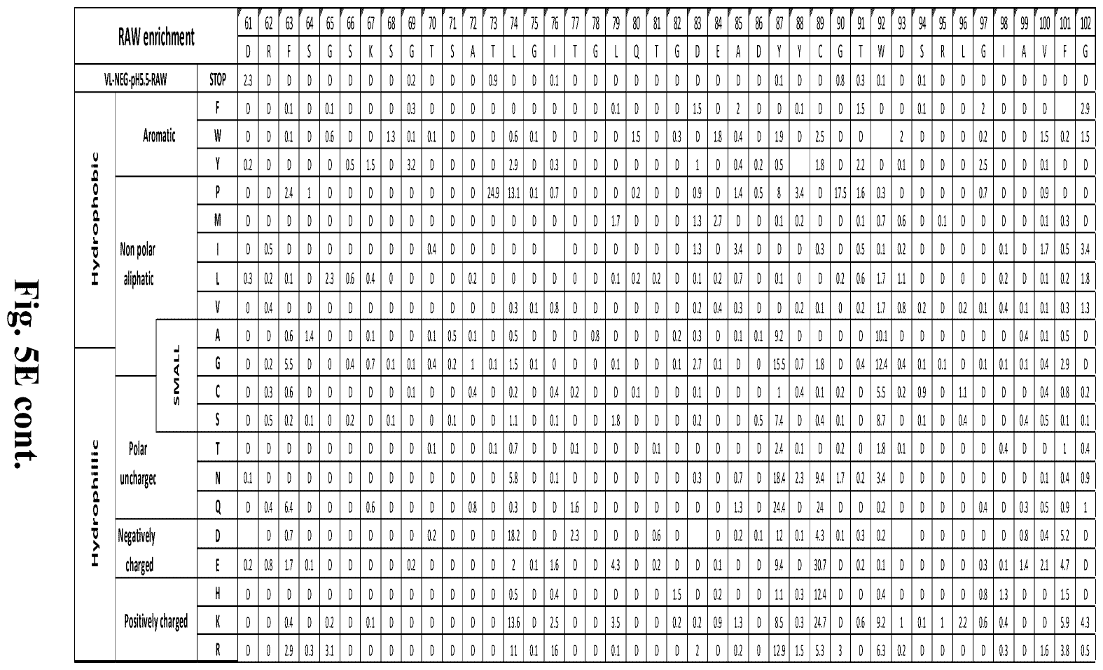

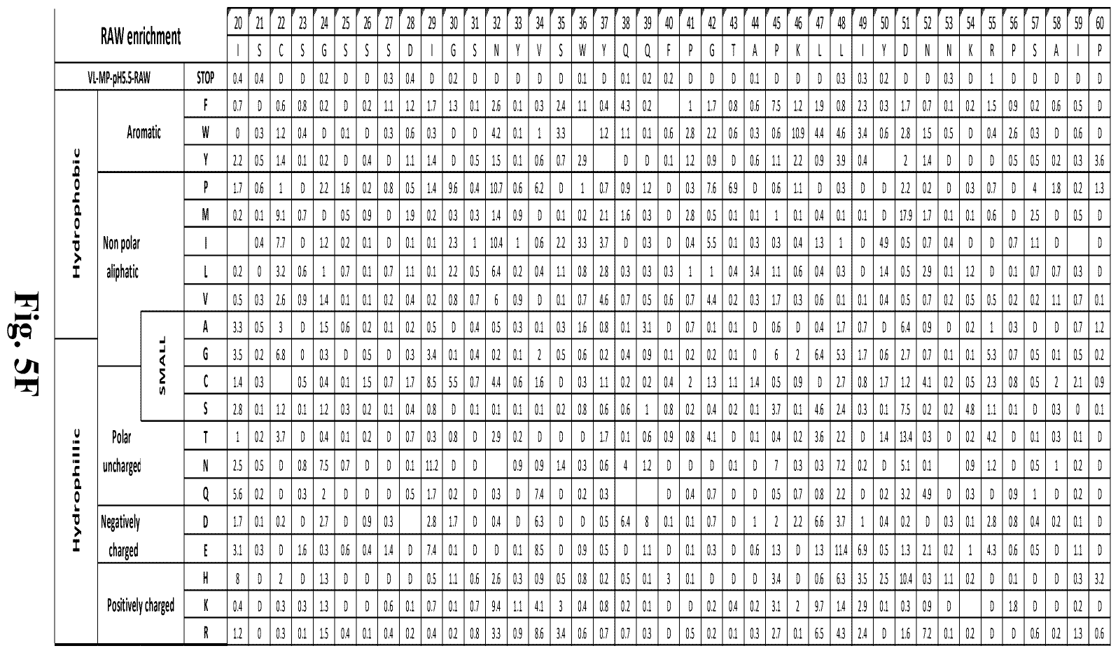

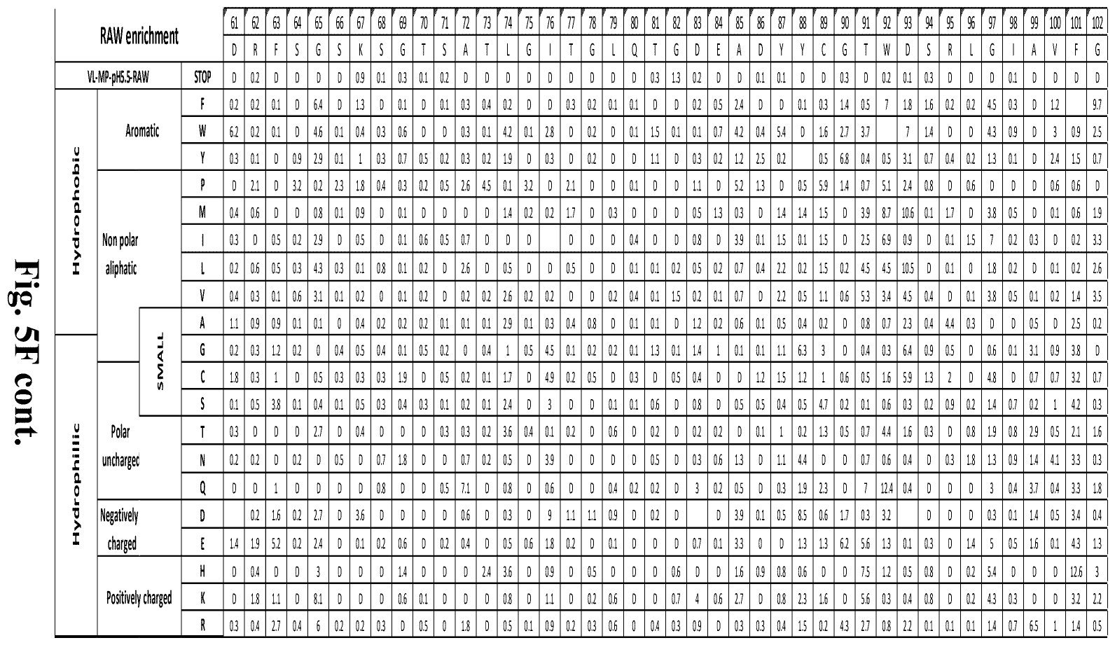

The raw enrichment of select variants was measured at pH 5.5 based on the different gating strategies described above. FIG. 5A and FIG. 5E depicts raw enrichment of specific mutations in the VH (FIG. 5A) and VL (FIG. 5E) paratope under pH 5.5 conditions under the negative gating. Higher enrichment values indicate that the mutation causes a strong loss of Fab recognition for its antigen or has a strong negative effect on the VH/VL pairing. FIG. 5B and FIG. 5F depicts raw enrichment of specific mutations in the VH (FIG. 5B) and VL (FIG. 5F) paratope under pH 5.5 conditions under MP gating. Higher enrichment values indicate that the mutation may considerably lower but not abolish affinity. FIG. 5C and FIG. 5G depicts raw enrichment of specific mutations in the VH (FIG. 5C) and VL (FIG. 5G) paratope under pH 5.5 conditions under positive gating. Higher enrichment values indicate that the mutation may improve affinity. Values at 3.5 or higher more strongly associate with improved affinity. FIG. 5D and FIG. 5H depicts raw enrichment of specific mutations in the VH (FIG. 5D) and VL (FIG. 5H) paratope under pH 5.5 conditions under pospos gating. Higher enrichment values indicate that the mutation may considerably improve affinity. Values at 5.0 or higher more strongly associate with improved affinity.

Based on the results of the affinity screen, any one or more of the following amino acid substitutions were identified to increase affinity to TSHR:
VH: L29M; D57F/W/M/S/T/H; Y108I
VL: S26Y/L/R; S27R; 198T

The recited VH and VL substitutions may be employed in the parental anti-TSHR antibody with a VH of SEQ ID NO: 1 and VL of SEQ ID NO: 2. The VH and VL substitutions may also be employed in the variant anti-TSHR antibody with a VH of SEQ ID NO: 3 and VL of SEQ ID NO: 4 or the variant anti-TSHR antibody with a VH of SEQ ID NO: 78 and VL of SEQ ID NO: 26.

### Thermostability

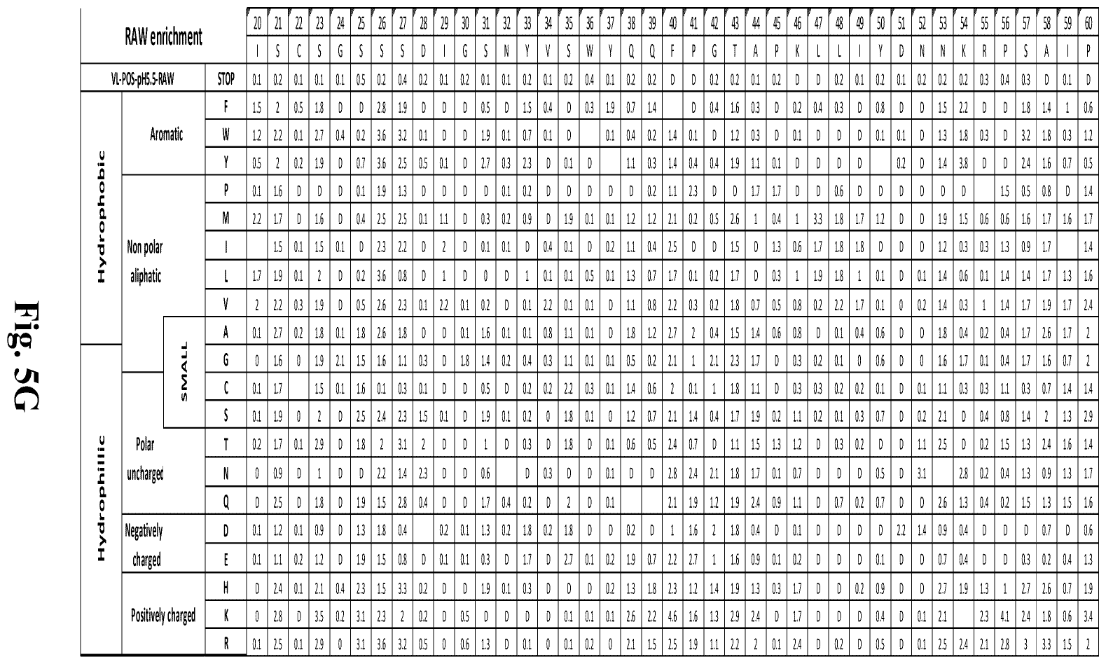

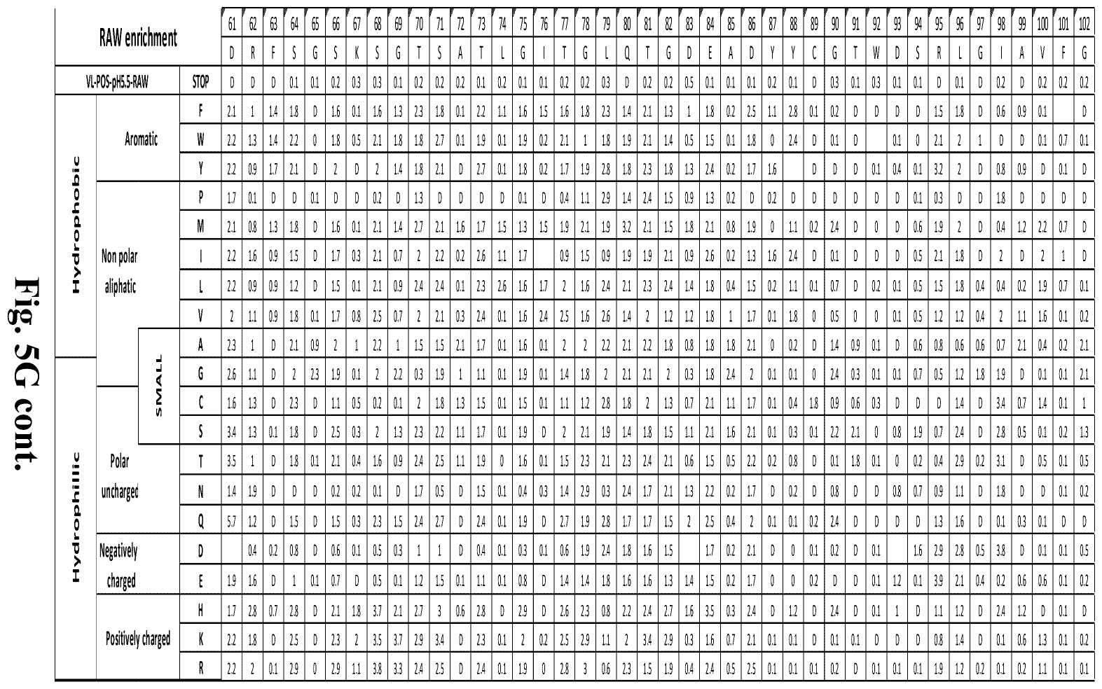

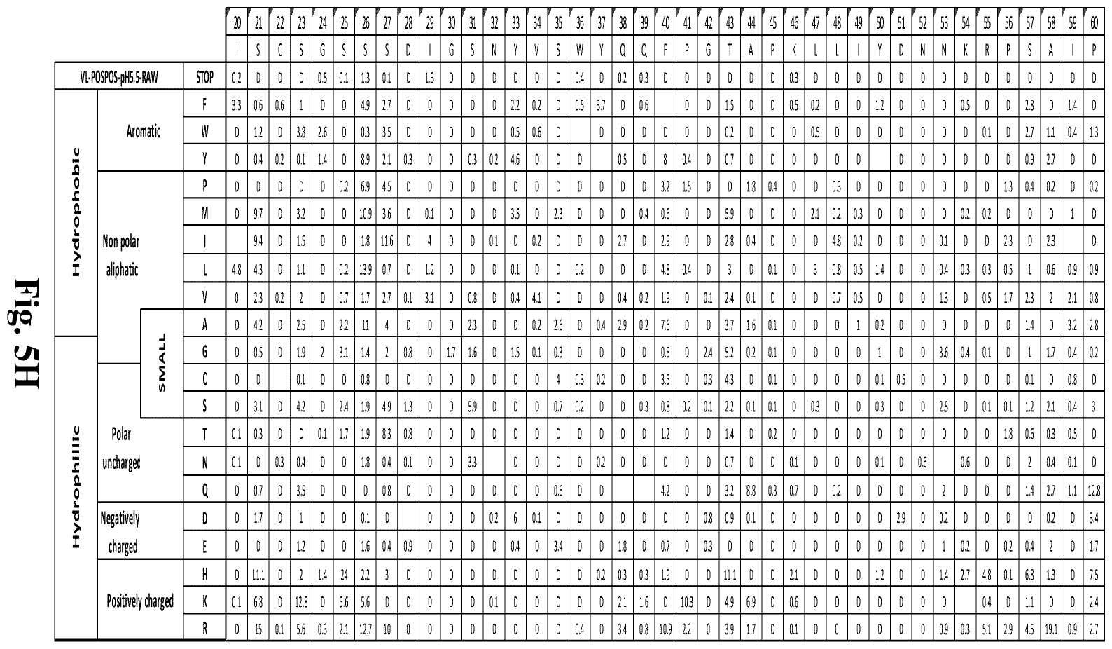

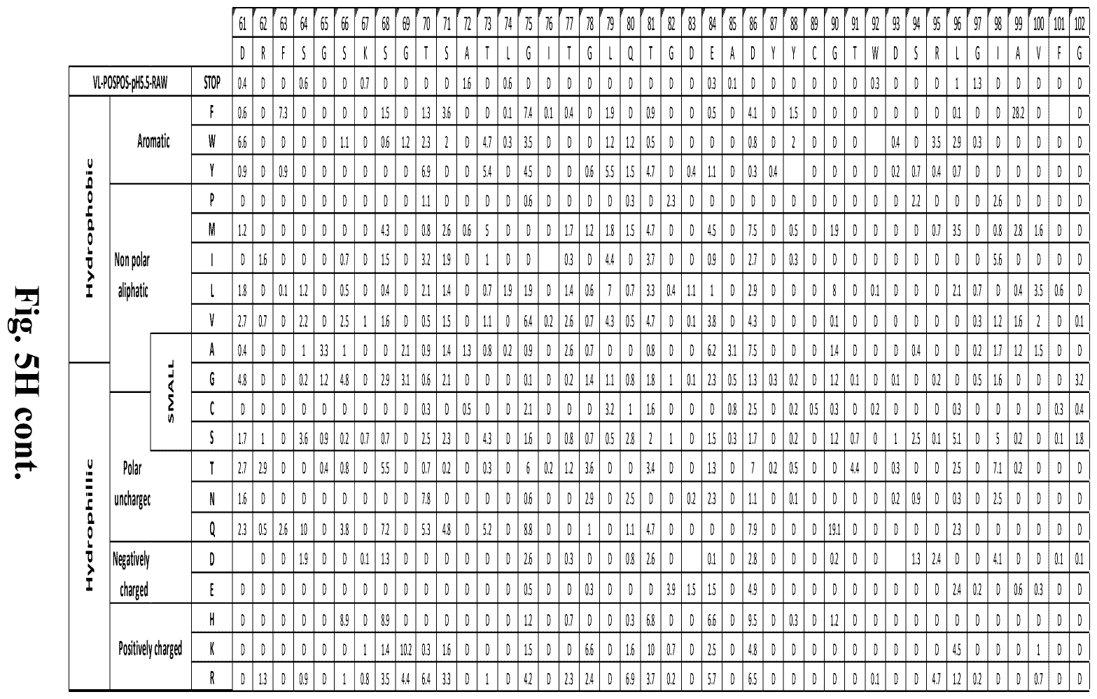

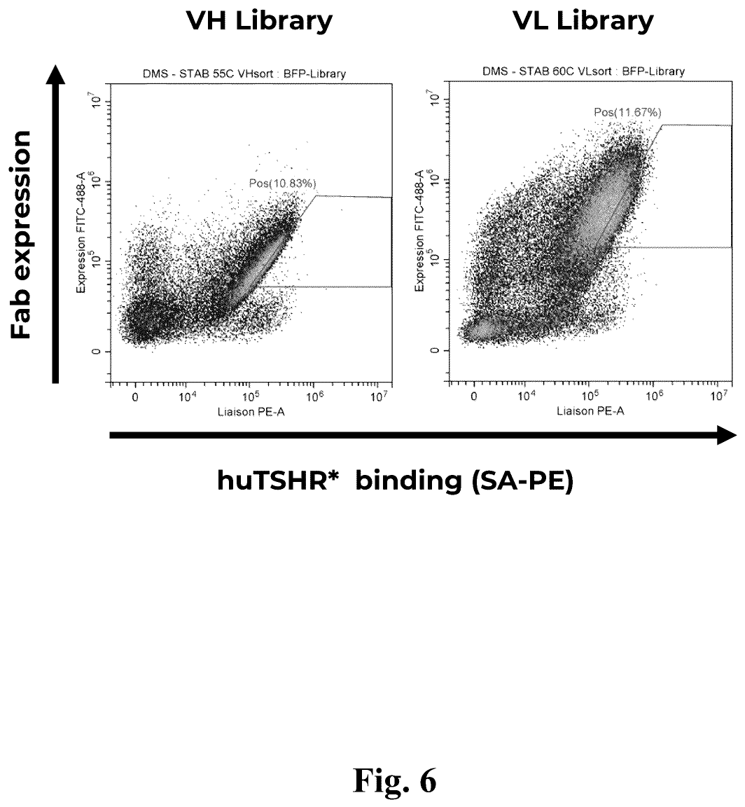

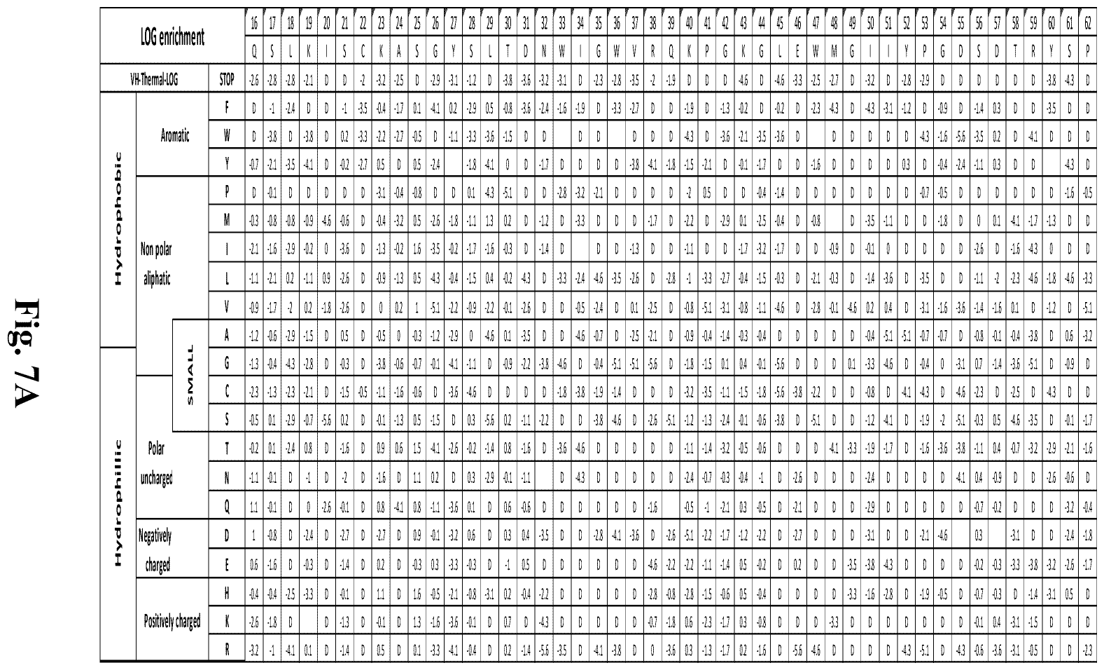

The VH paratope library and VL paratope library were screened for affinity changes to human TSHR relative to the parental antibody under thermal stress. Variant antibodies were heated at 55° C. (VH) or 60° C. (VL) for a 10-minute incubation at pH 7.5 with a human TSHR concentration of 25 nM. A depiction of the FACS gating is shown in FIG. 6. The thermal gate selected for variants with the same or better affinity as parental. Log2 enrichment for single amino acid substitutions were determined and reported in FIG. 7A (VH) and FIG. 7B (VL). Higher enrichment values indicate that the mutation may improve thermal stability. Values at 0.8 or higher more strongly associate with improved thermal stability. A value of “D” represents a mutant that is present in the unsorted library but absent in the sorted library and may indicate a loss of antigen binding and/or lower thermal stability.

### Polyreactivity

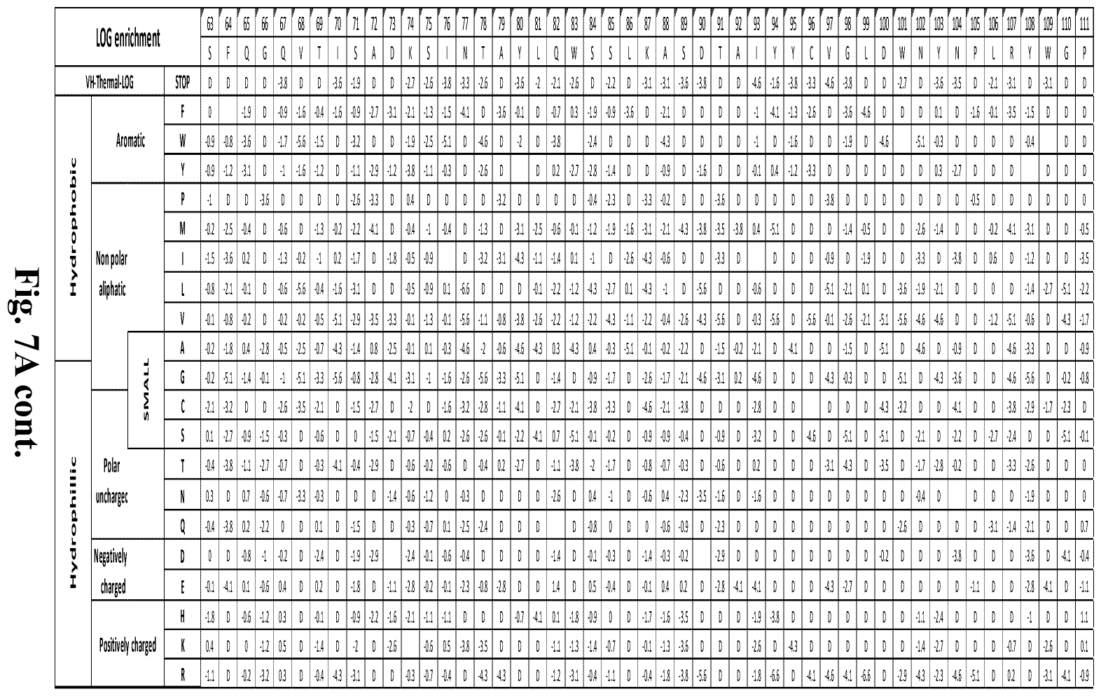

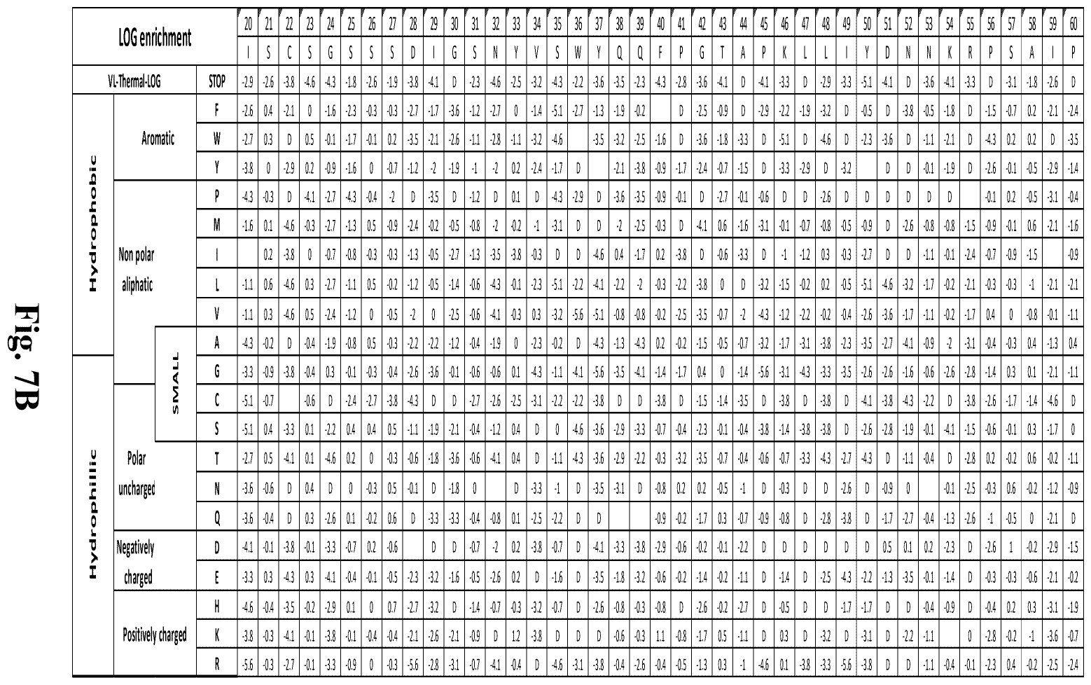

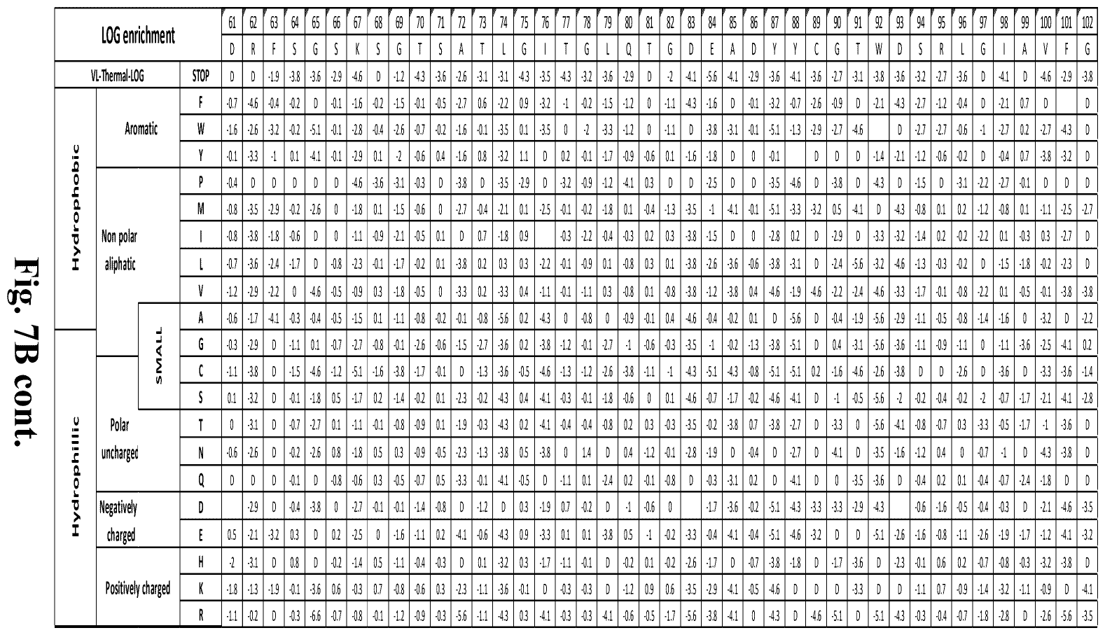

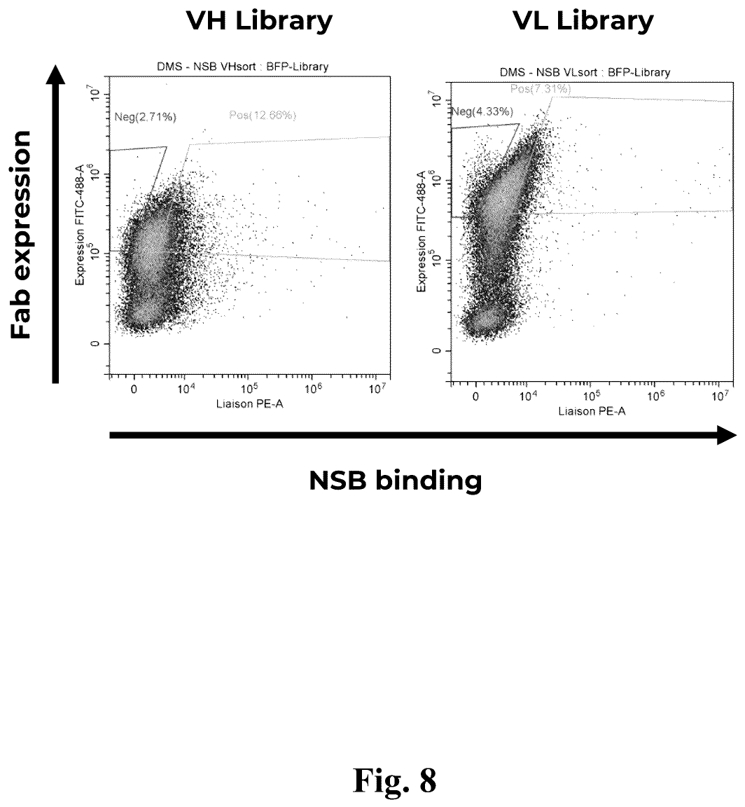

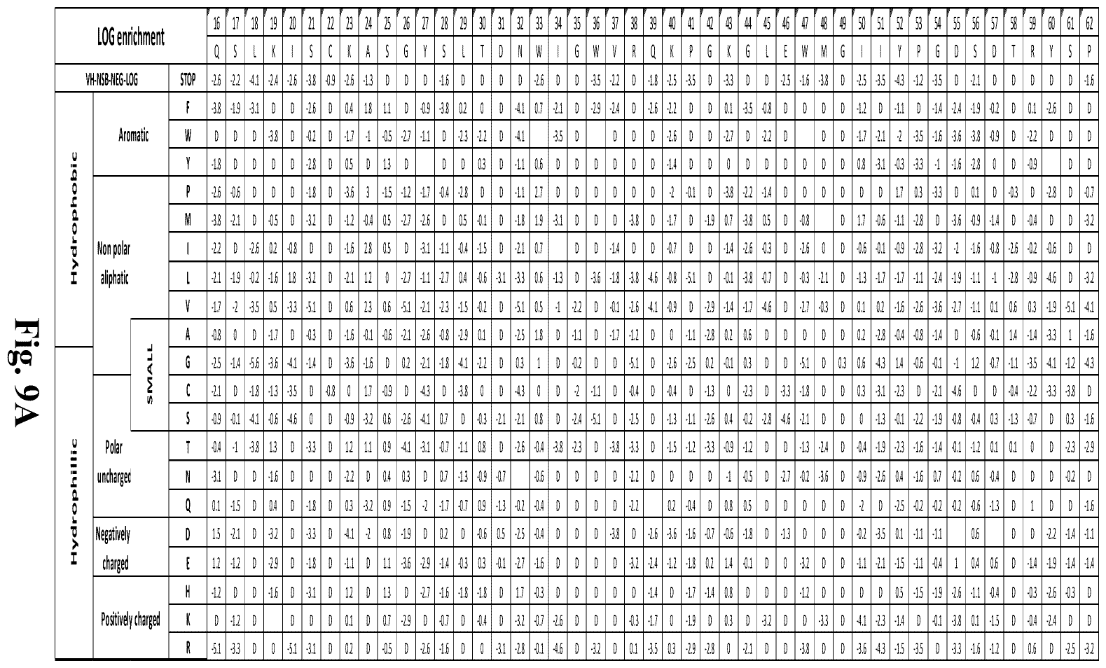

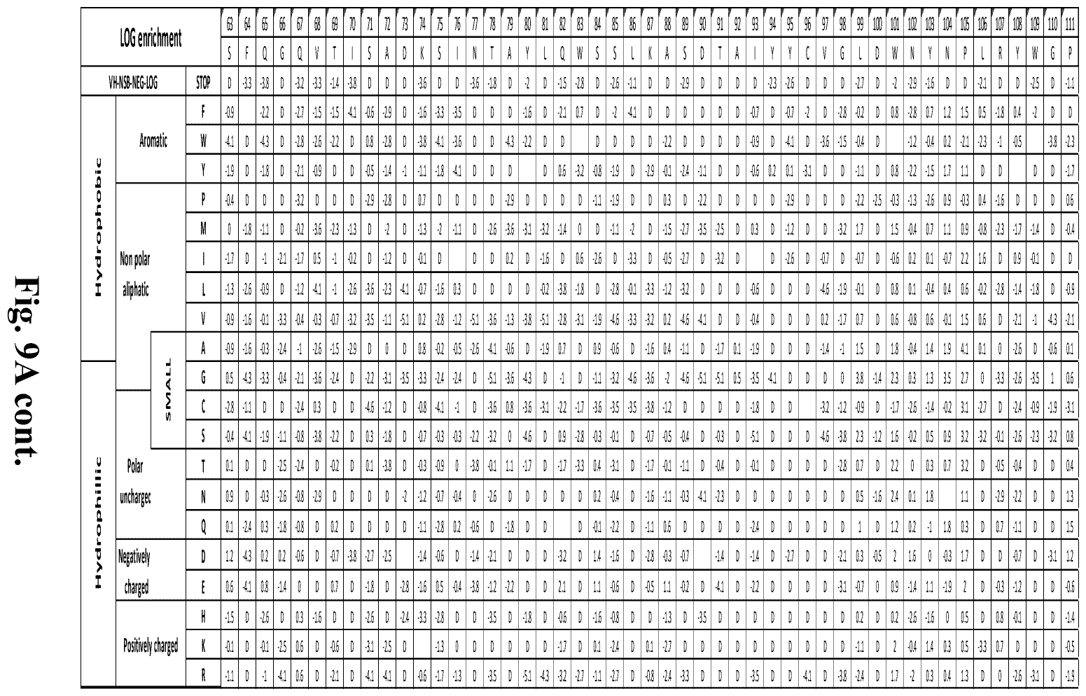

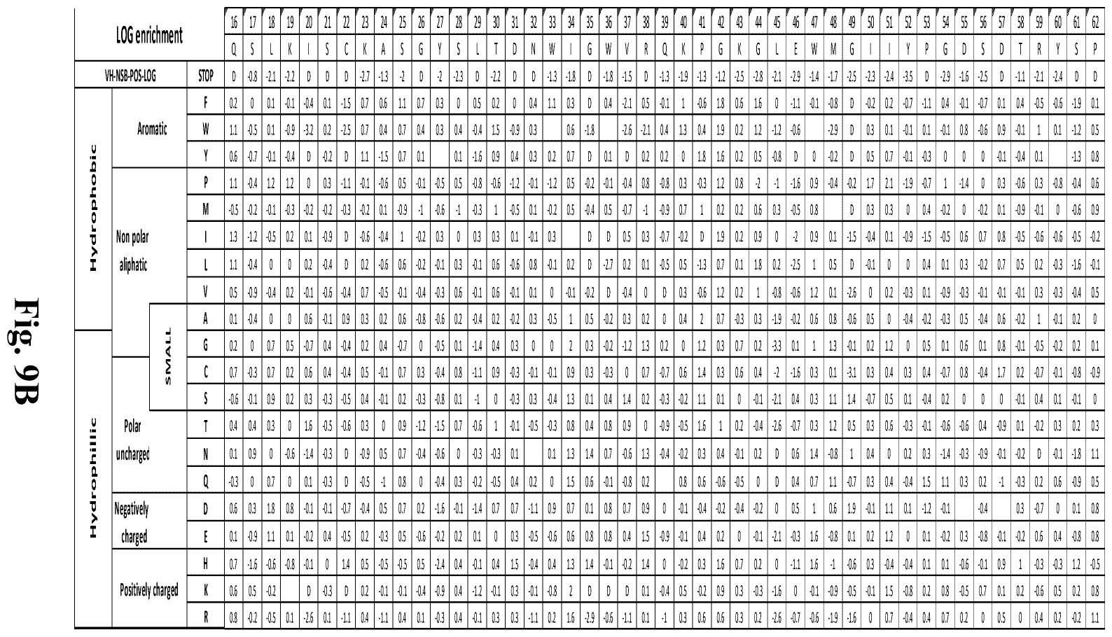

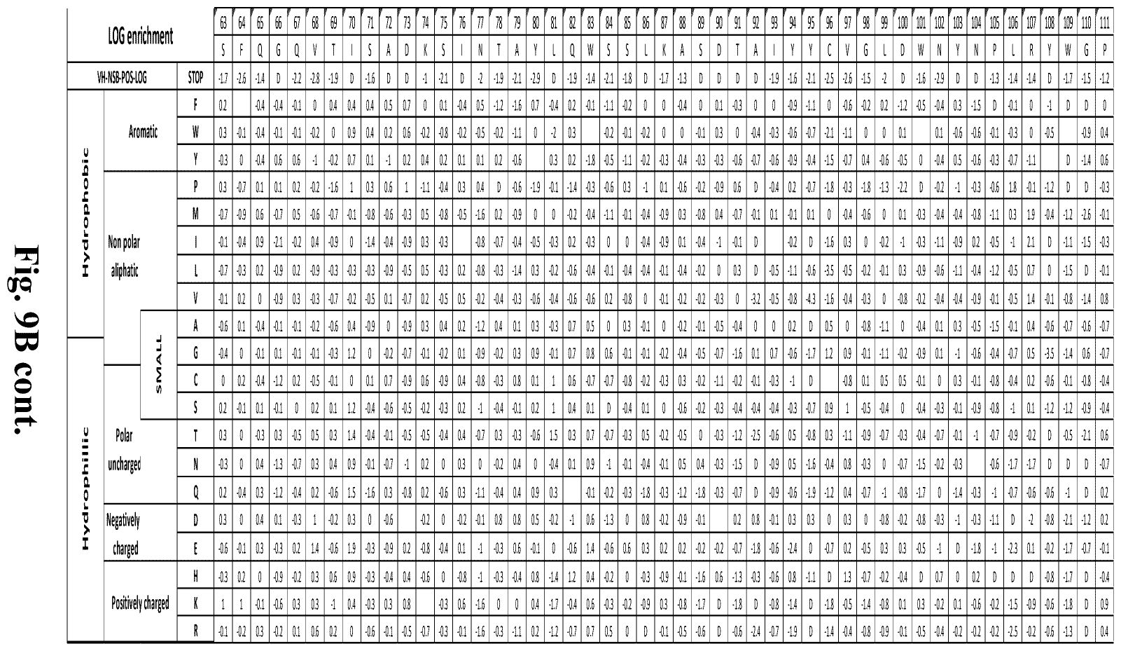

The VH paratope library and VL paratope library were screened for affinity to a non-specific binding (NSB) substrate relative to the parental antibody. The NSB substrate corresponds to a human cell lysate and is thus useful for determining an overall stickiness of the variant. A depiction of the FACS gating is shown in FIG. 8. A positive and negative binding gate was employed. FIG. 9A-9D depict enrichment heatmaps from FACS analysis of TSHR variant antibody fab domains based on expression and NSB binding. FIG. 9A depicts log enrichment of specific mutations in the VH paratope under negative gating and FIG. 9C depicts log enrichment of specific mutations in the VL paratope under negative gating. An enrichment score that is less than 0 indicates that the mutation may increase non-specific binding to the NSB substrate. An enrichment score above 0 indicates that the mutation may decrease non-specific binding to the NSB substrate. Values at 1.0 or higher more strongly associate with decreased non-specific binding.

FIG. 9B depicts log enrichment of specific mutations in the VH paratope under positive gating and FIG. 9D depicts log enrichment of specific mutations in the VL paratope under positive gating. An enrichment score that is less than 0 indicates that the mutation may decrease non-specific binding to the NSB substrate. An enrichment score above 0 indicates that the mutation may increase non-specific binding to the NSB substrate. Values at −0.8 or lower (i.e., −1.0, −1.5, −2.0, etc.) more strongly associate with decreased non-specific binding.

pH-Dependent Affinity to TSHR

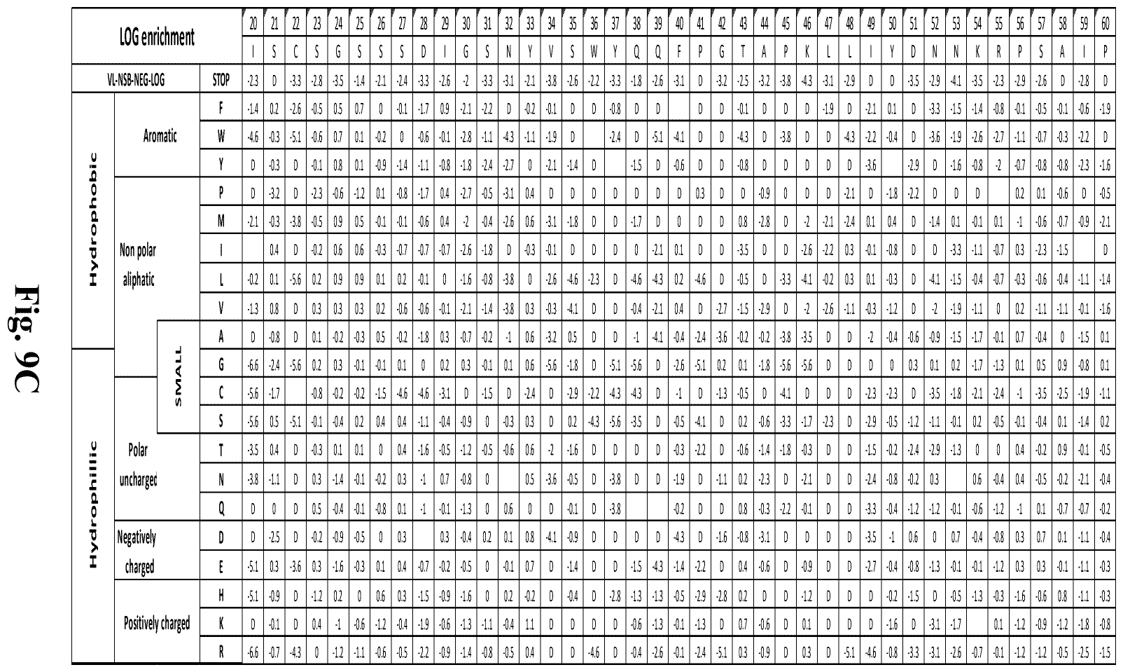

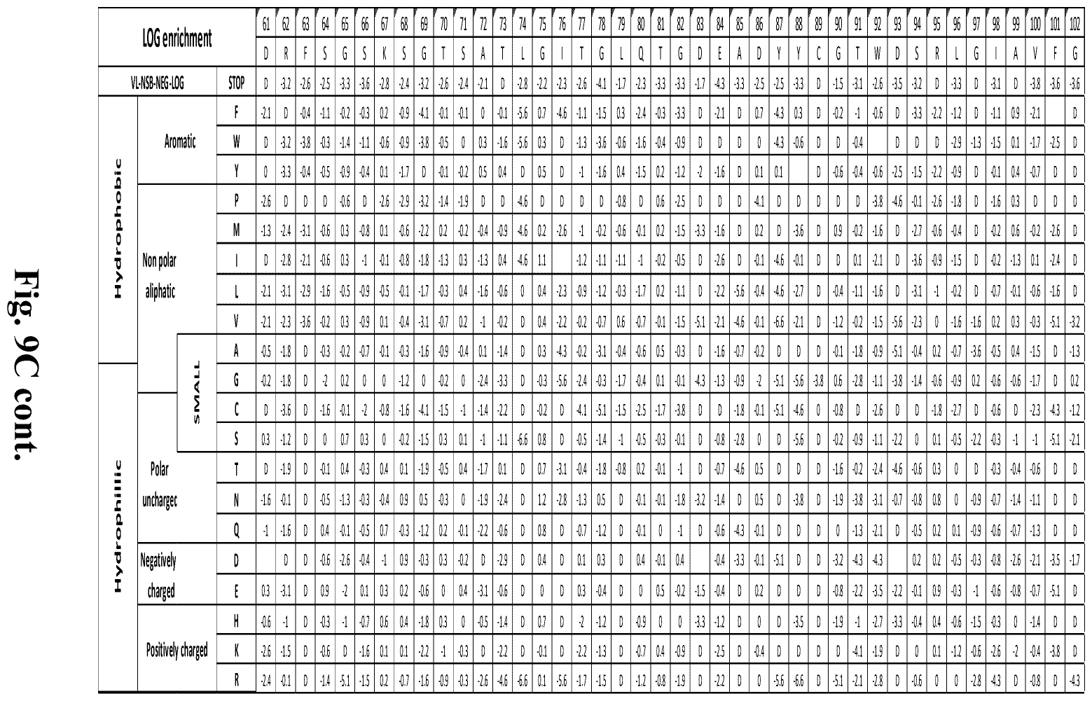

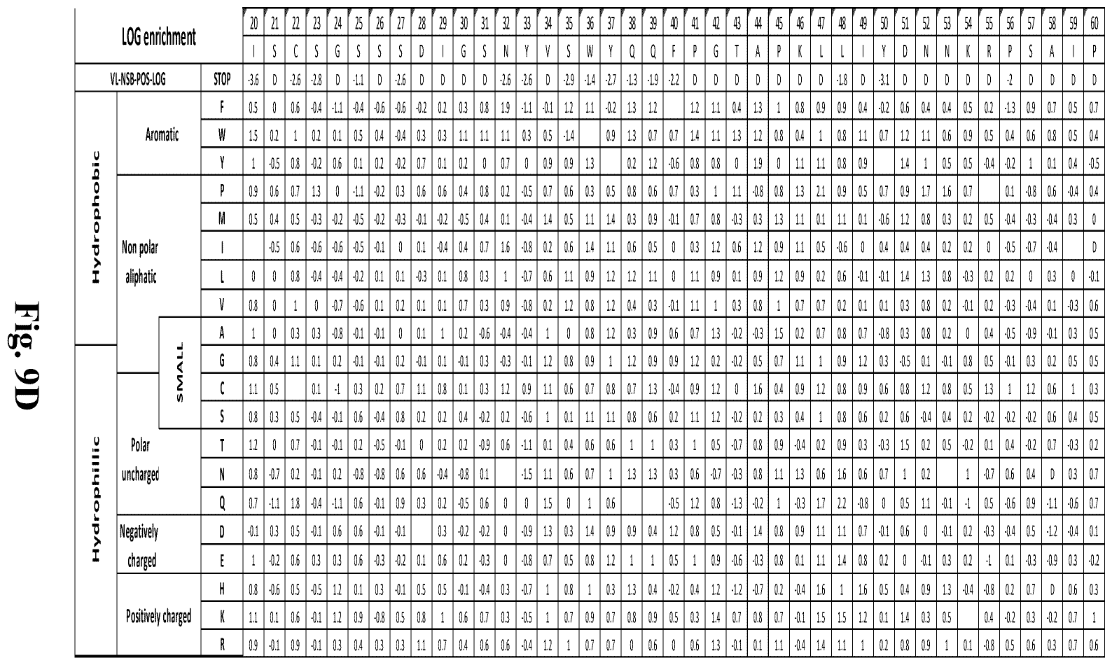

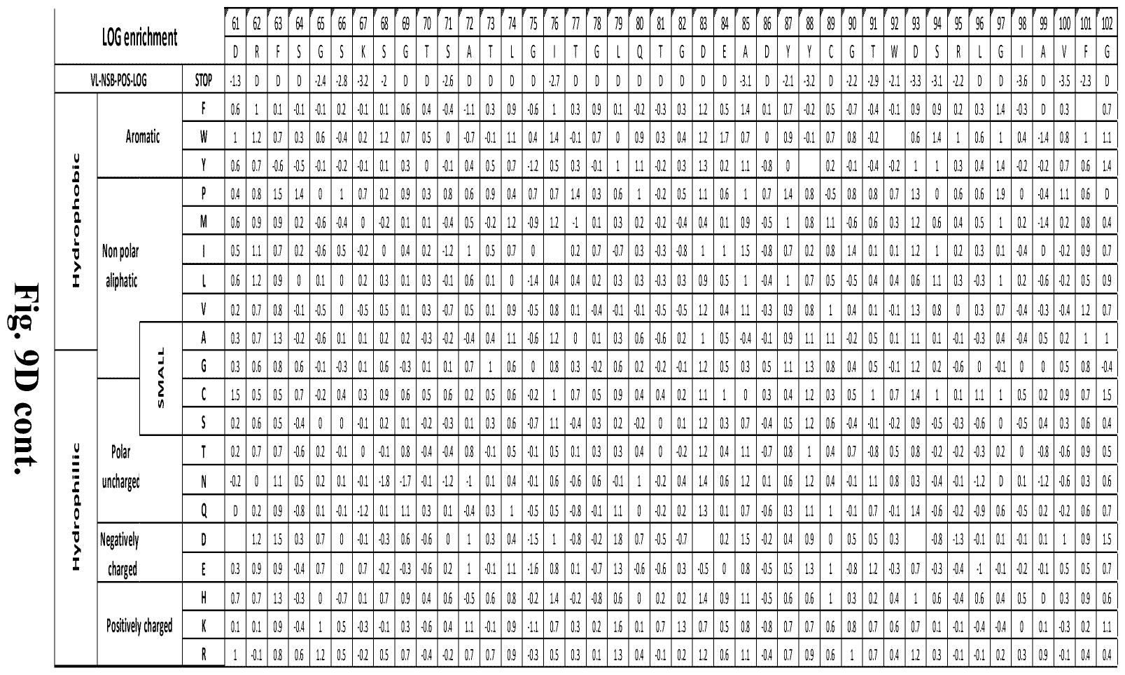

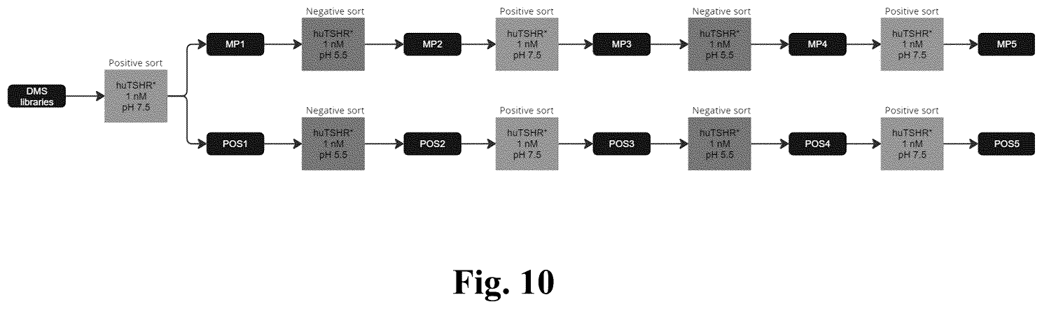

The VH paratope library and VL paratope library were screened for affinity changes to human TSHR relative to the parental antibody at pH 7.5 compared to pH 5.5. The yeast cells expressing either the VH or VL library were incubated with 1 nM human TSHR at pH 7.5 or pH 5.5. FIG. 10 depicts a scheme for selecting TSHR variant antibodies with differential biding at pH 7.5 and 5.5. MP refers to a “minus pos” population meaning a population of yeast cells expressing a Fab with slightly altered affinity compared to parental. Pos refers to a parental like yeast population expressing Fabs with comparable affinity compared to parental. The goal was to identify TSHR antibody variants that bind at pH 7.5 but dissociate from TSHR at pH 5.5.

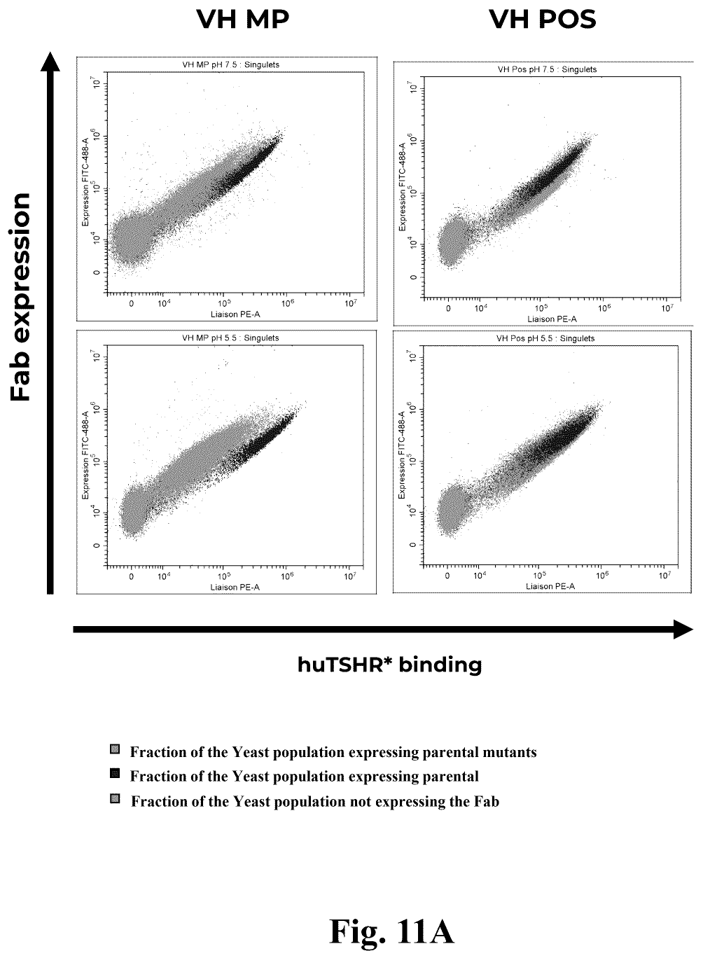

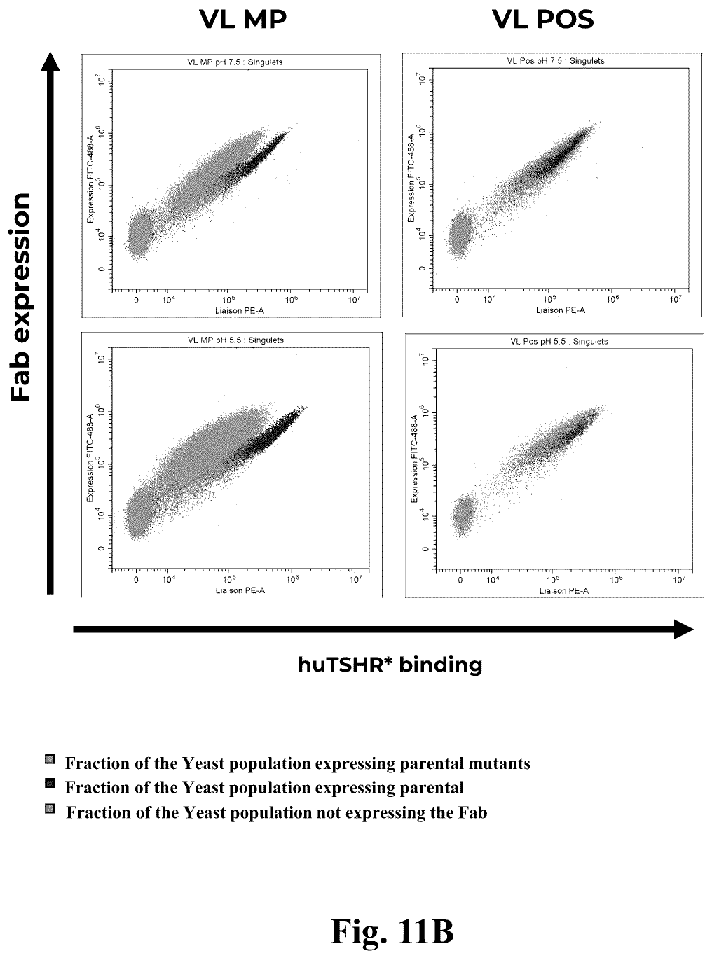

FIG. 11A-FIG. 11B depicts FACS analysis of TSHR variant antibody fab domains based on expression and TSHR binding. A MP and positive binding gate were employed. The top two graphs correspond to incubation at pH 7.5 with TSHR at 1 nM. The bottom two graphs correspond to incubation at pH 5.5 with TSHR at 1 nM. FIG. 11A corresponds to the VH library and FIG. 11B corresponds to the VL library.

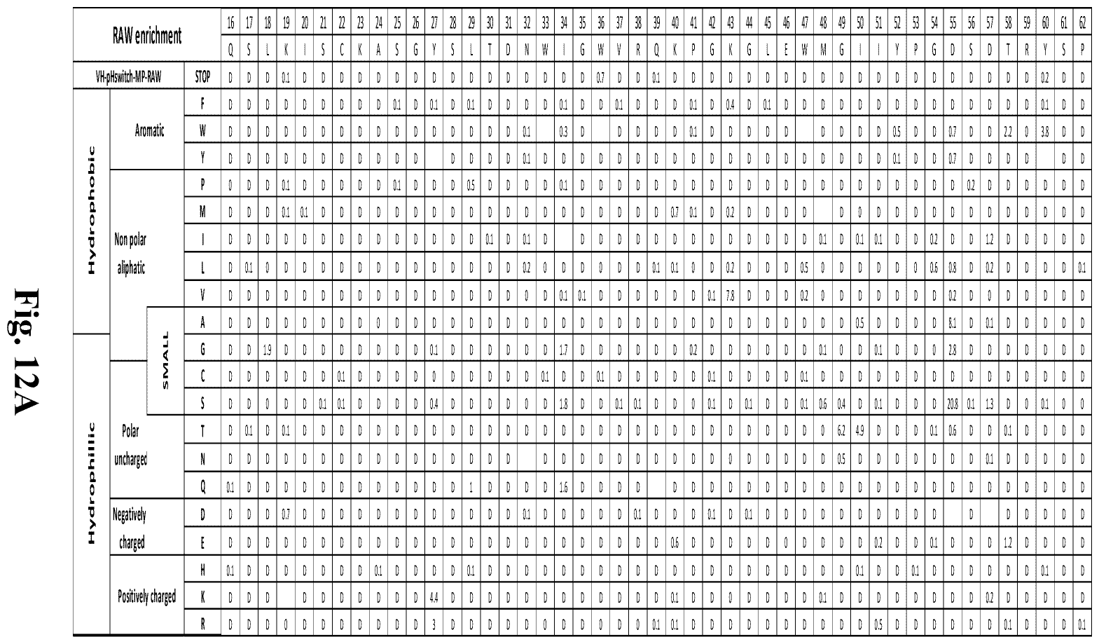

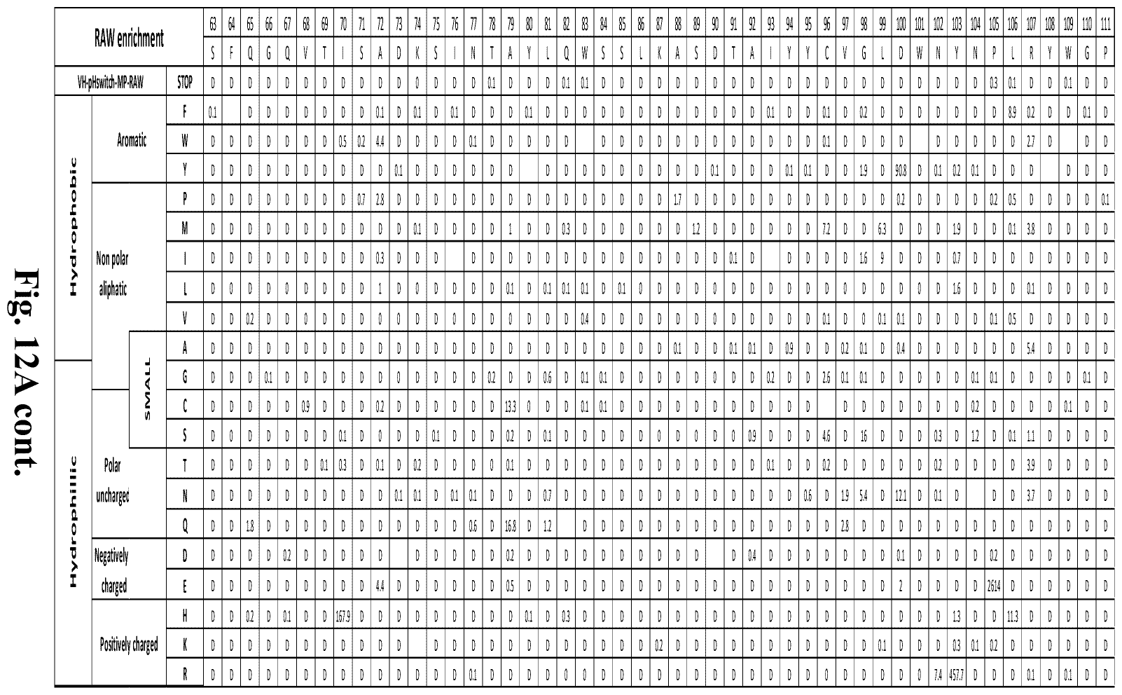

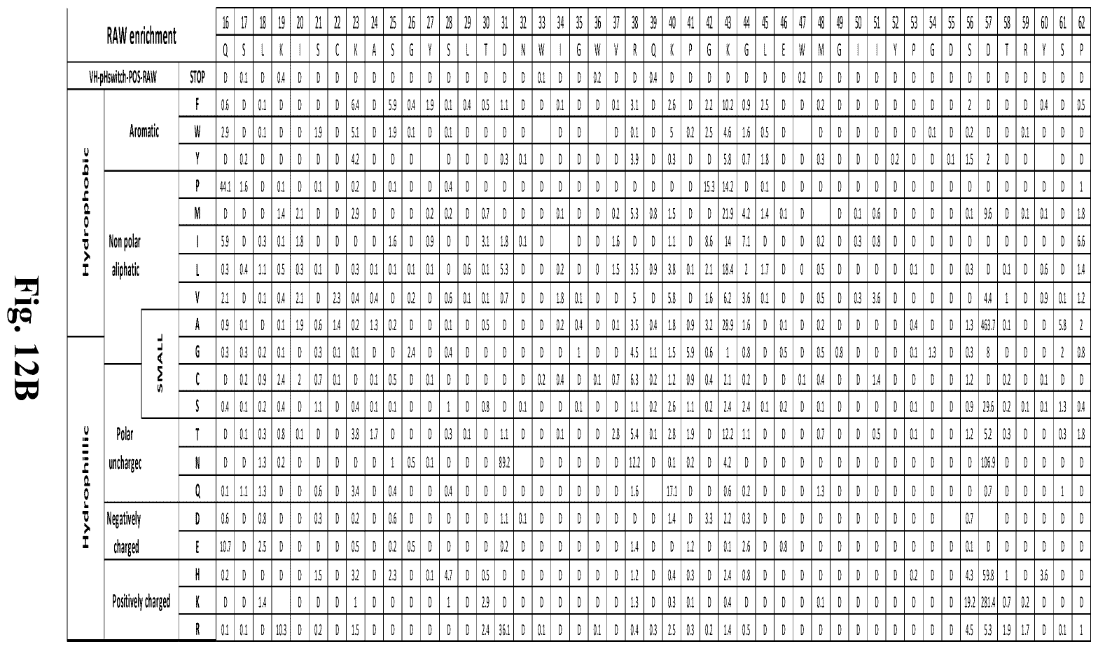

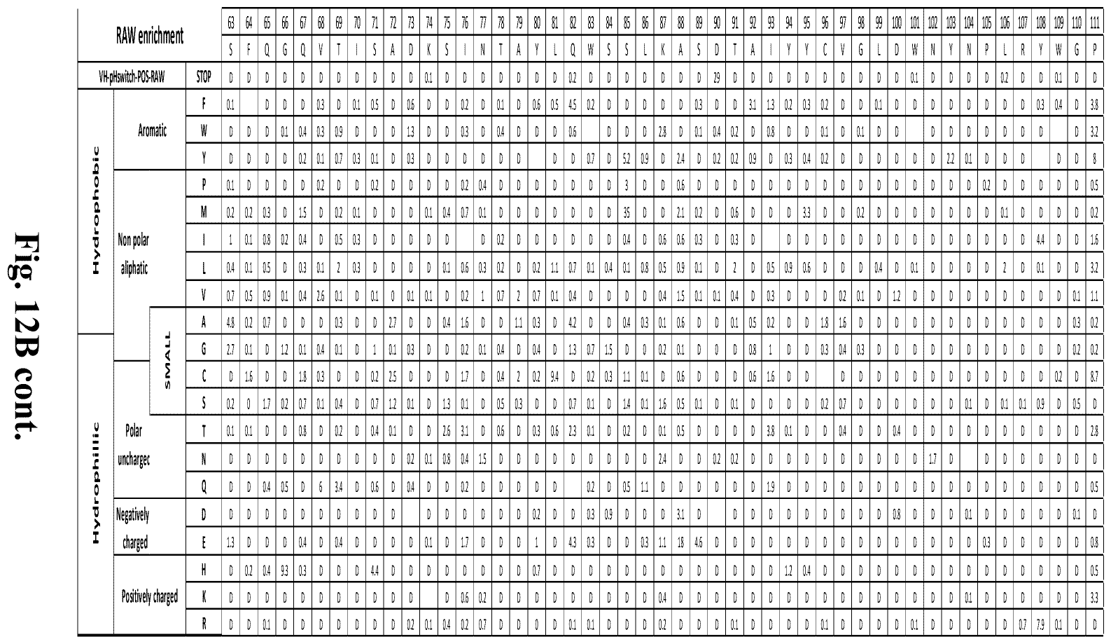

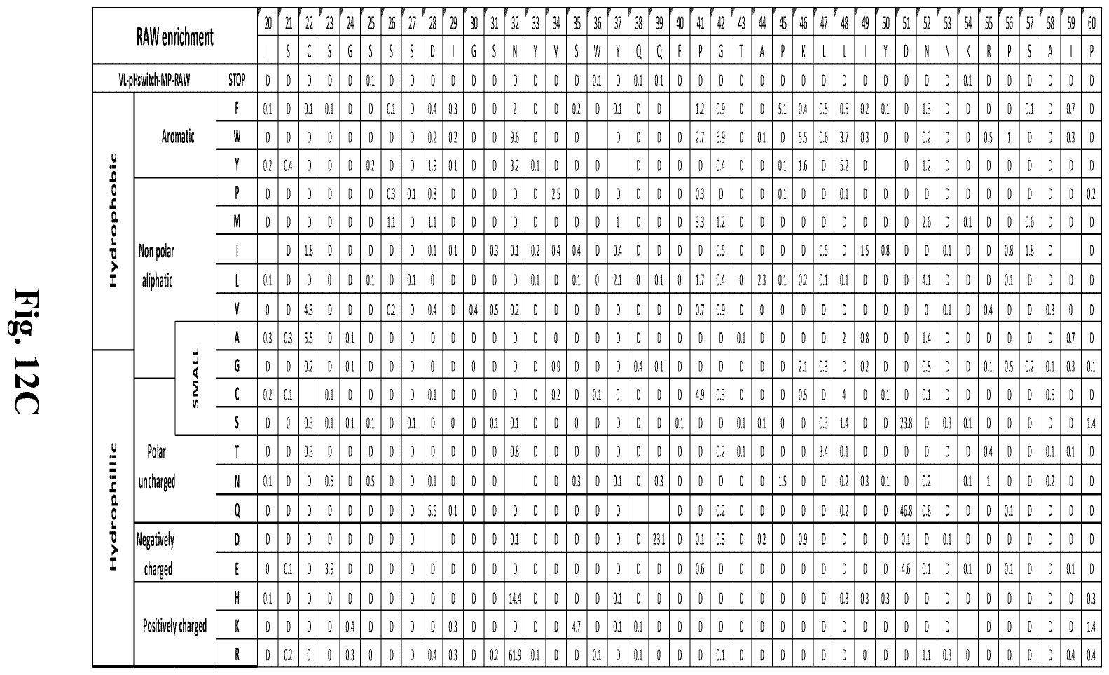

FIG. 12A-12D depict enrichment heatmaps from FACS analysis of TSHR variant antibody fab domains based on expression and TSHR binding. FIG. 12A and FIG. 12C depict raw enrichment of specific mutations in the VH (FIG. 12A) and VL (FIG. 12C) paratope under the pH switch condition under the MP gating. FIG. 12B and FIG. 12D depict raw enrichment of specific mutations in the VH (FIG. 12B) and VL (FIG. 12D) paratope under the pH switch condition under the positive gating. Higher enrichment values indicate that the mutation may improve pH-dependent binding (i.e., retained affinity at pH 7.5 and dissociation at pH 5.5). Values at 10.0 or higher more strongly associate with improved pH-dependent binding. For the MP gate, mutations with values of 40 or greater were particularly useful for pH-dependent binding. For the positive gate, mutations with values of 20 or greater were particularly useful for pH-dependent binding.

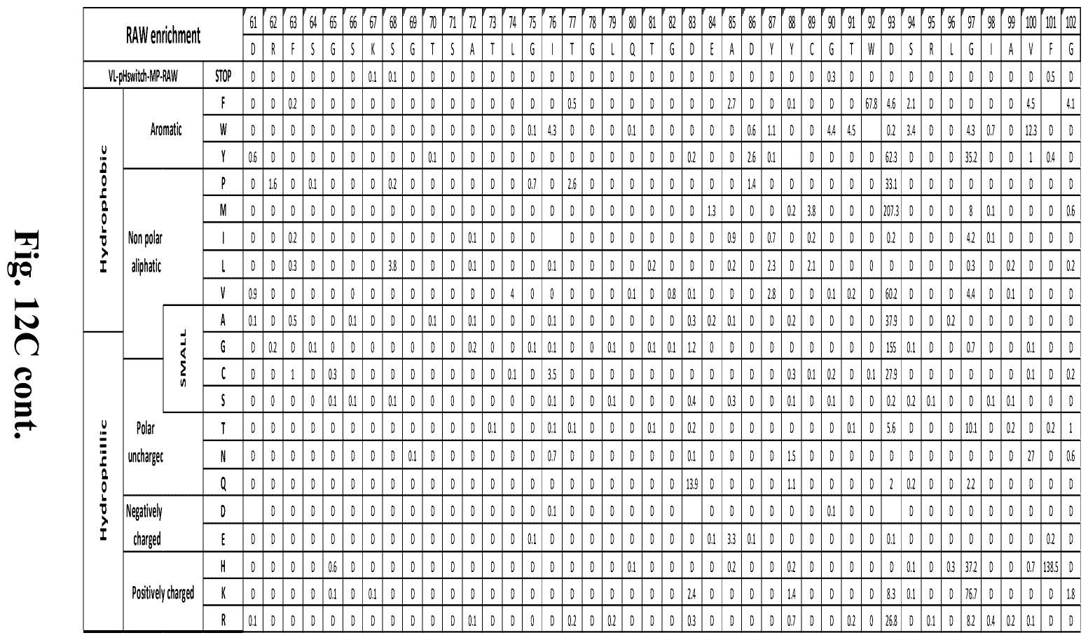

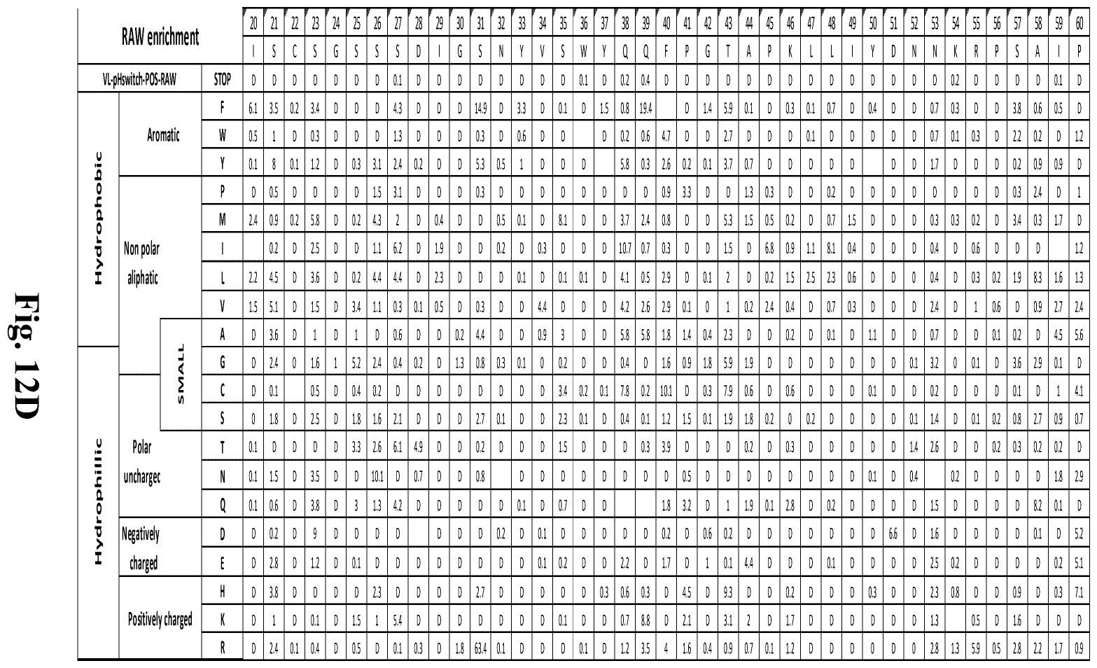

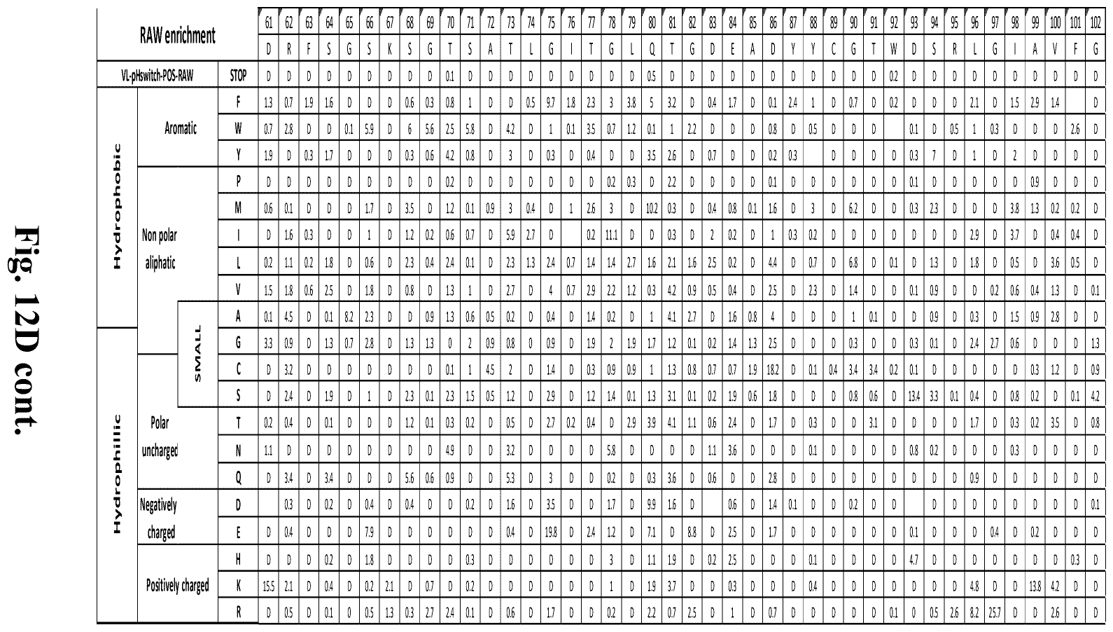

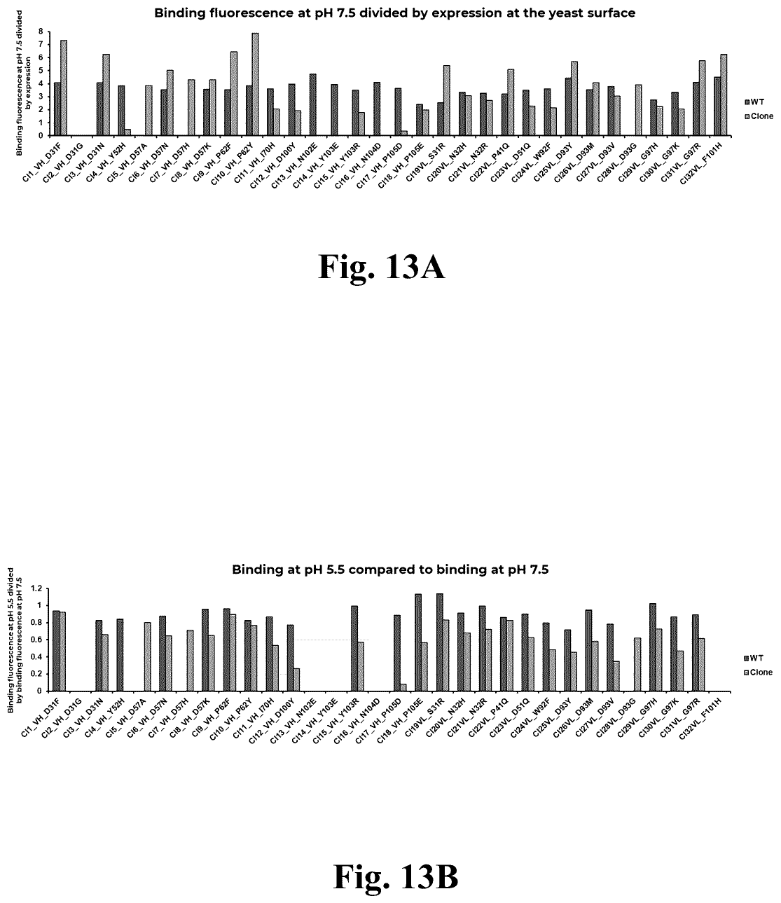

Based on the results of the FACS screen of single mutant variants, select variants were further tested. Yeast cells expressing the parental VH and VL or the 32 selected variants were induced for antibody expression. 5×105 yeast cells were washed twice with PBSF buffer (PBS, BSA 0.1%). Yeast cells were incubated for 2 hours with 5 nM of human TSHR at pH 7.5 or pH 5.5. Yeast cells were washed twice with PBSF buffer. Yeast cells were incubated with fluorescent reporters for 15 minutes on ice with anti-V5 488 (for Fab expression) and streptavidin PE (for TSHR binding detection). Cells were washed once and resuspended in PBSF buffer. Yeast cells were then analyzed on a Beckman CytoFlex S cytometer. The results of the focused analysis are shown in FIG. 13A-13B, which depicts binding fluorescence at pH 7.5 divided by expression at the yeast surface (FIG. 13A) and binding at pH 5.5 compared to binding at pH 7.5 (FIG. 13B). For each data point, the left bar corresponds to the parental antibody and the right bar corresponds to the variant.

Based on these results, the following VH substitutions were found to be useful for conferring pH-dependent biding to TSHR: Y52H, D57H or D57K, I70H, D100Y, Y103R, P105D or P105E.

Accordingly, the following anti-TSHR variant antibody VH domain, when paired with any one of the VL domains described herein, is a pH-dependent antibody that retains similar affinity as the parental TSHR antibody at pH 7.5 while dissociating at an acidic pH (e.g., pH 5.5).

pH-dependent anti-TSHR variant
antibody VH domain:
(SEQ ID NO: 144)
EVQLVQSGAEVKKPGQSLKISCKASGYSLTDNWIGWVRQKPGKGLE
WMGIIX₁PGDSX₂TRYSPSFQGQVTX₃SADKSINTAYLQWSSLKAS
DTAIYYCVGLX₄WNX₅NX₆LRYWGPGTLVTVSS

wherein:
X₁ corresponds to the amino acid H or Y, X₂ corresponds to the amino acid H, K, or Y, X₃ corresponds to the amino acid H or I, X₄ corresponds to the amino acid Y or D, X₅ corresponds to the amino acid R or Y, and X₆ corresponds to the amino acid D, E, or P; and
at least one of the following is present: X₁ is the amino acid H, X₂ is the amino acid H or K, X₃ is the amino acid H, X₄ is the amino acid Y, X₅ is the amino acid R, and X₆ is the amino acid D or E.

### Example 9. Paratope Mapping of a Variant Anti-TSHR Antibody T3

Paratope mapping of a variant of the parental anti-TSHR antibody having a VH domain of SEQ ID NO: 1 and a VL domain of SEQ ID NO: 2 was performed. The variant, designated T3, was a germlined version of the parental anti-TSHR antibody and had a VH domain of SEQ ID NO: 128 and a VL domain of SEQ ID NO: 129. Paratope mapping was performed as described in Example 8 for the parental VH and VL, specifically to identify amino acid substitutions that affected 1) affinity to TSHR, 2) thermal stability, 3) polyreactivity, and 4) pH-dependent affinity to TSHR. As with the parental VH and VL, DMS was employed. Similar gating strategies with yeast cells were employed, as described in Example 8.

From the DMS results, the following VH substitutions were found to be useful for conferring pH-dependent binding to TSHR: S28H, T30H, S31H, Y32H, D57K, and N102H relative to SEQ ID NO: 128. The following VL substitutions were found to be useful for conferring pH-dependent binding to TSHR: N31R, N32H, and S94H relative to SEQ ID NO: 129.

Following screening of the single amino acid variants of T3, the identified substitutions were tested as double, triple, and quadruple mutations. The following mutation combinations were tested:

| Mutant Type | ID | Substitutions |
|---|---|---|
| | T3-WT | T3_WT |
| Mono-Mutant | T3-1.1 | T3_VH_S28H |
| | T3-1.2 | T3_VH_T30H |
| | T3-1.3 | T3_VH_S31H |
| | T3-1.4 | T3_VH_Y32H |
| | T3-1.10 | T3_VH_N102H |
| | T3-1.16 | T3_VL_N31R |
| Double - mutant | T3-1.30 | T3_VH_S28H_VL_N31R |
| | T3-1.31 | T3_VH_T30H_VL_N31R |
| | T3-1.32 | T3_VH_S31H_VL_N31R |
| | T3-1.33 | T3_VH_Y32H_VL_N31R |
| | T3-1.34 | T3_VH_N102H_VL_N31R |
| | T3-1.35 | T3_VH_S28H_T30H |
| | T3-1.36 | T3_VH_S28H_S31H |
| | T3-1.37 | T3_VH_S28H_Y32H |
| | T3-1.38 | T3_VH_S28H_N102H |
| | T3-1.39 | T3_VH_T30H_S31H |
| | T3-1.40 | T3_VH_T30H_Y32H |
| | T3-1.41 | T3_VH_T30H_N102H |
| | T3-1.42 | T3_VH_S31H_Y32H |
| | T3-1.43 | T3_VH_S31H_N102H |
| | T3-1.44 | T3_VH_Y32H_N102H |
| Triple - mutant | T3-1.45 | T3_VH_S28H_T30H_VL_N31R |
| | T3-1.46 | T3_VH_S28H_S31H_VL_N31R |
| | T3-1.47 | T3_VH_S28H_Y32H_VL_N31R |
| | T3-1.48 | T3_VH_S28H_N102H_VL_N31R |
| | T3-1.49 | T3_VH_T30H_S31H_VL_N31R |
| | T3-1.50 | T3_VH_T30H_Y32H_VL_N31R |
| | T3-1.51 | T3_VH_T30H_N102H_VL_N31R |
| | T3-1.52 | T3_VH_S31H_Y32H_VL_N31R |
| | T3-1.53 | T3_VH_S31H_N102H_VL_N31R |
| | T3-1.54 | T3_VH_Y32H_N102H_VL_N31R |
| | T3-1.55 | T3_VH_S28H_T30H_S31H |
| | T3-1.56 | T3_VH_S28H_T30H_Y32H |
| | T3-1.57 | T3_VH_S28H_T30H_N102H |
| | T3-1.58 | T3_VH_S28H_S31H_Y32H |
| | T3-1.59 | T3_VH_S28H_S31H_N102H |
| | T3-1.60 | T3_VH_S28H_Y32H_N102H |
| | T3-1.61 | T3_VH_T30H_S31H_Y32H |
| | T3-1.62 | T3_VH_T30H_S31H_N102H |
| | T3-1.63 | T3_VH_T30H_Y32H_N102H |
| | T3-1.64 | T3_VH_S31H_Y32H_N102H |
| Quadruple mutant | T3-1.65 | T3_VH_S28H_T30H_S31H_Y32H |

To screen the various T3 variants, yeast cells expressing the WT T3 and the 42 T3 mutants were induced for antibody expression. 5×10⁵ yeast cells were washed twice with PBSF buffer (PBS, BSA 0.1%). Yeast cells were incubated 2 h with 10 nM of huTSHR at pH 7.5 or pH 5.5. Yeast cells were washed twice with PBSF buffer. Yeast cells were incubated with fluorescent reporters for 15 min on ice, anti-V5 488 (Fab expression) and streptavidin PE (huTSHR detection). Cells were washed once and resuspended in PBSF buffer. Yeast cells were then analyzed on a Beckman CytoFlex S cytometer.

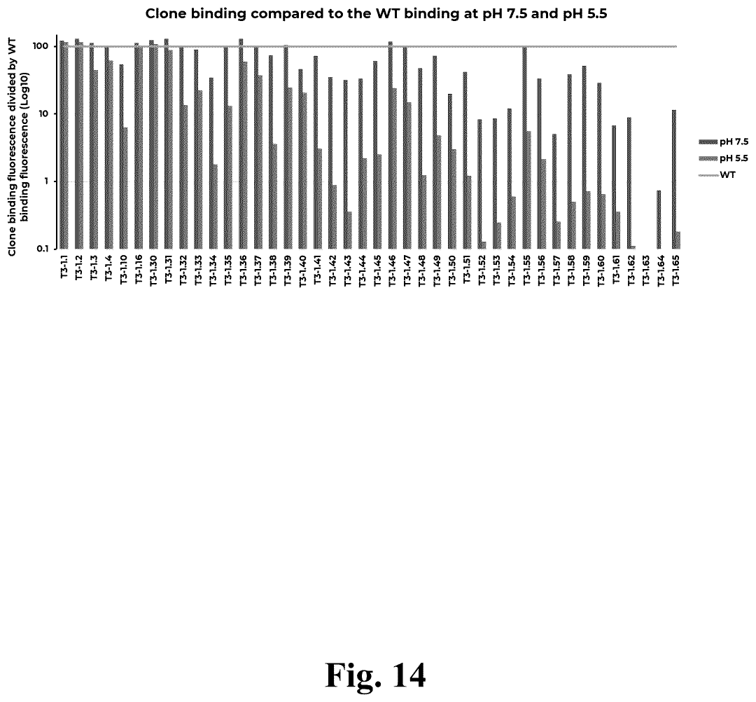

The results are shown in FIG. 14. Relative binding at pH 7.5 and pH 5.5 compared to WT was determined. From these results, the following T3 variants were selected for being pH sensitive:

| ID | Substituions |
|---|---|
| T3-1.32 | T3_VH_S31H_VL_N31R |
| T3-1.35 | T3_VH_S28H_T30H |
| T3-1.38 | T3_VH_S28H_N102H |
| T3-1.41 | T3_VH_T30H_N102H |
| T3-1.47 | T3_VH_S28H_Y32H_VL_N31R |
| T3-1.49 | T3_VH_T30H_S31H_VL_N31R |
| T3-1.55 | T3_VH_S28H_T30H_S31H |
| T3-1.66 | T3_VH_S28H_T30H_S31H_VL_N31R |

Based on the results of the pH sensitive screening in Example 8 with the parental antibody and Example 9 with the T3 variant, any one or more of the following pH sensitive amino acids substitutions may be utilized to enhance pH sensitive binding:
VH: S28H; T30H; D31H (in parental VH of SEQ ID NO: 1, or the variant VH of SEQ ID NO: 3 or 78), or S31H (in T3 VH of SEQ ID NO: 128), N32H (in parental VH of SEQ ID NO: 1, or the variant VH of SEQ ID NO: 3 or 78), or Y32H (in T3 VH of SEQ ID NO: 128); N102H
VL: S31R (in parental VL of SEQ ID NO: 2, or the variant VL of SEQ ID NO: 4 or 25), or N31R (in T3 VL of SEQ ID NO: 129); N32H; S94H

SEQUENCES

| | SEQ | |
|---|---|---|
| Antibody | ID NO | Sequence |
| Parental VH | 1 | EVQLVQSGAEVKKPGQSLKISCKASGYSLTDNWIGWVRQKPGKGLEWM |
| | | GIIYPGDSDTRYSPSFQGQVTISADKSINTAYLQWSSLKASDTAIYYC |
| | | VGLDWNYNPLRYWGPGTLVTVSS |
| Parental VL | 2 | QSVLTQPPSVSAAPGQKVTISCSGSSSDIGSNYVSWYQQFPGTAPKLL |
| | | IYDNNKRPSAIPDRESGSKSGTSATLGITGLQTGDEADYYCGTWDSRL |
| | | GIAVFGGGTQLTVL |
| 1-46_VH | 3 | EVQLVQSGAEVKKPGASVKVSCKASGYSLTDNWIGWVRQAPGQGLEWM |
| | | GIIYPGDSDTRYAQKFQGRVTMTADKSTSTVYMELSSLRSEDTAVYYC |
| | | VGLDWNYNPLRYWGQGTLVTVSS |
| 1-40_VL | 4 | QSVLTQPPSVSGAPGQRVTISCSGSSSDIGSNYVSWYQQLPGTAPKLL |
| | | IYDNNKRPSGVPDRESGSKSGTSASLAITGLQAEDEADYYCGTWDSRL |
| | | GIAVFGGGTKLTVL |
| IgG1 Heavy | 5 | ASTKGPSVFPLAPSSKSTSGGTAALGCLVKDYFPEPVTVSWNSGALTS |
| Chain constant | | GVHTFPAVLQSSGLYSLSSVVTVPSSSLGTQTYICNVNHKPSNTKVDK |
| region (YTE) | | KVEPKSCDKTHTCPPCPAPELLGGPSVFLFPPKPKDTLYITREPEVTC |
| | | VVVDVSHEDPEVKENWYVDGVEVHNAKTKPREEQYNSTYRVVSVLTVL |
| | | HQDWLNGKEYKCKVSNKALPAPIEKTISKAKGQPREPQVYTLPPSRDE |
| | | LTKNQVSLTCLVKGFYPSDIAVEWESNGQPENNYKTTPPVLDSDGSFF |
| | | LYSKLTVDKSRWQQGNVFSCSVMHEALHNHYTQKSLSLSPGK |
| Lambda Light | 6 | GQPKAAPSVTLFPPSSEELQANKATLVCLVSDFYPGAVTVAWKADGSP |
| Chain constant | | VKVGVETTKPSKQSNNKYAASSYLSLTPEQWKSHRSYSCRVTHEGSTV |
| region | | EKTVAPTECS |
| GL_5- | 7 | EVQLVQSGAEVKKPGESLKISCKASGYELNDNWIGWVRQKPGKGLEWM |
| 51_VH1 | | GIIYPGDSDTRYSPSFKGQVTISADKSISTAYLQWSSLKASDTAIYYC |
| | | VGLEWNYNPLRYWGQGTLVTVSS |
| GL_5-51_ | 8 | EVQLVQSGAEVKKPGESLKISCKASGYQLNDNWIGWVRQKPGKGLEWM |
| VH2 | | GIIYPGDSDTRYSPSFKGQVTISADKSISTAYLQWSSLKASDTAIYYC |
| | | VGLEWNYNPLRYWGQGTLVTVSS |
| GL_5-51_ | 9 | EVQLVQSGAEVKKPGESLKISCKASGYELKDNWIGWVRQKPGKGLEWM |
| VH3 | | GIIYPGDSDTRYSPSFKGQVTISADKSISTAYLQWSSLKASDTAIYYC |
| | | VGLEWNYNPLRYWGQGTLVTVSS |
| GL_5-51_ | 10 | EVQLVQSGAEVKKPGESLKISCKASGYQLKDNWAGWVRQKPGKGLEWM |
| VH4 | | GIIYPGDSDTRYSPSFKGQVTISADKSISTAYLQWSSLKASDTAIYYC |
| | | VGLEWNYNPLRYWGQGTLVTVSS |
| GL_5-51_ | 11 | EVQLVQSGAEVKKPGESLKISCKASGYQLKDNWIGWVRQKPGKGLEWM |
| VH5 | | GIIYPGDSDTRYSPSFQGQVTISADKSISTAYLQWSSLKASDTAIYYC |
| | | VGLEWNYNPLRYWGQGTLVTVSS |
| GL_5-51_ | 12 | EVQLVQSGAEVKKPGESLKISCKASGYQLKDNWIGWVRQKPGKGLEWM |
| VH6 | | GIIYPGDSDTRYSPSFKGQVTISADKSISTAYLQWSSLKASDTAIYYC |
| | | VGLEWNYNPGRYWGQGTLVTVSS |
| GL_5-51_ | 13 | EVQLVQSGAEVKKPGESLKISCKASGYQLKDNWAGWVRQKPGKGLEWM |
| VH7 | | GIIYPGDSDTRYSPSFKGQVTISADKSISTAYLQWSSLKASDTAIYYC |
| | | VGLEWNYNPGRYWGQGTLVTVSS |
| GL_5-51_VH8 | 14 | EVQLVQSGAEVKKPGESLKISCKASGYQLKDNWIGWVRQKPGKGLEWM |
| | | GIIYPGDSDTRYSPSFQGQVTISADKSISTAYLQWSSLKASDTAIYYC |
| | | VGLEWNYNPVRYWGQGTLVTVSS |
| GL_5-51_VH9 | 15 | EVQLVQSGAEVKKPGESLKISCKASGYQLKDNWIGWVRQKPGKGLEWM |
| | | GIIYPGDSDTRYSPSFQGQVTISADKSISTAYLQWSSLKASDTAIYYC |
| | | VGGEWNYNPLRYWGQGTLVTVSS |
| 1-46_VH1 | 16 | EVQLVQSGAEVKKPGASVKVSCKASGYELNDNWIGWVRQAPGQGLEWM |
| | | GIIYPGDSDTRYAQKFQGRVTMTADKSTSTVYMELSSLRSEDTAVYYC |
| | | VGLEWNYNPLRYWGQGTLVTVSS |
| 1-46_VH2 | 17 | EVQLVQSGAEVKKPGASVKVSCKASGYQLNDNWIGWVRQAPGQGLEWM |
| | | GIIYPGDSDTRYAQKFQGRVTMTADKSTSTVYMELSSLRSEDTAVYYC |
| | | VGLEWNYNPLRYWGQGTLVTVSS |
| 1-46_VH3 | 18 | EVQLVQSGAEVKKPGASVKVSCKASGYELKDNWIGWVRQAPGQGLEWM |
| | | GIIYPGDSDTRYAQKFQGRVTMTADKSTSTVYMELSSLRSEDTAVYYC |
| | | VGLEWNYNPLRYWGQGTLVTVSS |
| 1-46_VH4 | 19 | EVQLVQSGAEVKKPGASVKVSCKASGYQLKDNWAGWVRQAPGQGLEWM |
| | | GIIYPGDSDTRYAQKFQGRVTMTADKSTSTVYMELSSLRSEDTAVYYC |
| | | VGLEWNYNPLRYWGQGTLVTVSS |
| 1-46_VH5 | 20 | EVQLVQSGAEVKKPGASVKVSCKASGYQLKDNWIGWVRQAPGQGLEWM |
| | | GIIYPGDSDTRYAQKFQGRVTMTADKSTSTVYMELSSLRSEDTAVYYC |
| | | VGLEWNYNPLRYWGQGTLVTVSS |
| 1-46_VH6 | 21 | EVQLVQSGAEVKKPGASVKVSCKASGYQLKDNWIGWVRQAPGQGLEWM |
| | | GIIYPGDSDTRYAQKFQGRVTMTADKSTSTVYMELSSLRSEDTAVYYC |
| | | VGLEWNYNPGRYWGQGTLVTVSS |
| 1-46_VH7 | 22 | EVQLVQSGAEVKKPGASVKVSCKASGYQLKDNWAGWVRQAPGQGLEWM |
| | | GIIYPGDSDTRYAQKFQGRVTMTADKSTSTVYMELSSLRSEDTAVYYC |
| | | VGLEWNYNPGRYWGQGTLVTVSS |
| 1-46_VH8 | 23 | EVQLVQSGAEVKKPGASVKVSCKASGYQLKDNWIGWVRQAPGQGLEWM |
| | | GIIYPGDSDTRYAQKFQGRVTMTADKSTSTVYMELSSLRSEDTAVYYC |
| | | VGLEWNYNPVRYWGQGTLVTVSS |
| 1-46_VH9 | 24 | EVQLVQSGAEVKKPGASVKVSCKASGYQLKDNWIGWVRQAPGQGLEWM |
| | | GIIYPGDSDTRYAQKFQGRVTMTADKSTSTVYMELSSLRSEDTAVYYC |
| | | VGGEWNYNPLRYWGQGTLVTVSS |
| GL_1-51_VL | 25 | QSVLTQPPSVSAAPGQKVTISCSGSSSDIGSNYVSWYQQLPGTAPKLL |
| | | IYDNNKRPSGIPDRESGSKSGTSATLGITGLQTGDEADYYCGTWDSRL |
| | | GIAVFGGGTQLTVL |
| GL_1-51_VL1 | 26 | QSVLTQPPSVSAAPGQKVTISCSGSSTNIGALWVSWYQQLPGTAPKLL |
| | | IYDNNKRPSGIPDRESGSKSGTSATLGITGLQTGDEADYYCATWDSRF |
| | | GIAVFGGGTQLTVL |
| GL_1-51_VL2 | 27 | QSVLTQPPSVSAAPGQKVTISCSGSSTNIGALWVSWYQQLPGTAPKLL |
| | | IYDNNKRPSGIPDRESGSKSGTSATLGITGLQTGDEADYYCATWDSRW |
| | | GIAVFGGGTQLTVL |
| GL_1-51_VL3 | 28 | QSVLTQPPSVSAAPGQKVTISCSGSSTNIGANYVSWYQQLPGTAPKLL |
| | | IYDNNKRPSGIPDRESGSKSGTSATLGITGLQTGDEADYYCATWDSRF |
| | | GIAVFGGGTQLTVL |
| GL_1-51_VL4 | 29 | QSVLTQPPSVSAAPGQKVTISCSGSSTNIGANYVSWYQQLPGTAPKLL |
| | | IYDNNKRPSGIPDRESGSKSGTSATLGITGLQTGDEADYYCATWDSRW |
| | | GIAVFGGGTQLTVL |
| GL_1-51_VL5 | 30 | QSVLTQPPSVSAAPGQKVTISCSGSSTDIGANYVSWYQQLPGTAPKLL |
| | | IYDNNKRPSGIPDRESGSKSGTSATLGITGLQTGDEADYYCATWDSRW |
| | | GIAVFGGGTQLTVL |
| GL_1-51_VL6 | 31 | QSVLTQPPSVSAAPGQKVTISCSGSSTNIGANYVSWYQQLPGTAPKLL |
| | | IYDNQKRPSGIPDRESGSKSGTSATLGITGLQTGDEADYYCATWDSRW |
| | | GIAVFGGGTQLTVL |
| 1-40_VL1 | 32 | QSVLTQPPSVSGAPGQRVTISCSGSSTNIGALWVSWYQQLPGTAPKLL |
| | | IYDNNKRPSGVPDRFSGSKSGTSASLAITGLQAEDEADYYCATWDSRF |
| | | GIAVFGGGTKLTVL |
| 1-40_VL2 | 33 | QSVLTQPPSVSGAPGQRVTISCSGSSTNIGALWVSWYQQLPGTAPKLL |
| | | IYDNNKRPSGVPDRESGSKSGTSASLAITGLQAEDEADYYCATWDSRW |
| | | GIAVFGGGTKLTVL |
| 1-40_VL3 | 34 | QSVLTQPPSVSGAPGQRVTISCSGSSTNIGANYVSWYQQLPGTAPKLL |
| | | IYDNNKRPSGVPDRESGSKSGTSASLAITGLQAEDEADYYCATWDSRF |
| | | GIAVFGGGTKLTVL |
| 1-40_VL4 | 35 | QSVLTQPPSVSGAPGQRVTISCSGSSTNIGANYVSWYQQLPGTAPKLL |
| | | IYDNNKRPSGVPDRFSGSKSGTSASLAITGLQAEDEADYYCATWDSRW |
| | | GIAVFGGGTKLTVL |
| 1-40_VL5 | 36 | QSVLTQPPSVSGAPGQRVTISCSGSSTDIGANYVSWYQQLPGTAPKLL |
| | | IYDNNKRPSGVPDRESGSKSGTSASLAITGLQAEDEADYYCATWDSRW |
| | | GIAVFGGGTKLTVL |
| 1-40_VL6 | 37 | QSVLTQPPSVSGAPGQRVTISCSGSSTNIGANYVSWYQQLPGTAPKLL |
| | | IYDNQKRPSGVPDRESGSKSGTSASLAITGLQAEDEADYYCATWDSRW |
| | | GIAVFGGGTKLTVL |
| Parental | 38 | GYSLTDNWIG |
| HCDR1 | | |
| Parental | 39 | IIYPGDSDTRYSPSFQG |
| HCDR2 | | |
| Parental | 40 | VGLDWNYNPLRY |
| HCDR3 | | |
| Parental | 41 | SGSSSDIGSNYVS |
| LCDR1 | | |
| Parental | 42 | DNNKRPS |
| LCDR2 | | |
| Parental | 43 | GTWDSRLGIAV |
| LCDR3 | | |
| GL_5-51_VH1 | 44 | GYELNDNWIG |
| HCDR1 | | |
| GL_5-51_VH2 | 45 | GYQLNDNWIG |
| HCDR1 | | |
| GL_5-51_VH3 | 46 | GYELKDNWIG |
| HCDR1 | | |
| GL_5-51_VH4 | 47 | GYQLKDNWAG |
| HCDR1 | | |
| GL_5-51_VH5 | 48 | GYQLKDNWIG |
| HCDR1 | | |
| GL_5-51_ | 49 | IIYPGDSDTRYSPSFKG |
| HCDR2 | | |
| GL_5-51_ | 50 | VGLEWNYNPLRY |
| HCDR3 | | |
| GL_5- | 51 | VGLEWNYNPGRY |
| 51_VH6, VH7 | | |
| HCDR3 | | |
| GL_5-51_VH8 | 52 | VGLEWNYNPVRY |
| HCDR3 | | |
| GL_5-51_VH9 | 53 | VGGEWNYNPLRY |
| HCDR3 | | |
| GL_1- | 54 | SGSSTNIGALWVS |
| 51_VL1, VL2 | | |
| LCDR1 | | |
| GL_1-51_VL3 | 55 | SGSSTNIGANYVS |
| LCDR1 | | |
| GL_1-51_VL5 | 56 | SGSSTDIGANYVS |
| LCDR1 | | |
| GL_1-51_VL6 | 57 | DNQKRPS |
| LCDR2 | | |
| GL_1-51_VL1 | 58 | ATWDSREGIAV |
| LCDR3 | | |
| GL_1-51_VL2 | 59 | ATWDSRWGIAV |
| LCDR3 | | |
| GL_5-51_VH | 60 | VGLDWNYDPLRY |
| MUT1 | | |
| HCDR3 | | |
| GL_5-51_VH | 61 | VGLEWNYNPLRY |
| MUT2 | | |
| HCDR3 | | |
| GL_5-51_VH | 62 | GYELTDNWIG |
| MUT3 | | |
| HCDR1 | | |
| GL_5- | 63 | GYQLTDNWIG |
| 51_VH_MUT4 | | |
| HCDR1 | | |
| GL_5- | 64 | GYSLNDNWIG |
| 51_VH_MUT5 | | |
| HCDR1 | | |
| GL_5- | 65 | GYSLKDNWIG |
| 51_VH_MUT6 | | |
| HCDR1 | | |
| GL_5- | 66 | GYSLTDNWTG |
| 51_VH_MUT8 | | |
| HCDR1 | | |
| GL_5-51_VH | 67 | IIYPGDSDTRYSPSFKGQ |
| MUT9 | | |
| HCDR2 | | |
| GL_5- | 68 | VGTDWNYNPLRY |
| 51_VH_MUT10 | | |
| HCDR3 | | |
| GL_1- | 69 | GTWDSREGIAV |
| 51_VL_MUT2 | | |
| LCDR3 | | |
| GL_1- | 70 | SGSSSDIGANYVS |
| 51_VL_MUT3 | | |
| LCDR1 | | |
| GL_1- | 71 | GTWDSRLGIAV |
| 51_VL_MUT3 | | |
| LCDR3 | | |
| GL_1- | 72 | SGSSSDIGSLYVS |
| 51_VL_MUT4 | | |
| LCDR1 | | |
| GL_1- | 73 | GTWDSRLGIAV |
| 51_VL_MUT4 | | |
| MUT5 | | |
| LCDR3 | | |
| GL_1- | 74 | SGSSTDIGSNYVS |
| 51_VL_MUT5 | | |
| LCDR1 | | |
| GL_1- | 75 | SGSSSDIGSNWVS |
| 51_VL_MUT6 | | |
| LCDR1 | | |
| GL_1- | 76 | SGSSSNIGSNYVS |
| 51_VL_MUT | | |
| 7 LCDR1 | | |
| GL_1- | 77 | ATWDSRLGIAV |
| 51_VL_MUT8 | | |
| LCDR3 | | |
| GL_5-51_VH | 78 | EVQLVQSGAEVKKPGESLKISCKASGYSLTDNWIGWVRQKPGKGLEWM |
| | | GIIYPGDSDTRYSPSFQGQVTISADKSISTAYLQWSSLKASDTAIYYC |
| | | VGLDWNYNPLRYWGQGTLVTVSS |
| GL_5- | 79 | EVQLVQSGAEVKKPGESLKISCKASGYSLTDNWIGWVRQKPGKGLEWM |
| 51_MUT1_VH | | GIIYPGDSDTRYSPSFQGQVTISADKSISTAYLQWSSLKASDTAIYYC |
| | | VGLDWNYDPLRYWGQGTLVTVSS |
| GL_5- | 80 | EVQLVQSGAEVKKPGESLKISCKASGYSLTDNWIGWVRQKPGKGLEWM |
| 51_VH_MUT2 | | GIIYPGDSDTRYSPSFQGQVTISADKSISTAYLQWSSLKASDTAIYYC |
| | | VGLEWNYNPLRYWGQGTLVTVSS |
| GL_5- | 81 | EVQLVQSGAEVKKPGESLKISCKASGYELTDNWIGWVRQKPGKGLEWM |
| 51_VH_MUT3 | | GIIYPGDSDTRYSPSFQGQVTISADKSISTAYLQWSSLKASDTAIYYC |
| | | VGLDWNYNPLRYWGQGTLVTVSS |
| GL_5- | 82 | EVQLVQSGAEVKKPGESLKISCKASGYQLTDNWIGWVRQKPGKGLEWM |
| 51_VH_MUT4 | | GIIYPGDSDTRYSPSFQGQVTISADKSISTAYLQWSSLKASDTAIYYC |
| | | VGLDWNYNPLRYWGQGTLVTVSS |
| GL_5- | 83 | EVQLVQSGAEVKKPGESLKISCKASGYSLNDNWIGWVRQKPGKGLEWM |
| 51_VH_MUT5 | | GIIYPGDSDTRYSPSFQGQVTISADKSISTAYLQWSSLKASDTAIYYC |
| | | VGLDWNYNPLRYWGQGTLVTVSS |
| GL_5- | 84 | EVQLVQSGAEVKKPGESLKISCKASGYSLKDNWIGWVRQKPGKGLEWM |
| 51_VH_MUT6 | | GIIYPGDSDTRYSPSFQGQVTISADKSISTAYLQWSSLKASDTAIYYC |
| | | VGLDWNYNPLRYWGQGTLVTVSS |
| GL_5- | 85 | EVQLVQSGAEVKKPGESLKISCKASGYSLTDNWIGWVRQKPGKGLEWM |
| 51_VH_MUT7 | | GIIYPGDSDTRYSPSFQGQVTISADKSISTAYLQWSSLKASDTAMYYC |
| | | VGLDWNYNPLRYWGQGTLVTVSS |
| GL_5- | 86 | EVQLVQSGAEVKKPGESLKISCKASGYSLTDNWTGWVRQKPGKGLEWM |
| 51_VH_MUT8 | | GIIYPGDSDTRYSPSFQGQVTISADKSISTAYLQWSSLKASDTAIYYC |
| | | VGLDWNYNPLRYWGQGTLVTVSS |
| GL_5- | 87 | EVQLVQSGAEVKKPGESLKISCKASGYSLTDNWIGWVRQKPGKGLEWM |
| 51_VH_MUT9 | | GIIYPGDSDTRYSPSFKGQVTISADKSISTAYLQWSSLKASDTAIYYC |
| | | VGLDWNYNPLRYWGQGTLVTVSS |
| GL_5- | 88 | EVQLVQSGAEVKKPGESLKISCKASGYSLTDNWIGWVRQKPGKGLEWM |
| 51_VH_MUT1 | | GIIYPGDSDTRYSPSFQGQVTISADKSISTAYLQWSSLKASDTAIYYC |
| 0 | | VGTDWNYNPLRYWGQGTLVTVSS |
| GL_1- | 89 | QSVLTQPPSVSAAPGQKVTISCSGSSSDIGSNYVSWYQQLPGTAPKLL |
| 51_VL_MUT1 | | IYDNNKRPSGIPDRESGSKSGTSATLGITGLQTGDEADYYCGTWDSRW |
| | | GIAVFGGGTQLTVL |
| GL_1- | 90 | QSVLTQPPSVSAAPGQKVTISCSGSSSDIGSNYVSWYQQLPGTAPKLL |
| 51_VL_MUT2 | | IYDNNKRPSGIPDRESGSKSGTSATLGITGLQTGDEADYYCGTWDSRF |
| | | GIAVFGGGTQLTVL |
| GL_1- | 91 | QSVLTQPPSVSAAPGQKVTISCSGSSSDIGANYVSWYQQLPGTAPKLL |
| 51_VL_MUT3 | | IYDNNKRPSGIPDRESGSKSGTSATLGITGLQTGDEADYYCGTWDSRL |
| | | GIAVFGGGTQLTVL |
| GL_1- | 92 | QSVLTQPPSVSAAPGQKVTISCSGSSSDIGSLYVSWYQQLPGTAPKLL |
| 51_VL_MUT4 | | IYDNNKRPSGIPDRFSGSKSGTSATLGITGLQTGDEADYYCGTWDSRL |
| | | GIAVFGGGTQLTVL |
| GL_1- | 93 | QSVLTQPPSVSAAPGQKVTISCSGSSTDIGSNYVSWYQQLPGTAPKLL |
| 51_VL_MUT5 | | IYDNNKRPSGIPDRESGSKSGTSATLGITGLQTGDEADYYCGTWDSRL |
| | | GIAVFGGGTQLTVL |
| GL_1- | 94 | QSVLTQPPSVSAAPGQKVTISCSGSSSDIGSNWVSWYQQLPGTAPKLL |
| 51_VL_MUT6 | | IYDNNKRPSGIPDRFSGSKSGTSATLGITGLQTGDEADYYCGTWDSRL |
| | | GIAVFGGGTQLTVL |
| GL_1- | 95 | QSVLTQPPSVSAAPGQKVTISCSGSSSNIGSNYVSWYQQLPGTAPKLL |
| 51_VL_MUT7 | | IYDNNKRPSGIPDRESGSKSGTSATLGITGLQTGDEADYYCGTWDSRL |
| | | GIAVFGGGTQLTVL |
| GL_1-51_ | 96 | QSVLTQPPSVSAAPGQKVTISCSGSSSDIGSNYVSWYQQLPGTAPKLL |
| VL_MUT8 | | IYDNNKRPSGIPDRFSGSKSGTSATLGITGLQTGDEADYYCATWDSRL |
| | | GIAVFGGGTQLTVL |
| GL_1- | 97 | GTWDSRWGIAV |
| 51_VL_MUT1 | | |
| LCDR3 | | |
| GL_5-51 | 98 | EVQLVQSGAEVKKPGESLKISCKAS |
| (ETY-2) HFR1 | | |
| GL_5-51 | 99 | WVRQKPGKGLEWMG |
| (ETY-2) HFR2 | | |
| GL_5-51 | 100 | GIPDRESGSKSGTSATLGITGLQTGDEADYYC |
| (ETY-2) HFR3 | | |
| GL_5-51 | 101 | FGGGTQLTVL |
| (ETY-2) HFR4 | | |
| GL_5-51 | 102 | QSVLTQPPSVSAAPGQKVTISC |
| (ETY-2) LFR1 | | |
| GL_5-51 | 103 | WYQQLPGTAPKLLIY |
| (ETY-2) LFR2 | | |
| GL_5-51 | 104 | GIPDRFSGSKSGTSATLGITGLQTGDEADYYC |
| (ETY-2) LFR3 | | |
| GL_5-51 | 105 | WGQGTLVTVSS |
| (ETY-2) LFR4 | | |
| 1-46 (ETY-3) | 106 | GYSLTDNWIG |
| HCDR1 | | |
| 1-46 (ETY-3) | 107 | IIYPGDSDTRYAQKFQG |
| HCDR2 | | |
| 1-46 (ETY-3) | 108 | VGLDWNYNPLRY |
| HCDR3 | | |
| 1-46 (ETY-3) | 109 | SGSSSDIGSNYVS |
| LCDR1 | | |
| 1-46 (ETY-3) | 110 | DNNKRPS |
| LCDR2 | | |
| 1-46 (ETY-3) | 111 | GTWDSRLGIAV |
| LCDR3 | | |
| 1-46 (ETY-3) | 112 | EVQLVQSGAEVKKPGASVKVSCKAS |
| HFR1 | | |
| 1-46 (ETY-3) | 113 | WVRQAPGQGLEWMG |
| HFR2 | | |
| 1-46 (ETY-3) | 114 | RVTMTADKSTSTVYMELSSLRSEDTAVYYC |
| HFR3 | | |
| 1-46 (ETY-3) | 115 | WGQGTLVTVSS |
| HFR4 | | |
| 1-46 (ETY-3) | 116 | QSVLTQPPSVSGAPGQRVTISC |
| LFR1 | | |
| 1-46 (ETY-3) | 117 | WYQQLPGTAPKLLIY |
| LFR2 | | |
| 1-46 (ETY-3) | 118 | GVPDRESGSKSGTSASLAITGLQAEDEADYYC |
| LFR3 | | |
| 1-46 (ETY-3) | 119 | FGGGTKLTVL |
| LFR4 | | |
| pH-dependent | 120 | EVQLVQSGAEVKKPGQSLKISCKASGYSLTDNWIGWVRQKPGKGLEWM |
| VH variant 1- | | GIIHPGDSDTRYSPSFQGQVTISADKSINTAYLQWSSLKASDTAIYYC |
| Y52H | | VGLDWNYNPLRYWGPGTLVTVSS |
| pH-dependent | 121 | EVQLVQSGAEVKKPGQSLKISCKASGYSLTDNWIGWVRQKPGKGLEWM |
| VH variant 2- | | GIIYPGDSHTRYSPSFQGQVTISADKSINTAYLQWSSLKASDTAIYYC |
| D57H | | VGLDWNYNPLRYWGPGTLVTVSS |
| pH-dependent | 122 | EVQLVQSGAEVKKPGQSLKISCKASGYSLTDNWIGWVRQKPGKGLEWM |
| VH variant 3- | | GIIYPGDSKTRYSPSFQGQVTISADKSINTAYLQWSSLKASDTAIYYC |
| D57K | | VGLDWNYNPLRYWGPGTLVTVSS |
| pH-dependent | 123 | EVQLVQSGAEVKKPGQSLKISCKASGYSLTDNWIGWVRQKPGKGLEWM |
| VH variant 4- | | GIIYPGDSDTRYSPSFQGQVTISADKSINTAYLQWSSLKASDTAIYYC |
| I70H | | VGLDWNYNPLRYWGPGTLVTVSS |
| pH-dependent | 124 | EVQLVQSGAEVKKPGQSLKISCKASGYSLTDNWIGWVRQKPGKGLEWM |
| VH variant 5- | | GIIYPGDSDTRYSPSFQGQVTISADKSINTAYLQWSSLKASDTAIYYC |
| D100Y | | VGLYWNYNPLRYWGPGTLVTVSS |
| pH-dependent | 125 | EVQLVQSGAEVKKPGQSLKISCKASGYSLTDNWIGWVRQKPGKGLEWM |
| VH variant 6- | | GIIYPGDSDTRYSPSFQGQVTISADKSINTAYLQWSSLKASDTAIYYC |
| Y103R | | VGLDWNRNPLRYWGPGTLVTVSS |
| pH-dependent | 126 | EVQLVQSGAEVKKPGQSLKISCKASGYSLTDNWIGWVRQKPGKGLEWM |
| VH variant 7- | | GIIYPGDSDTRYSPSFQGQVTISADKSINTAYLQWSSLKASDTAIYYC |
| P105D | | VGLDWNYNDLRYWGPGTLVTVSS |
| pH-dependent | 127 | EVQLVQSGAEVKKPGQSLKISCKASGYSLTDNWIGWVRQKPGKGLEWM |
| VH variant 8- | | GIIYPGDSDTRYSPSFQGQVTISADKSINTAYLQWSSLKASDTAIYYC |
| P105E | | VGLDWNYNELRYWGPGTLVTVSS |
| T3 VH | 128 | EVQLVQSGAEVKKPGESLKISCKGSGYSFTSYWIGWVRQMPGKGLEWM |
| | | GIIYPGDTDTRYSPSFQGQVTISADKSISTAYLQWSSLKASDTAVYYC |
| | | VGLDWNYNPLRYWGPGTLVTVSS |
| T3 VL | 129 | QSVLTQPPSVSAAPGQKVTISCSGSSSDIGNNYVSWYQQLPGTAPKLL |
| | | IYDNNKRPSGIPDRESGSKSGTSATLGITGLQTGDEADYYCGTWESRL |
| | | GIAVFGGGTQLTVL |
| T3 HCDR1 | 130 | GYSFTSYWIG |
| T3 HCDR2 | 131 | IIYPGDTDTRYSPSFQG |
| T3 HCDR3 | 132 | VGLDWNYNPLRY |
| T3 LCDR1 | 133 | SGSSSDIGNNYVS |
| T3 LCDR2 | 134 | DNNKRPS |
| T3 LCDR3 | 135 | GTWESRLGIAV |
| T3 VH 1.32 | 136 | EVQLVQSGAEVKKPGESLKISCKGSGYSFTHYWIGWVRQMPGKGLEWM |
| | | GIIYPGDTDTRYSPSFQGQVTISADKSISTAYLQWSSLKASDTAVYYC |
| | | VGLDWNYNPLRYWGPGTLVTVSS |
| T3 VL1.32 | 137 | QSVLTQPPSVSAAPGQKVTISCSGSSSDIGRNYVSWYQQLPGTAPKLL |
| | | IYDNNKRPSGIPDRESGSKSGTSATLGITGLQTGDEADYYCGTWESRL |
| | | GIAVFGGGTQLTVL |
| T3 VH 1.35 | 138 | EVQLVQSGAEVKKPGESLKISCKGSGYHFHSYWIGWVRQMPGKGLEWM |
| | | GIIYPGDTDTRYSPSFQGQVTISADKSISTAYLQWSSLKASDTAVYYC |
| | | VGLDWNYNPLRYWGPGTLVTVSS |
| T3 VH 1.38 | 139 | EVQLVQSGAEVKKPGESLKISCKGSGYHFTSYWIGWVRQMPGKGLEWM |
| | | GIIYPGDTDTRYSPSFQGQVTISADKSISTAYLQWSSLKASDTAVYYC |
| | | VGLDWHYNPLRYWGPGTLVTVSS |
| T3 VH 1.41 | 140 | EVQLVQSGAEVKKPGESLKISCKGSGYSFHSYWIGWVRQMPGKGLEWM |
| | | GIIYPGDTDTRYSPSFQGQVTISADKSISTAYLQWSSLKASDTAVYYC |
| | | VGLDWHYNPLRYWGPGTLVTVSS |
| T3 VH 1.47 | 141 | EVQLVQSGAEVKKPGESLKISCKGSGYHFTSHWIGWVRQMPGKGLEWM |
| | | GIIYPGDTDTRYSPSFQGQVTISADKSISTAYLQWSSLKASDTAVYYC |
| | | VGLDWNYNPLRYWGPGTLVTVSS |
| T3 VH 1.49 | 142 | EVQLVQSGAEVKKPGESLKISCKGSGYSFHHYWIGWVRQMPGKGLEWM |
| | | GIIYPGDTDTRYSPSFQGQVTISADKSISTAYLQWSSLKASDTAVYYC |
| | | VGLDWNYNPLRYWGPGTLVTVSS |
| T3 VH 1.55 | 143 | EVQLVQSGAEVKKPGESLKISCKGSGYHFHHYWIGWVRQMPGKGLEWM |
| | | GIIYPGDTDTRYSPSFQGQVTISADKSISTAYLQWSSLKASDTAVYYC |
| | | VGLDWNYNPLRYWGPGTLVTVSS |

1. An antigen binding protein or an antigen binding fragment thereof which specifically binds thyroid-stimulating hormone receptor (TSHR), wherein the antigen binding protein or an antigen binding fragment thereof comprises a heavy chain variable (VH) domain and a light chain variable (VL) domain, wherein:
a. the VH domain comprises:
i. a HCDR1 sequence comprising the amino acid sequence of SEQ ID NO: 44, a HCDR2 sequence comprising the amino acid sequence of SEQ ID NO: 49 or SEQ ID NO: 39, and a HCDR3 sequence comprising the amino acid sequence of SEQ ID NO: 50;
ii. a HCDR1 sequence comprising the amino acid sequence of SEQ ID NO: 45, a HCDR2 sequence comprising the amino acid sequence of SEQ ID NO: 49 or SEQ ID NO: 39, and a HCDR3 sequence comprising the amino acid sequence of SEQ ID NO: 50;
iii. a HCDR1 sequence comprising the amino acid sequence of SEQ ID NO: 46, a HCDR2 sequence comprising the amino acid sequence of SEQ ID NO: 49 or SEQ ID NO: 39, and a HCDR3 sequence comprising the amino acid sequence of SEQ ID NO: 50;
iv. a HCDR1 sequence comprising the amino acid sequence of SEQ ID NO: 47, a HCDR2 sequence comprising the amino acid sequence of SEQ ID NO: 49 or SEQ ID NO: 39, and a HCDR3 sequence comprising the amino acid sequence of SEQ ID NO: 50;
v. a HCDR1 sequence comprising the amino acid sequence of SEQ ID NO: 48, a HCDR2 sequence comprising the amino acid sequence of SEQ ID NO: 49 or SEQ ID NO: 39, and a HCDR3 sequence comprising the amino acid sequence of SEQ ID NO: 50;
vi. a HCDR1 sequence comprising the amino acid sequence of SEQ ID NO: 48, a HCDR2 sequence comprising the amino acid sequence of SEQ ID NO: 49 or SEQ ID NO: 39, and a HCDR3 sequence comprising the amino acid sequence of SEQ ID NO: 51;
vii. a HCDR1 sequence comprising the amino acid sequence of SEQ ID NO: 47, a HCDR2 sequence comprising the amino acid sequence of SEQ ID NO: 49 or SEQ ID NO: 39, and a HCDR3 sequence comprising the amino acid sequence of SEQ ID NO: 51;
viii. a HCDR1 sequence comprising the amino acid sequence of SEQ ID NO: 48, a HCDR2 sequence comprising the amino acid sequence of SEQ ID NO: 39, and a HCDR3 sequence comprising the amino acid sequence of SEQ ID NO: 52;
ix. a HCDR1 sequence comprising the amino acid sequence of SEQ ID NO: 48, a HCDR2 sequence comprising the amino acid sequence of SEQ ID NO: 39, and a HCDR3 sequence comprising the amino acid sequence of SEQ ID NO: 53;
x. a HCDR1 sequence comprising the amino acid sequence of SEQ ID NO: 38, a HCDR2 sequence comprising the amino acid sequence of SEQ ID NO: 39, and a HCDR3 sequence comprising the amino acid sequence of SEQ ID NO: 60;
xi. a HCDR1 sequence comprising the amino acid sequence of SEQ ID NO: 38, a HCDR2 sequence comprising the amino acid sequence of SEQ ID NO: 39, and a HCDR3 sequence comprising the amino acid sequence of SEQ ID NO: 61;
xii. a HCDR1 sequence comprising the amino acid sequence of SEQ ID NO: 62, a HCDR2 sequence comprising the amino acid sequence of SEQ ID NO: 39, and a HCDR3 sequence comprising the amino acid sequence of SEQ ID NO: 40;
xiii. a HCDR1 sequence comprising the amino acid sequence of SEQ ID NO: 63, a HCDR2 sequence comprising the amino acid sequence of SEQ ID NO: 39, and a HCDR3 sequence comprising the amino acid sequence of SEQ ID NO: 40;
xiv. a HCDR1 sequence comprising the amino acid sequence of SEQ ID NO: 64, a HCDR2 sequence comprising the amino acid sequence of SEQ ID NO: 39, and a HCDR3 sequence comprising the amino acid sequence of SEQ ID NO: 40;
xv. a HCDR1 sequence comprising the amino acid sequence of SEQ ID NO: 65, a HCDR2 sequence comprising the amino acid sequence of SEQ ID NO: 39, and a HCDR3 sequence comprising the amino acid sequence of SEQ ID NO: 40;
xvi. a HCDR1 sequence comprising the amino acid sequence of SEQ ID NO: 66, a HCDR2 sequence comprising the amino acid sequence of SEQ ID NO: 39, and a HCDR3 sequence comprising the amino acid sequence of SEQ ID NO: 40;
xvii. a HCDR1 sequence comprising the amino acid sequence of SEQ ID NO: 38, a HCDR2 sequence comprising the amino acid sequence of SEQ ID NO: 67, and a HCDR3 sequence comprising the amino acid sequence of SEQ ID NO: 40;
xviii. a HCDR1 sequence comprising the amino acid sequence of SEQ ID NO: 38, a HCDR2 sequence comprising the amino acid sequence of SEQ ID NO: 39, and a HCDR3 sequence comprising the amino acid sequence of SEQ ID NO: 68; and
b. the VL domain comprises:
i. a LCDR1 sequence comprising the amino acid sequence of SEQ ID NO: 54, a LCDR2 sequence comprising the amino acid sequence of SEQ ID NO: 42, and a LCDR3 sequence comprising the amino acid sequence of SEQ ID NO: 58 or SEQ ID NO: 59;
ii. a LCDR1 sequence comprising the amino acid sequence of SEQ ID NO: 55, a LCDR2 sequence comprising the amino acid sequence of SEQ ID NO: 42, and a LCDR3 sequence comprising the amino acid sequence of SEQ ID NO: 58;
iii. a LCDR1 sequence comprising the amino acid sequence of SEQ ID NO: 55, a LCDR2 sequence comprising the amino acid sequence of SEQ ID NO: 42, and a LCDR3 sequence comprising the amino acid sequence of SEQ ID NO: 59;
iv. a LCDR1 sequence comprising the amino acid sequence of SEQ ID NO: 56, a LCDR2 sequence comprising the amino acid sequence of SEQ ID NO: 42, and a LCDR3 sequence comprising the amino acid sequence of SEQ ID NO: 59;
v. a LCDR1 sequence comprising the amino acid sequence of SEQ ID NO: 55, a LCDR2 sequence comprising the amino acid sequence of SEQ ID NO: 57, and a LCDR3 sequence comprising the amino acid sequence of SEQ ID NO: 59;
vi. a LCDR1 sequence comprising the amino acid sequence of SEQ ID NO: 41, a LCDR2 sequence comprising the amino acid sequence of SEQ ID NO: 42, and a LCDR3 sequence comprising the amino acid sequence of SEQ ID NO: 97;
vii. a LCDR1 sequence comprising the amino acid sequence of SEQ ID NO: 41, a LCDR2 sequence comprising the amino acid sequence of SEQ ID NO: 42, and a LCDR3 sequence comprising the amino acid sequence of SEQ ID NO: 69;
viii. a LCDR1 sequence comprising the amino acid sequence of SEQ ID NO: 70, a LCDR2 sequence comprising the amino acid sequence of SEQ ID NO: 42, and a LCDR3 sequence comprising the amino acid sequence of SEQ ID NO: 71;
ix. a LCDR1 sequence comprising the amino acid sequence of SEQ ID NO: 72, a LCDR2 sequence comprising the amino acid sequence of SEQ ID NO: 42, and a LCDR3 sequence comprising the amino acid sequence of SEQ ID NO: 71;
x. a LCDR1 sequence comprising the amino acid sequence of SEQ ID NO: 74, a LCDR2 sequence comprising the amino acid sequence of SEQ ID NO: 42, and a LCDR3 sequence comprising the amino acid sequence of SEQ ID NO: 73;
xi. a LCDR1 sequence comprising the amino acid sequence of SEQ ID NO: 75, a LCDR2 sequence comprising the amino acid sequence of SEQ ID NO: 42, and a LCDR3 sequence comprising the amino acid sequence of SEQ ID NO: 73;
xii. a LCDR1 sequence comprising the amino acid sequence of SEQ ID NO: 76, a LCDR2 sequence comprising the amino acid sequence of SEQ ID NO: 42, and a LCDR3 sequence comprising the amino acid sequence of SEQ ID NO: 73; or
xiii. a LCDR1 sequence of SEQ ID NO: 41, a LCDR2 sequence comprising the amino acid sequence of SEQ ID NO: 42, and a LCDR3 sequence comprising the amino acid sequence of SEQ ID NO: 77.

2. The antigen binding protein or antigen binding fragment thereof of claim 1, wherein;
the VH comprises an amino acid sequence at least 90% identical to SEQ ID NO: 1 and the VL comprises an amino acid sequence at least 90% identical to SEQ ID NO: 2; or
the VH comprises an amino acid sequence at least 95% identical to SEQ ID NO: 3 and the VL comprises an amino acid sequence at least 90% identical to SEQ ID NO: 4.

3. (canceled)

4. The antigen binding protein or the antigen binding fragment thereof of claim 1, wherein:
a. the VH comprises the amino acid sequence of SEQ ID NO: 3, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 78, SEQ ID NO: 79, SEQ ID NO: 80, SEQ ID NO: 81, SEQ ID NO: 82, SEQ ID NO: 83, SEQ ID NO: 84, SEQ ID NO: 85, SEQ ID NO: 86, SEQ ID NO: 87, or SEQ ID NO: 88; and
b. the VL comprises the amino acid sequence of SEQ ID NO: 4, SEQ ID NO: 25, SEQ ID NO: 26, SEQ ID NO: 27, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 35, SEQ ID NO: 36, SEQ ID NO: 37, SEQ ID NO: 89, SEQ ID NO: 90, SEQ ID NO: 91, SEQ ID NO: 92, SEQ ID NO: 93, SEQ ID NO: 94, SEQ ID NO: 95, or SEQ ID NO: 96.

5. The antigen binding protein or the antigen binding fragment thereof of claim 1, wherein:
a. the VH comprises an amino acid sequence of SEQ ID NO: 78 and the VL comprises an amino acid sequence of SEQ ID NO: 25;
b. the VH comprises an amino acid sequence of SEQ ID NO: 78 and the VL comprises an amino acid sequence of SEQ ID NO: 32;
c. the VH comprises an amino acid sequence of SEQ ID NO: 16 and the VL comprises an amino acid sequence of SEQ ID NO: 25;
d. the VH comprises an amino acid sequence of SEQ ID NO: 16 and the VL comprises an amino acid sequence of SEQ ID NO: 32;
e. the VH comprises an amino acid sequence of SEQ ID NO: 7 and the VL comprises an amino acid sequence of SEQ ID NO: 29;
f. the VH comprises an amino acid sequence of SEQ ID NO: 7 and the VL comprises an amino acid sequence of SEQ ID NO: 35;
g. the VH comprises an amino acid sequence of SEQ ID NO: 7 and the VL comprises an amino acid sequence of SEQ ID NO: 30;
h. the VH comprises an amino acid sequence of SEQ ID NO: 7 and the VL comprises an amino acid sequence of SEQ ID NO: 36;
i. the VH comprises an amino acid sequence of SEQ ID NO: 8 and the VL comprises an amino acid sequence of SEQ ID NO: 29;
j. the VH comprises an amino acid sequence of SEQ ID NO: 8 and the VL comprises an amino acid sequence of SEQ ID NO: 35;
k. the VH comprises an amino acid sequence of SEQ ID NO: 8 and the VL comprises an amino acid sequence of SEQ ID NO: 30;
l. the VH comprises an amino acid sequence of SEQ ID NO: 8 and the VL comprises an amino acid sequence of SEQ ID NO: 36;
m. the VH comprises an amino acid sequence of SEQ ID NO: 9 and the VL comprises an amino acid sequence of SEQ ID NO: 29;
n. the VH comprises an amino acid sequence of SEQ ID NO: 9 and the VL comprises an amino acid sequence of SEQ ID NO: 35;
o. the VH comprises an amino acid sequence of SEQ ID NO: 9 and the VL comprises an amino acid sequence of SEQ ID NO: 30;
p. the VH comprises an amino acid sequence of SEQ ID NO: 9 and the VL comprises an amino acid sequence of SEQ ID NO: 36;
q. the VH comprises an amino acid sequence of SEQ ID NO: 10 and the VL comprises an amino acid sequence of SEQ ID NO: 29;
r. the VH comprises an amino acid sequence of SEQ ID NO: 10 and the VL comprises an amino acid sequence of SEQ ID NO: 35;
s. the VH comprises an amino acid sequence of SEQ ID NO: 10 and the VL comprises an amino acid sequence of SEQ ID NO: 30;
t. the VH comprises an amino acid sequence of SEQ ID NO: 10 and the VL comprises an amino acid sequence of SEQ ID NO: 36;
u. the VH comprises an amino acid sequence of SEQ ID NO: 11 and the VL comprises an amino acid sequence of SEQ ID NO: 29;
v. the VH comprises an amino acid sequence of SEQ ID NO: 11 and the VL comprises an amino acid sequence of SEQ ID NO: 35;
w. the VH comprises an amino acid sequence of SEQ ID NO: 11 and the VL comprises an amino acid sequence of SEQ ID NO: 30;
x. the VH comprises an amino acid sequence of SEQ ID NO: 11 and the VL comprises an amino acid sequence of SEQ ID NO: 36;
y. the VH comprises an amino acid sequence of SEQ ID NO: 12 and the VL comprises an amino acid sequence of SEQ ID NO: 29;
z. the VH comprises an amino acid sequence of SEQ ID NO: 12 and the VL comprises an amino acid sequence of SEQ ID NO: 35;
aa. the VH comprises an amino acid sequence of SEQ ID NO: 12 and the VL comprises an amino acid sequence of SEQ ID NO: 30;
bb. the VH comprises an amino acid sequence of SEQ ID NO: 12 and the VL comprises an amino acid sequence of SEQ ID NO: 36;
cc. the VH comprises an amino acid sequence of SEQ ID NO: 13 and the VL comprises an amino acid sequence of SEQ ID NO: 29;
dd. the VH comprises an amino acid sequence of SEQ ID NO: 12 and the VL comprises an amino acid sequence of SEQ ID NO: 35;
ee. the VH comprises an amino acid sequence of SEQ ID NO: 12 and the VL comprises an amino acid sequence of SEQ ID NO: 30;
ff. the VH comprises an amino acid sequence of SEQ ID NO: 12 and the VL comprises an amino acid sequence of SEQ ID NO: 36;
gg. the VH comprises an amino acid sequence of SEQ ID NO: 16 and the VL comprises an amino acid sequence of SEQ ID NO: 29;
hh. the VH comprises an amino acid sequence of SEQ ID NO: 16 and the VL comprises an amino acid sequence of SEQ ID NO: 35;
ii. the VH comprises an amino acid sequence of SEQ ID NO: 16 and the VL comprises an amino acid sequence of SEQ ID NO: 30;
jj. the VH comprises an amino acid sequence of SEQ ID NO: 16 and the VL comprises an amino acid sequence of SEQ ID NO: 36;
kk. the VH comprises an amino acid sequence of SEQ ID NO: 17 and the VL comprises an amino acid sequence of SEQ ID NO: 29;
ll. the VH comprises an amino acid sequence of SEQ ID NO: 17 and the VL comprises an amino acid sequence of SEQ ID NO: 35;
mm. the VH comprises an amino acid sequence of SEQ ID NO: 17 and the VL comprises an amino acid sequence of SEQ ID NO: 30;
nn. the VH comprises an amino acid sequence of SEQ ID NO: 17 and the VL comprises an amino acid sequence of SEQ ID NO: 36;
oo. the VH comprises an amino acid sequence of SEQ ID NO: 18 and the VL comprises an amino acid sequence of SEQ ID NO: 29;
pp. the VH comprises an amino acid sequence of SEQ ID NO: 18 and the VL comprises an amino acid sequence of SEQ ID NO: 35;
qq. the VH comprises an amino acid sequence of SEQ ID NO: 18 and the VL comprises an amino acid sequence of SEQ ID NO: 30;
rr. the VH comprises an amino acid sequence of SEQ ID NO: 18 and the VL comprises an amino acid sequence of SEQ ID NO: 36;
ss. the VH comprises an amino acid sequence of SEQ ID NO: 19 and the VL comprises an amino acid sequence of SEQ ID NO: 29;
tt. the VH comprises an amino acid sequence of SEQ ID NO: 19 and the VL comprises an amino acid sequence of SEQ ID NO: 35;
uu. the VH comprises an amino acid sequence of SEQ ID NO: 19 and the VL comprises an amino acid sequence of SEQ ID NO: 30;
vv. the VH comprises an amino acid sequence of SEQ ID NO: 19 and the VL comprises an amino acid sequence of SEQ ID NO: 36;
ww. the VH comprises an amino acid sequence of SEQ ID NO: 20 and the VL comprises an amino acid sequence of SEQ ID NO: 29;
xx. the VH comprises an amino acid sequence of SEQ ID NO: 20 and the VL comprises an amino acid sequence of SEQ ID NO: 35;
yy. the VH comprises an amino acid sequence of SEQ ID NO: 20 and the VL comprises an amino acid sequence of SEQ ID NO: 30;
zz. the VH comprises an amino acid sequence of SEQ ID NO: 20 and the VL comprises an amino acid sequence of SEQ ID NO: 36;
aaa. the VH comprises an amino acid sequence of SEQ ID NO: 21 and the VL comprises an amino acid sequence of SEQ ID NO: 29;
bbb. the VH comprises an amino acid sequence of SEQ ID NO: 21 and the VL comprises an amino acid sequence of SEQ ID NO: 35;
ccc. the VH comprises an amino acid sequence of SEQ ID NO: 21 and the VL comprises an amino acid sequence of SEQ ID NO: 30;
ddd. the VH comprises an amino acid sequence of SEQ ID NO: 21 and the VL comprises an amino acid sequence of SEQ ID NO: 36;
eee. the VH comprises an amino acid sequence of SEQ ID NO: 22 and the VL comprises an amino acid sequence of SEQ ID NO: 29;
fff. the VH comprises an amino acid sequence of SEQ ID NO: 22 and the VL comprises an amino acid sequence of SEQ ID NO: 35;
ggg. the VH comprises an amino acid sequence of SEQ ID NO: 22 and the VL comprises an amino acid sequence of SEQ ID NO: 30;
hhh. the VH comprises an amino acid sequence of SEQ ID NO: 22 and the VL comprises an amino acid sequence of SEQ ID NO: 36;
iii. the VH comprises an amino acid sequence of SEQ ID NO: 11 and the VL comprises an amino acid sequence of SEQ ID NO: 26;
jjj. the VH comprises an amino acid sequence of SEQ ID NO: 20 and the VL comprises an amino acid sequence of SEQ ID NO: 26;
kkk. the VH comprises an amino acid sequence of SEQ ID NO: 12 and the VL comprises an amino acid sequence of SEQ ID NO: 26;
lll. the VH comprises an amino acid sequence of SEQ ID NO: 21 and the VL comprises an amino acid sequence of SEQ ID NO: 26;
mmm. the VH comprises an amino acid sequence of SEQ ID NO: 11 and the VL comprises an amino acid sequence of SEQ ID NO: 27;
nnn. the VH comprises an amino acid sequence of SEQ ID NO: 20 and the VL comprises an amino acid sequence of SEQ ID NO: 27;
ooo. the VH comprises an amino acid sequence of SEQ ID NO: 12 and the VL comprises an amino acid sequence of SEQ ID NO: 27;
ppp. the VH comprises an amino acid sequence of SEQ ID NO: 21 and the VL comprises an amino acid sequence of SEQ ID NO: 27;
qqq. the VH comprises an amino acid sequence of SEQ ID NO: 11 and the VL comprises an amino acid sequence of SEQ ID NO: 28;
rrr. the VH comprises an amino acid sequence of SEQ ID NO: 20 and the VL comprises an amino acid sequence of SEQ ID NO: 28;
sss. the VH comprises an amino acid sequence of SEQ ID NO: 12 and the VL comprises an amino acid sequence of SEQ ID NO: 28;
ttt. the VH comprises an amino acid sequence of SEQ ID NO: 21 and the VL comprises an amino acid sequence of SEQ ID NO: 28;
uuu the VH comprises an amino acid sequence of SEQ ID NO: 11 and the VL comprises an amino acid sequence of SEQ ID NO: 31;
vvv. the VH comprises an amino acid sequence of SEQ ID NO: 20 and the VL comprises an amino acid sequence of SEQ ID NO: 31;
www. the VH comprises an amino acid sequence of SEQ ID NO: 12 and the VL comprises an amino acid sequence of SEQ ID NO: 31;
xxx. the VH comprises an amino acid sequence of SEQ ID NO: 21 and the VL comprises an amino acid sequence of SEQ ID NO: 31;
yyy. the VH comprises an amino acid sequence of SEQ ID NO: 14 and the VL comprises an amino acid sequence of SEQ ID NO: 30;
zzz. the VH comprises an amino acid sequence of SEQ ID NO: 23 and the VL comprises an amino acid sequence of SEQ ID NO: 30;
aaaa. the VH comprises an amino acid sequence of SEQ ID NO: 15 and the VL comprises an amino acid sequence of SEQ ID NO: 30;
bbbb. the VH comprises an amino acid sequence of SEQ ID NO: 24 and the VL comprises an amino acid sequence of SEQ ID NO: 30;
cccc. the VH comprises an amino acid sequence of SEQ ID NO: 11 and the VL comprises an amino acid sequence of SEQ ID NO: 32;
dddd. the VH comprises an amino acid sequence of SEQ ID NO: 20 and the VL comprises an amino acid sequence of SEQ ID NO: 32;
eeee. the VH comprises an amino acid sequence of SEQ ID NO: 12 and the VL comprises an amino acid sequence of SEQ ID NO: 32;
ffff. the VH comprises an amino acid sequence of SEQ ID NO: 21 and the VL comprises an amino acid sequence of SEQ ID NO: 32;
gggg. the VH comprises an amino acid sequence of SEQ ID NO: 11 and the VL comprises an amino acid sequence of SEQ ID NO: 33;
hhhh. the VH comprises an amino acid sequence of SEQ ID NO: 20 and the VL comprises an amino acid sequence of SEQ ID NO: 33;
iiii. the VH comprises an amino acid sequence of SEQ ID NO: 12 and the VL comprises an amino acid sequence of SEQ ID NO: 33;
jjjj. the VH comprises an amino acid sequence of SEQ ID NO: 21 and the VL comprises an amino acid sequence of SEQ ID NO: 33;
kkkk. the VH comprises an amino acid sequence of SEQ ID NO: 11 and the VL comprises an amino acid sequence of SEQ ID NO: 34;
llll. the VH comprises an amino acid sequence of SEQ ID NO: 20 and the VL comprises an amino acid sequence of SEQ ID NO: 34;
mmmm. the VH comprises an amino acid sequence of SEQ ID NO: 12 and the VL comprises an amino acid sequence of SEQ ID NO: 34;
nnnn. the VH comprises an amino acid sequence of SEQ ID NO: 21 and the VL comprises an amino acid sequence of SEQ ID NO: 34;
oooo. the VH comprises an amino acid sequence of SEQ ID NO: 11 and the VL comprises an amino acid sequence of SEQ ID NO: 37;
pppp. the VH comprises an amino acid sequence of SEQ ID NO: 20 and the VL comprises an amino acid sequence of SEQ ID NO: 37;
qqqq. the VH comprises an amino acid sequence of SEQ ID NO: 12 and the VL comprises an amino acid sequence of SEQ ID NO: 37;
rrrr. the VH comprises an amino acid sequence of SEQ ID NO: 21 and the VL comprises an amino acid sequence of SEQ ID NO: 37;
ssss. the VH comprises an amino acid sequence of SEQ ID NO: 14 and the VL comprises an amino acid sequence of SEQ ID NO: 36;
tttt. the VH comprises an amino acid sequence of SEQ ID NO: 23 and the VL comprises an amino acid sequence of SEQ ID NO: 36;
uuuu. the VH comprises an amino acid sequence of SEQ ID NO: 15 and the VL comprises an amino acid sequence of SEQ ID NO: 36; or
vvvv. the VH comprises an amino acid sequence of SEQ ID NO: 24 and the VL comprises an amino acid sequence of SEQ ID NO: 36.

6. An antigen binding protein or the antigen binding fragment thereof which specifically binds thyroid-stimulating hormone receptor (TSHR), wherein the antigen binding protein or an antigen binding fragment thereof comprises:
a. a VH comprising an amino acid sequence of SEQ ID NO: 78 and a VL comprising an amino acid sequence of SEQ ID NO: 25;
b. a VH comprising an amino acid sequence of SEQ ID NO: 78 and a VL comprising an amino acid sequence of SEQ ID NO: 32;
c. a VH comprising an amino acid sequence of SEQ ID NO: 16 and a VL comprising an amino acid sequence of SEQ ID NO: 25;
d. a VH comprising an amino acid sequence of SEQ ID NO: 16 and a VL comprising an amino acid sequence of SEQ ID NO: 32;
e. a VH comprising an amino acid sequence of SEQ ID NO: 7 and a VL comprising an amino acid sequence of SEQ ID NO: 29;
f. a VH comprising an amino acid sequence of SEQ ID NO: 7 and a VL comprising an amino acid sequence of SEQ ID NO: 35;
g. a VH comprising an amino acid sequence of SEQ ID NO: 7 and a VL comprising an amino acid sequence of SEQ ID NO: 30;
h. a VH comprising an amino acid sequence of SEQ ID NO: 7 and a VL comprising an amino acid sequence of SEQ ID NO: 36;
i. a VH comprising an amino acid sequence of SEQ ID NO: 8 and a VL comprising an amino acid sequence of SEQ ID NO: 29;
j. a VH comprising an amino acid sequence of SEQ ID NO: 8 and a VL comprising an amino acid sequence of SEQ ID NO: 35;
k. a VH comprising an amino acid sequence of SEQ ID NO: 8 and a VL comprising an amino acid sequence of SEQ ID NO: 30;
l. a VH comprising an amino acid sequence of SEQ ID NO: 8 and a VL comprising an amino acid sequence of SEQ ID NO: 36;
m. a VH comprising an amino acid sequence of SEQ ID NO: 9 and a VL comp comprising rises an amino acid sequence of SEQ ID NO: 29;
n. a VH comprising an amino acid sequence of SEQ ID NO: 9 and a VL comprising an amino acid sequence of SEQ ID NO: 35;
o. a VH comprising an amino acid sequence of SEQ ID NO: 9 and a VL comprising an amino acid sequence of SEQ ID NO: 30;
p. a VH comprising an amino acid sequence of SEQ ID NO: 9 and a VL comprising an amino acid sequence of SEQ ID NO: 36;
q. a VH comprising an amino acid sequence of SEQ ID NO: 10 and a VL comprising an amino acid sequence of SEQ ID NO: 29;
r. a VH comprising an amino acid sequence of SEQ ID NO: 10 and a VL comprising an amino acid sequence of SEQ ID NO: 35;
s. a VH comprising an amino acid sequence of SEQ ID NO: 10 and a VL comprising an amino acid sequence of SEQ ID NO: 30;
t. a VH comprising an amino acid sequence of SEQ ID NO: 10 and a VL comprising an amino acid sequence of SEQ ID NO: 36;
u. a VH comprising an amino acid sequence of SEQ ID NO: 11 and a VL comprising an amino acid sequence of SEQ ID NO: 29;
v. a VH comprising an amino acid sequence of SEQ ID NO: 11 and a VL comprising an amino acid sequence of SEQ ID NO: 35;
w. a VH comprising an amino acid sequence of SEQ ID NO: 11 and a VL comprising an amino acid sequence of SEQ ID NO: 30;
x. a VH comprising an amino acid sequence of SEQ ID NO: 11 and a VL comprising an amino acid sequence of SEQ ID NO: 36;
y. a VH comprising an amino acid sequence of SEQ ID NO: 12 and a VL comprising an amino acid sequence of SEQ ID NO: 29;
z. a VH comprising an amino acid sequence of SEQ ID NO: 12 and a VL comprising an amino acid sequence of SEQ ID NO: 35;
aa. a VH comprising an amino acid sequence of SEQ ID NO: 12 and a VL comprising an amino acid sequence of SEQ ID NO: 30;
bb. a VH comprising an amino acid sequence of SEQ ID NO: 12 and a VL comprising an amino acid sequence of SEQ ID NO: 36;
cc. a VH comprising an amino acid sequence of SEQ ID NO: 13 and a VL comprising an amino acid sequence of SEQ ID NO: 29;
dd. a VH comprising an amino acid sequence of SEQ ID NO: 12 and a VL comprising an amino acid sequence of SEQ ID NO: 35;
ee. a VH comprising an amino acid sequence of SEQ ID NO: 12 and a VL comprising an amino acid sequence of SEQ ID NO: 30;
ff. a VH comprising an amino acid sequence of SEQ ID NO: 12 and a VL comprising an amino acid sequence of SEQ ID NO: 36;
gg. a VH comprising an amino acid sequence of SEQ ID NO: 16 and a VL comprising an amino acid sequence of SEQ ID NO: 29;
hh. a VH comprising an amino acid sequence of SEQ ID NO: 16 and a VL comprising an amino acid sequence of SEQ ID NO: 35;
ii. a VH comprising an amino acid sequence of SEQ ID NO: 16 and a VL comprising an amino acid sequence of SEQ ID NO: 30;
jj. a VH comprising an amino acid sequence of SEQ ID NO: 16 and a VL comprising an amino acid sequence of SEQ ID NO: 36;
kk. a VH comprising an amino acid sequence of SEQ ID NO: 17 and a VL comprising an amino acid sequence of SEQ ID NO: 29;
ll. a VH comprising an amino acid sequence of SEQ ID NO: 17 and a VL comprising an amino acid sequence of SEQ ID NO: 35;
mm. a VH comprising an amino acid sequence of SEQ ID NO: 17 and a VL comprising an amino acid sequence of SEQ ID NO: 30;
nn. a VH comprising an amino acid sequence of SEQ ID NO: 17 and a VL comprising an amino acid sequence of SEQ ID NO: 36;
oo. a VH comprising an amino acid sequence of SEQ ID NO: 18 and a VL comprising an amino acid sequence of SEQ ID NO: 29;
pp. a VH comprising an amino acid sequence of SEQ ID NO: 18 and a VL comprising an amino acid sequence of SEQ ID NO: 35;
qq. a VH comprising an amino acid sequence of SEQ ID NO: 18 and a VL comprising an amino acid sequence of SEQ ID NO: 30;
rr. a VH comprising an amino acid sequence of SEQ ID NO: 18 and a VL comprising an amino acid sequence of SEQ ID NO: 36;
ss. a VH comprising an amino acid sequence of SEQ ID NO: 19 and a VL comprising an amino acid sequence of SEQ ID NO: 29;
tt. a VH comprising an amino acid sequence of SEQ ID NO: 19 and a VL comprising an amino acid sequence of SEQ ID NO: 35;
uu. a VH comprising an amino acid sequence of SEQ ID NO: 19 and a VL comprising an amino acid sequence of SEQ ID NO: 30;
vv. a VH comprising an amino acid sequence of SEQ ID NO: 19 and a VL comprising an amino acid sequence of SEQ ID NO: 36;
ww. a VH comprising an amino acid sequence of SEQ ID NO: 20 and a VL comprising an amino acid sequence of SEQ ID NO: 29;
xx. a VH comprising an amino acid sequence of SEQ ID NO: 20 and a VL comprising an amino acid sequence of SEQ ID NO: 35;
yy. a VH comprising an amino acid sequence of SEQ ID NO: 20 and a VL comprising an amino acid sequence of SEQ ID NO: 30;
zz. a VH comprising an amino acid sequence of SEQ ID NO: 20 and a VL comprising an amino acid sequence of SEQ ID NO: 36;
aaa. a VH comprising an amino acid sequence of SEQ ID NO: 21 and a VL comprising an amino acid sequence of SEQ ID NO: 29;
bbb. a VH comprising an amino acid sequence of SEQ ID NO: 21 and a VL comprising an amino acid sequence of SEQ ID NO: 35;
ccc. a VH comprising an amino acid sequence of SEQ ID NO: 21 and a VL comprising an amino acid sequence of SEQ ID NO: 30;
ddd. a VH comprising an amino acid sequence of SEQ ID NO: 21 and a VL comprising an amino acid sequence of SEQ ID NO: 36;
eee. a VH comprising an amino acid sequence of SEQ ID NO: 22 and a VL comprising an amino acid sequence of SEQ ID NO: 29;
fff. a VH comprising an amino acid sequence of SEQ ID NO: 22 and a VL comprising an amino acid sequence of SEQ ID NO: 35;
ggg. a VH comprising an amino acid sequence of SEQ ID NO: 22 and a VL comprising an amino acid sequence of SEQ ID NO: 30;
hhh. a VH comprising an amino acid sequence of SEQ ID NO: 22 and a VL comprising an amino acid sequence of SEQ ID NO: 36;
iii. a VH comprising an amino acid sequence of SEQ ID NO: 11 and a VL comprising an amino acid sequence of SEQ ID NO: 26;
jjj. a VH comprising an amino acid sequence of SEQ ID NO: 20 and a VL comprising an amino acid sequence of SEQ ID NO: 26;
kkk. a VH comprising an amino acid sequence of SEQ ID NO: 12 and a VL comprising an amino acid sequence of SEQ ID NO: 26;
lll. a VH comprising an amino acid sequence of SEQ ID NO: 21 and a VL comprising an amino acid sequence of SEQ ID NO: 26;
mmm. a VH comprising an amino acid sequence of SEQ ID NO: 11 and a VL comprising an amino acid sequence of SEQ ID NO: 27;
nnn. a VH comprising an amino acid sequence of SEQ ID NO: 20 and a VL comprising an amino acid sequence of SEQ ID NO: 27;
ooo. a VH comprising an amino acid sequence of SEQ ID NO: 12 and a VL comprising an amino acid sequence of SEQ ID NO: 27;
ppp. a VH comprising an amino acid sequence of SEQ ID NO: 21 and a VL comprising an amino acid sequence of SEQ ID NO: 27;
qqq. a VH comprising an amino acid sequence of SEQ ID NO: 11 and a VL comprising an amino acid sequence of SEQ ID NO: 28;
rrr. a VH comprising an amino acid sequence of SEQ ID NO: 20 and a VL comprising an amino acid sequence of SEQ ID NO: 28;
sss. a VH comprising an amino acid sequence of SEQ ID NO: 12 and a VL comprising an amino acid sequence of SEQ ID NO: 28;
ttt. a VH comprising an amino acid sequence of SEQ ID NO: 21 and a VL comprising an amino acid sequence of SEQ ID NO: 28;
uuu. a VH comprising an amino acid sequence of SEQ ID NO: 11 and a VL comprising an amino acid sequence of SEQ ID NO: 31;
vvv. a VH comprising an amino acid sequence of SEQ ID NO: 20 and a VL comprising an amino acid sequence of SEQ ID NO: 31;
www. a VH comprising an amino acid sequence of SEQ ID NO: 12 and a VL comprising an amino acid sequence of SEQ ID NO: 31;
xxx a VH comprising an amino acid sequence of SEQ ID NO: 21 and a VL comprising an amino acid sequence of SEQ ID NO: 31;
yyy. a VH comprising an amino acid sequence of SEQ ID NO: 14 and a VL comprising an amino acid sequence of SEQ ID NO: 30;
zzz. a VH comprising an amino acid sequence of SEQ ID NO: 23 and a VL comprising an amino acid sequence of SEQ ID NO: 30;
aaaa. a VH comprising an amino acid sequence of SEQ ID NO: 15 and a VL comprising an amino acid sequence of SEQ ID NO: 30;
bbbb. a VH comprising an amino acid sequence of SEQ ID NO: 24 and a VL comprising an amino acid sequence of SEQ ID NO: 30;
cccc. a VH comprising an amino acid sequence of SEQ ID NO: 11 and a VL comprising an amino acid sequence of SEQ ID NO: 32;
dddd. a VH comprising an amino acid sequence of SEQ ID NO: 20 and a VL comprising an amino acid sequence of SEQ ID NO: 32;
eeee. a VH comprising an amino acid sequence of SEQ ID NO: 12 and a VL comprising an amino acid sequence of SEQ ID NO: 32;
ffff. a VH comprising an amino acid sequence of SEQ ID NO: 21 and a VL comprising an amino acid sequence of SEQ ID NO: 32;
gggg. a VH comprising an amino acid sequence of SEQ ID NO: 11 and a VL comprising an amino acid sequence of SEQ ID NO: 33;
hhhh. a VH comprising an amino acid sequence of SEQ ID NO: 20 and a VL comprising an amino acid sequence of SEQ ID NO: 33;
iiii. a VH comprising an amino acid sequence of SEQ ID NO: 12 and a VL comprising an amino acid sequence of SEQ ID NO: 33;
jjjj. a VH comprising an amino acid sequence of SEQ ID NO: 21 and a VL comprising an amino acid sequence of SEQ ID NO: 33;
kkkk. a VH comprising an amino acid sequence of SEQ ID NO: 11 and a VL comprising an amino acid sequence of SEQ ID NO: 34;
llll. the a VH comprising an amino acid sequence of SEQ ID NO: 20 and a VL comprising an amino acid sequence of SEQ ID NO: 34;
mmmm. a VH comprising an amino acid sequence of SEQ ID NO: 12 and a VL comprising an amino acid sequence of SEQ ID NO: 34;
nnnn. a VH comprising an amino acid sequence of SEQ ID NO: 21 and a VL comprising an amino acid sequence of SEQ ID NO: 34;
oooo. a VH comprising an amino acid sequence of SEQ ID NO: 11 and a VL comprising an amino acid sequence of SEQ ID NO: 37;
pppp. a VH comprising an amino acid sequence of SEQ ID NO: 20 and a VL comprising an amino acid sequence of SEQ ID NO: 37;
qqqq. a VH comprising an amino acid sequence of SEQ ID NO: 12 and a VL comprising an amino acid sequence of SEQ ID NO: 37;
rrrr. a VH comprising an amino acid sequence of SEQ ID NO: 21 and a VL comprising an amino acid sequence of SEQ ID NO: 37;
ssss. a VH comprising an amino acid sequence of SEQ ID NO: 14 and a VL comprising an amino acid sequence of SEQ ID NO: 36;
tttt. a VH comprising an amino acid sequence of SEQ ID NO: 23 and a VL comprising an amino acid sequence of SEQ ID NO: 36;
uuuu. a VH comprising an amino acid sequence of SEQ ID NO: 15 and a VL comprising an amino acid sequence of SEQ ID NO: 36;
vvvv. a VH comprising an amino acid sequence of SEQ ID NO: 24 and a VL comprising an amino acid sequence of SEQ ID NO: 36;
wwww. a VH comprising an amino acid sequence of SEQ ID NO: 79 and a VL comprising an amino acid sequence of SEQ ID NO: 2;
xxxx. a VH comprising an amino acid sequence of SEQ ID NO: 80 and a VL comprising an amino acid sequence of SEQ ID NO; 2;
yyyy. a VH comprising an amino acid sequence of SEQ ID NO: 81 and a VL comprising an amino acid sequence of SEQ ID NO: 2;
zzzz. a VH comprising an amino acid sequence of SEQ ID NO: 82 and a VL comprising an amino acid sequence of SEQ ID NO: 2;
aaaaa. a VH comprising an amino acid sequence of SEQ ID NO: 83 and a VL comprising an amino acid sequence of SEQ ID NO: 2;
bbbbb. a VH comprising an amino acid sequence of SEQ ID NO: 84 and a VL comprising an amino acid sequence of SEQ ID NO: 2;
ccccc. a VH comprising an amino acid sequence of SEQ ID NO: 85 and a VL comprising an amino acid sequence of SEQ ID NO: 2;
ddddd. a VH comprising an amino acid sequence of SEQ ID NO: 86 and a VL comprising an amino acid sequence of SEQ ID NO: 2;
eeeee. a VH comprising an amino acid sequence of SEQ ID NO: 87 and a VL comprising an amino acid sequence of SEQ ID NO: 2;
fffff. a VH comprising an amino acid sequence of SEQ ID NO: 88 and a VL comprising an amino acid sequence of SEQ ID NO: 2;
ggggg. a VH comprising an amino acid sequence of SEQ ID NO: 1 and a VL comprising an amino acid sequence of SEQ ID NO: 89;
hhhhh. a VH comprising an amino acid sequence of SEQ ID NO: 1 and a VL comprising an amino acid sequence of SEQ ID NO: 90;
iiiii. a VH comprising an amino acid sequence of SEQ ID NO: 1 and a VL comprising an amino acid sequence of SEQ ID NO: 91;
jjjjj. a VH comprising an amino acid sequence of SEQ ID NO: 1 and a VL comprising an amino acid sequence of SEQ ID NO: 92;
kkkkk. a VH comprising an amino acid sequence of SEQ ID NO: 1 and a VL comprising an amino acid sequence of SEQ ID NO: 93;
llll. a VH comprising an amino acid sequence of SEQ ID NO: 1 and a VL comprising an amino acid sequence of SEQ ID NO: 94;
mmmmm. a VH comprising an amino acid sequence of SEQ ID NO: 1 and a VL comprising an amino acid sequence of SEQ ID NO: 95; or
nnnnn. a VH comprising an amino acid sequence of SEQ ID NO: 1 and a VL comprising an amino acid sequence of SEQ ID NO: 96; or
wherein the antigen binding protein or an antigen binding fragment thereof comprises:
a. a VH comprising an amino acid sequence of SEQ ID NO: 120 and a VL comprising an amino acid sequence of SEQ ID NO: 2;
b. a VH comprising an amino acid sequence of SEQ ID NO: 121 and a VL comprising an amino acid sequence of SEQ ID NO: 2;
c. a VH comprising an amino acid sequence of SEQ ID NO: 122 and a VL comprising an amino acid sequence of SEQ ID NO: 2;
d. a VH comprising an amino acid sequence of SEQ ID NO: 123 and a VL comprising an amino acid sequence of SEQ ID NO: 2;
e. a VH comprising an amino acid sequence of SEQ ID NO: 124 and a VL comprising an amino acid sequence of SEQ ID NO: 2;
f. a VH comprising an amino acid sequence of SEQ ID NO: 125 and a VL comprising an amino acid sequence of SEQ ID NO: 2;
g. a VH comprising an amino acid sequence of SEQ ID NO: 126 and a VL comprising an amino acid sequence of SEQ ID NO: 2; or
h. a VH comprising an amino acid sequence of SEQ ID NO: 127 and a VL comprising an amino acid sequence of SEQ ID NO: 2; or
wherein the antigen binding protein or an antigen binding fragment thereof comprises:
a. a VH comprising an amino acid sequence of SEQ ID NO: 128 and a VL comprising an amino acid sequence of SEQ ID NO: 129;
b. a VH comprising an amino acid sequence of SEQ ID NO: 136 and a VL comprising an amino acid sequence of SEQ ID NO: 137;
c. a VH comprising an amino acid sequence of SEQ ID NO: 138 and a VL comprising an amino acid sequence of SEQ ID NO: 129;
d. a VH comprising an amino acid sequence of SEQ ID NO: 139 and a VL comprising an amino acid sequence of SEQ ID NO: 129;
e. a VH comprising an amino acid sequence of SEQ ID NO: 140 and a VL comprising an amino acid sequence of SEQ ID NO: 129;
f. a VH comprising an amino acid sequence of SEQ ID NO: 141 and a VL comprising an amino acid sequence of SEQ ID NO: 137;
g. a VH comprising an amino acid sequence of SEQ ID NO: 142 and a VL comprising an amino acid sequence of SEQ ID NO: 137;
h. a VH comprising an amino acid sequence of SEQ ID NO: 143 and a VL comprising an amino acid sequence of SEQ ID NO: 129; or
i. a VH comprising an amino acid sequence of SEQ ID NO: 143 and a VL comprising an amino acid sequence of SEQ ID NO: 137.

7. (canceled)

8. (canceled)

9. The antigen binding protein or the antigen binding fragment thereof of claim 1, wherein the protein further comprises an immunoglobulin Fc domain or variant thereof, optionally wherein the Fc domain or variant thereof comprises a first Fc heavy chain and a second Fc heavy chain.

10-25. (canceled)

26. An antigen binding protein or an antigen binding fragment thereof which specifically binds thyroid-stimulating hormone receptor (TSHR), wherein the antigen binding protein or an antigen binding fragment thereof comprises a heavy chain variable (VH) domain with at least 90% identity to SEQ ID NO: 78 with a glutamic acid (E) at position 16, a serine(S) at position 77, and a glutamine (Q) at position 111 relative to SEQ ID NO: 78; and a light chain variable (VL) domain with at least 90% identity to SEQ ID NO: 25 with a leucine (L) amino acid at position 40 and a glycine (G) amino acid at position 58 relative to SEQ ID NO: 25.

27. An antigen binding protein or an antigen binding fragment thereof which specifically binds thyroid-stimulating hormone receptor (TSHR), wherein the antigen binding protein or an antigen binding fragment thereof comprises:
a heavy chain variable (VH) domain with at least 90% identity to SEQ ID NO: 78 and comprising a heavy chain framework region 1 (HFR1) amino acid sequence of SEQ ID NO: 98, a heavy chain framework region 2 (HFR2) amino acid sequence of SEQ ID NO: 99, a heavy chain framework region 3 (HFR3) amino acid sequence of SEQ ID NO: 100, and a heavy chain framework region 4 (HFR4) amino acid sequence of SEQ ID NO: 101; and
a light chain variable (VL) domain with at least 90% identity to SEQ ID NO: 25 and comprising a light chain framework region 1 (LFR1) amino acid sequence of SEQ ID NO: 102, a light chain framework region 2 (LFR2) amino acid sequence of SEQ ID NO: 103, a light chain framework region 3 (LFR3) amino acid sequence of SEQ ID NO: 104, and a light chain framework region 4 (LFR4) amino acid sequence of SEQ ID NO: 105.

28. The antigen binding protein or an antigen binding fragment thereof of claim 26, comprising an HCDR1 sequence of SEQ ID NO: 38, an HCDR2 sequence of SEQ ID NO: 39, an HCDR3 sequence of SEQ ID NO: 40, an LCDR1 sequence of SEQ ID NO: 41, an LCDR2 sequence of SEQ ID NO: 42, and an LCDR3 sequence of SEQ ID NO: 43.

29. An antigen binding protein or an antigen binding fragment thereof which specifically binds thyroid-stimulating hormone receptor (TSHR), wherein the antigen binding protein or an antigen binding fragment thereof comprises:
a heavy chain variable (VH) domain with at least 90% identity to SEQ ID NO: 3 and comprising a heavy chain framework region 1 (HFR1) amino acid sequence of SEQ ID NO: 112, a heavy chain framework region 2 (HFR2) amino acid sequence of SEQ ID NO: 113, a heavy chain framework region 3 (HFR3) amino acid sequence of SEQ ID NO: 114, and a heavy chain framework region 4 (HFR4) amino acid sequence of SEQ ID NO: 115; and
a light chain variable (VL) domain with at least 90% identity to SEQ ID NO: 4 and comprising a light chain framework region 1 (LFR1) amino acid sequence of SEQ ID NO: 116, a light chain framework region 2 (LFR2) amino acid sequence of SEQ ID NO: 117, a light chain framework region 3 (LFR3) amino acid sequence of SEQ ID NO: 118, and a light chain framework region 4 (LFR4) amino acid sequence of SEQ ID NO: 119.

30. The antigen binding protein or an antigen binding fragment thereof of claim 29, comprising an HCDR1 sequence of SEQ ID NO: 106, an HCDR2 sequence of SEQ ID NO: 107, an HCDR3 sequence of SEQ ID NO: 108, an LCDR1 sequence of SEQ ID NO: 109, an LCDR2 sequence of SEQ ID NO: 110, and an LCDR3 sequence of SEQ ID NO: 111.

31. A pharmaceutical composition comprising the antigen binding protein or the antigen binding fragment thereof of claim 1 and a pharmaceutically acceptable carrier.

32. (canceled)

33. An isolated nucleic acid molecule encoding the antigen binding protein or the antigen binding fragment thereof of claim 1.

34. An expression vector comprising the nucleic acid molecule of claim 33.

35. A host cell comprising the expression vector of claim 34.

36. A method of treating or preventing a thyroid stimulating hormone receptor (TSHR)-related disease in a subject, comprising administering to a subject in need thereof the antigen binding protein or antigen binding fragment thereof of claim 1, optionally wherein:
the TSHR-related disease is an autoimmune disease, optionally Graves' disease;
the TSHR-related disease is thyrotoxicosis; or
the TSHR-related disease is cancer.

37-40. (canceled)

41. A method of treating an autoimmune disease associated with autoantibodies to thyroid stimulating hormone receptor (TSHR) in a subject, comprising administering to a subject in need thereof the antigen binding protein or antigen binding fragment thereof of claim 1.

42. A chimeric antigen receptor (CAR) comprising a TSHR-binding domain comprising the antigen binding protein or antigen binding fragment thereof of claim 1.

43. A cell comprising the CAR of claim 42.

44. An antibody drug conjugate (ADC) comprising the antigen binding protein or an antigen binding fragment thereof which specifically binds thyroid-stimulating hormone receptor (TSHR) of claim 1, optionally wherein the ADC is conjugated to a radioisotope or to a therapeutic small molecule.

45. (canceled)

46. A method of treating or preventing a disease or disorder comprising administration of the ADC of claim 44.

47. A method of diagnosing or detecting a disease or disorder comprising administration of the ADC of claim 44.

48. (canceled)

49. A nucleic acid library comprising a plurality of polynucleotide sequences, each polynucleotide sequence in the plurality encoding for a variant anti-TSHR antigen binding protein comprising one or both of:
a variant variable heavy chain (VH) comprising one or more amino acid substitutions in the amino acid sequence of SEQ ID NO: 1 or 128, and
a variant variable light chain (VL) comprising one or more amino acid substitutions in the amino acid sequence of SEQ ID NO: 2 or 129.

50. The nucleic acid library of claim 49, wherein;
each variant VH encoded the polynucleotide sequence in the plurality comprises or consists of one acid substitution in the amino acid sequence of SEQ ID NO: 1 or 128; and/or
each variant VL encoded the polynucleotide sequence in the plurality comprises or consists of one acid substitution in the amino acid sequence of SEQ ID NO: 2 or 129.

51. (canceled)

52. The nucleic acid library of claim 49, wherein:
the library comprises a diversity of at least about 100, 200, 300, 400, 500, 600, 700, 800, 900, 1000, 1500, or 2000 unique polynucleotide sequences;
the library comprises a diversity of about 100 to about 2000 unique polynucleotide sequences encoding variant VH amino acid sequences of SEQ ID NO: 1 or 128;
the library comprises a diversity of about 1920 unique polynucleotide sequences encoding variant VH amino acid sequences of SEQ ID NO: 1 or 128;
the library comprises a diversity of about 100 to about 2000 unique polynucleotide sequences encoding variant VL amino acid sequences of SEQ ID NO: 2 or 129; and/or
the library comprises a diversity of about 1660 unique polynucleotide sequences encoding variant VL amino acid sequences of SEQ ID NO: 2 or 129.

53-56. (canceled)

57. An isolated nucleic acid molecule encoding the antigen binding protein or the antigen binding fragment thereof of claim 26.

58. An isolated nucleic acid molecule encoding the antigen binding protein or the antigen binding fragment thereof of claim 29.

59. A method of treating or preventing a thyroid stimulating hormone receptor (TSHR)-related disease in a subject, comprising administering to a subject in need thereof the antigen binding protein or antigen binding fragment thereof of claim 26, optionally wherein:
the TSHR-related disease is an autoimmune disease, optionally Graves' disease;
the TSHR-related disease is thyrotoxicosis; or
the TSHR-related disease is cancer.

60. A method of treating or preventing a thyroid stimulating hormone receptor (TSHR)-related disease in a subject, comprising administering to a subject in need thereof the antigen binding protein or antigen binding fragment thereof of claim 29, optionally wherein:
the TSHR-related disease is an autoimmune disease, optionally Graves' disease;
the TSHR-related disease is thyrotoxicosis; or
the TSHR-related disease is cancer.


SEQUENCE LISTING (147 sequences)

SEQ ID NO: 1 - protein, 119 aa, synthetic construct [sequence omitted]

SEQ ID NO: 2 - protein, 110 aa, synthetic construct
QSVLTQPPSVSAAPGQKVTISCSGSSSDIGSNYVSWYQQFPGTAPKLLIYDNNKRPSAIPDRFSGSKSGTSATLGITGLQTGDEADYYCGTWDSRLGIAVFGGGTQLTVL

SEQ ID NO: 3 - protein, 119 aa, synthetic construct
EVQLVQSGAEVKKPGASVKVSCKASGYSLTDNWIGWVRQAPGQGLEWMGIIYPGDSDTRYAQKFQGRVTMTADKSTSTVYMELSSLRSEDTAVYYCVGLDWNYNPLRYWGQGTLVTVSS

SEQ ID NO: 4 - protein, 110 aa, synthetic construct
QSVLTQPPSVSGAPGQRVTISCSGSSSDIGSNYVSWYQQLPGTAPKLLIYDNNKRPSGVPDRFSGSKSGTSASLAITGLQAEDEADYYCGTWDSRLGIAVFGGGTKLTVL

SEQ ID NO: 5 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 6 - protein, 106 aa, synthetic construct
GQPKAAPSVTLFPPSSEELQANKATLVCLVSDFYPGAVTVAWKADGSPVKVGVETTKPSKQSNNKYAASSYLSLTPEQWKSHRSYSCRVTHEGSTVEKTVAPTECS

SEQ ID NO: 7 - protein, 119 aa, synthetic construct
EVQLVQSGAEVKKPGESLKISCKASGYELNDNWIGWVRQKPGKGLEWMGIIYPGDSDTRYSPSFKGQVTISADKSISTAYLQWSSLKASDTAIYYCVGLEWNYNPLRYWGQGTLVTVSS

SEQ ID NO: 8 - protein, 119 aa, synthetic construct
EVQLVQSGAEVKKPGESLKISCKASGYQLNDNWIGWVRQKPGKGLEWMGIIYPGDSDTRYSPSFKGQVTISADKSISTAYLQWSSLKASDTAIYYCVGLEWNYNPLRYWGQGTLVTVSS

SEQ ID NO: 9 - protein, 119 aa, synthetic construct
EVQLVQSGAEVKKPGESLKISCKASGYELKDNWIGWVRQKPGKGLEWMGIIYPGDSDTRYSPSFKGQVTISADKSISTAYLQWSSLKASDTAIYYCVGLEWNYNPLRYWGQGTLVTVSS

SEQ ID NO: 10 - protein, 119 aa, synthetic construct
EVQLVQSGAEVKKPGESLKISCKASGYQLKDNWAGWVRQKPGKGLEWMGIIYPGDSDTRYSPSFKGQVTISADKSISTAYLQWSSLKASDTAIYYCVGLEWNYNPLRYWGQGTLVTVSS

SEQ ID NO: 11 - protein, 119 aa, synthetic construct
EVQLVQSGAEVKKPGESLKISCKASGYQLKDNWIGWVRQKPGKGLEWMGIIYPGDSDTRYSPSFQGQVTISADKSISTAYLQWSSLKASDTAIYYCVGLEWNYNPLRYWGQGTLVTVSS

SEQ ID NO: 12 - protein, 119 aa, synthetic construct
EVQLVQSGAEVKKPGESLKISCKASGYQLKDNWIGWVRQKPGKGLEWMGIIYPGDSDTRYSPSFKGQVTISADKSISTAYLQWSSLKASDTAIYYCVGLEWNYNPGRYWGQGTLVTVSS

SEQ ID NO: 13 - protein, 119 aa, synthetic construct
EVQLVQSGAEVKKPGESLKISCKASGYQLKDNWAGWVRQKPGKGLEWMGIIYPGDSDTRYSPSFKGQVTISADKSISTAYLQWSSLKASDTAIYYCVGLEWNYNPGRYWGQGTLVTVSS

SEQ ID NO: 14 - protein, 119 aa, synthetic construct
EVQLVQSGAEVKKPGESLKISCKASGYQLKDNWIGWVRQKPGKGLEWMGIIYPGDSDTRYSPSFQGQVTISADKSISTAYLQWSSLKASDTAIYYCVGLEWNYNPVRYWGQGTLVTVSS

SEQ ID NO: 15 - protein, 119 aa, synthetic construct
EVQLVQSGAEVKKPGESLKISCKASGYQLKDNWIGWVRQKPGKGLEWMGIIYPGDSDTRYSPSFQGQVTISADKSISTAYLQWSSLKASDTAIYYCVGGEWNYNPLRYWGQGTLVTVSS

SEQ ID NO: 16 - protein, 119 aa, synthetic construct
EVQLVQSGAEVKKPGASVKVSCKASGYELNDNWIGWVRQAPGQGLEWMGIIYPGDSDTRYAQKFQGRVTMTADKSTSTVYMELSSLRSEDTAVYYCVGLEWNYNPLRYWGQGTLVTVSS

SEQ ID NO: 17 - protein, 119 aa, synthetic construct
EVQLVQSGAEVKKPGASVKVSCKASGYQLNDNWIGWVRQAPGQGLEWMGIIYPGDSDTRYAQKFQGRVTMTADKSTSTVYMELSSLRSEDTAVYYCVGLEWNYNPLRYWGQGTLVTVSS

SEQ ID NO: 18 - protein, 119 aa, synthetic construct
EVQLVQSGAEVKKPGASVKVSCKASGYELKDNWIGWVRQAPGQGLEWMGIIYPGDSDTRYAQKFQGRVTMTADKSTSTVYMELSSLRSEDTAVYYCVGLEWNYNPLRYWGQGTLVTVSS

SEQ ID NO: 19 - protein, 119 aa, synthetic construct
EVQLVQSGAEVKKPGASVKVSCKASGYQLKDNWAGWVRQAPGQGLEWMGIIYPGDSDTRYAQKFQGRVTMTADKSTSTVYMELSSLRSEDTAVYYCVGLEWNYNPLRYWGQGTLVTVSS

SEQ ID NO: 20 - protein, 119 aa, synthetic construct
EVQLVQSGAEVKKPGASVKVSCKASGYQLKDNWIGWVRQAPGQGLEWMGIIYPGDSDTRYAQKFQGRVTMTADKSTSTVYMELSSLRSEDTAVYYCVGLEWNYNPLRYWGQGTLVTVSS

SEQ ID NO: 21 - protein, 119 aa, synthetic construct
EVQLVQSGAEVKKPGASVKVSCKASGYQLKDNWIGWVRQAPGQGLEWMGIIYPGDSDTRYAQKFQGRVTMTADKSTSTVYMELSSLRSEDTAVYYCVGLEWNYNPGRYWGQGTLVTVSS

SEQ ID NO: 22 - protein, 119 aa, synthetic construct
EVQLVQSGAEVKKPGASVKVSCKASGYQLKDNWAGWVRQAPGQGLEWMGIIYPGDSDTRYAQKFQGRVTMTADKSTSTVYMELSSLRSEDTAVYYCVGLEWNYNPGRYWGQGTLVTVSS

SEQ ID NO: 23 - protein, 119 aa, synthetic construct
EVQLVQSGAEVKKPGASVKVSCKASGYQLKDNWIGWVRQAPGQGLEWMGIIYPGDSDTRYAQKFQGRVTMTADKSTSTVYMELSSLRSEDTAVYYCVGLEWNYNPVRYWGQGTLVTVSS

SEQ ID NO: 24 - protein, 119 aa, synthetic construct
EVQLVQSGAEVKKPGASVKVSCKASGYQLKDNWIGWVRQAPGQGLEWMGIIYPGDSDTRYAQKFQGRVTMTADKSTSTVYMELSSLRSEDTAVYYCVGGEWNYNPLRYWGQGTLVTVSS

SEQ ID NO: 25 - protein, 110 aa, synthetic construct
QSVLTQPPSVSAAPGQKVTISCSGSSSDIGSNYVSWYQQLPGTAPKLLIYDNNKRPSGIPDRFSGSKSGTSATLGITGLQTGDEADYYCGTWDSRLGIAVFGGGTQLTVL

SEQ ID NO: 26 - protein, 110 aa, synthetic construct
QSVLTQPPSVSAAPGQKVTISCSGSSTNIGALWVSWYQQLPGTAPKLLIYDNNKRPSGIPDRFSGSKSGTSATLGITGLQTGDEADYYCATWDSRFGIAVFGGGTQLTVL

SEQ ID NO: 27 - protein, 110 aa, synthetic construct
QSVLTQPPSVSAAPGQKVTISCSGSSTNIGALWVSWYQQLPGTAPKLLIYDNNKRPSGIPDRFSGSKSGTSATLGITGLQTGDEADYYCATWDSRWGIAVFGGGTQLTVL

SEQ ID NO: 28 - protein, 110 aa, synthetic construct
QSVLTQPPSVSAAPGQKVTISCSGSSTNIGANYVSWYQQLPGTAPKLLIYDNNKRPSGIPDRFSGSKSGTSATLGITGLQTGDEADYYCATWDSRFGIAVFGGGTQLTVL

SEQ ID NO: 29 - protein, 110 aa, synthetic construct
QSVLTQPPSVSAAPGQKVTISCSGSSTNIGANYVSWYQQLPGTAPKLLIYDNNKRPSGIPDRFSGSKSGTSATLGITGLQTGDEADYYCATWDSRWGIAVFGGGTQLTVL

SEQ ID NO: 30 - protein, 110 aa, synthetic construct
QSVLTQPPSVSAAPGQKVTISCSGSSTDIGANYVSWYQQLPGTAPKLLIYDNNKRPSGIPDRFSGSKSGTSATLGITGLQTGDEADYYCATWDSRWGIAVFGGGTQLTVL

SEQ ID NO: 31 - protein, 110 aa, synthetic construct
QSVLTQPPSVSAAPGQKVTISCSGSSTNIGANYVSWYQQLPGTAPKLLIYDNQKRPSGIPDRFSGSKSGTSATLGITGLQTGDEADYYCATWDSRWGIAVFGGGTQLTVL

SEQ ID NO: 32 - protein, 110 aa, synthetic construct
QSVLTQPPSVSGAPGQRVTISCSGSSTNIGALWVSWYQQLPGTAPKLLIYDNNKRPSGVPDRFSGSKSGTSASLAITGLQAEDEADYYCATWDSRFGIAVFGGGTKLTVL

SEQ ID NO: 33 - protein, 110 aa, synthetic construct
QSVLTQPPSVSGAPGQRVTISCSGSSTNIGALWVSWYQQLPGTAPKLLIYDNNKRPSGVPDRFSGSKSGTSASLAITGLQAEDEADYYCATWDSRWGIAVFGGGTKLTVL

SEQ ID NO: 34 - protein, 110 aa, synthetic construct
QSVLTQPPSVSGAPGQRVTISCSGSSTNIGANYVSWYQQLPGTAPKLLIYDNNKRPSGVPDRFSGSKSGTSASLAITGLQAEDEADYYCATWDSRFGIAVFGGGTKLTVL

SEQ ID NO: 35 - protein, 110 aa, synthetic construct
QSVLTQPPSVSGAPGQRVTISCSGSSTNIGANYVSWYQQLPGTAPKLLIYDNNKRPSGVPDRFSGSKSGTSASLAITGLQAEDEADYYCATWDSRWGIAVFGGGTKLTVL

SEQ ID NO: 36 - protein, 110 aa, synthetic construct
QSVLTQPPSVSGAPGQRVTISCSGSSTDIGANYVSWYQQLPGTAPKLLIYDNNKRPSGVPDRFSGSKSGTSASLAITGLQAEDEADYYCATWDSRWGIAVFGGGTKLTVL

SEQ ID NO: 37 - protein, 110 aa, synthetic construct
QSVLTQPPSVSGAPGQRVTISCSGSSTNIGANYVSWYQQLPGTAPKLLIYDNQKRPSGVPDRFSGSKSGTSASLAITGLQAEDEADYYCATWDSRWGIAVFGGGTKLTVL

SEQ ID NO: 38 - protein, 10 aa, synthetic construct
GYSLTDNWIG

SEQ ID NO: 39 - protein, 17 aa, synthetic construct
IIYPGDSDTRYSPSFQG

SEQ ID NO: 40 - protein, 12 aa, synthetic construct
VGLDWNYNPLRY

SEQ ID NO: 41 - protein, 13 aa, synthetic construct
SGSSSDIGSNYVS

SEQ ID NO: 42 - protein, 7 aa, synthetic construct
DNNKRPS

SEQ ID NO: 43 - protein, 11 aa, synthetic construct
GTWDSRLGIAV

SEQ ID NO: 44 - protein, 10 aa, synthetic construct
GYELNDNWIG

SEQ ID NO: 45 - protein, 10 aa, synthetic construct
GYQLNDNWIG

SEQ ID NO: 46 - protein, 10 aa, synthetic construct
GYELKDNWIG

SEQ ID NO: 47 - protein, 10 aa, synthetic construct
GYQLKDNWAG

SEQ ID NO: 48 - protein, 10 aa, synthetic construct
GYQLKDNWIG

SEQ ID NO: 49 - protein, 17 aa, synthetic construct
IIYPGDSDTRYSPSFKG

SEQ ID NO: 50 - protein, 12 aa, synthetic construct
VGLEWNYNPLRY

SEQ ID NO: 51 - protein, 12 aa, synthetic construct
VGLEWNYNPGRY

SEQ ID NO: 52 - protein, 12 aa, synthetic construct
VGLEWNYNPVRY

SEQ ID NO: 53 - protein, 12 aa, synthetic construct
VGGEWNYNPLRY

SEQ ID NO: 54 - protein, 13 aa, synthetic construct
SGSSTNIGALWVS

SEQ ID NO: 55 - protein, 13 aa, synthetic construct
SGSSTNIGANYVS

SEQ ID NO: 56 - protein, 13 aa, synthetic construct
SGSSTDIGANYVS

SEQ ID NO: 57 - protein, 7 aa, synthetic construct
DNQKRPS

SEQ ID NO: 58 - protein, 11 aa, synthetic construct
ATWDSRFGIAV

SEQ ID NO: 59 - protein, 11 aa, synthetic construct
ATWDSRWGIAV

SEQ ID NO: 60 - protein, 12 aa, synthetic construct
VGLDWNYDPLRY

SEQ ID NO: 61 - protein, 12 aa, synthetic construct
VGLEWNYNPLRY

SEQ ID NO: 62 - protein, 10 aa, synthetic construct
GYELTDNWIG

SEQ ID NO: 63 - protein, 10 aa, synthetic construct
GYQLTDNWIG

SEQ ID NO: 64 - protein, 10 aa, synthetic construct
GYSLNDNWIG

SEQ ID NO: 65 - protein, 10 aa, synthetic construct
GYSLKDNWIG

SEQ ID NO: 66 - protein, 10 aa, synthetic construct
GYSLTDNWTG

SEQ ID NO: 67 - protein, 18 aa, synthetic construct
IIYPGDSDTRYSPSFKGQ

SEQ ID NO: 68 - protein, 12 aa, synthetic construct
VGTDWNYNPLRY

SEQ ID NO: 69 - protein, 11 aa, synthetic construct
GTWDSRFGIAV

SEQ ID NO: 70 - protein, 13 aa, synthetic construct
SGSSSDIGANYVS

SEQ ID NO: 71 - protein, 11 aa, synthetic construct
GTWDSRLGIAV

SEQ ID NO: 72 - protein, 13 aa, synthetic construct
SGSSSDIGSLYVS

SEQ ID NO: 73 - protein, 11 aa, synthetic construct
GTWDSRLGIAV

SEQ ID NO: 74 - protein, 13 aa, synthetic construct
SGSSTDIGSNYVS

SEQ ID NO: 75 - protein, 13 aa, synthetic construct
SGSSSDIGSNWVS

SEQ ID NO: 76 - protein, 13 aa, synthetic construct
SGSSSNIGSNYVS

SEQ ID NO: 77 - protein, 11 aa, synthetic construct
ATWDSRLGIAV

SEQ ID NO: 78 - protein, 119 aa, synthetic construct
EVQLVQSGAEVKKPGESLKISCKASGYSLTDNWIGWVRQKPGKGLEWMGIIYPGDSDTRYSPSFQGQVTISADKSISTAYLQWSSLKASDTAIYYCVGLDWNYNPLRYWGQGTLVTVSS

SEQ ID NO: 79 - protein, 119 aa, synthetic construct
EVQLVQSGAEVKKPGESLKISCKASGYSLTDNWIGWVRQKPGKGLEWMGIIYPGDSDTRYSPSFQGQVTISADKSISTAYLQWSSLKASDTAIYYCVGLDWNYDPLRYWGQGTLVTVSS

SEQ ID NO: 80 - protein, 119 aa, synthetic construct
EVQLVQSGAEVKKPGESLKISCKASGYSLTDNWIGWVRQKPGKGLEWMGIIYPGDSDTRYSPSFQGQVTISADKSISTAYLQWSSLKASDTAIYYCVGLEWNYNPLRYWGQGTLVTVSS

SEQ ID NO: 81 - protein, 119 aa, synthetic construct
EVQLVQSGAEVKKPGESLKISCKASGYELTDNWIGWVRQKPGKGLEWMGIIYPGDSDTRYSPSFQGQVTISADKSISTAYLQWSSLKASDTAIYYCVGLDWNYNPLRYWGQGTLVTVSS

SEQ ID NO: 82 - protein, 119 aa, synthetic construct
EVQLVQSGAEVKKPGESLKISCKASGYQLTDNWIGWVRQKPGKGLEWMGIIYPGDSDTRYSPSFQGQVTISADKSISTAYLQWSSLKASDTAIYYCVGLDWNYNPLRYWGQGTLVTVSS

SEQ ID NO: 83 - protein, 119 aa, synthetic construct
EVQLVQSGAEVKKPGESLKISCKASGYSLNDNWIGWVRQKPGKGLEWMGIIYPGDSDTRYSPSFQGQVTISADKSISTAYLQWSSLKASDTAIYYCVGLDWNYNPLRYWGQGTLVTVSS

SEQ ID NO: 84 - protein, 119 aa, synthetic construct
EVQLVQSGAEVKKPGESLKISCKASGYSLKDNWIGWVRQKPGKGLEWMGIIYPGDSDTRYSPSFQGQVTISADKSISTAYLQWSSLKASDTAIYYCVGLDWNYNPLRYWGQGTLVTVSS

SEQ ID NO: 85 - protein, 119 aa, synthetic construct
EVQLVQSGAEVKKPGESLKISCKASGYSLTDNWIGWVRQKPGKGLEWMGIIYPGDSDTRYSPSFQGQVTISADKSISTAYLQWSSLKASDTAMYYCVGLDWNYNPLRYWGQGTLVTVSS

SEQ ID NO: 86 - protein, 119 aa, synthetic construct
EVQLVQSGAEVKKPGESLKISCKASGYSLTDNWTGWVRQKPGKGLEWMGIIYPGDSDTRYSPSFQGQVTISADKSISTAYLQWSSLKASDTAIYYCVGLDWNYNPLRYWGQGTLVTVSS

SEQ ID NO: 87 - protein, 119 aa, synthetic construct
EVQLVQSGAEVKKPGESLKISCKASGYSLTDNWIGWVRQKPGKGLEWMGIIYPGDSDTRYSPSFKGQVTISADKSISTAYLQWSSLKASDTAIYYCVGLDWNYNPLRYWGQGTLVTVSS

SEQ ID NO: 88 - protein, 119 aa, synthetic construct
EVQLVQSGAEVKKPGESLKISCKASGYSLTDNWIGWVRQKPGKGLEWMGIIYPGDSDTRYSPSFQGQVTISADKSISTAYLQWSSLKASDTAIYYCVGTDWNYNPLRYWGQGTLVTVSS

SEQ ID NO: 89 - protein, 110 aa, synthetic construct
QSVLTQPPSVSAAPGQKVTISCSGSSSDIGSNYVSWYQQLPGTAPKLLIYDNNKRPSGIPDRFSGSKSGTSATLGITGLQTGDEADYYCGTWDSRWGIAVFGGGTQLTVL

SEQ ID NO: 90 - protein, 110 aa, synthetic construct
QSVLTQPPSVSAAPGQKVTISCSGSSSDIGSNYVSWYQQLPGTAPKLLIYDNNKRPSGIPDRFSGSKSGTSATLGITGLQTGDEADYYCGTWDSRFGIAVFGGGTQLTVL

SEQ ID NO: 91 - protein, 110 aa, synthetic construct
QSVLTQPPSVSAAPGQKVTISCSGSSSDIGANYVSWYQQLPGTAPKLLIYDNNKRPSGIPDRFSGSKSGTSATLGITGLQTGDEADYYCGTWDSRLGIAVFGGGTQLTVL

SEQ ID NO: 92 - protein, 110 aa, synthetic construct
QSVLTQPPSVSAAPGQKVTISCSGSSSDIGSLYVSWYQQLPGTAPKLLIYDNNKRPSGIPDRFSGSKSGTSATLGITGLQTGDEADYYCGTWDSRLGIAVFGGGTQLTVL

SEQ ID NO: 93 - protein, 110 aa, synthetic construct
QSVLTQPPSVSAAPGQKVTISCSGSSTDIGSNYVSWYQQLPGTAPKLLIYDNNKRPSGIPDRFSGSKSGTSATLGITGLQTGDEADYYCGTWDSRLGIAVFGGGTQLTVL

SEQ ID NO: 94 - protein, 110 aa, synthetic construct
QSVLTQPPSVSAAPGQKVTISCSGSSSDIGSNWVSWYQQLPGTAPKLLIYDNNKRPSGIPDRFSGSKSGTSATLGITGLQTGDEADYYCGTWDSRLGIAVFGGGTQLTVL

SEQ ID NO: 95 - protein, 110 aa, synthetic construct
QSVLTQPPSVSAAPGQKVTISCSGSSSNIGSNYVSWYQQLPGTAPKLLIYDNNKRPSGIPDRFSGSKSGTSATLGITGLQTGDEADYYCGTWDSRLGIAVFGGGTQLTVL

SEQ ID NO: 96 - protein, 110 aa, synthetic construct
QSVLTQPPSVSAAPGQKVTISCSGSSSDIGSNYVSWYQQLPGTAPKLLIYDNNKRPSGIPDRFSGSKSGTSATLGITGLQTGDEADYYCATWDSRLGIAVFGGGTQLTVL

SEQ ID NO: 97 - protein, 11 aa, synthetic construct
GTWDSRWGIAV

SEQ ID NO: 98 - protein, 25 aa, synthetic construct
EVQLVQSGAEVKKPGESLKISCKAS

SEQ ID NO: 99 - protein, 14 aa, synthetic construct
WVRQKPGKGLEWMG

SEQ ID NO: 100 - protein, 32 aa, synthetic construct
GIPDRFSGSKSGTSATLGITGLQTGDEADYYC

SEQ ID NO: 101 - protein, 10 aa, synthetic construct
FGGGTQLTVL

SEQ ID NO: 102 - protein, 22 aa, synthetic construct
QSVLTQPPSVSAAPGQKVTISC

SEQ ID NO: 103 - protein, 15 aa, synthetic construct
WYQQLPGTAPKLLIY

SEQ ID NO: 104 - protein, 32 aa, synthetic construct; identical to SEQ ID NO: 100

SEQ ID NO: 105 - protein, 11 aa, synthetic construct
WGQGTLVTVSS

SEQ ID NO: 106 - protein, 10 aa, synthetic construct
GYSLTDNWIG

SEQ ID NO: 107 - protein, 17 aa, synthetic construct
IIYPGDSDTRYAQKFQG

SEQ ID NO: 108 - protein, 12 aa, synthetic construct
VGLDWNYNPLRY

SEQ ID NO: 109 - protein, 13 aa, synthetic construct
SGSSSDIGSNYVS

SEQ ID NO: 110 - protein, 7 aa, synthetic construct
DNNKRPS

SEQ ID NO: 111 - protein, 11 aa, synthetic construct
GTWDSRLGIAV

SEQ ID NO: 112 - protein, 25 aa, synthetic construct
EVQLVQSGAEVKKPGASVKVSCKAS

SEQ ID NO: 113 - protein, 14 aa, synthetic construct
WVRQAPGQGLEWMG

SEQ ID NO: 114 - protein, 30 aa, synthetic construct
RVTMTADKSTSTVYMELSSLRSEDTAVYYC

SEQ ID NO: 115 - protein, 11 aa, synthetic construct
WGQGTLVTVSS

SEQ ID NO: 116 - protein, 22 aa, synthetic construct
QSVLTQPPSVSGAPGQRVTISC

SEQ ID NO: 117 - protein, 15 aa, synthetic construct
WYQQLPGTAPKLLIY

SEQ ID NO: 118 - protein, 32 aa, synthetic construct
GVPDRFSGSKSGTSASLAITGLQAEDEADYYC

SEQ ID NO: 119 - protein, 10 aa, synthetic construct
FGGGTKLTVL

SEQ ID NO: 120 - protein, 119 aa, synthetic construct
EVQLVQSGAEVKKPGQSLKISCKASGYSLTDNWIGWVRQKPGKGLEWMGIIHPGDSDTRYSPSFQGQVTISADKSINTAYLQWSSLKASDTAIYYCVGLDWNYNPLRYWGPGTLVTVSS

SEQ ID NO: 121 - protein, 119 aa, synthetic construct
EVQLVQSGAEVKKPGQSLKISCKASGYSLTDNWIGWVRQKPGKGLEWMGIIYPGDSHTRYSPSFQGQVTISADKSINTAYLQWSSLKASDTAIYYCVGLDWNYNPLRYWGPGTLVTVSS

SEQ ID NO: 122 - protein, 119 aa, synthetic construct
EVQLVQSGAEVKKPGQSLKISCKASGYSLTDNWIGWVRQKPGKGLEWMGIIYPGDSKTRYSPSFQGQVTISADKSINTAYLQWSSLKASDTAIYYCVGLDWNYNPLRYWGPGTLVTVSS

SEQ ID NO: 123 - protein, 119 aa, synthetic construct [sequence omitted]

SEQ ID NO: 124 - protein, 119 aa, synthetic construct
EVQLVQSGAEVKKPGQSLKISCKASGYSLTDNWIGWVRQKPGKGLEWMGIIYPGDSDTRYSPSFQGQVTISADKSINTAYLQWSSLKASDTAIYYCVGLYWNYNPLRYWGPGTLVTVSS

SEQ ID NO: 125 - protein, 119 aa, synthetic construct
EVQLVQSGAEVKKPGQSLKISCKASGYSLTDNWIGWVRQKPGKGLEWMGIIYPGDSDTRYSPSFQGQVTISADKSINTAYLQWSSLKASDTAIYYCVGLDWNRNPLRYWGPGTLVTVSS

SEQ ID NO: 126 - protein, 119 aa, synthetic construct
EVQLVQSGAEVKKPGQSLKISCKASGYSLTDNWIGWVRQKPGKGLEWMGIIYPGDSDTRYSPSFQGQVTISADKSINTAYLQWSSLKASDTAIYYCVGLDWNYNDLRYWGPGTLVTVSS

SEQ ID NO: 127 - protein, 119 aa, synthetic construct
EVQLVQSGAEVKKPGQSLKISCKASGYSLTDNWIGWVRQKPGKGLEWMGIIYPGDSDTRYSPSFQGQVTISADKSINTAYLQWSSLKASDTAIYYCVGLDWNYNELRYWGPGTLVTVSS

SEQ ID NO: 128 - protein, 119 aa, synthetic construct
EVQLVQSGAEVKKPGESLKISCKGSGYSFTSYWIGWVRQMPGKGLEWMGIIYPGDTDTRYSPSFQGQVTISADKSISTAYLQWSSLKASDTAVYYCVGLDWNYNPLRYWGPGTLVTVSS

SEQ ID NO: 129 - protein, 110 aa, synthetic construct
QSVLTQPPSVSAAPGQKVTISCSGSSSDIGNNYVSWYQQLPGTAPKLLIYDNNKRPSGIPDRFSGSKSGTSATLGITGLQTGDEADYYCGTWESRLGIAVFGGGTQLTVL

SEQ ID NO: 130 - protein, 10 aa, synthetic construct
GYSFTSYWIG

SEQ ID NO: 131 - protein, 17 aa, synthetic construct
IIYPGDTDTRYSPSFQG

SEQ ID NO: 132 - protein, 12 aa, synthetic construct
VGLDWNYNPLRY

SEQ ID NO: 133 - protein, 13 aa, synthetic construct
SGSSSDIGNNYVS

SEQ ID NO: 134 - protein, 7 aa, synthetic construct
DNNKRPS

SEQ ID NO: 135 - protein, 11 aa, synthetic construct
GTWESRLGIAV

SEQ ID NO: 136 - protein, 119 aa, synthetic construct
EVQLVQSGAEVKKPGESLKISCKGSGYSFTHYWIGWVRQMPGKGLEWMGIIYPGDTDTRYSPSFQGQVTISADKSISTAYLQWSSLKASDTAVYYCVGLDWNYNPLRYWGPGTLVTVSS

SEQ ID NO: 137 - protein, 110 aa, synthetic construct
QSVLTQPPSVSAAPGQKVTISCSGSSSDIGRNYVSWYQQLPGTAPKLLIYDNNKRPSGIPDRFSGSKSGTSATLGITGLQTGDEADYYCGTWESRLGIAVFGGGTQLTVL

SEQ ID NO: 138 - protein, 119 aa, synthetic construct
EVQLVQSGAEVKKPGESLKISCKGSGYHFHSYWIGWVRQMPGKGLEWMGIIYPGDTDTRYSPSFQGQVTISADKSISTAYLQWSSLKASDTAVYYCVGLDWNYNPLRYWGPGTLVTVSS

SEQ ID NO: 139 - protein, 119 aa, synthetic construct
EVQLVQSGAEVKKPGESLKISCKGSGYHFTSYWIGWVRQMPGKGLEWMGIIYPGDTDTRYSPSFQGQVTISADKSISTAYLQWSSLKASDTAVYYCVGLDWHYNPLRYWGPGTLVTVSS

SEQ ID NO: 140 - protein, 119 aa, synthetic construct
EVQLVQSGAEVKKPGESLKISCKGSGYSFHSYWIGWVRQMPGKGLEWMGIIYPGDTDTRYSPSFQGQVTISADKSISTAYLQWSSLKASDTAVYYCVGLDWHYNPLRYWGPGTLVTVSS

SEQ ID NO: 141 - protein, 119 aa, synthetic construct
EVQLVQSGAEVKKPGESLKISCKGSGYHFTSHWIGWVRQMPGKGLEWMGIIYPGDTDTRYSPSFQGQVTISADKSISTAYLQWSSLKASDTAVYYCVGLDWNYNPLRYWGPGTLVTVSS

SEQ ID NO: 142 - protein, 119 aa, synthetic construct
EVQLVQSGAEVKKPGESLKISCKGSGYSFHHYWIGWVRQMPGKGLEWMGIIYPGDTDTRYSPSFQGQVTISADKSISTAYLQWSSLKASDTAVYYCVGLDWNYNPLRYWGPGTLVTVSS

SEQ ID NO: 143 - protein, 119 aa, synthetic construct
EVQLVQSGAEVKKPGESLKISCKGSGYHFHHYWIGWVRQMPGKGLEWMGIIYPGDTDTRYSPSFQGQVTISADKSISTAYLQWSSLKASDTAVYYCVGLDWNYNPLRYWGPGTLVTVSS

SEQ ID NO: 144 - protein, 119 aa, synthetic construct
  VARIANT 52: note = X can be H or Y
  VARIANT 57: note = X can be H, K or Y
  VARIANT 70: note = X can be H or I
  VARIANT 100: note = X can be Y or D
  VARIANT 103: note = X can be R or Y
  VARIANT 105: note = X can be D, E or P
EVQLVQSGAEVKKPGQSLKISCKASGYSLTDNWIGWVRQKPGKGLEWMGIIXPGDSXTRYSPSFQGQVTXSADKSINTAYLQWSSLKASDTAIYYCVGLXWNXNXLRYWGPGTLVTVSS

SEQ ID NO: 145 - protein, 96 aa, synthetic construct
QSLKISCKASGYSLTDNWIGWVRQKPGKGLEWMGIIYPGDSDTRYSPSFQGQVTISADKSINTAYLQWSSLKASDTAIYYCVGLDWNYNPLRYWGP

SEQ ID NO: 146 - protein, 83 aa, synthetic construct
ISCSGSSSDIGSNYVSWYQQFPGTAPKLLIYDNNKRPSAIPDRFSGSKSGTSATLGITGLQTGDEADYYCGTWDSRLGIAVFG

SEQ ID NO: 147 - protein, 6 aa, synthetic construct
KSCDKT